(19) United States
(12) Patent Application Publication
(10) Pub. No.: US 2024/0390349 A1
(43) Pub. Date: Nov. 28, 2024

(54) DOSING REGIMEN OF CAPSID INHIBITOR

(71) Applicant: Gilead Sciences, Inc., Foster City, CA (US)

(72) Inventors: Rebecca R. Begley, Palo Alto, CA (US); Polina German, Campbell, CA (US); Justin Lutz, Newark, CA (US); Rameshraja Palaparthy, Foster City, CA (US); Abdul Naveed Shaik, Union City, CA (US); Martin S. Rhee, Foster City, CA (US); Renu Singh, Silver Spring, MD (US)

(21) Appl. No.: 18/639,097

(22) Filed: Apr. 18, 2024

Related U.S. Application Data
(60) Provisional application No. 63/497,168, filed on Apr. 19, 2023.

(51) Int. Cl. A61K 31/4439; A61K 45/06; A61P 31/18

(52) U.S. Cl. CPC A61K 31/4439; A61K 45/06; A61P 31/18

(57) ABSTRACT

The present disclosure relates to dosing regimens of an HIV capsid inhibitor and methods for the treatment or prevention of a human immunodeficiency virus (HIV) infection in a patient.

### CROSS-REFERENCE TO RELATED APPLICATIONS

This application claims the benefit of U.S. Provisional Application No. 63/497,168, filed on Apr. 19, 2023, the entire contents of which is hereby incorporated by reference in its entirety.

### TECHNICAL FIELD

The present disclosure relates to dosing regimens of an HIV capsid inhibitor and methods for the treatment or prevention of a human immunodeficiency virus (HIV) infection in a patient.

### BACKGROUND

The viral capsid protein (CA) is essential for multiple stages of the HIV life cycle. During viral maturation following the processing of Gag polyprotein by the HIV protease, CA self-assembles into the conical shaped core characteristic of mature HIV-1 virions. Contained within this capsid core are the viral RNA, nucleocapsid, reverse transcriptase, and integrase. Failure to generate a suitable core precludes infectivity. In addition, CA contributes to multiple essential processes during the early stages of HIV replication, including important roles in regulating proper capsid core disassembly (uncoating) kinetics to ensure efficient and productive viral DNA synthesis via coupled reverse transcription, and contributes to the active transport of pre-integration complexes into the nuclear compartment to support viral DNA integration into transcriptionally active loci. Defects in the proper function of capsid ultimately inhibit efficient nuclear uptake and integration of viral DNA into the host genome.

Human immunodeficiency virus type 1 infection is a life-threatening and serious disease of major public health significance, with approximately 38 million people infected worldwide and approximately 26 million on antiretroviral (ARV) treatment (UNAIDS. Global HIV & AIDS statistics, 2020 fact sheet). Advances in combination ARV therapy for HIV have led to significant improvements in morbidity and mortality by suppressing viral replication, preserving immunologic function, and averting disease progression to AIDS.

### SUMMARY

The present disclosure provides a method of treating or preventing HIV in a patient, comprising administering to the patient a compound of Formula Ia:

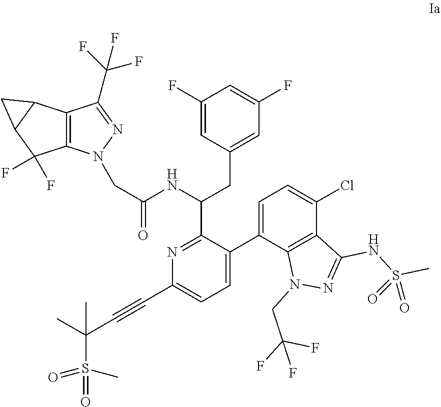 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(a) administering to the patient an initiation dosage of the compound of Formula Ia, or a pharmaceutically acceptable salt thereof, for a first period of time; and
(b) administering to the patient one or more maintenance dosages of the compound of Formula Ia, or a pharmaceutically acceptable salt thereof, for a second period of time, wherein the second period of time occurs after the first period of time; and
wherein if the patient misses or will miss a maintenance dosage, the method further comprises orally administering to the patient a bridging dosage of about 250 mg to about 650 mg of the compound of Formula Ia, or a pharmaceutically acceptable salt thereof, once per week until the patient resumes administration of the one or more maintenance dosages.

The present disclosure further provides the compound of Formula Ia, or a pharmaceutically acceptable salt thereof, for use in any of the methods described herein.

The present disclosure further provides use of the compound of Formula Ia, or a pharmaceutically acceptable salt thereof, for the preparation of a medicament for use in any of the methods described herein.

### BRIEF DESCRIPTION OF THE DRAWINGS

FIG. 1 shows simulated pharmacokinetic profile of oral weekly bridging of lenacapavir (300 mg) prior to resuming subcutaneous injection.

FIG. 2 shows simulated pharmacokinetic profile of oral weekly bridging of lenacapavir (300 mg) after resuming subcutaneous injection.

### DETAILED DESCRIPTION

Lenacapavir, a human immunodeficiency virus type 1 (HIV-1) capsid inhibitor, is indicated for the treatment of HIV-1 infection (e.g., in heavily treatment-experienced adults with multidrug resistant HIV-1 infection failing their current antiretroviral regimen due to resistance, intolerance, or safety considerations). The recommended dosage of lenacapavir comprises an initiation dosage (e.g., an initiation dosing as described herein) followed by maintenance dosing (e.g., maintenance dosing as described herein). Patients receiving lenacapavir may miss or anticipate missing a subcutaneous (SC) injection window (e.g., within 26 to 28 weeks of the previous lenacapavir dose) during the maintenance dosing period. If patients miss or anticipate missing a subcutaneous injection, the patients may receive an oral bridging dosage as provided herein, until they can receive their next SC injection.

### Methods

Accordingly, the present disclosure relates to a method of treating or preventing HIV in a patient, comprising administering to the patient a compound of Formula Ia:

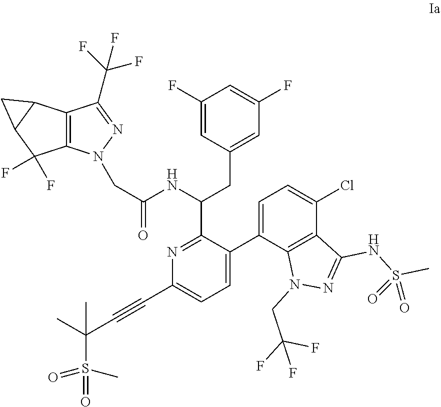 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(a) administering to the patient an initiation dosage of the compound of Formula Ia, or a pharmaceutically acceptable salt thereof, for a first period of time; and
(b) administering to the patient one or more maintenance dosages of the compound of Formula Ia, or a pharmaceutically acceptable salt thereof, for a second period of time, wherein the second period of time occurs after the first period of time; and
wherein if the patient misses or will miss a maintenance dosage, the method further comprises orally administering to the patient a bridging dosage of about 250 mg to about 650 mg of the compound of Formula Ia, or a pharmaceutically acceptable salt thereof, once per week until the patient resumes administration of the one or more maintenance dosages.

In any of the embodiments provided herein, the compound of Formula Ia is a compound of Formula Ib:

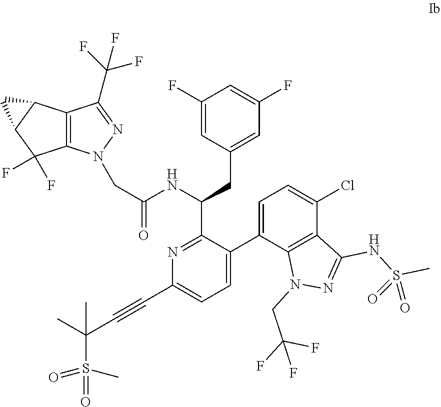 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof. The compound of Formula Ib may also be referred to as lenacapavir (or “LEN”) or N—((S)-1-(3-(4-chloro-3-(methylsulfonamido)-1-(2,2,2-trifluoroethyl)-1H-indazol-7-yl)-6-(3-methyl-3-(methylsulfonyl) but-1-yn-1-yl) pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa [3,4] cyclopenta [1,2-c]pyrazol-1-yl) acetamide.

In some embodiments, the compound of Formula Ia or Ib, or a pharmaceutically acceptable salt thereof, is administered as a monotherapy (i.e., in the absence of an additional therapeutic agent). In some embodiments, the compound of Formula Ia, or a pharmaceutically acceptable salt thereof, is administered in combination with one or more additional therapeutic agents, such as anti-HIV agents.

In some embodiments, the method comprises administering the compound of Formula Ia, or a pharmaceutically acceptable salt thereof. In some embodiments, the method comprises administering the compound of Formula Ib, or a pharmaceutically acceptable salt thereof.

Synthesis and characterization of the compounds of Formula Ia and Formula Ib, and salts thereof, are described, for example, in US 20180051005 and US 20190300505, the contents of each of which are hereby incorporated by reference in their entireties. Various forms and/or uses of the compounds of Formula Ia and Ib are disclosed, for example, in US 20190083478, US20190084963, US 20200038389A1, and US20210188815, the contents of each of which are hereby incorporated by reference in their entireties.

In some embodiments, the compound of Formula Ia is administered as the sodium salt. In some embodiments, the compound of Formula Ib is administered as the sodium salt.

In the absence of a specific reference to a particular pharmaceutically acceptable salt and/or solvate of the compound of Formula Ia or Ib, any dosages, whether expressed in milligrams or as % by weight, should be understood as referring to the amount of the free acid, i.e., the compound of Formula Ia or Formula Ib. For example, a reference to “50 mg” of Formula Ia, or a pharmaceutically acceptable salt thereof, refers to an amount of the compound of Formula Ia, or a pharmaceutically acceptable salt thereof, which provides the same amount of the compound of Formula Ia as 50 mg of the compound of Formula Ia free acid. In some embodiments, a dosage referring to 50 mg of Formula Ia contains about 51.1 mg of Formula Ia sodium salt.

In some embodiments, the compound provided herein (i.e., the compound of Formula Ia or Ib), or a pharmaceutically acceptable salt thereof, is administered orally in the form of one or more tablets as described herein.

In some embodiments, the compound provided herein (i.e., the compound of Formula Ia or Ib), or a pharmaceutically acceptable salt thereof, is administered subcutaneously in the form of one or more solutions as described herein.

In some embodiments, the compound provided herein (i.e., the compound of Formula Ia or Ib), or a pharmaceutically acceptable salt thereof, is administered intramuscularly in the form of one or more solutions as described herein.

In some embodiments, the first period of time is about one day to about two weeks. In some embodiments, the first period of time is about one day to about fifteen days. In some embodiments, the first period of time is two days. In some embodiments, the first period of time is about two weeks. In some embodiments, the first period of time is fifteen days.

In some embodiments, the initiation dosage provided herein comprises one or more oral administrations of the compound of Formula Ia or Ib, or a pharmaceutically acceptable salt thereof.

In some embodiments, the initiation dosage provided herein comprises one or more intramuscular or subcutaneous administrations of the compound of Formula Ia or Ib, or a pharmaceutically acceptable salt thereof.

In some embodiments, the initiation dosage provided herein comprises one or more subcutaneous administrations of the compound of Formula Ia or Ib, or a pharmaceutically acceptable salt thereof.

In some embodiments, the initiation dosage provided herein comprises one or more intramuscular administrations of the compound of Formula Ia or Ib, or a pharmaceutically acceptable salt thereof.

In some embodiments, the initiation dosage provided herein comprises one or more oral administrations of the compound of Formula Ia or Ib, or a pharmaceutically acceptable salt thereof; and one or more intramuscular or subcutaneous administrations of the compound of Formula Ia or Ib, or a pharmaceutically acceptable salt thereof.

In some embodiments, the initiation dosage provided herein comprises one or more oral administrations of the compound of Formula Ia or Ib, or a pharmaceutically acceptable salt thereof; and one or more subcutaneous administrations of the compound of Formula Ia or Ib, or a pharmaceutically acceptable salt thereof.

In some embodiments, the initiation dosage provided herein comprises one or more oral administrations of the compound of Formula Ia or Ib, or a pharmaceutically acceptable salt thereof; and one or more intramuscular administrations of the compound of Formula Ia or Ib, or a pharmaceutically acceptable salt thereof.

In some embodiments, the initiation dosage comprises:
subcutaneously administering the compound of Formula Ia or Ib, or a pharmaceutically acceptable salt thereof, at a concentration of about 309 mg/mL, and orally administering about 500 mg to about 700 mg of the compound of Formula Ia or Ib, or a pharmaceutically acceptable salt thereof, on the first day; and
orally administering about 500 mg to about 700 mg of the compound of Formula Ia or Ib, or a pharmaceutically acceptable salt thereof, on the second day.

In some embodiments, the first period of time is two days and the initiation dosage comprises:
subcutaneously administering the compound of Formula Ia or Ib, or a pharmaceutically acceptable salt thereof, at a concentration of about 309 mg/mL, and orally administering about 500 mg to about 700 mg of the compound of Formula Ia or Ib, or a pharmaceutically acceptable salt thereof, on the first day; and
orally administering about 500 mg to about 700 mg of the compound of Formula Ia or Ib, or a pharmaceutically acceptable salt thereof, on the second day.

In some embodiments, the subcutaneous administration is administered as two subcutaneous injections of about 309 mg/mL, on the first day. In some embodiments, the subcutaneous administration comprises administering about 927 mg of the compound of Formula Ia or Ib, or a pharmaceutically acceptable salt thereof, as two subcutaneous injections of about 309 mg/mL (e.g., two subcutaneous injections of 1.5 mL per injection, each comprising about 309 mg/mL of the compound of Formula Ia or Ib, or a pharmaceutically acceptable salt thereof).

In some embodiments, the oral administrations are administered as one or two tablets (e.g., one or two tablets on the first day; one or two tablets on the second day; 1 or 2 tablets on the first day and 1 or 2 tablets on the second day; and the like). In some embodiments, the oral administrations are administered as two tablets.

In some embodiments, the oral administrations are administered as two tablets, each comprising about 200 to about 400 mg of the compound of Formula Ia or Ib, or a pharmaceutically acceptable salt thereof, on the first day and on the second day.

In some embodiments, the oral administrations are administered as two tablets each comprising about 200 to about 400 mg of the compound of Formula Ia or Ib, or a pharmaceutically acceptable salt thereof, on the first day; and as two tablets each comprising about 200 to about 400 mg of the compound of Formula Ia or Ib, or a pharmaceutically acceptable salt thereof, on the second day.

In some embodiments, the oral administrations are administered as two tablets, each comprising about 200 to about 400 mg of the compound of Formula Ia or Ib, or a pharmaceutically acceptable salt thereof, on the first day; and as two tablets, each comprising about 200 to about 400 mg of the compound of Formula Ia or Ib, or a pharmaceutically acceptable salt thereof, on the second day.

In some embodiments, the oral administrations are administered as two tablets, each comprising about 300 mg of the compound of Formula Ia or Ib, or a pharmaceutically acceptable salt thereof, on the first day and on the second day.

In some embodiments, the oral administrations are administered as two tablets, each comprising about 300 mg of the compound of Formula Ia or Ib, or a pharmaceutically acceptable salt thereof, on the first day and on the second day.

In some embodiments, the oral administrations are administered as two tablets, each comprising about 300 mg of the compound of Formula Ia or Ib, or a pharmaceutically acceptable salt thereof, on the first day; and as two tablets, each comprising about 300 mg of the compound of Formula Ia or Ib, or a pharmaceutically acceptable salt thereof, on the second day.

In some embodiments, the initiation dosage comprises:
subcutaneously administering 927 mg of the compound of Formula Ia or Ib, or a pharmaceutically acceptable salt thereof, and orally administering about 600 mg of the compound of Formula Ia or Ib, or a pharmaceutically acceptable salt thereof, on the first day; and
orally administering about 600 mg of the compound of Formula Ia or Ib, or a pharmaceutically acceptable salt thereof, on the second day.

In some embodiments, the initiation dosage comprises:
subcutaneously administering 927 mg of the compound of Formula Ia or Ib, or a pharmaceutically acceptable salt thereof, and orally administering two tablets, each comprising about 300 mg of the compound of Formula Ia, or a pharmaceutically acceptable salt thereof, on the first day; and
orally administering two tablets, each comprising about 300 mg of the compound of Formula Ia or Ib, or a pharmaceutically acceptable salt thereof, on the second day.

In some embodiments, the initiation dosage comprises:
orally administering about 500 mg to about 700 mg of the compound of Formula Ia or Ib, or a pharmaceutically acceptable salt thereof, on the first day and on the second day;
orally administering about 200 mg to about 400 mg of the compound of Formula Ia or Ib, or a pharmaceutically acceptable salt thereof, on the eighth day; and
subcutaneously administering the compound of Formula Ia or Ib, or a pharmaceutically acceptable salt thereof, at a concentration of about 309 mg/mL, on the fifteenth day.

In some embodiments, the first period of time is fifteen days and the initiation dosage comprises:
orally administering about 500 mg to about 700 mg of the compound of Formula Ia or Ib, or a pharmaceutically acceptable salt thereof, on the first day and on the second day;
orally administering about 200 mg to about 400 mg of the compound of Formula Ia or Ib, or a pharmaceutically acceptable salt thereof, on the eighth day; and
subcutaneously administering the compound of Formula Ia or Ib, or a pharmaceutically acceptable salt thereof, at a concentration of about 309 mg/mL, on the fifteenth day.

In some embodiments, the oral administrations of the first and second days are each administered as two tablets, each comprising about 200 to about 400 mg of the compound of Formula Ia or Ib, or a pharmaceutically acceptable salt thereof.

In some embodiments, the oral administrations on the first and second days are administered as two tablets, each comprising about 200 to about 400 mg of the compound of Formula Ia or Ib, or a pharmaceutically acceptable salt thereof, on the first day; and as two tablets, each comprising about 200 to about 400 mg of the compound of Formula Ia or Ib, or a pharmaceutically acceptable salt thereof, on the second day.

In some embodiments, the oral administration of the eighth day is administered as one tablet comprising about 200 to about 400 mg of the compound of Formula Ia or Ib, or a pharmaceutically acceptable salt thereof. In some embodiments, the oral administration of the eighth day is administered as one tablet comprising about 300 mg of the compound of Formula Ia or Ib, or a pharmaceutically acceptable salt thereof.

In some embodiments, the oral administrations of the first and second days are administered as two tablets, each comprising about 200 to about 400 mg of the compound of Formula Ia or Ib, or a pharmaceutically acceptable salt thereof; and
the oral administration of the eighth day is administered as one tablet comprising about 200 to about 400 mg of the compound of Formula Ia or Ib, or a pharmaceutically acceptable salt thereof.

In some embodiments:
the oral administration on the first day is administered as two tablets, each comprising about 200 to about 400 mg of the compound of Formula Ia or Ib, or a pharmaceutically acceptable salt thereof;
the oral administration on the second day is administered as two tablets, each comprising about 200 to about 400 mg of the compound of Formula Ia or Ib, or a pharmaceutically acceptable salt thereof; and
the oral administration on the eighth day is administered as one tablet comprising about 200 to about 400 mg of the compound of Formula Ia or Ib, or a pharmaceutically acceptable salt thereof.

In some embodiments, oral administrations of the first and second days are administered as two tablets, each comprising about 300 mg of the compound of Formula Ia or Ib, or a pharmaceutically acceptable salt thereof; and
the oral administration of the eighth day is administered as one tablet comprising about 300 mg of the compound of Formula Ia or Ib, or a pharmaceutically acceptable salt thereof.

In some embodiments:
the oral administration on the first day is administered as two tablets, each comprising about 300 mg of the compound of Formula Ia or Ib, or a pharmaceutically acceptable salt thereof;
the oral administration on the second day is administered as two tablets, each comprising about 300 mg of the compound of Formula Ia or Ib, or a pharmaceutically acceptable salt thereof; and
the oral administration on the eighth day is administered as one tablet comprising about 300 mg of the compound of Formula Ia or Ib, or a pharmaceutically acceptable salt thereof.

In some embodiments, the subcutaneous administration is administered as two subcutaneous injections of about 309 mg/mL, on the fifteenth day. In some embodiments, the subcutaneous administration comprises administering about 927 mg of the compound of Formula Ia or Ib, or a pharmaceutically acceptable salt thereof, as two subcutaneous injections of about 309 mg/mL (e.g., two subcutaneous injections of 1.5 mL per injection, each comprising about 309 mg/mL of the compound of Formula Ia or Ib, or a pharmaceutically acceptable salt thereof).

In some embodiments, the initiation dosage comprises:
orally administering about 600 mg of the compound of Formula Ia or Ib, or a pharmaceutically acceptable salt thereof, on the first day and the second day;
orally administering about 300 mg of the compound of Formula Ia or Ib, or a pharmaceutically acceptable salt thereof, on the eighth day; and
subcutaneously administering 927 mg of the compound of Formula Ia or Ib, or a pharmaceutically acceptable salt thereof, on the fifteenth day.

In some embodiments, the initiation dosage comprises:
orally administering two tablets, each comprising about 300 mg of the compound of Formula Ia or Ib, or a pharmaceutically acceptable salt thereof, on the first day;
orally administering two tablets, each comprising about 300 mg of the compound of Formula Ia or Ib, or a pharmaceutically acceptable salt thereof, on the second day;
orally administering one tablet comprising about 300 mg of the compound of Formula Ia or Ib, or a pharmaceutically acceptable salt thereof, on the eighth day; and
subcutaneously administering 927 mg of the compound of Formula Ia or Ib, or a pharmaceutically acceptable salt thereof, on the fifteenth day.

In some embodiments, the initiation dosage comprises:
orally administering two tablets, each comprising about 300 mg of the compound of Formula Ia or Ib, or a pharmaceutically acceptable salt thereof, on the first day;
orally administering two tablets, each comprising about 300 mg of the compound of Formula Ia or Ib, or a pharmaceutically acceptable salt thereof, on the second day;
orally administering one tablet comprising about 300 mg of the compound of Formula Ia or Ib, or a pharmaceutically acceptable salt thereof, on the eighth day; and
subcutaneously administering two injections of 1.5 mL per injection, each comprising about 309 mg/mL of the compound of Formula Ia or Ib, or a pharmaceutically acceptable salt thereof, on the fifteenth day.

In some embodiments, the second period of time begins about 24 weeks to about 28 weeks after the final administration of the initiation dosage described herein, e.g., about 24 weeks, about 25 weeks, about 26 weeks, about 27 weeks, or about 28 weeks after the final administration of the initiation dosage described herein. In some embodiments, the second period of time begins about 26 weeks after the final administration of the initiation dosage described herein.

In some embodiments, the second period of time begins about 24 weeks to about 28 weeks after the final subcutaneous administration of the initiation dosage provided herein, e.g., about 24 weeks, about 25 weeks, about 26 weeks, about 27 weeks, or about 28 weeks after the final subcutaneous administration of the initiation dosage described herein. In some embodiments, the second period of time begins about 26 weeks after the final subcutaneous administration of the initiation dosage.

In some embodiments, each maintenance dosage comprises subcutaneously administering the compound of Formula Ia or Ib, or a pharmaceutically acceptable salt thereof, at a concentration of about 309 mg/mL, every 24 weeks to 28 weeks.

In some embodiments, each maintenance dosage comprises two subcutaneous injections of about 309 mg/mL, every 24 weeks to 28 weeks.

In some embodiments, each maintenance dosage comprises subcutaneously administering 927 mg of the compound of Formula Ia or Ib, or a pharmaceutically acceptable salt thereof, every 24 weeks to 28 weeks (e.g., two subcutaneous injections of 1.5 mL per injection, each comprising about 309 mg/mL of the compound of Formula Ia or Ib, or a pharmaceutically acceptable salt thereof).

In some embodiments, each maintenance dosage comprises administering two subcutaneous injections of 1.5 mL per injection, each comprising about 309 mg/mL of the compound of Formula Ia or Ib, or a pharmaceutically acceptable salt thereof, every 24 weeks to 28 weeks. In some embodiments, each maintenance dosage comprises subcutaneously administering the compound of Formula Ia, or a pharmaceutically acceptable salt thereof, at a concentration of about 309 mg/mL, every 24 weeks to 28 weeks.

In some embodiments, each maintenance dosage comprises subcutaneously administering about 927 mg of the compound of Formula Ia or Ib, or a pharmaceutically acceptable salt thereof, as two subcutaneous injections of about 309 mg/mL, every 24 weeks to 28 weeks.

In some embodiments, each maintenance dosage comprises subcutaneously administering the compound of Formula Ia or Ib, or a pharmaceutically acceptable salt thereof, at a concentration of about 309 mg/mL, every 26 weeks.

In some embodiments, each maintenance dosage comprises two subcutaneous injections of about 309 mg/mL, every 26 weeks.

In some embodiments, each maintenance dosage comprises subcutaneously administering 927 mg of the compound of Formula Ia or Ib, or a pharmaceutically acceptable salt thereof, every 26 weeks (e.g., two subcutaneous injections of 1.5 mL per injection, each comprising about 309 mg/mL of the compound of Formula Ia or Ib, or a pharmaceutically acceptable salt thereof).

In some embodiments, each maintenance dosage comprises administering two subcutaneous injections of 1.5 mL per injection, each comprising about 309 mg/mL of the compound of Formula Ia or Ib, or a pharmaceutically acceptable salt thereof, every 26 weeks. In some embodiments, each maintenance dosage comprises subcutaneously administering the compound of Formula Ia or Ib, or a pharmaceutically acceptable salt thereof, at a concentration of about 309 mg/mL, every 26 weeks.

In some embodiments, each maintenance dosage comprises subcutaneously administering about 927 mg of the compound of Formula Ia or Ib, or a pharmaceutically acceptable salt thereof, as two subcutaneous injections of about 309 mg/mL, every 26 weeks.

In some embodiments, the bridging dosage is administered for about 1 week to about 5 months and 3 weeks, e.g., about 1 week, about 2 weeks, about 3 weeks, about 4 weeks (i.e., about 1 month), about 5 weeks, about 6 weeks, about 7 weeks, about 8 weeks (i.e., about 2 months), about 9 weeks, about 10 weeks, about 11 weeks, about 12 weeks (i.e., about 3 months), about 13 weeks, about 14 weeks, about 15 weeks, about 16 weeks (i.e., about 4 months), about 17 weeks, about 18 weeks, about 19 weeks, about 20 weeks (i.e., about 5 months), about 21 weeks, about 22 weeks, about 23 weeks (i.e., about 5 months and 3 weeks).

In some embodiments, the bridging dosage is administered for about 1 week. In some embodiments, the bridging dosage is administered for about 4 weeks (i.e., about 1 month). In some embodiments, the bridging dosage is administered for about 8 weeks (i.e., about 2 months). In some embodiments, the bridging dosage is administered for about 12 weeks (i.e., about 3 months). In some embodiments, the bridging dosage is administered for about 16 weeks (i.e., about 4 months). In some embodiments, the bridging dosage is administered for about 20 weeks (i.e., about 5 months). In some embodiments, the bridging dosage is administered for about 23 weeks (i.e., about 5 months and 3 weeks).

In some embodiments, the patient is identified (or has been identified) as knowingly missing or anticipating missing the one or more maintenance dosages prior to administering the one or more bridging dosages. In some embodiments, the patient is identified (or has been identified) as knowingly missing the one or more maintenance dosages prior to administering the one or more bridging dosages. In some embodiments, the patient is identified (or has been identified) as anticipating missing the one or more maintenance dosages prior to administering the one or more bridging dosages. In some embodiments, the methods provided herein comprise administering the one or more bridging dosages, wherein the patient is identified (or has been identified) as knowingly missing or anticipating missing one or more of the maintenance dosages. For example, intentionally missing or anticipating missing one or more of the maintenance dosages may include, but is not limited to, missing a maintenance dosage due to travel that would prevent the patient from receiving the one or more maintenance dosages. In some embodiments, the methods provided herein comprise administering the one or more bridging dosages, wherein the patient is identified (or has been identified) as anticipating missing one or more of the maintenance dosages. In some embodiments the patient is identified (or has been identified) as anticipating missing one or more of the maintenance dosages during the maintenance dosing period. In some embodiments, the first bridging dosage is administered to the patient on the date of the next scheduled maintenance dosage. For example, if a patient receives a first maintenance dosage and subsequently anticipates missing a second maintenage dosage, then the first bridging dosage can be administered to the patient at the scheduled date of the second maintenance dosage (e.g., about 24 weeks to about 28 weeks after the first maintenance dosage, such as about 26 weeks after the first maintenance dosage).

In some embodiments, if the patient misses one or more of the bridging dosages, the method further comprises orally administering to the patient a dosage of about 250 mg to about 650 mg of the compound of Formula Ia or Ib, or a pharmaceutically acceptable salt thereof, as soon as possible after the missed bridging dose, followed by once-weekly oral administration of the bridging dosage (e.g., once-weekly oral administration of about 300 mg of the compound of Formula Ia or Ib, or a pharmaceutically acceptable salt thereof), until the patient resumes administration of the one or more maintenance dosages.

In some embodiments, if the patient misses one bridging dosage, the method further comprises orally administering to the patient a dosage of about 250 mg to about 350 mg of the compound of Formula Ia or Ib, or a pharmaceutically acceptable salt thereof, as soon as possible after the missed bridging dose, followed by once-weekly oral administration of the bridging dosage (e.g., once-weekly oral administration of about 300 mg of the compound of Formula Ia or Ib, or a pharmaceutically acceptable salt thereof), until the patient resumes administration of the one or more maintenance dosages.

In some embodiments, if the patient misses one bridging dosage, the method further comprises orally administering to the patient a dosage of about 300 mg of the compound of Formula Ia or Ib, or a pharmaceutically acceptable salt thereof, as soon as possible after the missed bridging dose, followed by once-weekly oral administration of the bridging dosage (e.g., once-weekly oral administration of about 300 mg of the compound of Formula Ia or Ib, or a pharmaceutically acceptable salt thereof), until the patient resumes administration of the one or more maintenance dosages.

In some embodiments, if the patient misses two bridging dosages, the method further comprises orally administering to the patient a dosage of about 500 mg to about 700 mg of the compound of Formula Ia or Ib, or a pharmaceutically acceptable salt thereof, as soon as possible after the second missed bridging dose, followed by once-weekly oral administration of the bridging dosage (e.g., once-weekly oral administration of about 300 mg of the compound of Formula Ia or Ib, or a pharmaceutically acceptable salt thereof), until the patient resumes administration of the one or more maintenance dosages.

In some embodiments, if the patient misses two bridging dosages, the method further comprises orally administering to the patient a dosage of about 600 mg of the compound of Formula Ia or Ib, or a pharmaceutically acceptable salt thereof, as soon as possible after the missed bridging dose, followed by once-weekly oral administration of the bridging dosage (e.g., once-weekly oral administration of about 300 mg of the compound of Formula Ia or Ib, or a pharmaceutically acceptable salt thereof), until the patient resumes administration of the one or more maintenance dosages.

In some embodiments, during the maintenance period, if a patient plans to miss a scheduled 6-month injection visit by more than 2 weeks, the compound of Formula Ia or Ib, or a pharmaceutically acceptable salt thereof, may be used for oral bridging on an interim basis for up to 6 months until injections resume.

In some embodiments, the recommended bridging dosage is about 300 mg of the compound of Formula Ia or Ib, or a pharmaceutically acceptable salt thereof.

In some embodiments, the recommended bridging dosage is about 300 mg of the compound of Formula Ia, or a pharmaceutically acceptable salt thereof.

In some embodiments, the recommended bridging dosage is about 300 mg of the compound of Formula Ib, or a pharmaceutically acceptable salt thereof.

In some embodiments, the recommended bridging dosage dosage is about 300 mg taken orally once every 7 days. In some embodiments, the maintenance dosage is administered within 7 days after the last oral bridging dose.

In some embodiments, during the maintenance period, if a patient plans to miss a scheduled 6-month injection visit by more than 2 weeks, SUNLENCA (lenacapavir) tablets may be used for oral bridging on an interim basis for up to 6 months until injections resume, wherein the recommended bridging dosage is 300 mg taken orally once every 7 days; and wherein the maintenance injection dosage is resumed within 7 days after the last oral dose.

In some embodiments, during the maintenance period, if a scheduled injection is to be missed by more than two weeks, SUNLENCA (lenacapavir) tablets may be used for oral bridging for up to 6 months until injections resume, wherein the recommended bridging dosage is 300 mg orally once every 7 days.

In some embodiments, the present disclosure further provides a method of treating or preventing HIV in a patient, comprising administering to the patient a compound of Formula Ia:

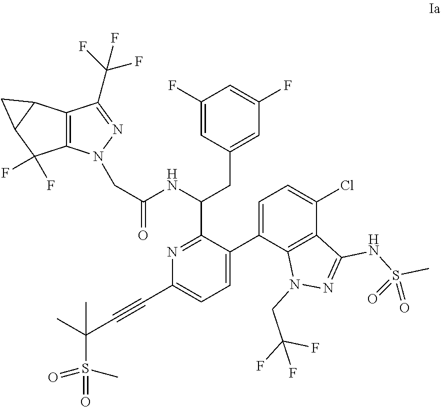 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(a) administering to the patient an initiation dosage comprising:
   (i) subcutaneously administering to the patient a dosage of 927 mg of the compound of Formula Ia, or a pharmaceutically acceptable salt thereof, and orally administering to the patient a dosage of 600 mg of the compound of Formula Ia, or a pharmaceutically acceptable salt thereof, on the first day;
   (ii) orally administering to the patient a dosage of 600 mg of the compound of Formula Ia, or a pharmaceutically acceptable salt thereof, on the second day; and
(b) administering to the patient one or more maintenance dosages of the compound of Formula Ia, or a pharmaceutically acceptable salt thereof, wherein each maintenance dosage comprises:
   (i) subcutaneously administering to the patient a dosage of 927 mg of the compound of Formula Ia, or a pharmaceutically acceptable salt thereof, once every 24 to 28 weeks from the date of the last subcutaneous administration;
wherein if the patient misses or will miss a maintenance dosage of step (b) (i), the method further comprises orally administering to the patient a bridging dosage of about 300 mg of the compound of Formula Ia, or a pharmaceutically acceptable salt thereof, once per week until the patient resumes administration of the one or more maintenance dosages.

In some embodiments, the present disclosure further provides a method of treating or preventing HIV in a patient, comprising administering to the patient a compound of Formula Ib:

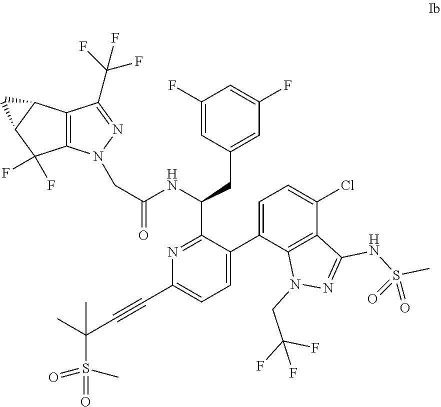 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(a) administering to the patient an initiation dosage comprising:
   (i) subcutaneously administering to the patient a dosage of 927 mg of the compound of Formula Ib, or a pharmaceutically acceptable salt thereof, and orally administering to the patient a dosage of 600 mg of the compound of Formula Ib, or a pharmaceutically acceptable salt thereof, on the first day;
   (ii) orally administering to the patient a dosage of 600 mg of the compound of Formula Ib, or a pharmaceutically acceptable salt thereof, on the second day; and
(b) administering to the patient one or more maintenance dosages of the compound of Formula Ib, or a pharmaceutically acceptable salt thereof, wherein each maintenance dosage comprises:
   (i) subcutaneously administering to the patient a dosage of 927 mg of the compound of Formula Ib, or a pharmaceutically acceptable salt thereof, once every 24 to 28 weeks from the date of the last subcutaneous administration;
wherein if the patient misses or will miss a maintenance dosage of step (b) (i), the method further comprises orally administering to the patient a bridging dosage of about 300 mg of the compound of Formula Ib, or a pharmaceutically acceptable salt thereof, once per week until the patient resumes administration of the one or more maintenance dosages.

In some embodiments, the present disclosure further provides a method of treating or preventing HIV in a patient, comprising administering to the patient a compound of Formula Ia:

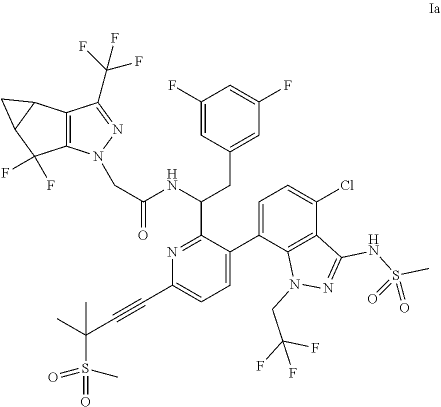 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(a) administering to the patient an initiation dosage comprising:
   (i) orally administering to the patient a dosage of 600 mg of the compound of Formula Ia, or a pharmaceutically acceptable salt thereof, on the first day and on the second day;
   (ii) orally administering to the patient a dosage of 300 mg of the compound of Formula Ia, or a pharmaceutically acceptable salt thereof, on the eighth day, wherein step (ii) occurs after step (i);
   (iii) subcutaneously administering to the patient a dosage of 927 mg of the compound of Formula Ia, or a pharmaceutically acceptable salt thereof, on the fifteenth day; and
(b) administering to the patient one or more maintenance dosages of the compound of Formula Ia, or a pharmaceutically acceptable salt thereof, wherein each maintenance dosage comprises:
   (i) subcutaneously administering to the patient a dosage of 927 mg of the compound of Formula Ia, or a pharmaceutically acceptable salt thereof, once every 24 to 28 weeks from the date of the last subcutaneous administration;
wherein if the patient misses or will miss a maintenance dosage of step (b) (1), the method further comprising orally administering to the patient a bridging dosage of about 300 mg of the compound of Formula Ia, or a pharmaceutically acceptable salt thereof, once per week until the patient resumes administration of the one or more maintenance dosages.

In some embodiments, the present disclosure further provides a method of treating or preventing HIV in a patient, comprising administering to the patient a compound of Formula Ib:

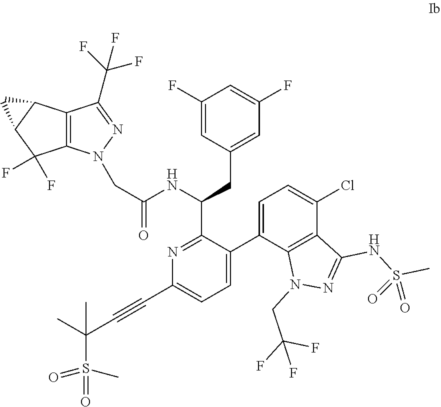 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(a) administering to the patient an initiation dosage comprising:
   (i) orally administering to the patient a dosage of 600 mg of the compound of Formula Ib, or a pharmaceutically acceptable salt thereof, on the first day and second day;
   (ii) orally administering to the patient a dosage of 300 mg of the compound of Formula Ib, or a pharmaceutically acceptable salt thereof, on the eighth day, wherein step (ii) occurs after step (i);
   (iii) subcutaneously administering to the patient a dosage of 927 mg of the compound of Formula Ib, or a pharmaceutically acceptable salt thereof, on the fifteenth day; and
(b) administering to the patient one or more maintenance dosages of the compound of Formula Ib, or a pharmaceutically acceptable salt thereof, wherein each maintenance dosage comprises:
   (i) subcutaneously administering to the patient a dosage of 927 mg of the compound of Formula Ib, or a pharmaceutically acceptable salt thereof, once every 24 to 28 weeks from the date of the last subcutaneous administration;
wherein if the patient misses or will miss a maintenance dosage of step (b) (1), the method further comprising orally administering to the patient a bridging dosage of about 300 mg of the compound of Formula Ib, or a pharmaceutically acceptable salt thereof, once per week until the patient resumes administration of the one or more maintenance dosages.

In some embodiments, the compound provided herein (e.g., the compound of Formula Ia or Ib, or a pharmaceutically acceptable salt thereof), is administered as a monotherapy (i.e., in the absence of an additional therapeutic agent). In some embodiments, the compound of Formula Ia or Ib, or a pharmaceutically acceptable salt thereof, is administered in combination with one or more additional therapeutic agents, such as anti-HIV agents.

In some embodiments, the methods provided herein comprise preventing an human immunodeficiency virus (HIV) infection in the patient. In some embodiments, the patient may have or be at risk of contracting an HIV infection. In some embodiments, the patient has been identified as an individual who is at risk of sexual transmission of HIV. In some embodiments, the individual has been identified as a man (e.g., who has sexual intercourse with a man or a woman), transgender man, transgender woman, a woman (e.g., who has sexual intercourse with a man or a woman), and/or a sex worker. In some embodiments, the individual has been identified as:
having anal sex with at least two different sexual partners and no consistent condom use over the last 6 months; and/or
having history of sexually transmitted diseases (STDs) during the last 12 months (e.g., syphilis, gonorrhea, chlamydiae, HBV or HCV infection); and/or
using psycho-active drugs during sexual intercourses (e.g., cocaine, gammahydroxybutyric acid (GHB), methylenedioxymethamphetamine (MDMA), mephedrone); and/or
having sexual intercourse with one or more partners originating from a region with high prevalence of HIV infection (>1%) (e.g., South America, Sub-Saharan Africa, South-East Asia, Eastern Europe, French Guyana) and no consistent condom use; and/or
a sex worker; and/or
having a sexual partner who is an intravenous drug user sharing injection material; and/or
having an HIV-infected sexual partner with a detectable plasma viral load (e.g., >50 copies (cp)/milliliter (mL)); and/or
a cisgender woman; and/or
a person who injects drugs, including, for example, but not limited to people who injects opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the patient is HIV-negative. In some embodiments, the HIV is HIV-1. In some embodiments, the HIV is HIV-2. In some embodiments, the HIV is HIV-1 and HIV-2.

As used herein, “HIV” or “Human Immunodeficiency Virus” refers to HIV-1 and/or to HIV-2.

The term “patient” is meant to refer to a human who is in need of therapeutic or preventative treatment for a viral infection, such as HIV infection.

As used herein, the terms “prevention” or “preventing” refers to the administration of a compound, pharmaceutically acceptable salt thereof, or composition comprising the compound or the pharmaceutically acceptable salt thereof according to the present disclosure pre- or post-exposure of the patient to the virus but before the appearance of symptoms of the disease, and/or prior to the detection of the virus in the blood. The terms also refer to prevention of the appearance of symptoms of the disease and/or to prevent the virus from reaching detectable levels in the blood. The terms include both pre-exposure prophylaxis (PrEP), as well as post-exposure prophylaxis (PEP) and event driven or “on demand” prophylaxis. The terms also refer to prevention of perinatal transmission of HIV from mother to baby by administration of a compound, pharmaceutically acceptable salt thereof, or composition comprising the compound or the pharmaceutically acceptable salt thereof according to the present disclosure to the mother before giving birth and to the child within the first days of life. The term also refers to prevention of transmission of HIV through blood transfusion.

As used herein the term “Cₜₐᵤ” refers to the observed drug concentration at the end of the dosing interval.

As used herein, the term “period of exposure” refers to a period of time, ranging from a single event or to multiple events over an extended period of time, in which a patient is exposed to HIV. For example, a patient who engages in one sexual intercourse event with a partner who is HIV-positive has a period of exposure that is limited to the time and duration of that one sexual intercourse event with that partner. As another example, a patient who has sexual intercourse with a partner who is HIV-positive on multiple occasions over an extended period of time (e.g., days, weeks, months, or years) has a period of exposure that ranges from the first instance to the last instance of sexual intercourse with that partner.

In some embodiments, the methods disclosed herein may comprise event driven administration of the compound of Formula Ia or Ib, or a pharmaceutically acceptable salt thereof, to the patient. As used herein, the terms “event driven” or “event driven administration” refer to administration of the compound of Formula Ia or Ib, or a pharmaceutically acceptable salt thereof, (1) prior to an event (e.g., 2 hours, 1 day, 2 days, 5 days, 7 days, 10 days, 14 days, 28 days (i.e., one month), or more days prior to the event) that would expose the patient to HIV (or that would otherwise increase the patient's risk of acquiring HIV); and/or (2) during an event (or more than one recurring event) that would expose the patient to HIV (or that would otherwise increase the patient's risk of acquiring HIV); and/or (3) after an event (or after the final event in a series of recurring events) that would expose the patient to HIV (or that would otherwise increase the patient's risk of acquiring HIV). In some embodiments, the event driven administration is performed pre-exposure of the patient to the HIV. In some embodiments, the event driven administration is performed during exposure of the patient to the HIV. In some embodiments, the event driven administration is performed post-exposure of the patient to the HIV.

In some embodiments, the event driven administration is performed pre-exposure of the patient to the HIV and during exposure of the patient to the HIV.

In some embodiments, the event driven administration is performed pre-exposure of the patient to the HIV and post-exposure of the patient to the HIV.

In some embodiments, the event driven administration is performed during exposure of the patient to the HIV and post-exposure of the patient to the HIV.

In certain embodiments, the methods disclosed herein involve administration prior to and/or after an event that would expose the patient to HIV or that would otherwise increase the patient's risk of acquiring HIV, e.g., as pre-exposure prophylaxis (PrEP) and/or as post-exposure prophylaxis (PEP). Examples of events that could increase a patient's risk of acquiring HIV include, without limitation, no condom use during anal intercourse with an HIV positive partner or a partner of unknown HIV status; anal intercourse with more than 3 sex partners; exchange of money, gifts, shelter or drugs for anal sex; sex with male partner and diagnosis of sexually transmitted infection; and no consistent use of condoms with sex partner known to be HIV positive. In some embodiments, the methods disclosed herein comprise pre-exposure prophylaxis (PrEP). In some embodiments, methods disclosed herein comprise post-exposure prophylaxis (PEP). In some embodiments, the methods disclosed herein comprise pre-exposure prophylaxis (PrEP) and post-exposure prophylaxis (PEP).

In some embodiments, the compound of Formula Ia or Ib, or a pharmaceutically acceptable salt thereof, is administered before exposure of the patient to the HIV.

In some embodiments, the compound of Formula Ia or Ib, or a pharmaceutically acceptable salt thereof, is administered during the period of exposure of the patient to the HIV.

In some embodiments, the compound of Formula Ia or Ib, or a pharmaceutically acceptable salt thereof, is administered after final exposure of the patient to the HIV.

In some embodiments, the compound of Formula Ia or Ib, or a pharmaceutically acceptable salt thereof, is administered before and during exposure of the patient to the HIV.

In some embodiments, the compound of Formula Ia or Ib, or a pharmaceutically acceptable salt thereof, is administered before and after exposure of the patient to the HIV.

In some embodiments, the compound of Formula Ia or Ib, or a pharmaceutically acceptable salt thereof, is administered during and after exposure of the patient to the HIV.

In some embodiments, the compound of Formula Ia or Ib, or a pharmaceutically acceptable salt thereof, is administered before, during, and after exposure of the patient to the HIV.

In some embodiments, the dose of the compound of Formula Ia or Ib, or a pharmaceutically acceptable salt thereof, administered during each period (i.e., before, during, and after exposure) may be different, i.e, independently selected from any of the doses disclosed herein.

In certain embodiments, e.g., when administered as PrEP, the compound of Formula Ia or Ib, or a pharmaceutically acceptable salt thereof, is administered 1 hour to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 24 hours, or 1 hour to 12 hours prior to an event that would increase the patient's risk of acquiring HIV (e.g., prior to sexual activity) prior to an event that would increase the patient's risk of acquiring HIV (e.g., prior to sexual intercourse or other exposure to the HIV). In some embodiments, the compound of Formula Ia or Ib, or a pharmaceutically acceptable salt thereof, is administered within 14 days, 13 days, 12 days, 11 days, 10 days, 9 days, 8 days, 7 days, 6 days, 5 days, 4 days, 3 days, 2 days, or 1 day prior to an event that would increase the patient's risk of acquiring HIV (e.g., prior to sexual intercourse or other exposure to the HIV). In some embodiments, the compound of Formula Ia or Ib, or a pharmaceutically acceptable salt thereof, is administered within 72 hours, 60 hours, 48 hours, 24 hours, 12 hours, 9 hours, 6 hours, 4 hours, 3 hours, 2 hours, or 1 hour prior to an event that would increase the patient's risk of acquiring HIV (e.g., prior to sexual intercourse or other exposure to the HIV). In certain embodiments, when the compound of Formula Ia or Ib, or a pharmaceutically acceptable salt thereof, is administered prior to an event that would increase the patient' risk of acquiring HIV, it is administered daily prior to the event (e.g., sexual activity). In certain embodiments, when the compound of Formula Ia or Ib, or a pharmaceutically acceptable salt thereof, is administered prior to an event that would increase the patient's risk of acquiring HIV, it is administered one to three times prior to the event. In certain embodiments, when the compound of Formula Ia or Ib, or a pharmaceutically acceptable salt thereof, is administered prior to an event that would increase the patient's risk of acquiring HIV, it is administered one time (i.e., once) prior to the event.

In some embodiments, the compound of Formula Ia or Ib, or a pharmaceutically acceptable salt thereof, is administered from about 14 days to about one day before exposure of the patient to the HIV. In some embodiments, the compound of Formula Ia or Ib, or a pharmaceutically acceptable salt thereof, is administered once from about 14 days to about one day before exposure of the patient to the HIV.

In some embodiments, the compound of Formula Ia or Ib, or a pharmaceutically acceptable salt thereof, is administered from about 10 days to about 5 days before exposure of the patient to the HIV. In some embodiments, the compound of Formula Ia or Ib, or a pharmaceutically acceptable salt thereof, is administered once from about 10 days to about 5 days before exposure of the patient to the HIV.

In some embodiments, the compound of Formula Ia or Ib, or a pharmaceutically acceptable salt thereof, is administered from about 8 days to about 6 days before exposure of the patient to the HIV. In some embodiments, the compound of Formula Ia or Ib, or a pharmaceutically acceptable salt thereof, is administered once from about 8 days to about 6 days before exposure of the patient to the HIV.

In some embodiments, the compound of Formula Ia or Ib, or a pharmaceutically acceptable salt thereof, is administered about 7 days before exposure of the patient to the HIV. In some embodiments, the compound of Formula Ia or Ib, or a pharmaceutically acceptable salt thereof, is administered once about 7 days before exposure of the patient to the HIV.

In some embodiments, the compound of Formula Ia or Ib, or a pharmaceutically acceptable salt thereof, is administered from about 72 hours to about 1 hour before exposure of the patient to the HIV. In some embodiments, the compound of Formula Ia or Ib, or a pharmaceutically acceptable salt thereof, is administered once from about 72 hours to about 1 hour before exposure of the patient to the HIV.

In some embodiments of the methods provided herein, the pre-exposure prophylaxis (PrEP) comprises continuous PrEP.

In certain embodiments where the compound of Formula Ia or Ib, or a pharmaceutically acceptable salt thereof, is administered before exposure of the patient to the HIV, the methods disclosed herein further comprise administering one or more additional doses of the compound of Formula Ia or Ib, or a pharmaceutically acceptable salt thereof, during, and/or after exposure of the patient to the HIV.

In some embodiments, e.g., when administered as part of a PrEP regimen or as part of a PEP regimen, the compound of Formula Ia or Ib, or a pharmaceutically acceptable salt thereof, is administered during the period of exposure of the patient to the HIV. In certain embodiments wherein the compound of Formula Ia or Ib, or a pharmaceutically acceptable salt thereof, is administered before HIV exposure, the compound of Formula Ia or Ib, or a pharmaceutically acceptable salt thereof, is administered about every 7 days, about every 14 days, about every 21 days, about every 28 days, about every 35 days, about every 42 days, or about every 6 months, or about every 12 months (e.g., as a single dose) during the time of HIV exposure (e.g., during the time period of sexual activity with sex partner known to be HIV positive). In some embodiments, the compound of Formula Ia or Ib, or a pharmaceutically acceptable salt thereof, is administered once about every 7 days, about every 14 days, about every 21 days, about every 28 days, about every 35 days, about every 42 days, about every 6 months, or about every 12 months during the period of exposure of the patient to the HIV.

In some embodiments, the compound of Formula Ia or Ib, or a pharmaceutically acceptable salt thereof, administered prior to exposure to the HIV is at a different dose than the compound of Formula Ia or Ib, or a pharmaceutically acceptable salt thereof, administered during and/or after exposure to the HIV. For example, in some embodiments, the dose of the compound of Formula Ia or Ib, or a pharmaceutically acceptable salt thereof, is increased, e.g., as a double dose, as a triple dose, and the like as compared to an earlier administered dose (e.g., a dose prior to exposure to the HIV). In some embodiments, the increased dose of the compound of Formula Ia or Ib, or a pharmaceutically acceptable salt thereof, is a double dose. In some embodiments, the dose of the compound of Formula Ia or Ib, or a pharmaceutically acceptable salt thereof, is decreased, e.g., a half dose as compared to an earlier administered dose (e.g., a dose prior to exposure to the HIV).

In some embodiments, the compound of Formula Ia or Ib, or a pharmaceutically acceptable salt thereof, is administered as a single dose from about 1 hour to about 10 days before exposure of the patient to the HIV.

Additional examples of PrEP and/or PEP can be found, for example, at the clinical trial summary titled “On Demand Antiretroviral Pre-exposure Prophylaxis for HIV Infection in Men Who Have Sex With Men” (Clinical Trial #NCT01473472); the clinical trial summary titled “Prevention of HIV in Île-de-France” (Clinical Trials #NCT03113123), and at Molina et al, N. Engl. J. Med. 2015, 353:2237-2246, the disclosure of each of which is incorporated herein by reference in its entirety.

In some embodiments, e.g., when administered as part of a PrEP regimen or as part of a PEP regimen, the compound of Formula Ia or Ib, or a pharmaceutically acceptable salt thereof, is administered 1 hour to 10 days, 1 hour to 7 days, 1 hour to 5 days, 1 to 72 hours, 1 to 48 hours, 1 to 36 hours, 1 to 24 hours, or 1 to 12 hours following an event that would increase the patient's risk of acquiring HIV (e.g., following sexual intercourse or other exposure to the HIV).

In certain embodiments, e.g., when administered as PEP, the compound of Formula Ia or Ib, or a pharmaceutically acceptable salt thereof, is administered for 7 days, 14 days, 21 days, 28 days, 30 days, or 45 days following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered for 30 days following an event that would increase the patient's risk of acquiring HIV (e.g., following sexual intercourse or other exposure to the HIV). In certain embodiments, the compound of Formula Ia or Ib, or a pharmaceutically acceptable salt thereof, is administered less than 1 hour, 2 hours, 3 hours, 4 hours, 5 hours, 6 hours, 7 hours, 8 hours, 9 hours, 12 hours, 18 hours, 24 hours, 36 hours, or 48 hours following an event that would increase the patient's risk of acquiring HIV (e.g., following sexual intercourse or other exposure to the HIV virus). In certain embodiments, the compound of Formula Ia or Ib, or a pharmaceutically acceptable salt thereof, is administered for 1 day, 2 days, 3 days, 4 days, or 5 days following an event that would increase the patient's risk of acquiring HIV (e.g., following sexual intercourse or other exposure to the HIV). In certain embodiments, when the compound of Formula Ia or Ib, or a pharmaceutically acceptable salt thereof, is administered following an event that would increase the patient's risk of acquiring HIV, it is administered daily following the event. In certain embodiments, when the compound of Formula Ia or Ib, or a pharmaceutically acceptable salt thereof, is administered following an event that would increase the patient's risk of acquiring HIV, it is administered one to three times following the event. In certain embodiments, when the compound of Formula Ia or Ib, or a pharmaceutically acceptable salt thereof, is administered following an event that would increase the patient's risk of acquiring HIV, it is administered once following the event.

In certain embodiments, e.g., when administered as PEP, the compound of Formula Ia or Ib, or a pharmaceutically acceptable salt thereof, is administered once about every week, once about every month, once about every 2 months, once about every 3 months, once about every 4 months, once about every 5 months, once about every 6 months, or once about every 12 months following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every week following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every month following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every 2 months following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every 3 months following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every 4 months following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every 5 months following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every 6 months following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every 12 months following an event that would increase the patient's risk of acquiring HIV (e.g., following sexual intercourse or other exposure to the HIV).

In certain embodiments, e.g., when administered as PEP, the compound of Formula Ia or Ib, or a pharmaceutically acceptable salt thereof, is administered for one month, two months, three months, four months, five months, six months, or twelve months following an event that would increase the patient's risk of acquiring HIV (e.g., following sexual intercourse or other exposure to the HIV).

In certain embodiments, when the compound of Formula Ia or Ib, or a pharmaceutically acceptable salt thereof, is administered following an event that would increase the patient's risk of acquiring HIV, it is administered one to fifty times following the event. In certain embodiments, when the compound of Formula Ia or Ib, or a pharmaceutically acceptable salt thereof, is administered following an event that would increase the patient's risk of acquiring HIV, it is administered one to forty times following the event. In certain embodiments, when the compound of Formula Ia or Ib, or a pharmaceutically acceptable salt thereof, is administered following an event that would increase the patient's risk of acquiring HIV, it is administered one to thirty times following the event. In certain embodiments, when the compound of Formula Ia or Ib, or a pharmaceutically acceptable salt thereof, is administered following an event that would increase the patient's risk of acquiring HIV, it is administered one to twenty times following the event. In certain embodiments, when the compound of Formula Ia or Ib, or a pharmaceutically acceptable salt thereof, is administered following an event that would increase the patient's risk of acquiring HIV, it is administered one to fifteen times following the event. In certain embodiments, when the compound of Formula Ia or Ib, or a pharmaceutically acceptable salt thereof, is administered following an event that would increase the patient's risk of acquiring HIV, it is administered one to ten times following the event. In certain embodiments, when the compound of Formula Ia or Ib, or a pharmaceutically acceptable salt thereof, is administered following an event that would increase the patient's risk of acquiring HIV, it is administered one to five times following the event.

In some embodiments, the compound of Formula Ia or Ib, or a pharmaceutically acceptable salt thereof, is administered during exposure of the patient to the HIV (e.g., during a period of sexual activity with sex partner known to be HIV positive).

In some embodiments, the compound of Formula Ia or Ib, or a pharmaceutically acceptable salt thereof, is administered after exposure (e.g., after final exposure) of the patient to the HIV (e.g., after a period of sexual activity with sex partner known to be HIV positive). In some embodiments, the compound of Formula Ia or Ib, or a pharmaceutically acceptable salt thereof, is administered from about 1 hour to about 14 days after exposure (e.g., after final exposure) of the patient to the HIV. In some embodiments, the compound of Formula Ia or Ib, or a pharmaceutically acceptable salt thereof, is administered once from about 1 hour to about 14 days after exposure (e.g., after final exposure) of the patient to the HIV. In some embodiments, the compound of Formula Ia or Ib, or a pharmaceutically acceptable salt thereof, is administered from about 1 hour to about 7 days after exposure (e.g., after final exposure) of the patient to the HIV. In some embodiments, the compound of Formula Ia or Ib, or a pharmaceutically acceptable salt thereof, is administered once from about 1 hour to about 7 days after exposure (e.g., after final exposure) of the patient to the HIV. In some embodiments, the compound of Formula Ia, or Ib or a pharmaceutically acceptable salt thereof, is administered from about 1 hour to about 72 hours after exposure (e.g., after final exposure) of the patient to the HIV. In some embodiments, the compound of Formula Ia or Ib, or a pharmaceutically acceptable salt thereof, is administered once from about 1 hour to about 72 hours after exposure (e.g., after final exposure) of the patient to the HIV. In some embodiments, the compound of Formula Ia or Ib, or a pharmaceutically acceptable salt thereof, is administered from about 1 hour to about 24 hours after exposure (e.g., after final exposure) of the patient to the HIV. In some embodiments, the compound of Formula Ia or Ib, or a pharmaceutically acceptable salt thereof, is administered once from about 1 hour to about 24 hours after exposure (e.g., after final exposure) of the patient to the HIV. In some embodiments, the compound of Formula Ia or Ib, or a pharmaceutically acceptable salt thereof, is administered from about 24 hours to about 72 hours after exposure (e.g., after final exposure) of the patient to the HIV. In some embodiments, the compound of Formula Ia or Ib, or a pharmaceutically acceptable salt thereof, is administered once from about 24 hours to about 72 hours after exposure (e.g., after final exposure) of the patient to the HIV.

In some embodiments, e.g., when administered as PrEP, the compound of Formula Ia or Ib, or a pharmaceutically acceptable salt thereof, is administered prior to an event that would increase the patient's risk of acquiring HIV (e.g., prior to sexual activity), and following the event. For example, in certain embodiments, when administered as PrEP, the compound of Formula Ia or Ib, or a pharmaceutically acceptable salt thereof, is administered 1 to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 24 hours, or 1 hour to 12 hours prior to an event that would increase the patient's risk of acquiring HIV (e.g., prior to sexual activity) and 1 hour to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72, 1 hour to 48 hours, 1 hour to 36 hours, 1 hour to 24 hours, or 1 hour to 12 hours following the event. For example, in some embodiments, one or more (e.g., one, two, or three) dosages of the compound of Formula Ia or Ib, or a pharmaceutically acceptable salt thereof, are administered one to ten days (e.g., seven days) prior to an event that would increase the patient's risk of acquiring HIV (e.g., prior to sexual intercourse) and once during a period of one to ten days following the event. In some embodiments, the compound of Formula Ia or Ib, or a pharmaceutically acceptable salt thereof, is administered once per week, twice per week, three times per week, four times per week, or five times per week and one or more times (e.g., one, two, or three times) beginning 1 to 48 hours following an event that would increase the patient's risk of acquiring HIV (e.g., following sexual intercourse).

Also provided herein is a method of reducing the risk of acquiring HIV in a patient, comprising administering to the patient a compound of Formula Ia or Ib, or a pharmaceutically acceptable salt thereof.

In some embodiments, methods for reducing the risk of acquiring HIV (e.g., HIV-1 and/or HIV-2) comprise administration of the compound of Formula Ia or Ib, or a pharmaceutically acceptable salt thereof, to a patient in combination with safer sexual intercourse practices. In certain embodiments, methods for reducing the risk of acquiring HIV (e.g., HIV-1 and/or HIV-2) comprise administration of the compound of Formula Ia or Ib, or a pharmaceutically acceptable salt thereof, to a patient at risk of acquiring HIV. Examples of patients at high risk for acquiring HIV include, without limitation, a patient who is at risk of sexual transmission of HIV.

In some embodiments, the reduction in risk of acquiring HIV is at least about 40%, 50%, 60%, 70%, 80%, 90%, or 95% (compared to a patient having not been administered the compound of Formula Ia or Ib, or a pharmaceutically acceptable salt thereof according to any of the methods provided herein). In some embodiments, the reduction in risk of acquiring HIV is about 80%, 85%, or 90%. In some embodiments, the reduction in risk of acquiring HIV is at least about 75%. In some embodiments, the reduction in risk of acquiring HIV is at least about 80%. In some embodiments, the reduction in risk of acquiring HIV is at least about 85%. In some embodiments, the reduction in risk of acquiring HIV is at least about 90%.

In some embodiments, the patient is a heavily treatment-experienced patient. In some embodiments, the methods provided herein comprise treating human immunodeficiency virus (HIV) infection in a heavily treatment-experienced patient.

In some embodiments, the present disclosure relates to the use of the compound of Formula Ia or Ib, or a pharmaceutically acceptable salt thereof, in treating an infection caused by the HIV virus comprising administering a therapeutically effective amount to a patient in need thereof, where the patient is a heavily treatment-experienced patient that has a multidrug resistant HIV infection.

As used herein, a “heavily treatment-experienced patient” refers to an HIV-infected patient who has limited treatment options due to a multidrug resistant HIV infection. For example, in some embodiments, the “heavily treatment-experienced patient” is a patient with HIV who has developed resistance to an antiretroviral medication from at least one class of antiretroviral medications selected from the group consisting of NRTIs, NNRTIs, PIs, and INSTIs.

In some embodiments, “multidrug resistant HIV infection” means resistance to an antiretroviral medication from at least one class of antiretroviral medications selected from the group consisting of NRTIs, NNRTIs, PIs, and INSTIs. In some embodiments, “multidrug resistant HIV infection” means resistance to at least one antiretroviral medication from two classes of antiretroviral medications selected from the group consisting of NRTIs, NNRTIs, PIs, and INSTIs. In some embodiments, “multidrug resistant HIV infection” means resistance to at least one antiretroviral medication from three classes of antiretroviral medications selected from the group consisting of NRTIs, NNRTIs, PIs, and INSTIs. In some embodiments, “multidrug resistant HIV infection” means resistance to at least one antiretroviral medication from each of the four classes of antiretroviral medications selected from the group consisting of NRTIs, NNRTIs, PIs, and INSTIs.

As used herein, the term “NRTI(s)” refers to nucleoside reverse transcriptase inhibitor(s) or nucleotide reverse transcriptase inhibitor(s).

As used herein, the term “NNRTI(s)” refers to non-nucleoside reverse transcriptase inhibitor(s) or non-nucleotide reverse transcriptase inhibitor(s).

As used herein, the term “PI(s)” refers to protease inhibitor(s).

As used herein, the term “INSTI(s)” refers to integrase strand transfer inhibitor(s).

As used herein, the term “fail” or “failed” when referring to HIV therapy or an HIV treatment regimen means a treatment outcome which precludes the use of the same agent or class in the future in a patient with HIV. This could be due to inadequate initial viral response due to pre-existing viral resistance, viral rebound due to emergent viral resistance, or inability of a patient to continue a treatment due to intolerability or safety issues.

In the disclosed methods, the heavily treatment-experienced patient is infected with multidrug resistant HIV. In some embodiments, the heavily treatment-experienced patient has a multidrug resistant HIV infection and is on a failing HIV treatment regimen. In some embodiments, the heavily treatment-experienced patient has a viral load greater than about 1,000 copies of HIV RNA/mL.

In some embodiments, the HIV infection is an HIV-1 infection. In some embodiments, the HIV-1 infection is characterized by HIV-1 mutant resistance to an antiretroviral medication, for example, to one, two, three, four, or more classes of antiretroviral medications (e.g., PIs, NRTIs, NNRTIs, INSTIs, etc.). In some embodiments, the HIV-1 infection is characterized by HIV-1 mutant resistance to one or more classes of antiretroviral medications. In some embodiments, the HIV-1 infection is characterized by HIV-1 mutant resistance to two or more classes of antiretroviral medications. In some embodiments, the HIV-1 infection is characterized by HIV-1 mutant resistance to three or more classes of antiretroviral medications.

In some embodiments, the HIV-1 mutant is resistant to a protease inhibitor (PI), a nucleoside or nucleotide reverse transcriptase inhibitor (NRTI), a non-nucleoside or non-nucleotide reverse transcriptase inhibitor (NNRTI), or an integrase strand transfer inhibitor (INSTI).

In some embodiments, the HIV-1 infection is characterized by an HIV-1 mutant that includes, but is not limited to:
(a) an HIV-1 mutant resistant to a PI (e.g., 150V, 184V/L90M, G48V/V82A/L90M, G48V/V82S, etc.);
(b) an HIV-1 mutant resistant to an NRTI (e.g., K65R, M184V, 6TAMs, etc.);
(c) an HIV-1 mutant resistant to an NNRTI (e.g., K103N, Y181C, Y188L, L100I/K103N, K103N/Y181C, etc.); and/or
(d) an HIV-1 mutant resistant to an INSTI (Y143R, E138K/Q148K, G140S/Q148R, E92Q/N155H, N155H/Q148R, R263K/M50I, etc.).

In some embodiments, the HIV-1 mutant is resistant to a protease inhibitor is selected from 150V, 184V/L90M, G48V/V82A/L90M, and G48V/V82S.

In some embodiments, the HIV-1 mutant is resistant to a nucleoside or nucleotide reverse transcriptase inhibitor is selected from K65R, M184V, and 6TAMs.

In some embodiments, the HIV-1 mutant is resistant to a non-nucleoside or non-nucleotide reverse transcriptase inhibitor is selected from K103N, Y181C, Y188L, L100I/K103N, and K103N/Y181C.

In some embodiments, the HIV-1 mutant is resistant to a integrase strand transfer inhibitor is selected from Y143R, E138K/Q148K, G140S/Q148R, E92Q/N155H, N155H/Q148R, and R263K/M50I.

In some embodiments, the patient is infected with HIV-1 that is resistant to at least one antiretroviral medication. In some embodiments, the patient is infected with multidrug resistant HIV-1. In some embodiments, the patient is infected with multidrug resistant HIV-1 that is resistant to at least one, two, three, four, or more antiretroviral medications. In some embodiments, the patient is infected with multidrug resistant HIV-1 that is resistant to at least one antiretroviral medication from each of two different classes of antiretroviral medications. In some embodiments, the patient is infected with multidrug resistant HIV-1 that is resistant to at least one antiretroviral medication from each of three different classes of antiretroviral medications. In some embodiments, the different classes of antiretroviral medications are selected from a nucleoside reverse transcriptase inhibitor (NRTI), a non-nucleoside reverse transcriptase inhibitor (NNRTI), a protease inhibitor (PI), and an integrase strand transfer inhibitor (INSTI). In some embodiments, the different classes of antiretroviral medications are selected from an NRTI, an NNRTI, and a PI. In some embodiments, the patient is infected with multidrug resistant HIV-1 that is resistant to at least one NRTI and at least one NNRTI. In some embodiments, the patient is infected with multidrug resistant HIV-1 that is resistant to at least one NRTI and at least one PI. In some embodiments, the patient is infected with multidrug resistant HIV-1 that is resistant to at least one NRTI and at least one INSTI. In some embodiments, the patient is infected with multidrug resistant HIV-1 that is resistant to at least one NNRTI and at least one PI. In some embodiments, the patient is infected with multidrug resistant HIV-1 that is resistant to at least one NNRTI and at least one INSTI. In some embodiments, the patient is infected with multidrug resistant HIV-1 that is resistant to at least one PI and at least one INSTI. In some embodiments, the patient is infected with multidrug resistant HIV-1 that is resistant to at least one NRTI, at least one NNRTI, and at least one PI. In some embodiments, the patient is infected with multidrug resistant HIV-1 that is resistant to at least one NRTI, at least one NNRTI, and at least one INSTI. In some embodiments, the patient is infected with multidrug resistant HIV-1 that is resistant to at least one NRTI, at least one PI, and at least one INSTI. In some embodiments, the patient is infected with multidrug resistant HIV-1 that is resistant to at least one NNRTI, at least one PI, and at least one INSTI. In some embodiments, the patient is infected with multidrug resistant HIV-1 that is resistant to at least one NRTI, at least one NNRTI, at least one PI, and at least one INSTI.

In some embodiments, the patient is infected with multidrug resistant HIV-1 that is resistant to at least one antiretroviral medication that is an NRTI. Examples of NRTIs include, but are not limited to, emtricitabine (FTC; Emtriva®), lamivudine (3TC; Epivir®), zidovudine (azidothymidine (AZT); Retrovir®), didanosine (ddI; Videx-EC®), dideoxyinosine (Videx®), tenofovir, tenofovir alafenamide (Vemlidy®), tenofovir disoproxil fumarate (Viread®), stavudine (d4T; Zerit®), zalcitabine (dideoxycytidine, ddC; Hivid®), and abacavir (Ziagen®).

In some embodiments, the patient is infected with multidrug resistant HIV-1 that is resistant to at least one antiretroviral medication that is an NNRTI. Examples of NNRTIs include, but are not limited to, efavirenz (Sustiva®), etravirine (Intelence®), rilpivirine (Edurant®), nevirapine (Viramune®), and delavirdine (Rescriptor®).

In some embodiments, the patient is infected with multidrug resistant HIV-1 that is resistant to at least one antiretroviral medication that is a PI. Examples of PIs include, but are not limited to, amprenavir (Agenerase®), atazanavir (Reyataz®), darunavir (Prezista®), fosamprenavir (Telzir®, Lexiva®), indinavir (Crixivan®), lopinavir (Kaletra®), nelfinavir (Viracept®), ritonavir (Norvir®), saquinavir (Invirase®), and tipranavir (Aptivus®).

In some embodiments, the patient is infected with multidrug resistant HIV-1 that is resistant to at least one antiretroviral medication that is an INSTI. Examples of INSTIs include, but are not limited to, raltegravir (Isentress®), elvitegravir (Vitekta®), dolutegravir (Tivicay®), cabotegravir, and bictegravir.

In some embodiments, the patient is infected with multidrug resistant HIV-1 that is resistant to at least one antiretroviral medication that is a gp41 fusion inhibitor. Examples of gp41 fusion inhibitors include, but are not limited to, albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

In some embodiments, the patient is infected with multidrug resistant HIV-1 that is resistant to at least one antiretroviral medication that is a CCR5 co-receptor antagonist. Examples of CCR5 co-receptor antagonists include, but are not limited to, aplaviroc, vicriviroc, maraviroc, cenicriviroc, PRO-140, adaptavir (RAP-101), nifeviroc (TD-0232), anti-GP120/CD4 or CCR5 bispecific antibodies, B-07, MB-66, polypeptide C25P, TD-0680, and vMIP (Haimipu).

In some embodiments of the disclosed methods, the patient has been previously treated with at least one antiretroviral medication before being treated with a compound of Formula Ia or Ib, or a pharmaceutically acceptable salt thereof. In some embodiments, the patient has been previously treated with at least one antiretroviral medication for at least 3 months, such as at least 4 months, at least 5 months, at least 6 months, at least 7 months, at least 8 months, at least 9 months, at least 10 months, at least 11 months, at least 12 months, at least 18 months, or at least 24 months. In some embodiments, the patient has been previously treated with at least one antiretroviral medication for at least 3 months. In some embodiments, the patient has been previously treated with at least one antiretroviral medication for at least 6 months. In some embodiments, the patient has been previously treated with at least one antiretroviral medication for at least 9 months. In some embodiments, the patient has been previously treated with at least one antiretroviral medication for at least 12 months. In some embodiments, the patient has been previously treated with at least one antiretroviral medication for at least 18 months. In some embodiments, the patient has been previously treated with at least one antiretroviral medication for at least 24 months. In some embodiments, the patient has been previously treated with at least one antiretroviral medication for at least 30 months. In some embodiments, the patient has been previously treated with at least one antiretroviral medication for at least 36 months.

In some embodiments of the disclosed methods, the patient has failed a prior HIV treatment regimen before being treated with a compound of Formula Ia or Ib, or a pharmaceutically acceptable salt thereof. In some embodiments of the disclosed methods, the patient is failing an HIV treatment regimen at the time of beginning administration of the compound of Formula Ia or Ib, or a pharmaceutically acceptable salt thereof. In some embodiments, the prior HIV treatment regimen included administering at least one antiretroviral medication. In some embodiments, the patient infected with HIV has relapsed after an initial response to the prior HIV treatment regimen, for example, antiretroviral therapy. In some embodiments, the patient has a viral load of greater than about 50 copies of HIV RNA/mL after about 48 weeks of therapy, for example, antiretroviral therapy, before being treated with a compound of Formula Ia or Ib, or a pharmaceutically acceptable salt thereof.

In some embodiments, the prior treatment regimen includes administering at least one antiretroviral medication from each of two different classes of antiretroviral medications. In some embodiments, the prior treatment regimen includes administering at least one antiretroviral medication from each of three different classes of antiretroviral medications. In some embodiments, the different classes of antiretroviral medications are selected from a nucleoside reverse transcriptase inhibitor (NRTI), a non-nucleoside reverse transcriptase inhibitor (NNRTI), a protease inhibitor (PI), and an integrase strand transfer inhibitor (INSTI). In some embodiments, the different classes of antiretroviral medications are selected from an NRTI, an NNRTI, and a PI. In some embodiments, the prior treatment regimen includes administering at least one NRTI and at least one NNRTI. In some embodiments, the prior treatment regimen includes administering at least one NRTI and at least one PI. In some embodiments, the prior treatment regimen includes administering at least one NRTI and at least one INSTI. In some embodiments, the prior treatment regimen includes administering at least one NNRTI and at least one PI. In some embodiments, the prior treatment regimen includes administering at least one NNRTI and at least one INSTI. In some embodiments, the prior treatment regimen includes administering at least one PI and at least one INSTI. In some embodiments, the prior treatment regimen includes administering at least one NRTI, at least one NNRTI, and at least one PI. In some embodiments, the prior treatment regimen includes administering at least one NRTI, at least one NNRTI, and at least one INSTI. In some embodiments, the prior treatment regimen includes administering at least one NRTI, at least one PI, and at least one INSTI. In some embodiments, the prior treatment regimen includes administering at least one NNRTI, at least one PI, and at least one INSTI.

In some embodiments, the prior treatment regimen includes administering at least one antiretroviral medication that is a gp41 fusion inhibitor.

In some embodiments, the prior treatment regimen includes administering at least one antiretroviral medication that is a CCR5 co-receptor antagonist.

In some embodiments, the prior treatment regimen includes administering at least one antiretroviral medication that is an NRTI. Examples of NRTIs include, but are not limited to, emtricitabine (FTC; Emtriva®), lamivudine (3TC; Epivir®), zidovudine (azidothymidine (AZT); Retrovir®), didanosine (ddI; Videx-EC®), dideoxyinosine (Videx®), tenofovir, tenofovir alafenamide (Vemlidy®), tenofovir disoproxil fumarate (Viread®), stavudine (d4T; Zerit®), zalcitabine (dideoxycytidine, ddC; Hivid®), and abacavir (Ziagen®).

In some embodiments, the prior treatment regimen includes administering at least one antiretroviral medication that is an NNRTI. Examples of NNRTIs include, but are not limited to, efavirenz (Sustiva®), etravirine (Intelence®), rilpivirine (Edurant®), nevirapine (Viramune®), and delavirdine (Rescriptor®).

In some embodiments, the prior treatment regimen includes administering at least one antiretroviral medication that is a PI. Examples of PIs include, but are not limited to, amprenavir (Agenerase®), atazanavir (Reyataz®), darunavir (Prezista®), fosamprenavir (Telzir®, Lexiva®), indinavir (Crixivan®), lopinavir (Kaletra®), nelfinavir (Viracept®), ritonavir (Norvir®), saquinavir (Invirase®), and tipranavir (Aptivus®).

In some embodiments, the prior treatment regimen includes administering at least one antiretroviral medication that is an INSTI. Examples of INSTIs include, but are not limited to, raltegravir (Isentress®), elvitegravir (Vitekta®), dolutegravir (Tivicay®), cabotegravir, and bictegravir.

In some embodiments, the prior treatment regimen includes administering at least one antiretroviral medication that is a gp41 fusion inhibitor. Examples of gp41 fusion inhibitors include, but are not limited to, albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

In some embodiments, the prior treatment regimen includes administering at least one antiretroviral medication that is a CCR5 co-receptor antagonist. Examples of CCR5 co-receptor antagonists include, but are not limited to, aplaviroc, vicriviroc, maraviroc, cenicriviroc, PRO-140, adaptavir (RAP-101), nifeviroc (TD-0232), anti-GP120/CD4 or CCR5 bispecific antibodies, B-07, MB-66, polypeptide C25P, TD-0680, and vMIP (Haimipu).

In some embodiments of the disclosed methods, the heavily treatment-experienced patient infected with HIV has a viral load of about 200 copies of HIV-1 RNA/mL (c/mL) to about 1,000,000 c/mL at the time of beginning administration of the compound of Formula Ia or Ib, or a pharmaceutically acceptable salt thereof, such as a viral load of about 200 c/mL to about 500,000 c/mL, about 200 c/mL to about 250,000 c/mL, about 200 c/mL to about 100,000 c/mL, about 200 c/mL to about 50,000 c/mL, about 200 c/mL to about 25,000 c/mL, about 200 c/mL to about 10,000 c/mL, about 200 c/mL to about 5,000 c/mL, about 200 c/mL to about 3,000 c/mL, about 200 c/mL to about 2,000 c/mL, about 200 c/mL to about 1,000 c/mL, about 200 c/mL to about 750 c/mL, about 200 c/mL to about 500 c/mL, about 500 c/mL to about 1,000,000 c/mL, about 500 c/mL to about 500,000 c/mL, about 500 c/mL to about 250,000 c/mL, about 500 c/mL to about 100,000 c/mL, about 500 c/mL to about 50,000 c/mL, about 500 c/mL to about 25,000 c/mL, about 500 c/mL to about 10,000 c/mL, about 500 c/mL to about 5,000 c/mL, about 500 c/mL to about 3,000 c/mL, about 500 c/mL to about 2,000 c/mL, about 500 c/mL to about 1,000 c/mL, about 500 c/mL to about 750 c/mL, about 750 c/mL to about 1,000,000 c/mL, about 750 c/mL to about 500,000 c/mL, about 750 c/mL to about 250,000 c/mL, about 750 c/mL to about 100,000 c/mL, about 750 c/mL to about 50,000 c/mL, about 750 c/mL to about 25,000 c/mL, about 750 c/mL to about 10,000 c/mL, about 750 c/mL to about 5,000 c/mL, about 750 c/mL to about 3,000 c/mL, about 750 c/mL to about 2,000 c/mL, about 750 c/mL to about 1,000 c/mL, about 1,000 c/mL to about 1,000,000 c/mL, about 1,000 c/mL to about 500,000 c/mL, about 1,000 c/mL to about 250,000 c/mL, about 1,000 c/mL to about 100,000 c/mL, about 1,000 c/mL to about 50,000 c/mL, about 1,000 c/mL to about 25,000 c/mL, about 1,000 c/mL to about 10,000 c/mL, about 1,000 c/mL to about 5,000 c/mL, about 1,000 c/mL to about 3,000 c/mL, about 1,000 c/mL to about 2,000 c/mL, about 2,000 c/mL to about 1,000,000 c/mL, about 2,000 c/mL to about 500,000 c/mL, about 2,000 c/mL to about 250,000 c/mL, about 2,000 c/mL to about 100,000 c/mL, about 2,000 c/mL to about 50,000 c/mL, about 2,000 c/mL to about 25,000 c/mL, about 2,000 c/mL to about 10,000 c/mL, about 2,000 c/mL to about 5,000 c/mL, about 2,000 c/mL to about 3,000 c/mL, about 3,000 c/mL to about 1,000,000 c/mL, about 3,000 c/mL to about 500,000 c/mL, about 3,000 c/mL to about 250,000 c/mL, about 3,000 c/mL to about 100,000 c/mL, about 3,000 c/mL to about 50,000 c/mL, about 3,000 c/mL to about 25,000 c/mL, about 3,000 c/mL to about 10,000 c/mL, about 3,000 c/mL to about 5,000 c/mL, about 5,000 c/mL to about 1,000,000 c/mL, about 5,000 c/mL to about 500,000 c/mL, about 5,000 c/mL to about 250,000 c/mL, about 5,000 c/mL to about 100,000 c/mL, about 5,000 c/mL to about 50,000 c/mL, about 5,000 c/mL to about 25,000 c/mL, about 5,000 c/mL to about 10,000 c/mL, about 10,000 c/mL to about 1,000,000 c/mL, about 10,000 c/mL to about 500,000 c/mL, about 10,000 c/mL to about 250,000 c/mL, about 10,000 c/mL to about 100,000 c/mL, about 10,000 c/mL to about 50,000 c/mL, about 10,000 c/mL to about 25,000 c/mL, about 25,000 c/mL to about 1,000,000 c/mL, about 25,000 c/mL to about 500,000 c/mL, about 25,000 c/mL to about 250,000 c/mL, about 25,000 c/mL to about 100,000 c/mL, about 25,000 c/mL to about 50,000 c/mL, about 50,000 c/mL to about 1,000,000 c/mL, about 50,000 c/mL to about 500,000 c/mL, about 50,000 c/mL to about 250,000 c/mL, about 50,000 c/mL to about 100,000 c/mL, about 100,000 c/mL to about 1,000,000 c/mL, about 100,000 c/mL to about 500,000 c/mL, about 100,000 c/mL to about 250,000 c/mL, about 250,000 c/mL to about 1,000,000 c/mL, about 250,000 c/mL to about 500,000 c/mL, or about 500,000 c/mL to about 1,000,000 c/mL.

In some embodiments, the patient has a viral load of greater than about 200 copies of HIV-1 RNA/mL (c/mL) at the time of beginning administration of the compound of Formula Ia or Ib, or a pharmaceutically acceptable salt thereof, such as a viral load greater than about 500 c/mL, about 750 c/mL, about 1,000 c/mL, about 2,000 c/mL, about 3,000 c/mL, about 5,000 c/mL, about 10,000 c/mL, about 25,000 c/mL, about 50,000 c/mL, about 100,000 c/mL, about 250,000 c/mL, about 500,000 c/mL, or greater than about 1,000,000 c/mL at the time of beginning administration of the compound of Formula Ia or Ib, or a pharmaceutically acceptable salt thereof. In some embodiments, the patient has a viral load of greater than about 200 c/mL at the time of beginning administration of the compound of Formula Ia or Ib, or a pharmaceutically acceptable salt thereof. In some embodiments, the patient has a viral load of greater than about 500 c/mL at the time of beginning administration of the compound of Formula Ia or Ib, or a pharmaceutically acceptable salt thereof. In some embodiments, the patient has a viral load of greater than about 750 c/mL at the time of beginning administration of the compound of Formula Ia or Ib, or a pharmaceutically acceptable salt thereof. In some embodiments, the patient has a viral load of greater than about 1,000 c/mL at the time of beginning administration of the compound of Formula Ia or Ib, or a pharmaceutically acceptable salt thereof. In some embodiments, the patient has a viral load of greater than about 2,000 c/mL at the time of beginning administration of the compound of Formula Ia or Ib, or a pharmaceutically acceptable salt thereof.

In some embodiments of the disclosed methods, administration of the compound of Formula Ia or Ib, or a pharmaceutically acceptable salt thereof, results in a decrease in the viral load in the patient. In some embodiments, the viral load is decreased by about 0.5 log₁₀ to about 2.5 log₁₀ after administration of the compound of Formula Ia or Ib, or a pharmaceutically acceptable salt thereof, for a certain amount of time as compared to the viral load at the time of beginning administration of the compound of Formula Ia or Ib, or a pharmaceutically acceptable salt thereof. For example, the viral load is decreased by about 0.5 log 10, about 1 log₁₀, about 1.5 log 10, about 2 log 10, or about 2.5 log₁₀ after administration of the compound of Formula Ia or Ib, or a pharmaceutically acceptable salt thereof, for a certain amount of time as compared to the viral load at the time of beginning administration of the compound of Formula Ia or Ib, or a pharmaceutically acceptable salt thereof. In some embodiments, the viral load is decreased by about 0.5 log₁₀ after administration of the compound of Formula Ia or Ib, or a pharmaceutically acceptable salt thereof, for about 24 weeks as compared to the viral load at the time of beginning administration of the compound of Formula Ia or Ib, or a pharmaceutically acceptable salt thereof. In some embodiments, the viral load is decreased by about 1 log₁₀ after administration of the compound of Formula Ia or Ib, or a pharmaceutically acceptable salt thereof, for about 24 weeks as compared to the viral load at the time of beginning administration of the compound of Formula Ia or Ib, or a pharmaceutically acceptable salt thereof. In some embodiments, the viral load is decreased by about 1.5 log₁₀ after administration of the compound of Formula Ia or Ib, or a pharmaceutically acceptable salt thereof, for about 24 weeks as compared to the viral load at the time of beginning administration of the compound of Formula Ia or Ib, or a pharmaceutically acceptable salt thereof. In some embodiments, the viral load is decreased by about 2 log₁₀ after administration of the compound of Formula Ia or Ib, or a pharmaceutically acceptable salt thereof, for about 24 weeks as compared to the viral load at the time of beginning administration of the compound of Formula Ia or Ib, or a pharmaceutically acceptable salt thereof. In some embodiments, the viral load is decreased by about 2.5 log₁₀ after administration of the compound of Formula Ia or Ib, or a pharmaceutically acceptable salt thereof, for about 24 weeks as compared to the viral load at the time of beginning administration of the compound of Formula Ia or Ib, or a pharmaceutically acceptable salt thereof.

In some embodiments of the disclosed methods, the viral load in the patient is about 200 c/mL or less after administration of the compound of Formula Ia or Ib, or a pharmaceutically acceptable salt thereof, for a certain amount of time, such as about 175 c/mL or less, about 150 c/mL or less, about 125 c/mL or less, about 100 c/mL or less, about 75 c/mL or less, or about 50 c/mL or less. In some embodiments, the viral load in the patient is about 200 c/mL or less after administration of the compound of Formula Ia or Ib, or a pharmaceutically acceptable salt thereof, for about 24 weeks. In some embodiments, the viral load in the patient is about 200 c/mL or less after administration of the compound of Formula Ia or Ib, or a pharmaceutically acceptable salt thereof, for about 24 weeks. In some embodiments, the viral load in the patient is about 100 c/mL or less after administration of the compound of Formula Ia or Ib, or a pharmaceutically acceptable salt thereof, for about 24 weeks. In some embodiments, the viral load in the patient is about 50 c/mL or less after administration of the compound of Formula Ia or Ib, or a pharmaceutically acceptable salt thereof, for about 24 weeks.

In certain embodiments of the disclosed methods, the heavily treatment-experienced patient is concurrently being treated with at least one additional antiretroviral medication. In some embodiments, the antiretroviral medication is selected from an NRTI, an NNRTI, a PI, an INSTI, a gp41 fusion inhibitor, and a CCR5 co-receptor antagonist.

In some embodiments, the patient is concurrently being treated with at least one NRTI. Examples of NRTIs include, but are not limited to, emtricitabine (FTC; Emtriva®), lamivudine (3TC; Epivir®), zidovudine (azidothymidine (AZT); Retrovir®), didanosine (ddI; Videx-EC®), dideoxyinosine (Videx®), tenofovir, tenofovir alafenamide (Vemlidy®), tenofovir disoproxil fumarate (Viread®), stavudine (d4T; Zerit®), zalcitabine (dideoxycytidine, ddC; Hivid®), and abacavir (Ziagen®).

In some embodiments, the patient is concurrently being treated with at least one NNRTI. Examples of NNRTIs include, but are not limited to, efavirenz (Sustiva®), etravirine (Intelence®), rilpivirine (Edurant®), nevirapine (Viramune®), and delavirdine (Rescriptor®).

In some embodiments, the patient is concurrently being treated with at least one PI. Examples of PIs include, but are not limited to, amprenavir (Agenerase®), atazanavir (Reyataz®), darunavir (Prezista®), fosamprenavir (Telzir®, Lexiva®), indinavir (Crixivan®), lopinavir (Kaletra®), nelfinavir (Viracept®), ritonavir (Norvir®), saquinavir (Invirase®), and tipranavir (Aptivus®).

In some embodiments, the patient is concurrently being treated with at least one INSTI. Examples of INSTIs include, but are not limited to, raltegravir (Isentress®), elvitegravir (Vitekta®), dolutegravir (Tivicay®), cabortegravir, and bictegravir.

In some embodiments, the patient is concurrently being treated with at least one gp41 fusion inhibitor. Examples of gp41 fusion inhibitors include, but are not limited to, albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer, and sifuvirtide.

In some embodiments, the patient is concurrently being treated with at least one CCR5 co-receptor antagonist. Examples of CCR5 co-receptor antagonists include, but are not limited to, aplaviroc, vicriviroc, maraviroc, cenicriviroc, PRO-140, adaptavir (RAP-101), nifeviroc (TD-0232), anti-GP120/CD4 or CCR5 bispecific antibodies, B-07, MB-66, polypeptide C25P, TD-0680, and vMIP (Haimipu).

Also provided in this disclosure is a method of treating an HIV-1 infection in a heavily treatment-experienced patient with multidrug resistant HIV-1 that includes administering a therapeutically effective amount of a compound of Formula Ia or Ib, or a pharmaceutically acceptable salt thereof, to a patient that had been previously treated with an HIV treatment regimen that includes the administration of at least one antiretroviral medication and had failed the treatment regimen. In some embodiments, the HIV treatment regimen includes administration of at least one antiretroviral medication such as those described herein. In some embodiments of the method, administration of the compound or Formula Ia or Ib, or a pharmaceutically acceptable salt thereof, results in a reduction in HIV viral load in the patient.

Also disclosed is a method of treating an HIV-1 infection in a heavily treatment-experienced patient with multidrug resistant HIV-1 that includes administering a therapeutically effective amount of a compound of Formula Ia or Ib, or a pharmaceutically acceptable salt thereof, to a patient that had been previously treated with an HIV treatment regimen that includes the administration of at least one antiretroviral medication and had failed the treatment regimen, where the multidrug resistant HIV-1 is resistant to at least one antiretroviral medication from each of two different classes of antiretroviral medications. In some embodiments, the different classes of antiretroviral medications are selected from an NRTI, an NNRTI, a PI, and an INSTI. In some embodiments, the patient has a viral load of greater than about 200 c/mL at the time of beginning administration of the compound of Formula Ia or Ib, or a pharmaceutically acceptable salt thereof, and administration of the compound results in a reduction in HIV viral load in the patient.

In some embodiments, disclosed is a method of treating an HIV-1 infection in a heavily treatment-experienced patient with multidrug resistant HIV-1 that includes administering a therapeutically effective amount of a compound of Formula Ia or Ib, or a pharmaceutically acceptable salt thereof, to a patient that had been previously treated with an HIV treatment regimen that includes the administration of at least one antiretroviral medication, and had failed the treatment regimen, where the multidrug resistant HIV-1 is resistant to at least one antiretroviral medication from each of three different classes of antiretroviral medications. In some embodiments, the different classes of antiretroviral medications are selected from an NRTI, an NNRTI, a PI, and an INSTI. In some embodiments, the patient has a viral load of greater than about 200 c/mL at the time of beginning administration of the compound of Formula Ia or Ib, or a pharmaceutically acceptable salt thereof, and administration of the compound results in a reduction in HIV viral load in the patient.

### Salts and Compositions

The present methods comprise administration of salts of the compound of Formula Ia or Ib, such as pharmaceutically acceptable salts. A salt generally refers to a derivative of a disclosed compound wherein the parent compound is modified by converting an existing acid or base moiety to its salt form. A pharmaceutically acceptable salt is one that, within the scope of sound medical judgment, is suitable for use in contact with the tissues of human beings and animals without excessive toxicity, irritation, allergic response, or other problem or complication, commensurate with a reasonable benefit/risk ratio. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety. In some embodiments, the salt is a sodium salt.

The compound of Formula Ia or Ib, or a salt thereof, can be present in a composition (such as a pharmaceutical composition or formulation) where the composition includes at least one compound other than the compound of Formula Ia or Ib or a salt thereof.

In some embodiments, the composition comprises the compound of Formula Ia or Ib, or a salt thereof, and one or more additional compounds (e.g., one or more additional therapeutic compounds), or salts thereof. In some embodiments, the composition comprises the compound of Formula Ia or Ib, or a salt thereof; bictegravir, or a salt thereof; and one or more additional compounds (e.g., one or more additional therapeutic compounds such as tenofovir alafenamide, or a pharmaceutically acceptable salt thereof), or salts thereof.

Compositions can include mixtures containing the compound of Formula Ia or Ib, or salt thereof, and one or more solvents, substrates, carriers, etc. In some embodiments, the composition comprises the compound of Formula Ia or Ib, or salt thereof, in an amount greater than about 25% by weight, for example, greater than about 25% by weight, greater than about 50% by weight, greater than about 75% by weight, greater than about 80% by weight, greater than about 90% by weight, or greater than about 95% by weight.

The present disclosure further includes pharmaceutical compositions comprising the compound of Formula Ia or Ib, or a pharmaceutically acceptable salt thereof, and at least one pharmaceutically acceptable carrier. As used herein, “pharmaceutically acceptable carrier” is meant to refer to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Administration of the compound of Formula Ia or Ib, or a pharmaceutically acceptable salt thereof, can be carried out via any of the accepted modes of administration of agents for serving similar utilities. The pharmaceutical compositions of the disclosure can be prepared by combining the compound of Formula Ia or Ib, or a pharmaceutically acceptable salt thereof, with an appropriate pharmaceutically acceptable carrier and, in specific embodiments, are formulated into preparations in solid, semi solid, liquid or gaseous forms, such as tablets, capsules, powders, granules, ointments, solutions, suppositories, injections, inhalants, gels, microspheres, and aerosols. Exemplary routes of administering such pharmaceutical compositions include, without limitation, oral, topical, transdermal, inhalation, parenteral, sublingual, buccal, rectal, vaginal, and intranasal. In some embodiments, pharmaceutical compositions of the disclosure are tablets. In some embodiments, pharmaceutical compositions of the disclosure are injection (e.g., intramuscular (IM) or intraperitoneal (IP)). Pharmaceutical compositions of the disclosure are formulated so as to allow the active ingredients contained therein to be bioavailable upon administration of the composition to a patient. Compositions that will be administered to a patient take the form of one or more dosage units, where for example, a tablet may be a single dosage unit, and a container of the compound of Formula Ia or Ib, or a pharmaceutically acceptable salt thereof, in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of the compound of Formula Ia or Ib, or a pharmaceutically acceptable salt thereof, for prevention of an HIV infection or reducing the risk of acquiring HIV, as described herein.

In some embodiments, the compound of Formula Ia or Ib, or a pharmaceutically acceptable salt thereof, is administered parenterally. Parenteral administration includes, but is not limited to, intravenous, intraarterial, subcutaneous, intraperitoneal, intramuscular, intracranial, transdermal, and vaginal administration. Parenteral administration can be administered, for example, in the form of a single bolus dose or by a continuous perfusion pump. In some embodiments, the compound of Formula Ia or Ib, or a pharmaceutically acceptable salt thereof, is administered to the patient through a medical device. Exemplary medical devices include, but are not limited to, a patch (e.g., a transdermal patch), an implantable device (e.g., an implantable device for metered or sustained release of an active agent; a subdermal device), a syringe, a contraceptive device (e.g., a vaginal ring, an intrauterine device), and the like.

In some embodiments, formulations suitable for parenteral administration (for example, intramuscular (IM) and subcutaneous (SC) administration) will include one or more excipients. Excipients should be compatible with the other ingredients of the formulation and physiologically innocuous to the recipient thereof. Examples of suitable excipients are well known to the person skilled in the art of parenteral formulation and can be found, for example, in the Handbook of Pharmaceutical Excipients (eds. Rowe, Sheskey & Quinn), 6th edition 2009.

Examples of solubilizing excipients in a parenteral formulation (for example, an SC or IM formulation) include, but are not limited to, polysorbates (such as polysorbate 20 or 80) and poloxamers (such as poloxamer 338, 188, or 207). In some embodiments, disclosed herein is a parenteral administration (for example, an SC or IM formulation) that comprises a compound of Formula Ia or Ib, or a pharmaceutically acceptable salt thereof, and a poloxamer. In some embodiments, the poloxamer is poloxamer 338. In some embodiments, the poloxamer is poloxamer 188. In some embodiments, the amount of poloxamer in a parenteral administration disclosed herein is less than about 10%, such as less than about 9%, about 8%, about 7%, about 6%, about 5%, about 4%, about 3%, about 2%, about 1%, or about 0.5%. In some embodiments, the amount of poloxamer in a parenteral administration disclosed herein is less than about 5%, such as less than about 3%, about 2%, about 1%, or about 0.5%.

In certain embodiments, excipients include N-methyl-2-pyrrolidone (NMP), dimethyl sulfoxide, polyethylene glycol and/or tetraglycol/glycofurol.

In general, poloxamers are synthetic non-ionic triblock of linear copolymers having a central hydrophobic chain of polyoxypropylene adjacent to two hydrophilic polypropylene oxide, in certain instances in a 4:2:4 weight ratio. Accordingly, in certain embodiments, the compositions disclosed herein include a compound of Formula Ia or Ib, or a pharmaceutically acceptable salt thereof, and a block copolymer comprised of one polyoxypropylene segment and two hydrophilic polypropylene oxide segments. In certain embodiments, the ratio of the polyoxypropylene segment to the two hydrophilic polypropylene oxide segments is 4:2:4 (hydrophilic polypropylene oxide:polyoxypropylene:hydrophilic polypropylene oxide). Poloxamers are generally understood to have the following structure:

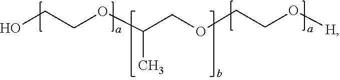

where a and b are integers. For example, a is between about 2 and about 130 and b is between about 15 and about 67. Poloxamer 188, for example, is understood to have a molecular weight from about 7680 to about 9510 Daltons (where a is about 80 and b is about 27) (see, for example, International Journal of PharmTech Research, Vol. 1, No. 2, pp 299-303, April-June 2009). In some instances, poloxamer 188 has an average molecular weight of about 8400 Daltons. Poloxamer 338 has a molecular weight in the range of from about 12700 Da to about 17400 Da (where a is about 141 and b is about 44).

In some embodiments, the pharmaceutical compositions disclosed herein comprise a compound of Formula Ia or Ib, or a pharmaceutically acceptable salt thereof, a poloxamer, and a pharmaceutically acceptable excipient. In some embodiments, the pharmaceutical compositions disclosed herein comprise a compound of Formula Ia or Ib, or a pharmaceutically acceptable salt thereof, poloxamer 188, and a pharmaceutically acceptable excipient. In some embodiments, the pharmaceutical compositions disclosed herein do not comprise a poloxamer. In some embodiments, the pharmaceutical compositions disclosed herein do not compromise poloxamer 188. In some embodiments, the pharmaceutical compositions disclosed herein are solutions that comprise a compound of Formula Ia or Ib, or a pharmaceutically acceptable salt thereof, and a pharmaceutically acceptable excipient. In some embodiments, the pharmaceutical compositions disclosed herein are solutions that comprise a compound of Formula Ia or Ib, or a pharmaceutically acceptable salt thereof, a poloxamer, and a pharmaceutically acceptable excipient. In some embodiments, the pharmaceutical compositions disclosed herein are solutions that comprise a compound of Formula Ia or Ib, or a pharmaceutically acceptable salt thereof, poloxamer 188, and a pharmaceutically acceptable excipient. In some embodiments, the pharmaceutical compositions disclosed herein are solutions that do not comprise a poloxamer. In some embodiments, the pharmaceutical compositions disclosed herein are solutions that do not comprise poloxamer 188.

Examples of excipients in a parenteral formulation (for example, an SC or an IM formulation) include polyethylene glycol. In general, polyethylene glycol (PEG) is a polyether having a general formula H—(O—CH₂—CH₂)ₙ—OH. In certain embodiments, the PEG may be “capped” by an alkyl group. In those embodiments, the capped PEG is of the formula alkyl-(O—CH₂—CH₂) n-O-alkyl (for example, CH₃—(O—CH₂—CH₂)ₙ—OCH₃). The pharmaceutical compositions of the present disclosure can include PEG having an average molecular weight of about 100 to about 1000. In some embodiments, the average molecular weight of PEG within the pharmaceutical composition is about 100 to about 800. In some embodiments, the average molecular weight of PEG within the pharmaceutical composition is about 200 to about 600. In some embodiments, the average molecular weight of PEG within the pharmaceutical composition is about 400. In some embodiments, the average molecular weight of PEG within the pharmaceutical composition is about 300. In some embodiments, the average molecular weight of PEG within the pharmaceutical composition is about 200. In some embodiments of the pharmaceutical composition, different molecular weight PEG can be combined to obtain a desired property or properties (for example, viscosity). Specific examples of PEG include, but are not limited to, PEG 100, PEG 200, PEG 300, PEG 400, PEG 500, and PEG 600. PEG 100, for example, refers to a polyethylene glycol with an average molecular weight of about 100.

The pharmaceutical compositions of the present disclosure can be in the form of a sterile injectable preparation, such as a solution or sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that can be employed are water, Ringer's solution, and isotonic sodium chloride solution. In addition, sterile fixed oils can conventionally be employed as a solvent or suspending medium. For this purpose any bland fixed oil can be employed, including synthetic mono- or diglycerides. In addition, fatty acids such as oleic acid can likewise be used in the preparation of injectables.

In some embodiments, the sterile injectable preparation disclosed herein can also be a sterile injectable solution or suspension prepared from a reconstituted lyophilized powder in a non-toxic parenterally acceptable diluent or solvent, such as a solution in 1,3-butane-diol. Among the acceptable vehicles and solvents that can be employed are water, Ringer's solution and isotonic sodium chloride solution. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. Formulations suitable for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents. In certain embodiments, the suspension is a microsuspension. In certain embodiments, the suspension is a nanosuspension.

In some embodiments, the pharmaceutical compositions of the present disclosure can be in the form of a solution formulation. In some embodiments, the solution comprises N-methyl-2-pyrrolidone (NMP), polyethylene glycol (PEG), water and/or glycofurol. In some embodiments, the solution comprises PEG 200, ethanol, and water. In some embodiments, the solution comprises PEG 300 and water. In some embodiments, the amount of water in the solution comprising PEG 300 and water is about 25 w/w %, about 23 w/w %, about 20 w/w %, about 17 w/w %, about 15 w/w %, about 10 w/w %, about 9 w/w %, about 8 w/w %, or about 5 w/w %. In some embodiments, the amount of PEG 300 in the solution comprising PEG 300 and water is about 90 w/w %, about 85 w/w %, about 80 w/w %, about 75 w/w %, about 70 w/w %, about 65 w/w %, about 60 w/w %, about 55 w/w %, about 50 w/w %, or about 45 w/w %. In some embodiments, the solution comprising PEG 300 and water comprises about 85 w/w % PEG 300 and about 15 w/w % water. In some embodiments, the solution comprising PEG 300 and water further comprises a base. In some embodiments, the solution comprising PEG 300 and water further comprises an inorganic base. In some embodiments, the inorganic base is sodium hydroxide or sodium ethoxide. In some embodiments, the sodium hydroxide or sodium ethoxide is in an amount of about 3 w/w %, about 2 w/w %, about 1 w/w %, or about 0.5 w/w %.

In some embodiments, the compound of Formula Ia or Ib, or a pharmaceutically acceptable salt thereof, is administered orally, subcutaneously, intramuscularly, or intravenously.

In some embodiments, the compound of Formula Ia or Ib, or a pharmaceutically acceptable salt thereof, is administered subcutaneously or intramuscularly.

In some embodiments, the compound of Formula Ia or Ib, or a pharmaceutically acceptable salt thereof, is administered subcutaneously at a concentration of about 10 mg/mL to about 600 mg/mL. In some embodiments, the compound of Formula Ia or Ib, or a pharmaceutically acceptable salt thereof, is administered subcutaneously at a concentration of about 10 mg/mL to about 500 mg/mL. In some embodiments, the compound of Formula Ia or Ib, or a pharmaceutically acceptable salt thereof, is administered subcutaneously at a concentration of about 50 mg/mL. In some embodiments, the compound of Formula Ia or Ib, or a pharmaceutically acceptable salt thereof, is administered subcutaneously at a concentration of about 100 mg/mL. In some embodiments, the compound of Formula Ia or Formula Ib, or a pharmaceutically acceptable salt thereof, is administered subcutaneously at a concentration of about 125 mg/mL. In some embodiments, the compound of Formula Ia or Ib, or a pharmaceutically acceptable salt thereof, is administered subcutaneously at a concentration of about 150 mg/mL. In some embodiments, the compound of Formula Ia or Formula Ib, or a pharmaceutically acceptable salt thereof, is administered subcutaneously at a concentration of about 175 mg/mL. In some embodiments, the compound of Formula Ia or Ib, or a pharmaceutically acceptable salt thereof, is administered subcutaneously at a concentration of about 300 mg/mL. In some embodiments, the compound of Formula Ia or Ib, or a pharmaceutically acceptable salt thereof, is administered subcutaneously at a concentration of about 309 mg/mL. In some embodiments, the compound of Formula Ia or Ib, or a pharmaceutically acceptable salt thereof, is administered subcutaneously at a concentration of about 400 mg/mL. In some embodiments, the compound of Formula Ia or Ib, or a pharmaceutically acceptable salt thereof, is administered subcutaneously at a concentration of about 500 mg/mL. In some embodiments, the compound of Formula Ia or Ib, or a pharmaceutically acceptable salt thereof, is administered subcutaneously at a concentration of about 600 mg/mL.

In some embodiments, the compound of Formula Ia or Ib, or a pharmaceutically acceptable salt thereof, is administered intramuscularly at a concentration of about 10 mg/mL to about 600 mg/mL. In some embodiments, the compound of Formula Ia or Ib, or a pharmaceutically acceptable salt thereof, is administered intramuscularly at a concentration of about 10 mg/mL to about 500 mg/mL. In some embodiments, the compound of Formula Ia or Ib, or a pharmaceutically acceptable salt thereof, is administered intramuscularly at a concentration of about 200 mg/mL to about 500 mg/mL. In some embodiments, the compound of Formula Ia or Ib, or a pharmaceutically acceptable salt thereof, is administered intramuscularly at a concentration of about 100 mg/mL. In some embodiments, the compound of Formula Ia or Ib, or a pharmaceutically acceptable salt thereof, is administered intramuscularly at a concentration of about 200 mg/mL. In some embodiments, the compound of Formula Ia or Ib, or a pharmaceutically acceptable salt thereof, is administered intramuscularly at a concentration of about 300 mg/mL. In some embodiments, the compound of Formula Ia or Ib, or a pharmaceutically acceptable salt thereof, is administered intramuscularly at a concentration of about 400 mg/mL. In some embodiments, the compound of Formula Ia or Ib, or a pharmaceutically acceptable salt thereof, is administered intramuscularly at a concentration of about 500 mg/mL. In some embodiments, the compound of Formula Ia or Ib, or a pharmaceutically acceptable salt thereof, is administered intramuscularly at a concentration of about 600 mg/mL.

In some embodiments of the methods provided herein, the compound of Formula Ia or Ib is administered to the patient as a solution for injection (e.g., for subcutaneous or intramuscular administration).

In some embodiments, the solution comprises a compound of Formula Ia or Ib, or a pharmaceutically acceptable salt thereof, PEG 300, and optionally one, two, or three additional components selected from water, poloxamer 188, and ethanol. In some embodiments, the solution comprises a compound of Formula Ia, or a pharmaceutically acceptable salt thereof, PEG 300, and optionally one, two, or three additional components selected from water, poloxamer 188, and ethanol. In some embodiments, the solution comprises a compound of Formula Ib, or a pharmaceutically acceptable salt thereof, PEG 300, and optionally one, two, or three additional components selected from water, poloxamer 188, and ethanol. In some embodiments, the solution comprises a compound of Formula Ia, PEG 300, and optionally one, two, or three additional components selected from water, poloxamer 188, and ethanol. In some embodiments, the solution comprises a sodium salt of the compound of Formula Ia, PEG 300, and optionally one, two, or three additional components selected from water, poloxamer 188, and ethanol. In some embodiments, the solution comprises a compound of Formula Ib, PEG 300, and optionally one, two, or three additional components selected from water, poloxamer 188, and ethanol. In some embodiments, the solution comprises a sodium salt of the compound of Formula Ib, PEG 300, and optionally one, two, or three additional components selected from water, poloxamer 188, and ethanol.

In some embodiments, the solution comprises a compound of Formula Ia or Ib, PEG 300, and optionally one, two, or three additional components selected from water, poloxamer 188, and ethanol. In some embodiments, the concentration of the compound of Formula Ia or Ib in the solution comprising a compound of Formula Ia or Ib, PEG 300, and optionally one, two, or three additional components selected from water, poloxamer 188, and ethanol is about 200 mg/ml to about 600 mg/ml. In some embodiments, the concentration of the compound of Formula Ia or Ib in the solution comprising a compound of Formula Ia or Ib, PEG 300, and optionally one, two, or three additional components selected from water, poloxamer 188, and ethanol is about 200 mg/ml. In some embodiments, the concentration of the compound of Formula Ia or Ib in the solution comprising a compound of Formula Ia or Ib, PEG 300, and optionally one, two, or three additional components selected from water, poloxamer 188, and ethanol is about 300 mg/ml. In some embodiments, the concentration of the compound of Formula Ia or Ib in the solution comprising a compound of Formula Ia or Ib, PEG 300, and optionally one, two, or three additional components selected from water, poloxamer 188, and ethanol is about 400 mg/ml. In some embodiments, the concentration of the compound of Formula Ia or Ib in the solution comprising a compound of Formula Ia or Ib, PEG 300, and optionally one, two, or three additional components selected from water, poloxamer 188, and ethanol is about 500 mg/ml. In some embodiments, the concentration of the compound of Formula Ia or Ib in the solution comprising a compound of Formula Ia or Ib, PEG 300, and optionally one, two, or three additional components selected from water, poloxamer 188, and ethanol is about 600 mg/ml.

In some embodiments, the amount of the compound of Formula Ia or Ib in the solution comprising a compound of Formula Ia or Ib, PEG 300, and optionally one, two, or three additional components selected from water, poloxamer 188, and ethanol is about 1 w/w % to about 75 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 9 w/w % to about 75 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 15 w/w % to about 65 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 20 w/w % to about 55 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 24 w/w % to about 50 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 20 w/w % to about 45 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 20 w/w % to about 30 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 25.2 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 25.20 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 25.7 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 25.71 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 33.87 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 33.9 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 41.0 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 40.92 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 41.08 w/w %. In some embodiments, the amount of the compound of Formula Ia or Ib in the solution comprising a compound of Formula Ia or Ib, PEG 300, and optionally one, two, or three additional components selected from water, poloxamer 188, and ethanol is about 41.1 w/w %.

In some embodiments, the amount of PEG 300 in the solution comprising a compound of Formula Ia or Ib, PEG 300, and optionally one, two, or three additional components selected from water, poloxamer 188, and ethanol is about 15 w/w % to about 90 w/w %. In some embodiments, the amount of PEG 300 in the solution comprising a compound of Formula Ia or Ib, PEG 300, and optionally one, two, or three additional components selected from water, poloxamer 188, and ethanol is about 25 w/w % to about 80 w/w %. In some embodiments, the amount of PEG 300 in the solution comprising a compound of Formula Ia or Ib, PEG 300, and optionally one, two, or three additional components selected from water, poloxamer 188, and ethanol is about 35 w/w % to about 70 w/w %. In some embodiments, the amount of PEG 300 in the solution comprising a compound of Formula Ia or Ib, PEG 300, and optionally one, two, or three additional components selected from water, poloxamer 188, and ethanol is about 40 w/w % to about 70 w/w %. In some embodiments, the amount of PEG 300 in the solution comprising a compound of Formula Ia or Ib, PEG 300, and optionally one, two, or three additional components selected from water, poloxamer 188, and ethanol is about 49 w/w % to about 59 w/w %. In some embodiments, the amount of PEG 300 in the solution comprising a compound of Formula Ia or Ib, PEG 300, and optionally one, two, or three additional components selected from water, poloxamer 188, and ethanol is about 40 w/w % to about 65 w/w %. In some embodiments, the amount of PEG 300 in the solution comprising a compound of Formula Ia or Ib, PEG 300, and optionally one, two, or three additional components selected from water, poloxamer 188, and ethanol is about 45.97 w/w %. In some embodiments, the amount of PEG 300 in the solution comprising a compound of Formula Ia or Ib, PEG 300, and optionally one, two, or three additional components selected from water, poloxamer 188, and ethanol is about 45.83 w/w %. In some embodiments, the amount of PEG 300 in the solution comprising a compound of Formula Ia or Ib, PEG 300, and optionally one, two, or three additional components selected from water, poloxamer 188, and ethanol is about 45.8 w/w %. In some embodiments, the amount of PEG 300 in the solution comprising a compound of Formula Ia or Ib, PEG 300, and optionally one, two, or three additional components selected from water, poloxamer 188, and ethanol is about 46.0 w/w %. In some embodiments, the amount of PEG 300 in the solution comprising a compound of Formula Ia or Ib, PEG 300, and optionally one, two, or three additional components selected from water, poloxamer 188, and ethanol is about 49.3 w/w %. In some embodiments, the amount of PEG 300 in the solution comprising a compound of Formula Ia or Ib, PEG 300, and optionally one, two, or three additional components selected from water, poloxamer 188, and ethanol is about 49.34 w/w %. In some embodiments, the amount of PEG 300 in the solution comprising a compound of Formula Ia or Ib, PEG 300, and optionally one, two, or three additional components selected from water, poloxamer 188, and ethanol is about 51.96 w/w %. In some embodiments, the amount of PEG 300 in the solution comprising a compound of Formula Ia or Ib, PEG 300, and optionally one, two, or three additional components selected from water, poloxamer 188, and ethanol is about 52.0 w/w %. In some embodiments, the amount of PEG 300 in the solution comprising a compound of Formula Ia or Ib, PEG 300, and optionally one, two, or three additional components selected from water, poloxamer 188, and ethanol is about 54.9 w/w %. In some embodiments, the amount of PEG 300 in the solution comprising a compound of Formula Ia or Ib, PEG 300, and optionally one, two, or three additional components selected from water, poloxamer 188, and ethanol is about 54.92 w/w %. In some embodiments, the amount of PEG 300 in the solution comprising a compound of Formula Ia or Ib, PEG 300, and optionally one, two, or three additional components selected from water, poloxamer 188, and ethanol is about 62.4 w/w %. In some embodiments, the amount of PEG 300 in the solution comprising a compound of Formula Ia or Ib, PEG 300, and optionally one, two, or three additional components selected from water, poloxamer 188, and ethanol is about 62.39 w/w %.

In some embodiments, the amount of water in the solution comprising a compound of Formula Ia or Ib, PEG 300, and optionally one, two, or three additional components selected from water, poloxamer 188, and ethanol is about 0 w/w % to about 40 w/w %. In some embodiments, the amount of water in the solution comprising a compound of Formula Ia or Ib, PEG 300, and optionally one, two, or three additional components selected from water, poloxamer 188, and ethanol is about 0 w/w % to about 30 w/w %. In some embodiments, the amount of water in the solution comprising a compound of Formula Ia or Ib, PEG 300, and optionally one, two, or three additional components selected from water, poloxamer 188, and ethanol is about 0 w/w % to about 20 w/w %. In some embodiments, the amount of water in the solution comprising a compound of Formula Ia or Ib, PEG 300, and optionally one, two, or three additional components selected from water, poloxamer 188, and ethanol is about 0 w/w % to about 15 w/w %. In some embodiments, the amount of water in the solution comprising a compound of Formula Ia or Ib, PEG 300, and optionally one, two, or three additional components selected from water, poloxamer 188, and ethanol is about 3 w/w % to about 15 w/w %. In some embodiments, the amount of water in the solution comprising a compound of Formula Ia or Ib, PEG 300, and optionally one, two, or three additional components selected from water, poloxamer 188, and ethanol is about 4 w/w % to about 14 w/w %. In some embodiments, the amount of water in the solution comprising a compound of Formula Ia or Ib, PEG 300, and optionally one, two, or three additional components selected from water, poloxamer 188, and ethanol is about 0 w/w %. In some embodiments, the amount of water in the solution comprising a compound of Formula Ia or Ib, PEG 300, and optionally one, two, or three additional components selected from water, poloxamer 188, and ethanol is about 8.09 w/w %. In some embodiments, the amount of water in the solution comprising a compound of Formula Ia or Ib, PEG 300, and optionally one, two, or three additional components selected from water, poloxamer 188, and ethanol is about 8.1 w/w %. In some embodiments, the amount of water in the solution comprising a compound of Formula Ia or Ib, PEG 300, and optionally one, two, or three additional components selected from water, poloxamer 188, and ethanol is about 8.11 w/w %. In some embodiments, the amount of water in the solution comprising a compound of Formula Ia or Ib, PEG 300, and optionally one, two, or three additional components selected from water, poloxamer 188, and ethanol is about 8.7 w/w %. In some embodiments, the amount of water in the solution comprising a compound of Formula Ia or Ib, PEG 300, and optionally one, two, or three additional components selected from water, poloxamer 188, and ethanol is about 8.71 w/w %. In some embodiments, the amount of water in the solution comprising a compound of Formula Ia or Ib, PEG 300, and optionally one, two, or three additional components selected from water, poloxamer 188, and ethanol is about 9.17 w/w %. In some embodiments, the amount of water in the solution comprising a compound of Formula Ia or Ib, PEG 300, and optionally one, two, or three additional components selected from water, poloxamer 188, and ethanol is about 9.2 w/w %. In some embodiments, the amount of water in the solution comprising a compound of Formula Ia or Ib, PEG 300, and optionally one, two, or three additional components selected from water, poloxamer 188, and ethanol is about 9.4 w/w %. In some embodiments, the amount of water in the solution comprising a compound of Formula Ia or Ib, PEG 300, and optionally one, two, or three additional components selected from water, poloxamer 188, and ethanol is about 9.41 w/w %. In some embodiments, the amount of water in the solution comprising a compound of Formula Ia or Ib, PEG 300, and optionally one, two, or three additional components selected from water, poloxamer 188, and ethanol is about 9.7 w/w %. In some embodiments, the amount of water in the solution comprising a compound of Formula Ia or Ib, PEG 300, and optionally one, two, or three additional components selected from water, poloxamer 188, and ethanol is about 9.70 w/w %.

In some embodiments, the amount of poloxamer 188 in the solution comprising a compound of Formula Ia or Ib, PEG 300, and optionally one, two, or three additional components selected from water, poloxamer 188, and ethanol is about 0 w/w % to about 35 w/w %. In some embodiments, the amount of poloxamer 188 in the solution comprising a compound of Formula Ia or Ib, PEG 300, and optionally one, two, or three additional components selected from water, poloxamer 188, and ethanol is about 0 w/w % to about 25 w/w %. In some embodiments, the amount of poloxamer 188 in the solution comprising a compound of Formula Ia or Ib, PEG 300, and optionally one, two, or three additional components selected from water, poloxamer 188, and ethanol is about 0 w/w % to about 15 w/w %. In some embodiments, the amount of poloxamer 188 in the solution comprising a compound of Formula Ia or Ib, PEG 300, and optionally one, two, or three additional components selected from water, poloxamer 188, and ethanol is about 0 w/w % to about 10 w/w %. In some embodiments, the amount of poloxamer 188 in the solution comprising a compound of Formula Ia or Ib, PEG 300, and optionally one, two, or three additional components selected from water, poloxamer 188, and ethanol is about 0 w/w % to about 8 w/w %. In some embodiments, the amount of poloxamer 188 in the solution comprising a compound of Formula Ia or Ib, PEG 300, and optionally one, two, or three additional components selected from water, poloxamer 188, and ethanol is about 1 w/w % to about 8 w/w %. In some embodiments, the amount of poloxamer 188 in the solution comprising a compound of Formula Ia or Ib, PEG 300, and optionally one, two, or three additional components selected from water, poloxamer 188, and ethanol is about 0 w/w % to about 5 w/w %. In some embodiments, the amount of poloxamer 188 in the solution comprising a compound of Formula Ia or Ib, PEG 300, and optionally one, two, or three additional components selected from water, poloxamer 188, and ethanol is about 0 w/w % to about 3 w/w %. In some embodiments, the amount of poloxamer 188 in the solution comprising a compound of Formula Ia or Ib, PEG 300, and optionally one, two, or three additional components selected from water, poloxamer 188, and ethanol is about 0 w/w %. In some embodiments, the amount of poloxamer 188 in the solution comprising a compound of Formula Ia or Ib, PEG 300, and optionally one, two, or three additional components selected from water, poloxamer 188, and ethanol is about 1 w/w %. In some embodiments, the amount of poloxamer 188 in the solution comprising a compound of Formula Ia or Ib, PEG 300, and optionally one, two, or three additional components selected from water, poloxamer 188, and ethanol is about 2 w/w %. In some embodiments, the amount of poloxamer 188 in the solution comprising a compound of Formula Ia or Ib, PEG 300, and optionally one, two, or three additional components selected from water, poloxamer 188, and ethanol is about 3 w/w %. In some embodiments, the amount of poloxamer 188 in the solution comprising a compound of Formula Ia or Ib, PEG 300, and optionally one, two, or three additional components selected from water, poloxamer 188, and ethanol is about 3.08 w/w %. In some embodiments, the amount of poloxamer 188 in the solution comprising a compound of Formula Ia or Ib, PEG 300, and optionally one, two, or three additional components selected from water, poloxamer 188, and ethanol is about 3.1 w/w %. In some embodiments, the amount of poloxamer 188 in the solution comprising a compound of Formula Ia or Ib, PEG 300, and optionally one, two, or three additional components selected from water, poloxamer 188, and ethanol is about 4 w/w %. In some embodiments, the amount of poloxamer 188 in the solution comprising a compound of Formula Ia or Ib, PEG 300, and optionally one, two, or three additional components selected from water, poloxamer 188, and ethanol is about 4.67 w/w %. In some embodiments, the amount of poloxamer 188 in the solution comprising a compound of Formula Ia or Ib, PEG 300, and optionally one, two, or three additional components selected from water, poloxamer 188, and ethanol is about 4.7 w/w %.

In some embodiments, the amount of ethanol in the solution comprising a compound of Formula Ia or Ib, PEG 300, and optionally one, two, or three additional components selected from water, poloxamer 188, and ethanol is about 0 w/w % to about 30 w/w %. In some embodiments, the amount of ethanol in the solution comprising a compound of Formula Ia or Ib, PEG 300, and optionally one, two, or three additional components selected from water, poloxamer 188, and ethanol is about 0 w/w % to about 25 w/w %. In some embodiments, the amount of ethanol in the solution comprising a compound of Formula Ia or Ib, PEG 300, and optionally one, two, or three additional components selected from water, poloxamer 188, and ethanol is about 0 w/w % to about 15 w/w %. In some embodiments, the amount of ethanol in the solution comprising a compound of Formula Ia or Ib, PEG 300, and optionally one, two, or three additional components selected from water, poloxamer 188, and ethanol is about 0 w/w % to about 10 w/w %. In some embodiments, the amount of ethanol in the solution comprising a compound of Formula Ia or Ib, PEG 300, and optionally one, two, or three additional components selected from water, poloxamer 188, and ethanol is about 0 w/w % to about 8 w/w %. In some embodiments, the amount of ethanol in the solution comprising a compound of Formula Ia or Ib, PEG 300, and optionally one, two, or three additional components selected from water, poloxamer 188, and ethanol is about 0 w/w % to about 5 w/w %. In some embodiments, the amount of ethanol in the solution comprising a compound of Formula Ia or Ib, PEG 300, and optionally one, two, or three additional components selected from water, poloxamer 188, and ethanol is about 1 w/w % to about 5 w/w %. In some embodiments, the amount of poloxamer 188 in the solution comprising a compound of Formula Ia or Ib, PEG 300, and optionally one, two, or three additional components selected from water, poloxamer 188, and ethanol is about 0 w/w %, about 1 w/w %, about 2 w/w %, about 3 w/w %, about 4 w/w %, or about 5 w/w %. In some embodiments, the amount of poloxamer 188 in the solution comprising a compound of Formula Ia or Ib, PEG 300, and optionally one, two, or three additional components selected from water, poloxamer 188, and ethanol is about 0 w/w %. In some embodiments, the amount of poloxamer 188 in the solution comprising a compound of Formula Ia or Ib, PEG 300, and optionally one, two, or three additional components selected from water, poloxamer 188, and ethanol is about 1 w/w %. In some embodiments, the amount of poloxamer 188 in the solution comprising a compound of Formula Ia or Ib, PEG 300, and optionally one, two, or three additional components selected from water, poloxamer 188, and ethanol is about 2 w/w %. In some embodiments, the amount of poloxamer 188 in the solution comprising a compound of Formula Ia or Ib, PEG 300, and optionally one, two, or three additional components selected from water, poloxamer 188, and ethanol is about 3 w/w %. In some embodiments, the amount of poloxamer 188 in the solution comprising a compound of Formula Ia or Ib, PEG 300, and optionally one, two, or three additional components selected from water, poloxamer 188, and ethanol is about 4 w/w %. In some embodiments, the amount of poloxamer 188 in the solution comprising a compound of Formula Ia or Ib, PEG 300, and optionally one, two, or three additional components selected from water, poloxamer 188, and ethanol is about 5 w/w %. In some embodiments, the amount of poloxamer 188 in the solution comprising a compound of Formula Ia or Ib, PEG 300, and optionally one, two, or three additional components selected from water, poloxamer 188, and ethanol is about 6 w/w %. In some embodiments, the amount of poloxamer 188 in the solution comprising a compound of Formula Ia or Ib, PEG 300, and optionally one, two, or three additional components selected from water, poloxamer 188, and ethanol is about 7 w/w %. In some embodiments, the amount of poloxamer 188 in the solution comprising a compound of Formula Ia or Ib, PEG 300, and optionally one, two, or three additional components selected from water, poloxamer 188, and ethanol is about 8 w/w %. In some embodiments, the amount of poloxamer 188 in the solution comprising a compound of Formula Ia or Ib, PEG 300, and optionally one, two, or three additional components selected from water, poloxamer 188, and ethanol is about 9 w/w %. In some embodiments, the amount of poloxamer 188 in the solution comprising a compound of Formula Ia or Ib, PEG 300, and optionally one, two, or three additional components selected from water, poloxamer 188, and ethanol is about 10 w/w %.

In some embodiments, the solution comprises about 20 w/w % to about 55 w/w % of the compound of Formula Ia or Ib, about 35 w/w % to about 70 w/w % of PEG 300, about 0 w/w % to about 20 w/w % of water, about 0 w/w % to about 10 w/w % of poloxamer 188, and about 0 w/w % to about 10 w/w % of ethanol. In some embodiments, the solution comprises about 24 w/w % to about 50 w/w % of the compound of Formula Ia or Ib, about 40 w/w % to about 65 w/w % of PEG 300, about 0 w/w % to about 15 w/w % of water, about 0 w/w % to about 8 w/w % of poloxamer 188, and about 0 w/w % to about 10 w/w % of ethanol. In some embodiments, the solution comprises about 24 w/w % to about 50 w/w % of the compound of Formula Ia or Ib, about 40 w/w % to about 65 w/w % of PEG 300, about 0 w/w % to about 15 w/w % of water, about 0 w/w % to about 8 w/w % of poloxamer 188, and about 0 w/w % to about 5 w/w % of ethanol.

In some embodiments, the solution comprises about 25.2 w/w % of the compound of Formula Ia or Ib, about 62.4 w/w % of PEG 300, about 9.4 w/w % of water, and about 3.0 w/w % of poloxamer 188. In some embodiments, the solution comprises about 25.20 w/w % of the compound of Formula Ia or Ib, about 62.39 w/w % of PEG 300, about 9.41 w/w % of water, and about 3.00 w/w % of poloxamer 188. In some embodiments, the solution comprises about 25.2 w/w % of the compound of Formula Ib, about 62.4 w/w % of PEG 300, about 9.4 w/w % of water, and about 3.0 w/w % of poloxamer 188. In some embodiments, the solution comprises about 25.20 w/w % of the compound of Formula Ib, about 62.39 w/w % of PEG 300, about 9.41 w/w % of water, and about 3.00 w/w % of poloxamer 188.

In some embodiments, the solution comprises about 20 w/w % to about 30 w/w % of the compound of Formula Ia or Ib, about 49 w/w % to about 59 w/w % of PEG 300, about 4 w/w % to about 14 w/w % of water, about 1 w/w % to about 8 w/w % of poloxamer 188, and about 1 w/w % to about 5 w/w % of ethanol. In some embodiments, the solution comprises about 25.20 w/w % of the compound of Formula Ia or Ib, about 62.39 w/w % of PEG 300, about 9.41 w/w % of water, and about 3.00 w/w % of poloxamer 188. In some embodiments, the solution comprises about 25.2 w/w % of the compound of Formula Ib, about 62.4 w/w % of PEG 300, about 9.4 w/w % of water, and about 3.0 w/w % of poloxamer 188. In some embodiments, the solution comprises about 25.20 w/w % of the compound of Formula Ib, about 62.39 w/w % of PEG 300, about 9.41 w/w % of water, and about 3.00 w/w % of poloxamer 188.

In some embodiments, the solution comprises about 25.7 w/w % of the compound of Formula Ia or Ib, about 54.9 w/w % of PEG 300, about 9.7 w/w % of water, about 4.7 w/w % of poloxamer 188, and about 5.0 w/w % of ethanol. In some embodiments, the solution comprises about 25.71 w/w % of the compound of Formula Ia or Ib, about 54.92 w/w % of PEG 300, about 9.70 w/w % of water, about 4.67 w/w % of poloxamer 188, and about 5.00 w/w % of ethanol. In some embodiments, the solution comprises about 25.7 w/w % of the compound of Formula Ib, about 54.9 w/w % of PEG 300, about 9.7 w/w % of water, about 4.7 w/w % of poloxamer 188, and about 5.0 w/w % of ethanol. In some embodiments, the solution comprises about 25.71 w/w % of the compound of Formula Ib, about 54.92 w/w % of PEG 300, about 9.70 w/w % of water, about 4.67 w/w % of poloxamer 188, and about 5.00 w/w % of ethanol.

In some embodiments, the solution comprises about 33.9 w/w % of the compound of Formula Ia or Ib, about 52.0 w/w % of PEG 300, about 9.2 w/w % of water, and about 5.0 w/w % of ethanol. In some embodiments, the solution comprises about 33.87 w/w % of the compound of Formula Ia or Ib, about 51.96 w/w % of PEG 300, about 9.17 w/w % of water, and about 5.00 w/w % of ethanol. In some embodiments, the solution comprises about 33.9 w/w % of the compound of Formula Ib, about 52.0 w/w % of PEG 300, about 9.2 w/w % of water, and about 5.0 w/w % of ethanol. In some embodiments, the solution comprises about 33.87 w/w % of the compound of Formula Ib, about 51.96 w/w % of PEG 300, about 9.17 w/w % of water, and about 5.00 w/w % of ethanol.

In some embodiments, the solution comprises about 33.9 w/w % of the compound of Formula Ia or Ib, about 49.3 w/w % of PEG 300, about 8.7 w/w % of water, about 3.1 w/w % of poloxamer 188, and about 5.0 w/w % of ethanol. In some embodiments, the solution comprises about 33.87 w/w % of the compound of Formula Ia or Ib, about 49.34 w/w % of PEG 300, about 8.71 w/w % of water, about 3.08 w/w % of poloxamer 188, and about 5.00 w/w % of ethanol. In some embodiments, the solution comprises about 33.9 w/w % of the compound of Formula Ib, about 49.3 w/w % of PEG 300, about 8.7 w/w % of water, about 3.1 w/w % of poloxamer 188, and about 5.0 w/w % of ethanol. In some embodiments, the solution comprises about 33.87 w/w % of the compound of Formula Ib, about 49.34 w/w % of PEG 300, about 8.71 w/w % of water, about 3.08 w/w % of poloxamer 188, and about 5.00 w/w % of ethanol.

In some embodiments, the solution comprises about 41.0 w/w % of the compound of Formula Ia or Ib, about 46.0 w/w % of PEG 300, about 8.1 w/w % of water, and about 5.0 w/w % of ethanol. In some embodiments, the solution comprises about 40.92 w/w % of the compound of Formula Ia or Ib, about 45.97 w/w % of PEG 300, about 8.11 w/w % of water, and about 5.00 w/w % of ethanol. In some embodiments, the solution comprises about 41.0 w/w % of the compound of Formula Ib, about 46.0 w/w % of PEG 300, about 8.1 w/w % of water, and about 5.0 w/w % of ethanol. In some embodiments, the solution comprises about 40.92 w/w % of the compound of Formula Ib, about 45.97 w/w % of PEG 300, about 8.11 w/w % of water, and about 5.00 w/w % of ethanol.

In some embodiments, the solution comprises about 41.1 w/w % of the compound of Formula Ia or Ib, about 45.8 w/w % of PEG 300, about 8.1 w/w % of water, and about 5.0 w/w % of ethanol. In some embodiments, the solution comprises about 41.08 w/w % of the compound of Formula Ia or Ib, about 45.83 w/w % of PEG 300, about 8.09 w/w % of water, and about 5.00 w/w % of ethanol. In some embodiments, the solution comprises about 41.1 w/w % of the compound of Formula Ib, about 45.8 w/w % of PEG 300, about 8.1 w/w % of water, and about 5.0 w/w % of ethanol. In some embodiments, the solution comprises about 41.08 w/w % of the compound of Formula Ib, about 45.83 w/w % of PEG 300, about 8.09 w/w % of water, and about 5.00 w/w % of ethanol.

In some embodiments, the solution comprises a sodium salt of the compound of Formula Ia or Ib, PEG 300, and optionally one, two, or three additional components selected from water, poloxamer 188, and ethanol. In some embodiments, the concentration of the compound of Formula Ia or Ib in the solution comprising a sodium salt of the compound of Formula Ia or Ib, PEG 300, and optionally one, two, or three additional components selected from water, poloxamer 188, and ethanol is about 200 mg/ml to about 600 mg/ml. In some embodiments, the concentration of the compound of Formula Ia or Ib in the solution comprising a sodium salt of the compound of Formula Ia or Ib, PEG 300, and optionally one, two, or three additional components selected from water, poloxamer 188, and ethanol is about 200 mg/ml. In some embodiments, the concentration of the compound of Formula Ia or Ib in the solution comprising a sodium salt of the compound of Formula Ia or Ib, PEG 300, and optionally one, two, or three additional components selected from water, poloxamer 188, and ethanol is about 300 mg/ml. In some embodiments, the concentration of the compound of Formula Ia or Ib in the solution comprising a sodium salt of the compound of Formula Ia or Ib, PEG 300, and optionally one, two, or three additional components selected from water, poloxamer 188, and ethanol is about 400 mg/ml. In some embodiments, the concentration of the compound of Formula Ia or Ib in the solution comprising a sodium salt of the compound of Formula Ia or Ib, PEG 300, and optionally one, two, or three additional components selected from water, poloxamer 188, and ethanol is about 500 mg/ml. In some embodiments, the concentration of the compound of Formula Ia or Ib in the solution comprising a sodium salt of the compound of Formula Ia or Ib, PEG 300, and optionally one, two, or three additional components selected from water, poloxamer 188, and ethanol is about 600 mg/ml.

In some embodiments, the amount of the sodium salt of the compound of Formula Ia or Ib in the solution comprising a sodium salt of the compound of Formula Ia or Ib, PEG 300, and optionally one, two, or three additional components selected from water, poloxamer 188, and ethanol is about 1 w/w % to about 75 w/w %. In some embodiments, the amount of the sodium salt of the compound of Formula Ia or Ib in the solution comprising a sodium salt of the compound of Formula Ia or Ib, PEG 300, and optionally one, two, or three additional components selected from water, poloxamer 188, and ethanol is about 9 w/w % to about 75 w/w %. In some embodiments, the amount of the sodium salt of the compound of Formula Ia or Ib in the solution comprising a sodium salt of the compound of Formula Ia or Ib, PEG 300, and optionally one, two, or three additional components selected from water, poloxamer 188, and ethanol is about 15 w/w % to about 65 w/w %. In some embodiments, the amount of the sodium salt of the compound of Formula Ia or Ib in the solution comprising a sodium salt of the compound of Formula Ia or Ib, PEG 300, and optionally one, two, or three additional components selected from water, poloxamer 188, and ethanol is about 20 w/w % to about 55 w/w %. In some embodiments, the amount of the sodium salt of the compound of Formula Ia or Ib in the solution comprising a sodium salt of the compound of Formula Ia or Ib, PEG 300, and optionally one, two, or three additional components selected from water, poloxamer 188, and ethanol is about 24 w/w % to about 50 w/w %. In some embodiments, the amount of the sodium salt of the compound of Formula Ia or Ib in the solution comprising a sodium salt of the compound of Formula Ia or Ib, PEG 300, and optionally one, two, or three additional components selected from water, poloxamer 188, and ethanol is about 20 w/w % to about 45 w/w %. In some embodiments, the amount of the sodium salt of the compound of Formula Ia or Ib in the solution comprising a sodium salt of the compound of Formula Ia or Ib, PEG 300, and optionally one, two, or three additional components selected from water, poloxamer 188, and ethanol is about 41.85 w/w %. In some embodiments, the amount of the sodium salt of the compound of Formula Ia or Ib in the solution comprising a sodium salt of the compound of Formula Ia or Ib, PEG 300, and optionally one, two, or three additional components selected from water, poloxamer 188, and ethanol is about 41.9 w/w %.

In some embodiments, the amount of PEG 300 in the solution comprising a sodium salt of the compound of Formula Ia or Ib, PEG 300, and optionally one, two, or three additional components selected from water, poloxamer 188, and ethanol is about 15 w/w % to about 90 w/w %. In some embodiments, the amount of PEG 300 in the solution comprising a sodium salt of the compound of Formula Ia or Ib, PEG 300, and optionally one, two, or three additional components selected from water, poloxamer 188, and ethanol is about 25 w/w % to about 80 w/w %. In some embodiments, the amount of PEG 300 in the solution comprising a sodium salt of the compound of Formula Ia or Ib, PEG 300, and optionally one, two, or three additional components selected from water, poloxamer 188, and ethanol is about 25 w/w % to about 70 w/w %. In some embodiments, the amount of PEG 300 in the solution comprising a sodium salt of the compound of Formula Ia or Ib, PEG 300, and optionally one, two, or three additional components selected from water, poloxamer 188, and ethanol is about 25 w/w % to about 60 w/w %. In some embodiments, the amount of PEG 300 in the solution comprising a sodium salt of the compound of Formula Ia or Ib, PEG 300, and optionally one, two, or three additional components selected from water, poloxamer 188, and ethanol is about 25 w/w % to about 55 w/w %. In some embodiments, the amount of PEG 300 in the solution comprising a sodium salt of the compound of Formula Ia or Ib, PEG 300, and optionally one, two, or three additional components selected from water, poloxamer 188, and ethanol is about 32.8 w/w %. In some embodiments, the amount of PEG 300 in the solution comprising a sodium salt of the compound of Formula Ia or Ib, PEG 300, and optionally one, two, or three additional components selected from water, poloxamer 188, and ethanol is about 32.82 w/w %.

In some embodiments, the amount of water in the solution comprising a sodium salt of the compound of Formula Ia or Ib, PEG 300, and optionally one, two, or three additional components selected from water, poloxamer 188, and ethanol is about 0 w/w % to about 40 w/w %. In some embodiments, the amount of water in the solution comprising a sodium salt of the compound of Formula Ia or Ib, PEG 300, and optionally one, two, or three additional components selected from water, poloxamer 188, and ethanol is about 0 w/w % to about 30 w/w %. In some embodiments, the amount of water in the solution comprising a sodium salt of the compound of Formula Ia or Ib, PEG 300, and optionally one, two, or three additional components selected from water, poloxamer 188, and ethanol is about 0 w/w % to about 25 w/w %. In some embodiments, the amount of water in the solution comprising a sodium salt of the compound of Formula Ia or Ib, PEG 300, and optionally one, two, or three additional components selected from water, poloxamer 188, and ethanol is about 0 w/w % to about 20 w/w %. In some embodiments, the amount of water in the solution comprising a sodium salt of the compound of Formula Ia or Ib, PEG 300, and optionally one, two, or three additional components selected from water, poloxamer 188, and ethanol is about 3 w/w % to about 20 w/w %. In some embodiments, the amount of water in the solution comprising a sodium salt of the compound of Formula Ia or Ib, PEG 300, and optionally one, two, or three additional components selected from water, poloxamer 188, and ethanol is about 15.3 w/w %. In some embodiments, the amount of water in the solution comprising a sodium salt of the compound of Formula Ia or Ib, PEG 300, and optionally one, two, or three additional components selected from water, poloxamer 188, and ethanol is about 15.33 w/w %.

In some embodiments, the amount of poloxamer 188 in the solution comprising a sodium salt of the compound of Formula Ia or Ib, PEG 300, and optionally one, two, or three additional components selected from water, poloxamer 188, and ethanol is about 0 w/w % to about 35 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 0 w/w % to about 25 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 0 w/w % to about 15 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 0 w/w % to about 10 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 0 w/w % to about 8 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 0 w/w % to about 5 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 0 w/w % to about 3 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 0 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 1 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 2 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 3 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 4 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 5 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 6 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 7 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 8 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 9 w/w %. In some embodiments, the amount of poloxamer 188 in the solution comprising a sodium salt of the compound of Formula Ia or Ib, PEG 300, and optionally one, two, or three additional components selected from water, poloxamer 188, and ethanol is about 10 w/w %.

In some embodiments, the amount of ethanol in the solution comprising a sodium salt of the compound of Formula Ia or Ib, PEG 300, and optionally one, two, or three additional components selected from water, poloxamer 188, and ethanol is about 0 w/w % to about 30 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 0 w/w % to about 25 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 0 w/w % to about 15 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 0 w/w % to about 10 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 0 w/w % to about 8 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 0 w/w % to about 5 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 0 w/w %, about 1 w/w %, about 2 w/w %, about 3 w/w %, about 4 w/w %, or about 5 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 0 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 1 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 2 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 3 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 4 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 5 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 6 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 7 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 8 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 9 w/w %. In some embodiments, the amount of ethanol in the solution comprising a sodium salt of the compound of Formula Ia or Ib, PEG 300, and optionally one, two, or three additional components selected from water, poloxamer 188, and ethanol is about 10 w/w %.

In some embodiments, the solution comprises about 20 w/w % to about 55 w/w % of the sodium salt of the compound of Formula Ia or Ib, about 25 w/w % to about 80 w/w % of PEG 300, about 0 w/w % to about 25 w/w % of water, about 0 w/w % to about 10 w/w % of poloxamer 188, and about 0 w/w % to about 10 w/w % of ethanol. In some embodiments, the solution comprises about 24 w/w % to about 50 w/w % of the sodium salt of the compound of Formula Ia or Ib, about 25 w/w % to about 60 w/w % of PEG 300, about 0 w/w % to about 20 w/w % of water, about 0 w/w % to about 8 w/w % of poloxamer 188, and about 0 w/w % to about 10 w/w % of ethanol. In some embodiments, the solution comprises about 24 w/w % to about 50 w/w % of the sodium salt of the compound of Formula Ia or Ib, about 25 w/w % to about 55 w/w % of PEG 300, about 0 w/w % to about 20 w/w % of water, about 0 w/w % to about 8 w/w % of poloxamer 188, and about 0 w/w % to about 10 w/w % of ethanol.

In some embodiments, the solution comprises about 41.9 w/w % of the sodium salt of the compound of Formula Ia or Ib, about 32.8 w/w % of PEG 300, about 15.3 w/w % of water, and about 10.0 w/w % of ethanol. In some embodiments, the solution comprises about 41.85 w/w % of the sodium salt of the compound of Formula Ia or Ib, about 32.82 w/w % of PEG 300, about 15.33 w/w % of water, and about 10.00 w/w % of ethanol. In some embodiments, the solution comprises about 41.9 w/w % of the sodium salt of the compound of Formula Ib, about 32.8 w/w % of PEG 300, about 15.3 w/w % of water, and about 10.0 w/w % of ethanol. In some embodiments, the solution comprises about 41.85 w/w % of the sodium salt of the compound of Formula Ib, about 32.82 w/w % of PEG 300, about 15.33 w/w % of water, and about 10.00 w/w % of ethanol.

In some embodiments, the solution comprises a compound of Formula Ia or Ib, or a pharmaceutically acceptable salt thereof, PEG 300, water, and optionally one or two additional components selected from poloxamer 188 and ethanol. In some embodiments, the solution comprises a compound of Formula Ia, or a pharmaceutically acceptable salt thereof, PEG 300, water, and optionally one or two additional components selected from poloxamer 188 and ethanol. In some embodiments, the solution comprises a compound of Formula Ib, or a pharmaceutically acceptable salt thereof, PEG 300, water, and optionally one or two additional components selected from poloxamer 188 and ethanol. In some embodiments, the solution comprises a compound of Formula Ia, PEG 300, water, and optionally one or two additional components selected from poloxamer 188 and ethanol. In some embodiments, the solution comprises a compound of Formula Ib, PEG 300, water, and optionally one or two additional components selected from poloxamer 188 and ethanol.

In some embodiments, the solution comprises a sodium salt of the compound of Formula Ia or Ib, PEG 300, water, and optionally one or two additional components selected from poloxamer 188 and ethanol. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 50 mg/ml to about 600 mg/ml. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 50 mg/ml to about 500 mg/ml. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 50 mg/ml to about 400 mg/ml. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 50 mg/ml to about 300 mg/ml. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 50 mg/ml. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 75 mg/ml. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 100 mg/ml. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 125 mg/ml. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 150 mg/ml. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 200 mg/ml. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 300 mg/ml. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 400 mg/ml. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 500 mg/ml. In some embodiments, the concentration of the compound of Formula Ia or Ib in the solution comprising a sodium salt of the compound of Formula Ia or Ib, PEG 300, water, and optionally one or two additional components selected from poloxamer 188 and ethanol is about 600 mg/ml.

In some embodiments, the amount of the sodium salt of the compound of Formula Ia or Ib in the solution comprising a sodium salt of the compound of Formula Ia or Ib, PEG 300, water, and optionally one or two additional components selected from poloxamer 188 and ethanol is about 0.5 w/w % to about 60 w/w %. In some embodiments, the amount of the sodium salt of the compound of Formula Ia or Ib in the solution comprising a sodium salt of the compound of Formula Ia or Ib, PEG 300, water, and optionally one or two additional components selected from poloxamer 188 and ethanol is about 2 w/w % to about 50 w/w %. In some embodiments, the amount of the sodium salt of the compound of Formula Ia or Ib in the solution comprising a sodium salt of the compound of Formula Ia or Ib, PEG 300, water, and optionally one or two additional components selected from poloxamer 188 and ethanol is about 2 w/w % to about 35 w/w %. In some embodiments, the amount of the sodium salt of the compound of Formula Ia or Ib in the solution comprising a sodium salt of the compound of Formula Ia or Ib, PEG 300, water, and optionally one or two additional components selected from poloxamer 188 and ethanol is about 2 w/w % to about 30 w/w %. In some embodiments, the amount of the sodium salt of the compound of Formula Ia or Ib in the solution comprising a sodium salt of the compound of Formula Ia or Ib, PEG 300, water, and optionally one or two additional components selected from poloxamer 188 and ethanol is about 4 w/w % to about 27 w/w %.

In some embodiments, the amount of PEG 300 in the solution comprising a sodium salt of the compound of Formula Ia or Ib, PEG 300, water, and optionally one or two additional components selected from poloxamer 188 and ethanol is about 5 w/w % to about 90 w/w %. In some embodiments, the amount of PEG 300 in the solution comprising a sodium salt of the compound of Formula Ia or Ib, PEG 300, water, and optionally one or two additional components selected from poloxamer 188 and ethanol is about 10 w/w % to about 80 w/w %. In some embodiments, the amount of PEG 300 in the solution comprising a sodium salt of the compound of Formula Ia or Ib, PEG 300, water, and optionally one or two additional components selected from poloxamer 188 and ethanol is about 20 w/w % to about 75 w/w %. In some embodiments, the amount of PEG 300 in the solution comprising a sodium salt of the compound of Formula Ia or Ib, PEG 300, water, and optionally one or two additional components selected from poloxamer 188 and ethanol is about 30 w/w % to about 65 w/w %.

In some embodiments, the amount of water in the solution comprising a sodium salt of the compound of Formula Ia or Ib, PEG 300, water, and optionally one or two additional components selected from poloxamer 188 and ethanol is about 0.5 w/w % to about 60 w/w %. In some embodiments, the amount of water in the solution comprising a sodium salt of the compound of Formula Ia or Ib, PEG 300, water, and optionally one or two additional components selected from poloxamer 188 and ethanol is about 2 w/w % to about 50 w/w %. In some embodiments, the amount of water in the solution comprising a sodium salt of the compound of Formula Ia or Ib, PEG 300, water, and optionally one or two additional components selected from poloxamer 188 and ethanol is about 5 w/w % to about 45 w/w %. In some embodiments, the amount of water in the solution comprising a sodium salt of the compound of Formula Ia or Ib, PEG 300, water, and optionally one or two additional components selected from poloxamer 188 and ethanol is about 10 w/w % to about 40 w/w %. In some embodiments, the amount of water in the solution comprising a sodium salt of the compound of Formula Ia or Ib, PEG 300, water, and optionally one or two additional components selected from poloxamer 188 and ethanol is about 14 w/w % to about 31 w/w %.

In some embodiments, the amount of poloxamer 188 in the solution comprising a sodium salt of the compound of Formula Ia or Ib, PEG 300, water, and optionally one or two additional components selected from poloxamer 188 and ethanol is about 0 w/w % to about 35 w/w %. In some embodiments, the amount of poloxamer 188 in the solution comprising a sodium salt of the compound of Formula Ia or Ib, PEG 300, water, and optionally one or two additional components selected from poloxamer 188 and ethanol is about 0 w/w % to about 25 w/w %. In some embodiments, the amount of poloxamer 188 in the solution comprising a sodium salt of the compound of Formula Ia or Ib, PEG 300, water, and optionally one or two additional components selected from poloxamer 188 and ethanol is about 0 w/w % to about 15 w/w %. In some embodiments, the amount of poloxamer 188 in the solution comprising a sodium salt of the compound of Formula Ia or Ib, PEG 300, water, and optionally one or two additional components selected from poloxamer 188 and ethanol is about 0 w/w % to about 10 w/w %. In some embodiments, the amount of poloxamer 188 in the solution comprising a sodium salt of the compound of Formula Ia or Ib, PEG 300, water, and optionally one or two additional components selected from poloxamer 188 and ethanol is about 0 w/w % to about 8 w/w %. In some embodiments, the amount of poloxamer 188 in the solution comprising a sodium salt of the compound of Formula Ia or Ib, PEG 300, water, and optionally one or two additional components selected from poloxamer 188 and ethanol is about 0 w/w % to about 5 w/w %. In some embodiments, the amount of poloxamer 188 in the solution comprising a sodium salt of the compound of Formula Ia or Ib, PEG 300, water, and optionally one or two additional components selected from poloxamer 188 and ethanol is about 0 w/w % to about 3 w/w %. In some embodiments, the amount of poloxamer 188 in the solution comprising a sodium salt of the compound of Formula Ia or Ib, PEG 300, water, and optionally one or two additional components selected from poloxamer 188 and ethanol is about 0 w/w %.

In some embodiments, the amount of ethanol in the solution comprising a sodium salt of the compound of Formula Ia or Ib, PEG 300, water, and optionally one or two additional components selected from poloxamer 188 and ethanol is about 0 w/w % to about 30 w/w %. In some embodiments, the amount of ethanol in the solution comprising a sodium salt of the compound of Formula Ia or Ib, PEG 300, water, and optionally one or two additional components selected from poloxamer 188 and ethanol is about 0 w/w % to about 25 w/w %. In some embodiments, the amount of ethanol in the solution comprising a sodium salt of the compound of Formula Ia or Ib, PEG 300, water, and optionally one or two additional components selected from poloxamer 188 and ethanol is about 0 w/w % to about 15 w/w %. In some embodiments, the amount of ethanol in the solution comprising a sodium salt of the compound of Formula Ia or Ib, PEG 300, water, and optionally one or two additional components selected from poloxamer 188 and ethanol is about 0 w/w % to about 10 w/w %. In some embodiments, the amount of ethanol in the solution comprising a sodium salt of the compound of Formula Ia or Ib, PEG 300, water, and optionally one or two additional components selected from poloxamer 188 and ethanol is about 0 w/w % to about 8 w/w %. In some embodiments, the amount of ethanol in the solution comprising a sodium salt of the compound of Formula Ia or Ib, PEG 300, water, and optionally one or two additional components selected from poloxamer 188 and ethanol is about 0 w/w % to about 5 w/w %. In some embodiments, the amount of ethanol in the solution comprising a sodium salt of the compound of Formula Ia or Ib, PEG 300, water, and optionally one or two additional components selected from poloxamer 188 and ethanol is about 0 w/w %, about 1 w/w %, about 2 w/w %, about 3 w/w %, about 4 w/w %, or about 5 w/w %. In some embodiments, the amount of ethanol in the solution comprising a sodium salt of the compound of Formula Ia or Ib, PEG 300, water, and optionally one or two additional components selected from poloxamer 188 and ethanol is about 0 w/w %. In some embodiments, the amount of ethanol in the solution comprising a sodium salt of the compound of Formula Ia or Ib, PEG 300, water, and optionally one or two additional components selected from poloxamer 188 and ethanol is about 1 w/w %. In some embodiments, the amount of ethanol in the solution comprising a sodium salt of the compound of Formula Ia or Ib, PEG 300, water, and optionally one or two additional components selected from poloxamer 188 and ethanol is about 2 w/w %. In some embodiments, the amount of ethanol in the solution comprising a sodium salt of the compound of Formula Ia or Ib, PEG 300, water, and optionally one or two additional components selected from poloxamer 188 and ethanol is about 3 w/w %. In some embodiments, the amount of ethanol in the solution comprising a sodium salt of the compound of Formula Ia or Ib, PEG 300, water, and optionally one or two additional components selected from poloxamer 188 and ethanol is about 4 w/w %. In some embodiments, the amount of ethanol in the solution comprising a sodium salt of the compound of Formula Ia or Ib, PEG 300, water, and optionally one or two additional components selected from poloxamer 188 and ethanol is about 5 w/w %. In some embodiments, the amount of ethanol in the solution comprising a sodium salt of the compound of Formula Ia or Ib, PEG 300, water, and optionally one or two additional components selected from poloxamer 188 and ethanol is about 6 w/w %. In some embodiments, the amount of ethanol in the solution comprising a sodium salt of the compound of Formula Ia or Ib, PEG 300, water, and optionally one or two additional components selected from poloxamer 188 and ethanol is about 7 w/w %. In some embodiments, the amount of ethanol in the solution comprising a sodium salt of the compound of Formula Ia or Ib, PEG 300, water, and optionally one or two additional components selected from poloxamer 188 and ethanol is about 8 w/w %. In some embodiments, the amount of ethanol in the solution comprising a sodium salt of the compound of Formula Ia or Ib, PEG 300, water, and optionally one or two additional components selected from poloxamer 188 and ethanol is about 9 w/w %. In some embodiments, the amount of ethanol in the solution comprising a sodium salt of the compound of Formula Ia or Ib, PEG 300, water, and optionally one or two additional components selected from poloxamer 188 and ethanol is about 10 w/w %.

In some embodiments, the solution comprises about about 2 w/w % to about 35 w/w % of the sodium salt of the compound of Formula Ia or Ib, about 20 w/w % to about 75 w/w % of PEG 300, about 10 w/w % to about 40 w/w % of water, about 0 w/w % to about 10 w/w % of poloxamer 188, and about 0 w/w % to about 10 w/w % of ethanol. In some embodiments, the solution comprises about about 2 w/w % to about 35 w/w % of the sodium salt of the compound of Formula Ia or Ib, about 20 w/w % to about 75 w/w % of PEG 300, about 10 w/w % to about 40 w/w % of water, about 0 w/w % to about 10 w/w % of poloxamer 188, and about 0 w/w % to about 10 w/w % of ethanol. In some embodiments, the solution comprises about about 4 w/w % to about 27 w/w % of the sodium salt of the compound of Formula Ia or Ib, about 30 w/w % to about 65 w/w % of PEG 300, about 14 w/w % to about 31 w/w % of water, about 0 w/w % to about 8 w/w % of poloxamer 188, and about 0 w/w % to about 10 w/w % of ethanol. In some embodiments, the solution comprises about about 4 w/w % to about 27 w/w % of the sodium salt of the compound of Formula Ia or Ib, about 30 w/w % to about 65 w/w % of PEG 300, about 14 w/w % to about 31 w/w % of water, about 0 w/w % to about 8 w/w % of poloxamer 188, and about 0 w/w % to about 5 w/w % of ethanol.

In some embodiments, the solution comprises a compound of Formula Ia or Ib, or a pharmaceutically acceptable salt thereof, PEG 300, and water. In some embodiments, the solution comprises a compound of Formula Ia or a pharmaceutically acceptable salt thereof, PEG 300, and water. In some embodiments, the solution comprises a compound of Formula Ib, or a pharmaceutically acceptable salt thereof, PEG 300, and water. In some embodiments, the solution comprises a compound of Formula Ia, PEG 300, and water. In some embodiments, the solution comprises a sodium salt of the compound of Formula Ia, PEG 300, and water. In some embodiments, the solution comprises a compound of Formula Ib, PEG 300, and water. In some embodiments, the solution comprises a sodium salt of a compound of Formula Ib, PEG 300, and water.

In some embodiments, the solution comprises a compound of Formula Ia or Ib, PEG 300, and water. In some embodiments, the concentration of the compound of Formula Ia or Ib in the solution comprising a compound of Formula Ia or Ib, PEG 300, and water is about 50 mg/ml to about 600 mg/ml. In some embodiments, the concentration of the compound of Formula Ia or Ib in the solution comprising a compound of Formula Ia or Ib, PEG 300, and water is about 50 mg/ml to about 500 mg/ml. In some embodiments, the concentration of the compound of Formula Ia or Ib in the solution comprising a compound of Formula Ia or Ib, PEG 300, and water is about 50 mg/ml to about 400 mg/ml. In some embodiments, the concentration of the compound of Formula Ia or Ib in the solution comprising a compound of Formula Ia or Ib, PEG 300, and water is about 50 mg/ml to about 300 mg/ml. In some embodiments, the concentration of the compound of Formula Ia or Ib in the solution comprising a compound of Formula Ia or Ib, PEG 300, and water is about 75 mg/ml to about 300 mg/ml. In some embodiments, the concentration of the compound of Formula Ia or Ib in the solution comprising a compound of Formula Ia or Ib, PEG 300, and water is about 50 mg/ml. In some embodiments, the concentration of the compound of Formula Ia or Ib in the solution comprising a compound of Formula Ia or Ib, PEG 300, and water is about 75 mg/ml. In some embodiments, the concentration of the compound of Formula Ia or Ib in the solution comprising a compound of Formula Ia or Ib, PEG 300, and water is about 100 mg/ml. In some embodiments, the concentration of the compound of Formula Ia or Ib in the solution comprising a compound of Formula Ia or Ib, PEG 300, and water is about 125 mg/ml. In some embodiments, the concentration of the compound of Formula Ia or Ib in the solution comprising a compound of Formula Ia or Ib, PEG 300, and water is about 150 mg/ml. In some embodiments, the concentration of the compound of Formula Ia or Ib in the solution comprising a compound of Formula Ia or Ib, PEG 300, and water is about 175 mg/ml. In some embodiments, the concentration of the compound of Formula Ia or Ib in the solution comprising a compound of Formula Ia or Ib, PEG 300, and water is about 200 mg/ml. In some embodiments, the concentration of the compound of Formula Ia or Ib in the solution comprising a compound of Formula Ia or Ib, PEG 300, and water is about 225 mg/ml. In some embodiments, the concentration of the compound of Formula Ia or Ib in the solution comprising a compound of Formula Ia or Ib, PEG 300, and water is about 250 mg/ml. In some embodiments, the concentration of the compound of Formula Ia or Ib in the solution comprising a compound of Formula Ia or Ib, PEG 300, and water is about 275 mg/ml. In some embodiments, the concentration of the compound of Formula Ia or Ib in the solution comprising a compound of Formula Ia or Ib, PEG 300, and water is about 300 mg/ml. In some embodiments, the concentration of the compound of Formula (Ia or Ib in the solution comprising a compound of Formula Ia or Ib, PEG 300, and water is about 325 mg/ml. In some embodiments, the concentration of the compound of Formula Ia or Ib in the solution comprising a compound of Formula Ia or Ib, PEG 300, and water is about 350 mg/ml. In some embodiments, the concentration of the compound of Formula Ia or Ib in the solution comprising a compound of Formula Ia or Ib, PEG 300, and water is about 375 mg/ml. In some embodiments, the concentration of the compound of Formula Ia or Ib in the solution comprising a compound of Formula Ia or Ib, PEG 300, and water is about 400 mg/ml. In some embodiments, the concentration of the compound of Formula Ia or Ib in the solution comprising a compound of Formula Ia or Ib, PEG 300, and water is about 425 mg/ml. In some embodiments, the concentration of the compound of Formula Ia or Ib in the solution comprising a compound of Formula Ia or Ib, PEG 300, and water is about 450 mg/ml. In some embodiments, the concentration of the compound of Formula Ia or Ib in the solution comprising a compound of Formula Ia or Ib, PEG 300, and water is about 475 mg/ml. In some embodiments, the concentration of the compound of Formula Ia or Ib in the solution comprising a compound of Formula Ia or Ib, PEG 300, and water is about 500 mg/ml. In some embodiments, the concentration of the compound of Formula Ia or Ib in the solution comprising a compound of Formula Ia or Ib, PEG 300, and water is about 600 mg/ml.

In some embodiments, the amount of water in the solution comprising a compound of Formula Ia or Ib, PEG 300, and water is about 5 w/w % to about 15 w/w %. In some embodiments, the amount of water in the solution comprising a compound of Formula Ia or Ib, PEG 300, and water is about 5 w/w % to about 10 w/w %. In some embodiments, the amount of water in the solution comprising a compound of Formula Ia or Ib, PEG 300, and water is about 8 w/w % to about 12 w/w %. In some embodiments, the amount of water in the solution comprising a compound of Formula Ia or Ib, PEG 300, and water is about 9 w/w % to about 10 w/w %. In some embodiments, the amount of water in the solution comprising a compound of Formula Ia or Ib, PEG 300, and water is about 8.0 w/w %, about 8.1 w/w %, about 8.2 w/w %, about 8.3 w/w %, about 8.4 w/w %, about 8.5 w/w %, about 8.6 w/w %, about 8.7 w/w %, about 8.8 w/w %, about 8.9 w/w %, about 9.0 w/w %, about 9.1 w/w %, about 9.2 w/w %, about 9.3 w/w %, about 9.4 w/w %, about 9.5 w/w %, about 9.6 w/w %, about 9.7 w/w %, about 9.8 w/w %, about 9.9 w/w %, about 10.0 w/w %, about 10.1 w/w %, about 10.2 w/w %, about 10.3 w/w %, about 10.4 w/w %, about 10.5 w/w %, about 10.6 w/w %, about 10.7 w/w %, about 10.8 w/w %, about 10.9 w/w %, about 11.0 w/w %, about 11.1 w/w %, about 11.2 w/w %, about 11.3 w/w %, about 11.4 w/w %, about 11.5 w/w %, about 11.6 w/w %, about 11.7 w/w %, about 11.8 w/w %, about 11.9 w/w %, or about 12.0 w/w %. In some embodiments, the amount of water in the solution comprising a compound of Formula Ia or Ib, PEG 300, and water is about 9.0 w/w %, about 9.1 w/w %, about 9.2 w/w %, about 9.3 w/w %, about 9.4 w/w %, about 9.5 w/w %, about 9.6 w/w %, about 9.7 w/w %, about 9.8 w/w %, about 9.9 w/w %, or about 10.0 w/w %. In some embodiments, the amount of water in the solution comprising a compound of Formula Ia or Ib, PEG 300, and water is about 9.5 w/w %, about 9.6 w/w %, about 9.7 w/w %, about 9.8 w/w %, about 9.9 w/w %, or about 10.0 w/w %. In some embodiments, the amount of water in the solution comprising a compound of Formula Ia or Ib, PEG 300, and water is about 9.8 w/w %. In some embodiments, the amount of water in the solution comprising a compound of Formula Ia or Ib, PEG 300, and water is about 10 w/w %.

In some embodiments, the amount of PEG 300 in the solution comprising a compound of Formula Ia or Ib, PEG 300, and water is about 50 w/w % to about 85 w/w %. In some embodiments, the amount of PEG 300 in the solution comprising a compound of Formula Ia or Ib, PEG 300, and water is about 60 w/w % to about 80 w/w %. In some embodiments, the amount of PEG 300 in the solution comprising a compound of Formula Ia or Ib, PEG 300, and water is about 60 w/w % to about 70 w/w %. In some embodiments, the amount of PEG 300 in the solution comprising a compound of Formula Ia or Ib, PEG 300, and water is about 40 w/w %, about 45 w/w %, about 50 w/w %, about 55 w/w %, about 60 w/w %, about 65 w/w %, about 70 w/w %, about 75 w/w %, about 80 w/w %, or about 85 w/w %. In some embodiments, the amount of PEG 300 in the solution comprising a compound of Formula Ia or Ib, PEG 300, and water is about 65 w/w %. In some embodiments, the amount of PEG 300 in the solution comprising a compound of Formula Ia or Ib, PEG 300, and water is about 65.0 w/w %.

In some embodiments, the amount of the compound of Formula Ia or Ib in the solution comprising a compound of Formula Ia or Ib, PEG 300, and water is about 15 w/w % to about 35 w/w %. In some embodiments, the amount of the compound of Formula Ia or Ib in the solution comprising a compound of Formula Ia or Ib, PEG 300, and water is about 20 w/w % to about 35 w/w %. In some embodiments, the amount of the compound of Formula Ia or Ib in the solution comprising a compound of Formula Ia or Ib, PEG 300, and water is about 24 w/w % to about 26 w/w %. In some embodiments, the amount of the compound of Formula Ia or Ib in the solution comprising a compound of Formula Ia or Ib, PEG 300, and water is about 24.5 w/w %, about 24.6 w/w %, about 24.7 w/w %, about 24.8 w/w %, about 24.9 w/w %, about 25.0 w/w %, about 25.1 w/w %, about 25.2 w/w %, about 25.3 w/w %, about 25.4 w/w %, or about 25.5 w/w %. In some embodiments, the amount of the compound of Formula Ia or Ib in the solution comprising a compound of Formula Ia or Ib, PEG 300, and water is about 25 w/w %. In some embodiments, the amount of the compound of Formula Ia or Ib in the solution comprising a compound of Formula Ia or Ib, PEG 300, and water is about 25.2 w/w %.

In some embodiments, the solution comprises about 5 w/w % to about 15 w/w % water, about 50 w/w % to about 85 w/w % PEG 300, and about 15 w/w % to about 35 w/w % of a compound of Formula Ia or Ib. In some embodiments, the solution comprises about 5 w/w % to about 10 w/w % water, about 60 w/w % to about 80 w/w % PEG 300, and about 20 w/w % to about 35 w/w % of a compound of Formula Ia or Ib. In some embodiments, the solution comprises about 8 w/w % to about 12 w/w % water, about 60 w/w % to about 70 w/w % PEG 300, and about 15 w/w % to about 35 w/w % of a compound of Formula Ia or Ib. In some embodiments, the solution comprises about 9 w/w % to about 10 w/w % water, about 60 w/w % to about 70 w/w % PEG 300, and about 24 w/w % to about 26 w/w % of a compound of Formula Ia or Ib. In some embodiments, the solution comprises about 9.8 w/w % water, about 65.0 w/w % PEG 300, and about 25.2 w/w % of a compound of Formula Ia. In some embodiments, the solution comprises about 10 w/w % water, about 65.0 w/w % PEG 300, and about 25 w/w % of a compound of Formula Ia. In some embodiments, the solution comprises about 9.8 w/w % water, about 65.0 w/w % PEG 300, and about 25.2 w/w % of a compound of Formula Ib. In some embodiments, the solution comprises about 10 w/w % water, about 65.0 w/w % PEG 300, and about 25 w/w % of a compound of Formula Ib.

In some embodiments, the solution comprises a sodium salt of the compound of Formula Ia or Ib, PEG 300, and water. In some embodiments, the concentration of the compound of Formula Ia or Ib in the solution comprising a sodium salt of the compound of Formula Ia or Ib, PEG 300, and water is about 50 mg/ml to about 600 mg/ml. In some embodiments, the concentration of the compound of Formula Ia or Ib in the solution comprising a sodium salt of the compound of Formula Ia or Ib, PEG 300, and water is about 50 mg/ml to about 500 mg/ml. In some embodiments, the concentration of the compound of Formula Ia or Ib in the solution comprising a sodium salt of the compound of Formula Ia or Ib, PEG 300, and water is about 50 mg/ml to about 300 mg/ml. In some embodiments, the concentration of the compound of Formula Ia or Ib in the solution comprising a sodium salt of the compound of Formula Ia or Ib, PEG 300, and water is about 75 mg/ml to about 300 mg/ml. In some embodiments, the concentration of the compound of Formula Ia or Ib in the solution comprising a sodium salt of the compound of Formula Ia or Ib, PEG 300, and water is about 50 mg/ml. In some embodiments, the concentration of the compound of Formula Ia or Ib in the solution comprising a sodium salt of the compound of Formula Ia or Ib, PEG 300, and water is about 75 mg/ml. In some embodiments, the concentration of the compound of Formula Ia or Ib in the solution comprising a sodium salt of the compound of Formula Ia or Ib, PEG 300, and water is about 100 mg/ml. In some embodiments, the concentration of the compound of Formula Ia or Ib in the solution comprising a sodium salt of the compound of Formula Ia or Ib, PEG 300, and water is about 125 mg/ml. In some embodiments, the concentration of the compound of Formula Ia or Ib in the solution comprising a sodium salt of the compound of Formula Ia or Ib, PEG 300, and water is about 150 mg/ml. In some embodiments, the concentration of the compound of Formula Ia or Ib in the solution comprising a sodium salt of the compound of Formula Ia or Ib, PEG 300, and water is about 175 mg/ml. In some embodiments, the concentration of the compound of Formula Ia or Ib in the solution comprising a sodium salt of the compound of Formula Ia or Ib, PEG 300, and water is about 200 mg/ml. In some embodiments, the concentration of the compound of Formula Ia or Ib in the solution comprising a sodium salt of the compound of Formula Ia or Ib PEG 300, and water is about 225 mg/ml. In some embodiments, the concentration of the compound of Formula Ia or Ib in the solution comprising a sodium salt of the compound of Formula Ia or Ib, PEG 300, and water is about 250 mg/ml. In some embodiments, the concentration of the compound of Formula Ia or Ib in the solution comprising a sodium salt of the compound of Formula Ia or Ib, PEG 300, and water is about 275 mg/ml. In some embodiments, the concentration of the compound of Formula Ia or Ib in the solution comprising a sodium salt of the compound of Formula Ia or Ib, PEG 300, and water is about 300 mg/ml. In some embodiments, the concentration of the compound of Formula Ia or Ib in the solution comprising a sodium salt of the compound of Formula Ia or Ib, PEG 300, and water is about 325 mg/ml. In some embodiments, the concentration of the compound of Formula Ia or Ib in the solution comprising a sodium salt of the compound of Formula Ia or Ib, PEG 300, and water is about 350 mg/ml. In some embodiments, the concentration of the compound of Formula Ia or Ib in the solution comprising a sodium salt of the compound of Formula Ia or Ib, PEG 300, and water is about 375 mg/ml. In some embodiments, the concentration of the compound of Formula Ia or Ib in the solution comprising a sodium salt of the compound of Formula Ia or Ib, PEG 300, and water is about 400 mg/ml. In some embodiments, the concentration of the compound of Formula Ia or Ib in the solution comprising a sodium salt of the compound of Formula Ia or Ib, PEG 300, and water is about 425 mg/ml. In some embodiments, the concentration of the compound of Formula Ia or Ib in the solution comprising a sodium salt of the compound of Formula Ia or Ib, PEG 300, and water is about 450 mg/ml. In some embodiments, the concentration of the compound of Formula Ia or Ib in the solution comprising a sodium salt of the compound of Formula Ia or Ib, PEG 300, and water is about 475 mg/ml. In some embodiments, the concentration of the compound of Formula Ia or Ib in the solution comprising a sodium salt of the compound of Formula Ia or Ib, PEG 300, and water is about 500 mg/ml. In some embodiments, the concentration of the compound of Formula Ia or Ib in the solution comprising a sodium salt of the compound of Formula Ia or Ib, PEG 300, and water is about 600 mg/ml.

In some embodiments, the concentration of the compound of Formula Ia or Ib in the solution comprising a sodium salt of the compound of Formula Ia or Ib, PEG 300, and water is about 309 mg/ml. In some embodiments, the concentration of the compound of Formula Ib in the solution comprising a sodium salt of the compound of Formula Ib, PEG 300, and water is about 309 mg/ml.

In some embodiments, the amount of water in the solution comprising a sodium salt of the compound of Formula Ia or Ib, PEG 300, and water is about 10 w/w % to about 40 w/w %. In some embodiments, the amount of water in the solution comprising a sodium salt of the compound of Formula Ia or Ib, PEG 300, and water is about 15 w/w % to about 35 w/w %. In some embodiments, the amount of water in the solution comprising a sodium salt of the compound of Formula Ia or Ib, PEG 300, and water is about 20 w/w % to about 30 w/w %. In some embodiments, the amount of water in the solution comprising a sodium salt of the compound of Formula Ia or Ib, PEG 300, and water is about 21 w/w % to about 29 w/w %. In some embodiments, the amount of water in the solution comprising a sodium salt of the compound of Formula Ia or Ib, PEG 300, and water is about 23.4 w/w % to about 27.5 w/w %. In some embodiments, the amount of water in the solution comprising a sodium salt of the compound of Formula Ia or Ib, PEG 300, and water is about 23.41 w/w % to about 27.47 w/w %. In some embodiments, the amount of water in the solution comprising a sodium salt of the compound of Formula Ia or Ib, PEG 300, and water is about 22.0 w/w %, about 22.1 w/w %, about 22.2 w/w %, about 22.3 w/w %, about 22.4 w/w %, about 22.5 w/w %, about 22.6 w/w %, about 22.7 w/w %, about 22.8 w/w %, about 22.9 w/w %, about 23.0 w/w %, about 23.1 w/w %, about 23.2 w/w %, about 23.3 w/w %, about 23.4 w/w %, about 23.5 w/w %, about 23.6 w/w %, about 23.7 w/w %, about 23.8 w/w %, about 23.9 w/w %, about 24.0 w/w %, about 24.1 w/w %, about 24.2 w/w %, about 24.3 w/w %, about 24.4 w/w %, about 24.5 w/w %, about 24.6 w/w %, about 24.7 w/w %, about 24.8 w/w %, about 24.9 w/w %, about 25.0 w/w %, about 25.1 w/w %, about 25.2 w/w %, about 25.3 w/w %, about 25.4 w/w %, about 25.5 w/w %, about 25.6 w/w %, about 25.7 w/w %, about 25.8 w/w %, about 25.9 w/w %, about 26.0 w/w %, about 26.1 w/w %, about 26.2 w/w %, about 26.3 w/w %, about 26.4 w/w %, about 26.5 w/w %, about 26.6 w/w %, about 26.7 w/w %, about 26.8 w/w %, about 26.9 w/w %, about 27.0 w/w %, about 27.1 w/w %, about 27.2 w/w %, about 27.3 w/w %, about 27.4 w/w %, about 27.5 w/w %, about 27.6 w/w %, about 27.7 w/w %, about 27.8 w/w %, about 27.9 w/w %, about 28.0 w/w %, about 28.1 w/w %, about 28.2 w/w %, about 28.3 w/w %, about 28.4 w/w %, about 28.5 w/w %, about 28.6 w/w %, about 28.7 w/w %, about 28.8 w/w %, about 28.9 w/w %, or about 29.0 w/w %. In some embodiments, the amount of water in the solution comprising a sodium salt of the compound of Formula Ia or Ib, PEG 300, and water is about 23.4 w/w %. In some embodiments, the amount of water in the solution comprising a sodium salt of the compound of Formula Ia or Ib, PEG 300, and water is about 23.41 w/w %. In some embodiments, the amount of water in the solution comprising a sodium salt of the compound of Formula Ia or Ib, PEG 300, and water is about 27.47 w/w %. In some embodiments, the amount of water in the solution comprising a sodium salt of the compound of Formula Ia or Ib, PEG 300, and water is about 27.5 w/w %.

In some embodiments, the amount of water in the solution comprising a sodium salt of the compound of Formula Ia or Ib, PEG 300, and water is about 21.1 w/w % to about 27.5 w/w %. In some embodiments, the amount of water in the solution comprising a sodium salt of the compound of Formula Ia or Ib, PEG 300, and water is about 21.13 w/w % to about 27.47 w/w %.

In some embodiments, the amount of water in the solution comprising a sodium salt of the compound of Formula Ia or Ib, PEG 300, and water is about 21.1 w/w %. In some embodiments, the amount of water in the solution comprising a sodium salt of the compound of Formula Ia or Ib, PEG 300, and water is about 21.13 w/w %.

In some embodiments, the amount of PEG 300 in the solution comprising a sodium salt of the compound of Formula Ia or Ib, PEG 300, and water is about 35 w/w % to about 75 w/w %. In some embodiments, the amount of PEG 300 in the solution comprising a sodium salt of the compound of Formula Ia or Ib, PEG 300, and water is about 45 w/w % to about 65 w/w %. In some embodiments, the amount of PEG 300 in the solution comprising a sodium salt of the compound of Formula Ia or Ib, PEG 300, and water is about 48 w/w % to about 60 w/w %. In some embodiments, the amount of PEG 300 in the solution comprising a sodium salt of the compound of Formula Ia or Ib, PEG 300, and water is about 50 w/w % to about 59 w/w %. In some embodiments, the amount of PEG 300 in the solution comprising a sodium salt of the compound of Formula Ia or Ib, PEG 300, and water is about 50.1 w/w % to about 58.8 w/w %. In some embodiments, the amount of PEG 300 in the solution comprising a sodium salt of the compound of Formula Ia or Ib, PEG 300, and water is about 50.13 w/w % to about 58.84 w/w %. In some embodiments, the amount of PEG 300 in the solution comprising a sodium salt of the compound of Formula Ia or Ib, PEG 300, and water is about 45 w/w %, about 46 w/w %, about 47 w/w %, about 48 w/w %, about 49 w/w %, about 50 w/w %, about 51 w/w %, about 52 w/w %, about 53 w/w %, about 54 w/w %, about 55 w/w %, about 56 w/w %, about 57 w/w %, about 58 w/w %, about 59 w/w %, about 60 w/w %, about 61 w/w %, about 62 w/w %, about 63 w/w %, about 64 w/w %, or about 65 w/w %. In some embodiments, the amount of PEG 300 in the solution comprising a sodium salt of the compound of Formula Ia or Ib, PEG 300, and water is about 50.1 w/w %. In some embodiments, the amount of PEG 300 in the solution comprising a sodium salt of the compound of Formula Ia or Ib, PEG 300, and water is about 50.13 w/w %. In some embodiments, the amount of PEG 300 in the solution comprising a sodium salt of the compound of Formula Ia or Ib, PEG 300, and water is about 58.8 w/w %. In some embodiments, the amount of PEG 300 in the solution comprising a sodium salt of the compound of Formula Ia or Ib, PEG 300, and water is about 58.84 w/w %.

In some embodiments, the amount of PEG 300 in the solution comprising a sodium salt of the compound of Formula Ia or Ib, PEG 300, and water is about 45.3 w/w % to about 58.8 w/w %. In some embodiments, the amount of PEG 300 in the solution comprising a sodium salt of the compound of Formula Ia or Ib, PEG 300, and water is about 45.25 w/w % to about 58.84 w/w %.

In some embodiments, the amount of PEG 300 in the solution comprising a sodium salt of the compound of Formula Ia or Ib, PEG 300, and water is about 45.25 w/w %. In some embodiments, the amount of PEG 300 in the solution comprising a sodium salt of the compound of Formula Ia or Ib, PEG 300, and water is about 45.3 w/w %.

In some embodiments, the amount of the sodium salt of the compound of Formula Ia or Ib in the solution comprising a sodium salt of the compound of Formula Ia or Ib, PEG 300, and water is about 5 w/w % to about 35 w/w %. In some embodiments, the amount of the sodium salt of the compound of Formula Ia or Ib in the solution comprising a sodium salt of the compound of Formula Ia or Ib, PEG 300, and water is about 10 w/w % to about 30 w/w %. In some embodiments, the amount of the sodium salt of the compound of Formula Ia or Ib in the solution comprising a sodium salt of the compound of Formula Ia or Ib, PEG 300, and water is about 11 w/w % to about 28 w/w %. In some embodiments, the amount of the sodium salt of the compound of Formula Ia or Ib in the solution comprising a sodium salt of the compound of Formula Ia or Ib, PEG 300, and water is about 13 w/w % to about 27 w/w %. In some embodiments, the amount of the sodium salt of the compound of Formula Ia or Ib in the solution comprising a sodium salt of the compound of Formula Ia or Ib, PEG 300, and water is about 13.69 w/w % to about 26.46 w/w %. In some embodiments, the amount of the sodium salt of the compound of Formula Ia or Ib in the solution comprising a sodium salt of the compound of Formula Ia or Ib, PEG 300, and water is about 13.7 w/w % to about 26.5 w/w %. In some embodiments, the amount of the sodium salt of the compound of Formula Ia or Ib in the solution comprising a sodium salt of the compound of Formula Ia or Ib, PEG 300, and water is about 13.0 w/w %, about 13.1 w/w %, about 13.2 w/w %, about 13.3 w/w %, about 13.4 w/w %, about 13.5 w/w %, about 13.6 w/w %, about 13.7 w/w %, about 13.8 w/w %, about 13.9 w/w %, about 14.0 w/w %, about 14.1 w/w %, about 14.2 w/w %, about 14.3 w/w %, about 14.4 w/w %, about 14.5 w/w %, about 14.6 w/w %, about 14.7 w/w %, about 14.8 w/w %, about 14.9 w/w %, about 15.0 w/w %, about 15.1 w/w %, about 15.2 w/w %, about 15.3 w/w %, about 15.4 w/w %, about 15.5 w/w %, about 15.6 w/w %, about 15.7 w/w %, about 15.8 w/w %, about 15.9 w/w %, about 16.0 w/w %, about 16.1 w/w %, about 16.2 w/w %, about 16.3 w/w %, about 16.4 w/w %, about 16.5 w/w %, about 16.6 w/w %, about 16.7 w/w %, about 16.8 w/w %, about 16.9 w/w %, about 17.0 w/w %, about 17.1 w/w %, about 17.2 w/w %, about 17.3 w/w %, about 17.4 w/w %, about 17.5 w/w %, about 17.6 w/w %, about 17.7 w/w %, about 17.8 w/w %, about 17.9 w/w %, about 18.0 w/w %, about 18.1 w/w %, about 18.2 w/w %, about 18.3 w/w %, about 18.4 w/w %, about 18.5 w/w %, about 18.6 w/w %, about 18.7 w/w %, about 18.8 w/w %, about 18.9 w/w %, about 19.0 w/w %, about 19.1 w/w %, about 19.2 w/w %, about 19.3 w/w %, about 19.4 w/w %, about 19.5 w/w %, about 19.6 w/w %, about 19.7 w/w %, about 19.8 w/w %, about 19.9 w/w %, about 20.0 w/w %, about 21.1 w/w %, about 21.2 w/w %, about 21.3 w/w %, about 21.4 w/w %, about 21.5 w/w %, about 21.6 w/w %, about 21.7 w/w %, about 21.8 w/w %, about 21.9 w/w %, about 22.0 w/w %, about 22.1 w/w %, about 22.2 w/w %, about 22.3 w/w %, about 22.4 w/w %, about 22.5 w/w %, about 22.6 w/w %, about 22.7 w/w %, about 22.8 w/w %, about 22.9 w/w %, about 23.0 w/w %, about 23.1 w/w %, about 23.2 w/w %, about 23.3 w/w %, about 23.4 w/w %, about 23.5 w/w %, about 23.6 w/w %, about 23.7 w/w %, about 23.8 w/w %, about 23.9 w/w %, about 24.0 w/w %, about 24.1 w/w %, about 24.2 w/w %, about 24.3 w/w %, about 24.4 w/w %, about 24.5 w/w %, about 24.6 w/w %, about 24.7 w/w %, about 24.8 w/w %, about 24.9 w/w %, about 25.0 w/w %, about 25.1 w/w %, about 25.2 w/w %, about 25.3 w/w %, about 25.4 w/w %, about 25.5 w/w %, about 25.6 w/w %, about 25.7 w/w %, about 25.8 w/w %, about 25.9 w/w %, about 26.0 w/w %, about 26.1 w/w %, about 26.2 w/w %, about 26.3 w/w %, about 26.4 w/w %, about 26.5 w/w %, about 26.6 w/w %, about 26.7 w/w %, about 26.8 w/w %, about 26.9 w/w %, about 27.0 w/w %, about 27.1 w/w %, about 27.2 w/w %, about 27.3 w/w %, about 27.4 w/w %, about 27.5 w/w %, about 27.6 w/w %, about 27.7 w/w %, about 27.8 w/w %, about 27.9 w/w %, about 28.0 w/w %, about 28.1 w/w %, about 28.2 w/w %, about 28.3 w/w %, about 28.4 w/w %, about 28.5 w/w %, about 28.6 w/w %, about 28.7 w/w %, about 28.8 w/w %, about 28.9 w/w %, or about 29.0 w/w %. In some embodiments, the amount of the sodium salt of the compound of Formula Ia or Ib in the solution comprising a sodium salt of the compound of Formula Ia or Ib, PEG 300, and water is about 13.69 w/w %. In some embodiments, the amount of the sodium salt of the compound of Formula Ia or Ib in the solution comprising a sodium salt of the compound of Formula Ia or Ib, PEG 300, and water is about 13.7 w/w %. In some embodiments, the amount of the sodium salt of the compound of Formula Ia or Ib in the solution comprising a sodium salt of the compound of Formula Ia or Ib, PEG 300, and water is about 26.46 w/w %. In some embodiments, the amount of the sodium salt of the compound of Formula Ia or Ib in the solution comprising a sodium salt of the compound of Formula Ia or Ib, PEG 300, and water is about 26.5 w/w %.

In some embodiments, the amount of the sodium salt of the compound of Formula Ia or Ib in the solution comprising a sodium salt of the compound of Formula Ia or Ib, PEG 300, and water is about 13.69 w/w % to about 33.61 w/w %. In some embodiments, the amount of the sodium salt of the compound of Formula Ia or Ib in the solution comprising a sodium salt of the compound of Formula Ia or Ib, PEG 300, and water is about 13.7 w/w % to about 33.6 w/w %.

In some embodiments, the amount of the sodium salt of the compound of Formula Ia or Ib in the solution comprising a sodium salt of the compound of Formula Ia or Ib, PEG 300, and water is about 33.61 w/w %. In some embodiments, the amount of the sodium salt of the compound of Formula Ia or Ib in the solution comprising a sodium salt of the compound of Formula Ia or Ib, PEG 300, and water is about 33.6 w/w %.

In some embodiments, the solution comprises about 10 w/w % to about 40 w/w % water, about 35 w/w % to about 75 w/w % PEG 300, and about 5 w/w % to about 35 w/w % of a sodium salt of the compound of Formula Ia or Ib. In some embodiments, the solution comprises about 15 w/w % to about 35 w/w % water, about 45 w/w % to about 65 w/w % PEG 300, and about 10 w/w % to about 30 w/w % of a sodium salt of the compound of Formula Ia or Ib. In some embodiments, the solution comprises about 20 w/w % to about 30 w/w % water, about 48 w/w % to about 60 w/w % PEG 300, and about 11 w/w % to about 28 w/w % of a sodium salt of the compound of Formula Ia or Ib. In some embodiments, the solution comprises about 21 w/w % to about 29 w/w % water, about 50 w/w % to about 59 w/w % PEG 300, and about 13 w/w % to about 27 w/w % of a sodium salt of the compound of Formula Ia or Ib. In some embodiments, the solution comprises about 23.4 w/w % to about 27.5 w/w % water, about 50.1 w/w % to about 58.8 w/w % PEG 300, and about 13.7 w/w % to about 26.5 w/w % of a sodium salt of the compound of Formula Ia or Ib. In some embodiments, the solution comprises about 23.41 w/w % to about 27.47 w/w % water, about 50.13 w/w % to about 58.84 w/w % PEG 300, and about 13.69 w/w % to about 26.46 w/w % of a sodium salt of the compound of Formula Ia or Ib. In some embodiments, the solution comprises about 27.5 w/w % water, about 58.8 w/w % PEG 300, and about 13.7 w/w % of a sodium salt of the compound of Formula Ia or Ib. In some embodiments, the solution comprises about 27.47 w/w % water, about 58.84 w/w % PEG 300, and about 13.69 w/w % of a sodium salt of the compound of Formula Ia or Ib. In some embodiments, the solution comprises about 23.4 w/w % water, about 50.1 w/w % PEG 300, and about 26.5 w/w % of a sodium salt of the compound of Formula Ia or Ib. In some embodiments, the solution comprises about 23.41 w/w % water, about 50.13 w/w % PEG 300, and about 26.46 w/w % of a sodium salt of the compound of Formula Ia or Ib. In some embodiments, the solution comprises about 27.5 w/w % water, about 58.8 w/w % PEG 300, and about 13.7 w/w % of a sodium salt of the compound of Formula Ib. In some embodiments, the solution comprises about 27.47 w/w % water, about 58.84 w/w % PEG 300, and about 13.69 w/w % of a sodium salt of the compound of Formula Ib. In some embodiments, the solution comprises about 23.4 w/w % water, about 50.1 w/w % PEG 300, and about 26.5 w/w % of a sodium salt of the compound of Formula Ib. In some embodiments, the solution comprises about 23.41 w/w % water, about 50.13 w/w % PEG 300, and about 26.46 w/w % of a sodium salt of the compound of Formula Ib.

In some embodiments, the solution comprises about 10 w/w % to about 40 w/w % water, about 35 w/w % to about 75 w/w % PEG 300, and about 5 w/w % to about 45 w/w % of a sodium salt of the compound of Formula Ia or Ib. In some embodiments, the solution comprises about 10 w/w % to about 30 w/w % water, about 35 w/w % to about 65 w/w % PEG 300, and about 5 w/w % to about 45 w/w % of a sodium salt of the compound of Formula Ia or Ib.

In some embodiments, the solution comprises about 21.1 w/w % to about 27.5 w/w % water, about 45.3 w/w % to about 58.8 w/w % PEG 300, and about 13.7 w/w % to about 33.6 w/w % of a sodium salt of the compound of Formula Ia or Ib. In some embodiments, the solution comprises about 21.13 w/w % to about 27.47 w/w % water, about 45.25 w/w % to about 58.84 w/w % PEG 300, and about 13.69 w/w % to about 33.61 w/w % of a sodium salt of the compound of Formula Ia or Ib.

In some embodiments, the solution comprises about 21.1 w/w % water, about 45.3 w/w % PEG 300, and about 33.6 w/w % of a sodium salt of a compound of Formula Ia or Ib. In some embodiments, the solution comprises about 21.13 w/w % water, about 45.25 w/w % PEG 300, and about 33.61 w/w % of a sodium salt of a compound of Formula Ia or Ib. In some embodiments, the solution comprises about 21.1 w/w % water, about 45.3 w/w % PEG 300, and about 33.6 w/w % of a sodium salt of a compound of Formula Ib. In some embodiments, the solution comprises about 21.13 w/w % water, about 45.25 w/w % PEG 300, and about 33.61 w/w % of a sodium salt of a compound of Formula Ib.

In some embodiments, the solution comprises a compound of Formula Ia or Ib, or a pharmaceutically acceptable salt thereof, PEG 300, water, and ethanol. In some embodiments, the solution comprises a compound of Formula Ia, or a pharmaceutically acceptable salt thereof, PEG 300, water, and ethanol. In some embodiments, the solution comprises a compound of Formula Ia, PEG 300, water, and ethanol. In some embodiments, the solution comprises a sodium salt of the compound of Formula Ia, PEG 300, water, and ethanol. In some embodiments, the solution comprises a compound of Formula Ib, or a pharmaceutically acceptable salt thereof, PEG 300, water, and ethanol. In some embodiments, the solution comprises a compound of Formula Ib, PEG 300, water, and ethanol. In some embodiments, the solution comprises a sodium salt of the compound of Formula Ib, PEG 300, water, and ethanol.

In some embodiments, the solution comprises a sodium salt of the compound of Formula Ia or Ib, PEG 300, water, and ethanol. In some embodiments, the concentration of the compound of Formula Ia or Ib in the solution comprising a sodium salt of the compound of Formula Ia or Ib, PEG 300, water, and ethanol is about 50 mg/ml to about 600 mg/ml. In some embodiments, the concentration of the compound of Formula Ia or Ib in the solution comprising a sodium salt of the compound of Formula Ia or Ib, PEG 300, water, and ethanol is about 50 mg/ml to about 500 mg/ml. In some embodiments, the concentration of the compound of Formula Ia or Ib in the solution comprising a sodium salt of the compound of Formula Ia or Ib, PEG 300, water, and ethanol is about 200 mg/ml to about 600 mg/ml. In some embodiments, the concentration of the compound of Formula Ia or Ib in the solution comprising a sodium salt of the compound of Formula Ia or Ib, PEG 300, water, and ethanol is about 50 mg/ml. In some embodiments, the concentration of the compound of Formula Ia or Ib in the solution comprising a sodium salt of the compound of Formula Ia or Ib, PEG 300, water, and ethanol is about 100 mg/ml. In some embodiments, the concentration of the compound of Formula Ia or Ib in the solution comprising a sodium salt of the compound of Formula Ia or Ib, PEG 300, water, and ethanol is about 150 mg/ml. In some embodiments, the concentration of the compound of Formula Ia or Ib in the solution comprising a sodium salt of the compound of Formula Ia or Ib, PEG 300, water, and ethanol is about 200 mg/ml. In some embodiments, the concentration of the compound of Formula Ia or Ib in the solution comprising a sodium salt of the compound of Formula Ia or Ib, PEG 300, water, and ethanol is about 250 mg/ml. In some embodiments, the concentration of the compound of Formula Ia or Ib in the solution comprising a sodium salt of the compound of Formula Ia or Ib, PEG 300, water, and ethanol is about 300 mg/ml. In some embodiments, the concentration of the compound of Formula Ia or Ib in the solution comprising a sodium salt of the compound of Formula Ia or Ib, PEG 300, water, and ethanol is about 350 mg/ml. In some embodiments, the concentration of the compound of Formula Ia or Ib in the solution comprising a sodium salt of the compound of Formula Ia or Ib, PEG 300, water, and ethanol is about 400 mg/ml. In some embodiments, the concentration of the compound of Formula Ia or Ib in the solution comprising a sodium salt of the compound of Formula Ia or Ib, PEG 300, water, and ethanol is about 450 mg/ml. In some embodiments, the concentration of the compound of Formula Ia or Ib in the solution comprising a sodium salt of the compound of Formula Ia or Ib, PEG 300, water, and ethanol is about 500 mg/ml. In some embodiments, the concentration of the compound of Formula Ia or Ib in the solution comprising a sodium salt of the compound of Formula Ia or Ib, PEG 300, water, and ethanol is about 550 mg/ml. In some embodiments, the concentration of the compound of Formula Ia or Ib in the solution comprising a sodium salt of the compound of Formula Ia or Ib, PEG 300, water, and ethanol is about 600 mg/ml.

In some embodiments, the amount of water in the solution comprising a sodium salt of the compound of Formula Ia or Ib, PEG 300, water, and ethanol is about 10 w/w % to about 20 w/w %. In some embodiments, the amount of water in the solution comprising a sodium salt of the compound of Formula Ia or Ib, PEG 300, water, and ethanol is about 12 w/w % to about 20 w/w %. In some embodiments, the amount of water in the solution comprising a sodium salt of the compound of Formula Ia or Ib, PEG 300, water, and ethanol is about 16.93 w/w %. In some embodiments, the amount of water in the solution comprising a sodium salt of the compound of Formula Ia or Ib, PEG 300, water, and ethanol is about 16.9 w/w %.

In some embodiments, the amount of PEG 300 in the solution comprising a sodium salt of the compound of Formula Ia or Ib, PEG 300, water, and ethanol is about 30 w/w % to about 40 w/w %. In some embodiments, the amount of PEG 300 in the solution comprising a sodium salt of the compound of Formula Ia or Ib, PEG 300, water, and ethanol is about 32 w/w % to about 40 w/w %. In some embodiments, the amount of PEG 300 in the solution comprising a sodium salt of the compound of Formula Ia or Ib, PEG 300, water, and ethanol is about 36.22 w/w %. In some embodiments, the amount of PEG 300 in the solution comprising a sodium salt of the compound of Formula Ia or Ib, PEG 300, water, and ethanol is about 36.2 w/w %.

In some embodiments, the amount of the sodium salt of the compound of Formula Ia or Ib in the solution comprising a sodium salt of the compound of Formula Ia or Ib, PEG 300, water, and ethanol is about 35 w/w % to about 45 w/w %. In some embodiments, the amount of the sodium salt of the compound of Formula Ia or Ib in the solution comprising a sodium salt of the compound of Formula Ia or Ib, PEG 300, water, and ethanol is about 37 w/w % to about 45 w/w %. In some embodiments, the amount of the sodium salt of the compound of Formula Ia or Ib in the solution comprising a sodium salt of the compound of Formula Ia or Ib, PEG 300, water, and ethanol is about 41.85 w/w %. In some embodiments, the amount of the sodium salt of the compound of Formula Ia or Ib in the solution comprising a sodium salt of the compound of Formula Ia or Ib, PEG 300, water, and ethanol is about 41.9 w/w %.

In some embodiments, the amount of ethanol in the solution comprising a sodium salt of the compound of Formula Ia or Ib, PEG 300, water, and ethanol is about 0.1 w/w % to about 10 w/w %. In some embodiments, the amount of ethanol in the solution comprising a sodium salt of the compound of Formula Ia or Ib, PEG 300, water, and ethanol is about 1 w/w % to about 9 w/w %. In some embodiments, the amount of ethanol in the solution comprising a sodium salt of the compound of Formula Ia or Ib, PEG 300, water, and ethanol is about 3 w/w % to about 8 w/w %. In some embodiments, the amount of ethanol in the solution comprising a sodium salt of the compound of Formula Ia or Ib, PEG 300, water, and ethanol is about 5.00 w/w %. In some embodiments, the amount of ethanol in the solution comprising a sodium salt of the compound of Formula Ia or Ib, PEG 300, water, and ethanol is about 5.0 w/w %.

In some embodiments, the solution comprises about 10 w/w % to about 40 w/w % water, about 20 w/w % to about 75 w/w % PEG 300, about 10 w/w % to about 70 w/w % of a sodium salt of the compound of Formula Ia or Ib, and about 1 w/w % to about 9 w/w % of ethanol. In some embodiments, the solution comprises about 10 w/w % to about 20 w/w % water, about 30 w/w % to about 40 w/w % PEG 300, about 37 w/w % to about 45 w/w % of a sodium salt of the compound of Formula Ia or Ib, and about 3 w/w % to about 8 w/w % of ethanol.

In some embodiments, the solution comprises about 16.93 w/w % water, about 36.22 w/w % PEG 300, about 41.85 w/w % of a sodium salt of the compound of Formula Ia or Ib, and about 5.00 w/w % ethanol. In some embodiments, the solution comprises about 16.9 w/w % water, about 36.2 w/w % PEG 300, about 41.9 w/w % of a sodium salt of the compound of Formula Ia or Ib, and about 5.0 w/w % ethanol. In some embodiments, the solution comprises about 16.93 w/w % water, about 36.22 w/w % PEG 300, about 41.85 w/w % of a sodium salt of the compound of Formula Ib, and about 5.00 w/w % ethanol. In some embodiments, the solution comprises about 16.9 w/w % water, about 36.2 w/w % PEG 300, about 41.9 w/w % of a sodium salt of the compound of Formula Ib, and about 5.0 w/w % ethanol.

In some embodiments, the solution comprises a compound of Formula Ia or Ib, or a pharmaceutically acceptable salt thereof, PEG 300, poloxamer 188, and water. In some embodiments, the solution comprises a compound of Formula Ia, or a pharmaceutically acceptable salt thereof, PEG 300, poloxamer 188, and water. In some embodiments, the solution comprises a compound of Formula Ib, or a pharmaceutically acceptable salt thereof, PEG 300, poloxamer 188, and water. In some embodiments, the solution comprises a compound of Formula Ia, PEG 300, poloxamer 188, and water. In some embodiments, the solution comprises a sodium salt of the compound of Formula Ia, PEG 300, poloxamer 188, and water. In some embodiments, the solution comprises a compound of Formula Ib, PEG 300, poloxamer 188, and water. In some embodiments, the solution comprises a sodium salt of the compound of Formula Ib, PEG 300, poloxamer 188, and water.

In some embodiments, the solution comprises a sodium salt of the compound of Formula Ia or Ib, PEG 300, poloxamer 188, and water. In some embodiments, the concentration of the compound of Formula Ia or Ib in the solution comprising a sodium salt of the compound of Formula Ia or Ib, PEG 300, poloxamer 188, and water is about 50 mg/ml to about 600 mg/ml. In some embodiments, the concentration of the compound of Formula Ia or Ib in the solution comprising a sodium salt of the compound of Formula Ia or Ib, PEG 300, poloxamer 188, and water is about 50 mg/ml to about 500 mg/ml. In some embodiments, the concentration of the compound of Formula Ia or Ib in the solution comprising a sodium salt of the compound of Formula Ia or Ib, PEG 300, poloxamer 188, and water is about 50 mg/ml to about 400 mg/ml. In some embodiments, the concentration of the compound of Formula Ia or Ib in the solution comprising a sodium salt of the compound of Formula Ia or Ib, PEG 300, poloxamer 188, and water is about 50 mg/ml to about 300 mg/ml. In some embodiments, the concentration of the compound of Formula Ia or Ib in the solution comprising a sodium salt of the compound of Formula Ia or Ib, PEG 300, poloxamer 188, and water is about 75 mg/ml to about 300 mg/ml. In some embodiments, the concentration of the compound of Formula Ia or Ib in the solution comprising a sodium salt of the compound of Formula Ia or Ib, PEG 300, poloxamer 188, and water is about 50 mg/ml. In some embodiments, the concentration of the compound of Formula Ia or Ib in the solution comprising a sodium salt of the compound of Formula Ia or Ib, PEG 300, poloxamer 188, and water is about 75 mg/ml. In some embodiments, the concentration of the compound of Formula Ia or Ib in the solution comprising a sodium salt of the compound of Formula Ia or Ib, PEG 300, poloxamer 188, and water is about 100 mg/ml. In some embodiments, the concentration of the compound of Formula Ia or Ib in the solution comprising a sodium salt of the compound of Formula Ia or Ib, PEG 300, poloxamer 188, and water is about 125 mg/ml. In some embodiments, the concentration of the compound of Formula Ia or Ib in the solution comprising a sodium salt of the compound of Formula Ia or Ib, PEG 300, poloxamer 188, and water is about 150 mg/ml. In some embodiments, the concentration of the compound of Formula Ia or Ib in the solution comprising a sodium salt of the compound of Formula Ia or Ib, PEG 300, poloxamer 188, and water is about 175 mg/ml. In some embodiments, the concentration of the compound of Formula Ia or Ib in the solution comprising a sodium salt of the compound of Formula Ia or Ib, PEG 300, poloxamer 188, and water is about 200 mg/ml. In some embodiments, the concentration of the compound of Formula Ia or Ib in the solution comprising a sodium salt of the compound of Formula Ia or Ib, PEG 300, poloxamer 188, and water is about 225 mg/ml. In some embodiments, the concentration of the compound of Formula Ia or Ib in the solution comprising a sodium salt of the compound of Formula Ia or Ib, PEG 300, poloxamer 188, and water is about 250 mg/ml. In some embodiments, the concentration of the compound of Formula Ia or Ib in the solution comprising a sodium salt of the compound of Formula Ia or Ib, PEG 300, poloxamer 188, and water is about 275 mg/ml. In some embodiments, the concentration of the compound of Formula Ia or Ib in the solution comprising a sodium salt of the compound of Formula Ia or Ib, PEG 300, poloxamer 188, and water is about 300 mg/ml. In some embodiments, the concentration of the compound of Formula Ia or Ib in the solution comprising a sodium salt of the compound of Formula Ia or Ib, PEG 300, poloxamer 188, and water is about 325 mg/ml. In some embodiments, the concentration of the compound of Formula Ia or Ib in the solution comprising a sodium salt of the compound of Formula Ia or Ib, PEG 300, poloxamer 188, and water is about 350 mg/ml. In some embodiments, the concentration of the compound of Formula Ia or Ib in the solution comprising a sodium salt of the compound of Formula Ia or Ib, PEG 300, poloxamer 188, and water is about 375 mg/ml. In some embodiments, the concentration of the compound of Formula Ia or Ib in the solution comprising a sodium salt of the compound of Formula Ia or Ib, PEG 300, poloxamer 188, and water is about 400 mg/ml. In some embodiments, the concentration of the compound of Formula Ia or Ib in the solution comprising a sodium salt of the compound of Formula Ia or Ib, PEG 300, poloxamer 188, and water is about 425 mg/ml. In some embodiments, the concentration of the compound of Formula Ia or Ib in the solution comprising a sodium salt of the compound of Formula Ia or Ib, PEG 300, poloxamer 188, and water is about 450 mg/ml. In some embodiments, the concentration of the compound of Formula Ia or Ib in the solution comprising a sodium salt of the compound of Formula Ia or Ib, PEG 300, poloxamer 188, and water is about 475 mg/ml. In some embodiments, the concentration of the compound of Formula Ia or Ib in the solution comprising a sodium salt of the compound of Formula Ia or Ib, PEG 300, poloxamer 188, and water is about 500 mg/ml. In some embodiments, the concentration of the compound of Formula Ia or Ib in the solution comprising a sodium salt of the compound of Formula Ia or Ib, PEG 300, poloxamer 188, and water is about 600 mg/ml.

In some embodiments, the amount of water in the solution comprising a sodium salt of the compound of Formula Ia or Ib, PEG 300, poloxamer 188, and water is about 10 w/w % to about 45 w/w %. In some embodiments, the amount of water in the solution comprising a sodium salt of the compound of Formula Ia or Ib, PEG 300, poloxamer 188, and water is about 15 w/w % to about 35 w/w %. In some embodiments, the amount of water in the solution comprising a sodium salt of the compound of Formula Ia or Ib, PEG 300, poloxamer 188, and water is about 20 w/w % to about 35 w/w %. In some embodiments, the amount of water in the solution comprising a sodium salt of the compound of Formula Ia or Ib, PEG 300, poloxamer 188, and water is about 20 w/w % to about 31 w/w %. In some embodiments, the amount of water in the solution comprising a sodium salt of the compound of Formula Ia or Ib, PEG 300, poloxamer 188, and water is about 21.9 w/w % to about 30.1 w/w %. In some embodiments, the amount of water in the solution comprising a sodium salt of the compound of Formula Ia or Ib, PEG 300, poloxamer 188, and water is about 21.87 w/w % to about 30.07 w/w %. In some embodiments, the amount of water in the solution comprising a sodium salt of the compound of Formula Ia or Ib, PEG 300, poloxamer 188, and water is about 19.0 w/w %, about 19.1 w/w %, about 19.2 w/w %, about 19.3 w/w %, about 19.4 w/w %, about 19.5 w/w %, about 19.6 w/w %, about 19.7 w/w %, about 19.8 w/w %, about 19.9 w/w %, about 20.0 w/w %, about 20.1 w/w %, about 20.2 w/w %, about 20.3 w/w %, about 20.4 w/w %, about 20.5 w/w %, about 20.6 w/w %, about 20.7 w/w %, about 20.8 w/w %, about 20.9 w/w %, about 21.0 w/w %, about 21.1 w/w %, about 21.2 w/w %, about 21.3 w/w %, about 21.4 w/w %, about 21.5 w/w %, about 21.6 w/w %, about 21.7 w/w %, about 21.8 w/w %, about 21.9 w/w %, about 22.0 w/w %, about 22.1 w/w %, about 22.2 w/w %, about 22.3 w/w %, about 22.4 w/w %, about 22.5 w/w %, about 22.6 w/w %, about 22.7 w/w %, about 22.8 w/w %, about 22.9 w/w %, about 23.0 w/w %, about 23.1 w/w %, about 23.2 w/w %, about 23.3 w/w %, about 23.4 w/w %, about 23.5 w/w %, about 23.6 w/w %, about 23.7 w/w %, about 23.8 w/w %, about 23.9 w/w %, about 24.0 w/w %, about 24.1 w/w %, about 24.2 w/w %, about 24.3 w/w %, about 24.4 w/w %, about 24.5 w/w %, about 24.6 w/w %, about 24.7 w/w %, about 24.8 w/w %, about 24.9 w/w %, about 25.0 w/w %, about 25.1 w/w %, about 25.2 w/w %, about 25.3 w/w %, about 25.4 w/w %, about 25.5 w/w %, about 25.6 w/w %, about 25.7 w/w %, about 25.8 w/w %, about 25.9 w/w %, about 26.0 w/w %, about 26.1 w/w %, about 26.2 w/w %, about 26.3 w/w %, about 26.4 w/w %, about 26.5 w/w %, about 26.6 w/w %, about 26.7 w/w %, about 26.8 w/w %, about 26.9 w/w %, about 27.0 w/w %, about 27.1 w/w %, about 27.2 w/w %, about 27.3 w/w %, about 27.4 w/w %, about 27.5 w/w %, about 27.6 w/w %, about 27.7 w/w %, about 27.8 w/w %, about 27.9 w/w %, about 28.0 w/w %, about 28.1 w/w %, about 28.2 w/w %, about 28.3 w/w %, about 28.4 w/w %, about 28.5 w/w %, about 28.6 w/w %, about 28.7 w/w %, about 28.8 w/w %, about 28.9 w/w %, about 29.0 w/w %, about 29.1 w/w %, about 29.2 w/w %, about 29.3 w/w %, about 29.4 w/w %, about 29.5 w/w %, about 29.6 w/w %, about 29.7 w/w %, about 29.8 w/w %, about 29.9 w/w %, about 30.0 w/w %, about 30.1 w/w %, about 30.2 w/w %, about 30.3 w/w %, about 30.4 w/w %, about 30.5 w/w %, about 30.6 w/w %, about 30.7 w/w %, about 30.8 w/w %, about 30.9 w/w %, about 31.0 w/w %, about 31.1 w/w %, about 31.2 w/w %, about 31.3 w/w %, about 31.4 w/w %, about 31.5 w/w %, about 31.6 w/w %, about 31.7 w/w %, about 31.8 w/w %, about 31.9 w/w %, about 32.0 w/w %, about 32.1 w/w %, about 32.2 w/w %, about 32.3 w/w %, about 32.4 w/w %, about 32.5 w/w %, about 32.6 w/w %, about 32.7 w/w %, about 32.8 w/w %, about 32.9 w/w %, or about 33.0 w/w %. In some embodiments, the amount of water in the solution comprising a sodium salt of the compound of Formula Ia or Ib, PEG 300, poloxamer 188, and water is about 21.87 w/w %. In some embodiments, the amount of water in the solution comprising a sodium salt of the compound of Formula Ia or Ib, PEG 300, poloxamer 188, and water is about 21.9 w/w %. In some embodiments, the amount of water in the solution comprising a sodium salt of the compound of Formula Ia or Ib, PEG 300, poloxamer 188, and water is about 26.68 w/w %. In some embodiments, the amount of water in the solution comprising a sodium salt of the compound of Formula Ia or Ib, PEG 300, poloxamer 188, and water is about 26.7 w/w %. In some embodiments, the amount of water in the solution comprising a sodium salt of the compound of Formula Ia or Ib, PEG 300, and poloxamer 188, and water is about 27.5 w/w %. In some embodiments, the amount of water in the solution comprising a sodium salt of the compound of Formula Ia or Ib, PEG 300, poloxamer 188, and water is about 27.51 w/w %. In some embodiments, the amount of water in the solution comprising a sodium salt of the compound of Formula Ia or Ib, PEG 300, poloxamer 188, and water is about 28.36 w/w %. In some embodiments, the amount of water in the solution comprising a sodium salt of the compound of Formula Ia or Ib, PEG 300, poloxamer 188, and water is about 28.4 w/w %. In some embodiments, the amount of water in the solution comprising a sodium salt of the compound of Formula Ia or Ib, PEG 300, poloxamer 188, and water is about 29.2 w/w %. In some embodiments, the amount of water in the solution comprising a sodium salt of the compound of Formula Ia or Ib, PEG 300, poloxamer 188, and water is about 29.21 w/w %. In some embodiments, the amount of water in the solution comprising a sodium salt of the compound of Formula Ia or Ib, PEG 300, poloxamer 188, and water is about 30.07 w/w %. In some embodiments, the amount of water in the solution comprising a sodium salt of the compound of Formula Ia or Ib, PEG 300, poloxamer 188, and water is about 30.1 w/w %.

In some embodiments, the amount of water in the solution comprising a sodium salt of the compound of Formula Ia or Ib, PEG 300, poloxamer 188, and water is about 19.18 w/w % to about 30.07 w/w %. In some embodiments, the amount of water in the solution comprising a sodium salt of the compound of Formula Ia or Ib, PEG 300, poloxamer 188, and water is about 19.2 w/w % to about 30.1 w/w %.

In some embodiments, the amount of water in the solution comprising a sodium salt of the compound of Formula Ia or Ib, PEG 300, poloxamer 188, and water is about 19.18 w/w %. In some embodiments, the amount of water in the solution comprising a sodium salt of the compound of Formula Ia or Ib, PEG 300, poloxamer 188, and water is about 19.2 w/w %. In some embodiments, the amount of water in the solution comprising a sodium salt of the compound of Formula Ia or Ib, PEG 300, poloxamer 188, and water is about 20.16 w/w %. In some embodiments, the amount of water in the solution comprising a sodium salt of the compound of Formula Ia or Ib, PEG 300, poloxamer 188, and water is about 20.2 w/w %. In some embodiments, the amount of water in the solution comprising a sodium salt of the compound of Formula Ia or Ib, PEG 300, poloxamer 188, and water is about 22.10 w/w %. In some embodiments, the amount of water in the solution comprising a sodium salt of the compound of Formula Ia or Ib, PEG 300, poloxamer 188, and water is about 22.1 w/w %. In some embodiments, the amount of water in the solution comprising a sodium salt of the compound of Formula Ia or Ib, PEG 300, poloxamer 188, and water is about 22.48 w/w %. In some embodiments, the amount of water in the solution comprising a sodium salt of the compound of Formula Ia or Ib, PEG 300, poloxamer 188, and water is about 22.5 w/w %. In some embodiments, the amount of water in the solution comprising a sodium salt of the compound of Formula Ia or Ib, PEG 300, poloxamer 188, and water is about 22.85 w/w %. In some embodiments, the amount of water in the solution comprising a sodium salt of the compound of Formula Ia or Ib, PEG 300, poloxamer 188, and water is about 22.9 w/w %. In some embodiments, the amount of water in the solution comprising a sodium salt of the compound of Formula Ia or Ib, PEG 300, poloxamer 188, and water is about 23.22 w/w %. In some embodiments, the amount of water in the solution comprising a sodium salt of the compound of Formula Ia or Ib, PEG 300, poloxamer 188, and water is about 23.2 w/w %. In some embodiments, the amount of water in the solution comprising a sodium salt of the compound of Formula Ia or Ib, PEG 300, poloxamer 188, and water is about 26.79 w/w %. In some embodiments, the amount of water in the solution comprising a sodium salt of the compound of Formula Ia or Ib, PEG 300, poloxamer 188, and water is about 26.8 w/w %. In some embodiments, the amount of water in the solution comprising a sodium salt of the compound of Formula Ia or Ib, PEG 300, poloxamer 188, and water is about 27.61 w/w %. In some embodiments, the amount of water in the solution comprising a sodium salt of the compound of Formula Ia or Ib, PEG 300, poloxamer 188, and water is about 27.6 w/w %. In some embodiments, the amount of water in the solution comprising a sodium salt of the compound of Formula Ia or Ib, PEG 300, poloxamer 188, and water is about 28.43 w/w %. In some embodiments, the amount of water in the solution comprising a sodium salt of the compound of Formula Ia or Ib, PEG 300, poloxamer 188, and water is about 28.4 w/w %.

In some embodiments, the amount of PEG 300 in the solution comprising a sodium salt of the compound of Formula Ia or Ib, PEG 300, poloxamer 188, and water is about 30 w/w % to about 85 w/w %. In some embodiments, the amount of PEG 300 in the solution comprising a sodium salt of the compound of Formula Ia or Ib, PEG 300, poloxamer 188, and water is about 35 w/w % to about 75 w/w %. In some embodiments, the amount of PEG 300 in the solution comprising a sodium salt of the compound of Formula Ia or Ib, PEG 300, poloxamer 188, and water is about 40 w/w % to about 70 w/w %. In some embodiments, the amount of PEG 300 in the solution comprising a sodium salt of the compound of Formula Ia or Ib, PEG 300, poloxamer 188, and water is about 45 w/w % to about 68 w/w %. In some embodiments, the amount of PEG 300 in the solution comprising a sodium salt of the compound of Formula Ia or Ib, PEG 300, poloxamer 188, and water is about 46.8 w/w % to about 64.4 w/w %. In some embodiments, the amount of PEG 300 in the solution comprising a sodium salt of the compound of Formula Ia or Ib, PEG 300, poloxamer 188, and water is about 46.84 w/w % to about 64.40 w/w %. In some embodiments, the amount of PEG 300 in the solution comprising a sodium salt of the compound of Formula Ia or Ib, PEG 300, poloxamer 188, and water is about 40 w/w %, about 41 w/w %, about 42 w/w %, about 43 w/w %, about 44 w/w %, about 45 w/w %, about 46 w/w %, about 47 w/w %, about 48 w/w %, about 49 w/w %, about 50 w/w %, about 51 w/w %, about 52 w/w %, about 53 w/w %, about 54 w/w %, about 55 w/w %, about 56 w/w %, about 57 w/w %, about 58 w/w %, about 59 w/w %, about 60 w/w %, about 61 w/w %, about 62 w/w %, about 63 w/w %, about 64 w/w %, about 65 w/w %, about 66 w/w %, about 67 w/w %, about 68 w/w %, about 69 w/w %, or about 70 w/w %. In some embodiments, the amount of PEG 300 in the solution comprising a sodium salt of the compound of Formula Ia or Ib, PEG 300, poloxamer 188, and water is about 46.8 w/w %. In some embodiments, the amount of PEG 300 in the solution comprising a sodium salt of the compound of Formula Ia or Ib, PEG 300, poloxamer 188, and water is about 46.84 w/w %. In some embodiments, the amount of PEG 300 in the solution comprising a sodium salt of the compound of Formula Ia or Ib, PEG 300, poloxamer 188, and water is about 57.1 w/w %. In some embodiments, the amount of PEG 300 in the solution comprising a sodium salt of the compound of Formula Ia or Ib, PEG 300, poloxamer 188, and water is about 57.13 w/w %. In some embodiments, the amount of PEG 300 in the solution comprising a sodium salt of the compound of Formula Ia or Ib, PEG 300, poloxamer 188, and water is about 58.9 w/w %. In some embodiments, the amount of PEG 300 in the solution comprising a sodium salt of the compound of Formula Ia or Ib, PEG 300, poloxamer 188, and water is about 58.92 w/w %. In some embodiments, the amount of PEG 300 in the solution comprising a sodium salt of the compound of Formula Ia or Ib, PEG 300, poloxamer 188, and water is about 60.7 w/w %. In some embodiments, the amount of PEG 300 in the solution comprising a sodium salt of the compound of Formula Ia or Ib, PEG 300, poloxamer 188, and water is about 60.73 w/w %. In some embodiments, the amount of PEG 300 in the solution comprising a sodium salt of the compound of Formula Ia or Ib, PEG 300, poloxamer 188, and water is about 62.55 w/w %. In some embodiments, the amount of PEG 300 in the solution comprising a sodium salt of the compound of Formula Ia or Ib, PEG 300, poloxamer 188, and water is about 62.6 w/w %. In some embodiments, the amount of PEG 300 in the solution comprising a sodium salt of the compound of Formula Ia or Ib, PEG 300, poloxamer 188, and water is about 64.4 w/w %. In some embodiments, the amount of PEG 300 in the solution comprising a sodium salt of the compound of Formula Ia or Ib, PEG 300, poloxamer 188, and water is about 64.40 w/w %.

In some embodiments, the amount of PEG 300 in the solution comprising a sodium salt of the compound of Formula Ia or Ib, PEG 300, poloxamer 188, and water is about 41.09 w/w % to about 64.40 w/w %. In some embodiments, the amount of PEG 300 in the solution comprising a sodium salt of the compound of Formula Ia or Ib, PEG 300, poloxamer 188, and water is about 41.1 w/w % to about 64.4 w/w %.

In some embodiments, the amount of PEG 300 in the solution comprising a sodium salt of the compound of Formula Ia or Ib, PEG 300, poloxamer 188, and water is about 41.09 w/w %. In some embodiments, the amount of PEG 300 in the solution comprising a sodium salt of the compound of Formula Ia or Ib, PEG 300, poloxamer 188, and water is about 41.1 w/w %. In some embodiments, the amount of PEG 300 in the solution comprising a sodium salt of the compound of Formula Ia or Ib, PEG 300, poloxamer 188, and water is about 43.17 w/w %. In some embodiments, the amount of PEG 300 in the solution comprising a sodium salt of the compound of Formula Ia or Ib, PEG 300, poloxamer 188, and water is about 43.2 w/w %. In some embodiments, the amount of PEG 300 in the solution comprising a sodium salt of the compound of Formula Ia or Ib, PEG 300, poloxamer 188, and water is about 47.33 w/w %. In some embodiments, the amount of PEG 300 in the solution comprising a sodium salt of the compound of Formula Ia or Ib, PEG 300, poloxamer 188, and water is about 47.3 w/w %. In some embodiments, the amount of PEG 300 in the solution comprising a sodium salt of the compound of Formula Ia or Ib, PEG 300, poloxamer 188, and water is about 48.13 w/w %. In some embodiments, the amount of PEG 300 in the solution comprising a sodium salt of the compound of Formula Ia or Ib, PEG 300, poloxamer 188, and water is about 48.1 w/w %. In some embodiments, the amount of PEG 300 in the solution comprising a sodium salt of the compound of Formula Ia or Ib, PEG 300, poloxamer 188, and water is about 48.94 w/w %. In some embodiments, the amount of PEG 300 in the solution comprising a sodium salt of the compound of Formula Ia or Ib, PEG 300, poloxamer 188, and water is about 48.9 w/w %. In some embodiments, the amount of PEG 300 in the solution comprising a sodium salt of the compound of Formula Ia or Ib, PEG 300, poloxamer 188, and water is about 49.73 w/w %. In some embodiments, the amount of PEG 300 in the solution comprising a sodium salt of the compound of Formula Ia or Ib, PEG 300, poloxamer 188, and water is about 49.7 w/w %. In some embodiments, the amount of PEG 300 in the solution comprising a sodium salt of the compound of Formula Ia or Ib, PEG 300, poloxamer 188, and water is about 57.38 w/w %. In some embodiments, the amount of PEG 300 in the solution comprising a sodium salt of the compound of Formula Ia or Ib, PEG 300, poloxamer 188, and water is about 57.4 w/w %. In some embodiments, the amount of PEG 300 in the solution comprising a sodium salt of the compound of Formula Ia or Ib, PEG 300, poloxamer 188, and water is about 59.13 w/w %. In some embodiments, the amount of PEG 300 in the solution comprising a sodium salt of the compound of Formula Ia or Ib, PEG 300, poloxamer 188, and water is about 59.1 w/w %. In some embodiments, the amount of PEG 300 in the solution comprising a sodium salt of the compound of Formula Ia or Ib, PEG 300, poloxamer 188, and water is about 60.90 w/w %. In some embodiments, the amount of PEG 300 in the solution comprising a sodium salt of the compound of Formula Ia or Ib, PEG 300, poloxamer 188, and water is about 60.9 w/w %.

In some embodiments, the amount of the sodium salt of the compound of Formula Ia or Ib in the solution comprising a sodium salt of the compound of Formula Ia or Ib, PEG 300, poloxamer 188, and water is about 0.5 w/w % to about 40 w/w %. In some embodiments, the amount of the sodium salt of the compound of Formula Ia or Ib in the solution comprising a sodium salt of the compound of Formula Ia or Ib, PEG 300, poloxamer 188, and water is about 1 w/w % to about 35 w/w %. In some embodiments, the amount of the sodium salt of the compound of Formula Ia or Ib in the solution comprising a sodium salt of the compound of Formula Ia or Ib, PEG 300, poloxamer 188, and water is about 1 w/w % to about 30 w/w %. In some embodiments, the amount of the sodium salt of the compound of Formula Ia or Ib in the solution comprising a sodium salt of the compound of Formula Ia or Ib, PEG 300, poloxamer 188, and water is about 3 w/w % to about 28 w/w %. In some embodiments, the amount of the sodium salt of the compound of Formula Ia or Ib in the solution comprising a sodium salt of the compound of Formula Ia or Ib, PEG 300, poloxamer 188, and water is about 4 w/w % to about 27 w/w %. In some embodiments, the amount of the sodium salt of the compound of Formula Ia or Ib in the solution comprising a sodium salt of the compound of Formula Ia or Ib, PEG 300, poloxamer 188, and water is about 4.68 w/w % to about 26.47 w/w %. In some embodiments, the amount of the sodium salt of the compound of Formula Ia or Ib in the solution comprising a sodium salt of the compound of Formula Ia or Ib, PEG 300, poloxamer 188, and water is about 4.7 w/w % to about 26.5 w/w %. In some embodiments, the amount of the sodium salt of the compound of Formula Ia or Ib in the solution comprising a sodium salt of the compound of Formula Ia or Ib, PEG 300, poloxamer 188, and water is about 3.0 w/w %, about 3.1 w/w %, about 3.2 w/w %, about 3.3 w/w %, about 3.4 w/w %, about 3.5 w/w %, about 3.6 w/w %, about 3.7 w/w %, about 3.8 w/w %, about 3.9 w/w %, about 4.0 w/w %, about 4.1 w/w %, about 4.2 w/w %, about 4.3 w/w %, about 4.4 w/w %, about 4.5 w/w %, about 4.6 w/w %, about 4.7 w/w %, about 4.8 w/w %, about 4.9 w/w %, about 5.0 w/w %, about 5.1 w/w %, about 5.2 w/w %, about 5.3 w/w %, about 5.4 w/w %, about 5.5 w/w %, about 5.6 w/w %, about 5.7 w/w %, about 5.8 w/w %, about 5.9 w/w %, about 6.0 w/w %, about 6.1 w/w %, about 6.2 w/w %, about 6.3 w/w %, about 6.4 w/w %, about 6.5 w/w %, about 6.6 w/w %, about 6.7 w/w %, about 6.8 w/w %, about 6.9 w/w %, about 7.0 w/w %, about 7.1 w/w %, about 7.2 w/w %, about 7.3 w/w %, about 7.4 w/w %, about 7.5 w/w %, about 7.6 w/w %, about 7.7 w/w %, about 7.8 w/w %, about 7.9 w/w %, about 8.0 w/w %, about 8.1 w/w %, about 8.2 w/w %, about 8.3 w/w %, about 8.4 w/w %, about 8.5 w/w %, about 8.6 w/w %, about 8.7 w/w %, about 8.8 w/w %, about 8.9 w/w %, about 9.0 w/w %, about 9.1 w/w %, about 9.2 w/w %, about 9.3 w/w %, about 9.4 w/w %, about 9.5 w/w %, about 9.6 w/w %, about 9.7 w/w %, about 9.8 w/w %, about 9.9 w/w %, about 10.0 w/w %, about 10.1 w/w %, about 10.2 w/w %, about 10.3 w/w %, about 10.4 w/w %, about 10.5 w/w %, about 10.6 w/w %, about 10.7 w/w %, about 10.8 w/w %, about 10.9 w/w %, about 11.0 w/w %, about 11.1 w/w %, about 11.2 w/w %, about 11.3 w/w %, about 11.4 w/w %, about 11.5 w/w %, about 11.6 w/w %, about 11.7 w/w %, about 11.8 w/w %, about 11.9 w/w %, about 12.0 w/w %, about 12.1 w/w %, about 12.2 w/w %, about 12.3 w/w %, about 12.4 w/w %, about 12.5 w/w %, about 12.6 w/w %, about 12.7 w/w %, about 12.8 w/w %, about 12.9 w/w %, about 13.0 w/w %, about 13.1 w/w %, about 13.2 w/w %, about 13.3 w/w %, about 13.4 w/w %, about 13.5 w/w %, about 13.6 w/w %, about 13.7 w/w %, about 13.8 w/w %, about 13.9 w/w %, about 14.0 w/w %, about 14.1 w/w %, about 14.2 w/w %, about 14.3 w/w %, about 14.4 w/w %, about 14.5 w/w %, about 14.6 w/w %, about 14.7 w/w %, about 14.8 w/w %, about 14.9 w/w %, about 15.0 w/w %, about 15.1 w/w %, about 15.2 w/w %, about 15.3 w/w %, about 15.4 w/w %, about 15.5 w/w %, about 15.6 w/w %, about 15.7 w/w %, about 15.8 w/w %, about 15.9 w/w %, about 16.0 w/w %, about 16.1 w/w %, about 16.2 w/w %, about 16.3 w/w %, about 16.4 w/w %, about 16.5 w/w %, about 16.6 w/w %, about 16.7 w/w %, about 16.8 w/w %, about 16.9 w/w %, about 17.0 w/w %, about 17.1 w/w %, about 17.2 w/w %, about 17.3 w/w %, about 17.4 w/w %, about 17.5 w/w %, about 17.6 w/w %, about 17.7 w/w %, about 17.8 w/w %, about 17.9 w/w %, about 18.0 w/w %, about 18.1 w/w %, about 18.2 w/w %, about 18.3 w/w %, about 18.4 w/w %, about 18.5 w/w %, about 18.6 w/w %, about 18.7 w/w %, about 18.8 w/w %, about 18.9 w/w %, about 19.0 w/w %, about 19.1 w/w %, about 19.2 w/w %, about 19.3 w/w %, about 19.4 w/w %, about 19.5 w/w %, about 19.6 w/w %, about 19.7 w/w %, about 19.8 w/w %, about 19.9 w/w %, about 20.0 w/w %, about 20.1 w/w %, about 20.2 w/w %, about 20.3 w/w %, about 20.4 w/w %, about 20.5 w/w %, about 20.6 w/w %, about 20.7 w/w %, about 20.8 w/w %, about 20.9 w/w %, about 21.0 w/w %, about 21.1 w/w %, about 21.2 w/w %, about 21.3 w/w %, about 21.4 w/w %, about 21.5 w/w %, about 21.6 w/w %, about 21.7 w/w %, about 21.8 w/w %, about 21.9 w/w %, about 22.0 w/w %, about 22.1 w/w %, about 22.2 w/w %, about 22.3 w/w %, about 22.4 w/w %, about 22.5 w/w %, about 22.6 w/w %, about 22.7 w/w %, about 22.8 w/w %, about 22.9 w/w %, about 23.0 w/w %, about 23.1 w/w %, about 23.2 w/w %, about 23.3 w/w %, about 23.4 w/w %, about 23.5 w/w %, about 23.6 w/w %, about 23.7 w/w %, about 23.8 w/w %, about 23.9 w/w %, about 24.0 w/w %, about 24.1 w/w %, about 24.2 w/w %, about 24.3 w/w %, about 24.4 w/w %, about 24.5 w/w %, about 24.6 w/w %, about 24.7 w/w %, about 24.8 w/w %, about 24.9 w/w %, about 25.0 w/w %, about 25.1 w/w %, about 25.2 w/w %, about 25.3 w/w %, about 25.4 w/w %, about 25.5 w/w %, about 25.6 w/w %, about 25.7 w/w %, about 25.8 w/w %, about 25.9 w/w %, about 26.0 w/w %, about 26.1 w/w %, about 26.2 w/w %, about 26.3 w/w %, about 26.4 w/w %, about 26.5 w/w %, about 26.6 w/w %, about 26.7 w/w %, about 26.8 w/w %, about 26.9 w/w %, about 27.0 w/w %, about 27.1 w/w %, about 27.2 w/w %, about 27.3 w/w %, about 27.4 w/w %, about 27.5 w/w %, about 27.6 w/w %, about 27.7 w/w %, about 27.8 w/w %, about 27.9 w/w %, about 28.0 w/w %, about 28.1 w/w %, about 28.2 w/w %, about 28.3 w/w %, about 28.4 w/w %, about 28.5 w/w %, about 28.6 w/w %, about 28.7 w/w %, about 28.8 w/w %, about 28.9 w/w %, or about 29.0 w/w %. In some embodiments, the amount of the sodium salt of the compound of Formula Ia or Ib in the solution comprising a sodium salt of the compound of Formula Ia or Ib, PEG 300, poloxamer 188, and water is about 4.68 w/w %. In some embodiments, the amount of the sodium salt of the compound of Formula Ia or Ib in the solution comprising a sodium salt of the compound of Formula Ia or Ib, PEG 300, poloxamer 188, and water is about 4.7 w/w %. In some embodiments, the amount of the sodium salt of the compound of Formula Ia in the solution comprising a sodium salt of the compound of Formula Ia or Ib, PEG 300, poloxamer 188, and water is about 6.97 w/w %. In some embodiments, the amount of the sodium salt of the compound of Formula Ia or Ib in the solution comprising a sodium salt of the compound of Formula Ia or Ib, PEG 300, poloxamer 188, and water is about 7 w/w %. In some embodiments, the amount of the sodium salt of the compound of Formula Ia or Ib in the solution comprising a sodium salt of the compound of Formula Ia or Ib, PEG 300, poloxamer 188, and water is about 9.2 w/w %. In some embodiments, the amount of the sodium salt of the compound of Formula Ia or Ib in the solution comprising a sodium salt of the compound of Formula Ia or Ib, PEG 300, poloxamer 188, and water is about 9.23 w/w %. In some embodiments, the amount of the sodium salt of the compound of Formula Ia or Ib in the solution comprising a sodium salt of the compound of Formula Ia or Ib, PEG 300, poloxamer 188, and water is about 11.48 w/w %. In some embodiments, the amount of the sodium salt of the compound of Formula Ia or Ib in the solution comprising a sodium salt of the compound of Formula Ia or Ib PEG 300, poloxamer 188, and water is about 11.5 w/w %. In some embodiments, the amount of the sodium salt of the compound of Formula Ia in the solution comprising a sodium salt of the compound of Formula Ia or Ib, PEG 300, poloxamer 188, and water is about 13.7 w/w %. In some embodiments, the amount of the sodium salt of the compound of Formula Ia or Ib in the solution comprising a sodium salt of the compound of Formula Ia or Ib, PEG 300, poloxamer 188, and water is about 13.70 w/w %. In some embodiments, the amount of the sodium salt of the compound of Formula Ia or Ib in the solution comprising a sodium salt of the compound of Formula Ia or Ib, PEG 300, poloxamer 188, and water is about 26.47 w/w %. In some embodiments, the amount of the sodium salt of the compound of Formula Ia or Ib in the solution comprising a sodium salt of the compound of Formula Ia or Ib, PEG 300, poloxamer 188, and water is about 26.5 w/w %.

In some embodiments, the amount of the sodium salt of the compound of Formula Ia or Ib in the solution comprising a sodium salt of the compound of Formula Ia or Ib, PEG 300, poloxamer 188, and water is about 4.68 w/w % to about 33.61 w/w %. In some embodiments, the amount of the sodium salt of the compound of Formula Ia or Ib in the solution comprising a sodium salt of the compound of Formula Ia or Ib, PEG 300, poloxamer 188, and water is about 4.7 w/w % to about 33.6 w/w %.

In some embodiments, the amount of the sodium salt of the compound of Formula Ia or Ib in the solution comprising a sodium salt of the compound of Formula Ia or Ib, PEG 300, poloxamer 188, and water is about 9.03 w/w %. In some embodiments, the amount of the sodium salt of the compound of Formula Ia or Ib in the solution comprising a sodium salt of the compound of Formula Ia or Ib, PEG 300, poloxamer 188, and water is about 9.0 w/w %. In some embodiments, the amount of the sodium salt of the compound of Formula Ia or Ib in the solution comprising a sodium salt of the compound of Formula Ia or Ib, PEG 300, poloxamer 188, and water is about 11.22 w/w %. In some embodiments, the amount of the sodium salt of the compound of Formula Ia or Ib in the solution comprising a sodium salt of the compound of Formula Ia or Ib, PEG 300, poloxamer 188, and water is about 11.2 w/w %. In some embodiments, the amount of the sodium salt of the compound of Formula Ia or Ib in the solution comprising a sodium salt of the compound of Formula Ia or Ib, PEG 300, poloxamer 188, and water is about 13.39 w/w %. In some embodiments, the amount of the sodium salt of the compound of Formula Ia or Ib in the solution comprising a sodium salt of the compound of Formula Ia or Ib, PEG 300, poloxamer 188, and water is about 13.4 w/w %. In some embodiments, the amount of the sodium salt of the compound of Formula Ia or Ib in the solution comprising a sodium salt of the compound of Formula Ia or Ib, PEG 300, poloxamer 188, and water is about 25.85 w/w %. In some embodiments, the amount of the sodium salt of the compound of Formula Ia or Ib in the solution comprising a sodium salt of the compound of Formula Ia or Ib, PEG 300, poloxamer 188, and water is about 25.87 w/w %. In some embodiments, the amount of the sodium salt of the compound of Formula Ia or Ib in the solution comprising a sodium salt of the compound of Formula Ia or Ib, PEG 300, poloxamer 188, and water is about 25.9 w/w %. In some embodiments, the amount of the sodium salt of the compound of Formula Ia or Ib in the solution comprising a sodium salt of the compound of Formula Ia or Ib, PEG 300, poloxamer 188, and water is about 33.61 w/w %. In some embodiments, the amount of the sodium salt of the compound of Formula Ia or Ib in the solution comprising a sodium salt of the compound of Formula Ia or Ib, PEG 300, poloxamer 188, and water is about 33.6 w/w %.

In some embodiments, the amount of poloxamer 188 in the solution comprising a sodium salt of the compound of Formula Ia or Ib, PEG 300, poloxamer 188, and water is about 0.1 w/w % to about 10 w/w %. In some embodiments, the amount of poloxamer 188 in the solution comprising a sodium salt of the compound of Formula Ia or Ib, PEG 300, poloxamer 188, and water is about 0.3 w/w % to about 8 w/w %. In some embodiments, the amount of poloxamer 188 in the solution comprising a sodium salt of the compound of Formula Ia or Ib, PEG 300, poloxamer 188, and water is about 0.5 w/w % to about 7 w/w %. In some embodiments, the amount of poloxamer 188 in the solution comprising a sodium salt of the compound of Formula Ia or Ib, PEG 300, poloxamer 188, and water is about 0.6 w/w % to about 7 w/w %. In some embodiments, the amount of poloxamer 188 in the solution comprising a sodium salt of the compound of Formula Ia or Ib, PEG 300, poloxamer 188, and water is about 0.85 w/w % to about 4.82 w/w %. In some embodiments, the amount of poloxamer 188 in the solution comprising a sodium salt of the compound of Formula Ia or Ib, PEG 300, poloxamer 188, and water is about 0.9 w/w % to about 4.8 w/w %. In some embodiments, the amount of poloxamer 188 in the solution comprising a sodium salt of the compound of Formula Ia or Ib, PEG 300, poloxamer 188, and water is about 0.5 w/w %, about 0.6 w/w %, about 0.7 w/w %, about 0.8 w/w %, about 0.9 w/w %, about 1.0 w/w %, about 1.1 w/w %, about 1.2 w/w %, about 1.3 w/w %, about 1.4 w/w %, about 1.5 w/w %, about 1.6 w/w %, about 1.7 w/w %, about 1.8 w/w %, about 1.9 w/w %, about 2.0 w/w %, about 2.1 w/w %, about 2.2 w/w %, about 2.3 w/w %, about 2.4 w/w %, about 2.5 w/w %, about 2.6 w/w %, about 2.7 w/w %, about 2.8 w/w %, about 2.9 w/w %, about 3.0 w/w %, about 3.1 w/w %, about 3.2 w/w %, about 3.3 w/w %, about 3.4 w/w %, about 3.5 w/w %, about 3.6 w/w %, about 3.7 w/w %, about 3.8 w/w %, about 3.9 w/w %, about 4.0 w/w %, about 4.1 w/w %, about 4.2 w/w %, about 4.3 w/w %, about 4.4 w/w %, about 4.5 w/w %, about 4.6 w/w %, about 4.7 w/w %, about 4.8 w/w %, about 4.9 w/w %, about 5.0 w/w %, about 5.1 w/w %, about 5.2 w/w %, about 5.3 w/w %, about 5.4 w/w %, about 5.5 w/w %, about 5.6 w/w %, about 5.7 w/w %, about 5.8 w/w %, about 5.9 w/w %, about 6.0 w/w %, about 6.1 w/w %, about 6.2 w/w %, about 6.3 w/w %, about 6.4 w/w %, about 6.5 w/w %, about 6.6 w/w %, about 6.7 w/w %, about 6.8 w/w %, about 6.9 w/w %, or about 7.0 w/w %. In some embodiments, the amount of poloxamer 188 in the solution comprising a sodium salt of the compound of Formula Ia or Ib, PEG 300, poloxamer 188, and water is about 0.85 w/w %. In some embodiments, the amount of poloxamer 188 in the solution comprising a sodium salt of the compound of Formula Ia or Ib, PEG 300, poloxamer 188, and water is about 0.9 w/w %. In some embodiments, the amount of poloxamer 188 in the solution comprising a sodium salt of the compound of Formula Ia or Ib, PEG 300, poloxamer 188, and water is about 1.27 w/w %. In some embodiments, the amount of poloxamer 188 in the solution comprising a sodium salt of the compound of Formula Ia or Ib, PEG 300, poloxamer 188, and water is about 1.3 w/w %. In some embodiments, the amount of poloxamer 188 in the solution comprising a sodium salt of the compound of Formula Ia or Ib, PEG 300, poloxamer 188, and water is about 1.68 w/w %. In some embodiments, the amount of poloxamer 188 in the solution comprising a sodium salt of the compound of Formula Ia or Ib, PEG 300, poloxamer 188, and water is about 1.7 w/w %. In some embodiments, the amount of poloxamer 188 in the solution comprising a sodium salt of the compound of Formula Ia or Ib, PEG 300, poloxamer 188, and water is about 2.09 w/w %. In some embodiments, the amount of poloxamer 188 in the solution comprising a sodium salt of the compound of Formula Ia or Ib, PEG 300, poloxamer 188, and water is about 2.1 w/w %. In some embodiments, the amount of poloxamer 188 in the solution comprising a sodium salt of the compound of Formula Ia or Ib, PEG 300, poloxamer 188, and water is about 2.49 w/w %. In some embodiments, the amount of poloxamer 188 in the solution comprising a sodium salt of the compound of Formula Ia or Ib, PEG 300, poloxamer 188, and water is about 2.5 w/w %. In some embodiments, the amount of poloxamer 188 in the solution comprising a sodium salt of the compound of Formula Ia or Ib, PEG 300, poloxamer 188, and water is about 4.8 w/w %. In some embodiments, the amount of poloxamer 188 in the solution comprising a sodium salt of the compound of Formula Ia or Ib, PEG 300, poloxamer 188, and water is about 4.82 w/w %.

In some embodiments, the amount of poloxamer 188 in the solution comprising a sodium salt of the compound of Formula Ia or Ib, PEG 300, poloxamer 188, and water is about 0.85 w/w % to about 6.12 w/w %. In some embodiments, the amount of poloxamer 188 in the solution comprising a sodium salt of the compound of Formula Ia or Ib, PEG 300, poloxamer 188, and water is about 0.9 w/w % to about 6.1 w/w %.

In some embodiments, the amount of poloxamer 188 in the solution comprising a sodium salt of the compound of Formula Ia or Ib, PEG 300, poloxamer 188, and water is about 1.18 w/w %. In some embodiments, the amount of poloxamer 188 in the solution comprising a sodium salt of the compound of Formula Ia or Ib, PEG 300, poloxamer 188, and water is about 1.2 w/w %. In some embodiments, the amount of poloxamer 188 in the solution comprising a sodium salt of the compound of Formula Ia or Ib, PEG 300, poloxamer 188, and water is about 1.64 w/w %. In some embodiments, the amount of poloxamer 188 in the solution comprising a sodium salt of the compound of Formula Ia or Ib, PEG 300, poloxamer 188, and water is about 1.6 w/w %. In some embodiments, the amount of poloxamer 188 in the solution comprising a sodium salt of the compound of Formula Ia or Ib, PEG 300, poloxamer 188, and water is about 2.04 w/w %. In some embodiments, the amount of poloxamer 188 in the solution comprising a sodium salt of the compound of Formula Ia or Ib, PEG 300, poloxamer 188, and water is about 2.0 w/w %. In some embodiments, the amount of poloxamer 188 in the solution comprising a sodium salt of the compound of Formula Ia or Ib, PEG 300, poloxamer 188, and water is about 2.36 w/w %. In some embodiments, the amount of poloxamer 188 in the solution comprising a sodium salt of the compound of Formula Ia or Ib, PEG 300, poloxamer 188, and water is about 2.44 w/w %. In some embodiments, the amount of poloxamer 188 in the solution comprising a sodium salt of the compound of Formula Ia or Ib, PEG 300, poloxamer 188, and water is about 2.4 w/w %. In some embodiments, the amount of poloxamer 188 in the solution comprising a sodium salt of the compound of Formula Ia or Ib, PEG 300, poloxamer 188, and water is about 3.06 w/w %. In some embodiments, the amount of poloxamer 188 in the solution comprising a sodium salt of the compound of Formula Ia or Ib, PEG 300, poloxamer 188, and water is about 3.1 w/w %. In some embodiments, the amount of poloxamer 188 in the solution comprising a sodium salt of the compound of Formula Ia or Ib, PEG 300, poloxamer 188, and water is about 3.54 w/w %. In some embodiments, the amount of poloxamer 188 in the solution comprising a sodium salt of the compound of Formula Ia or Ib, PEG 300, poloxamer 188, and water is about 3.5 w/w %. In some embodiments, the amount of poloxamer 188 in the solution comprising a sodium salt of the compound of Formula Ia or Ib, PEG 300, poloxamer 188, and water is about 4.72 w/w %. In some embodiments, the amount of poloxamer 188 in the solution comprising a sodium salt of the compound of Formula Ia or Ib, PEG 300, poloxamer 188, and water is about 4.7 w/w %. In some embodiments, the amount of poloxamer 188 in the solution comprising a sodium salt of the compound of Formula Ia or Ib, PEG 300, poloxamer 188, and water is about 6.12 w/w %. In some embodiments, the amount of poloxamer 188 in the solution comprising a sodium salt of the compound of Formula Ia or Ib, PEG 300, poloxamer 188, and water is about 6.1 w/w %.

In some embodiments, the solution comprises about 10 w/w % to about 45 w/w % water, about 30 w/w % to about 85 w/w % PEG 300, about 0.5 w/w % to about 40 w/w % of a sodium salt of a compound of Formula Ia or Ib, and about 0.1 w/w % to about 10 w/w % of poloxamer 188. In some embodiments, the solution comprises about 15 w/w % to about 35 w/w % water, about 35 w/w % to about 75 w/w % PEG 300, about 1 w/w % to about 35 w/w % of a sodium salt of a compound of Formula Ia or Ib, and about 0.3 w/w % to about 8 w/w % of poloxamer 188. In some embodiments, the solution comprises about 20 w/w % to about 35 w/w % water, about 40 w/w % to about 70 w/w % PEG 300, about 1 w/w % to about 30 w/w % of a sodium salt of a compound of Formula Ia or Ib, and about 0.5 w/w % to about 7 w/w % of poloxamer 188. In some embodiments, the solution comprises about 20 w/w % to about 31 w/w % water, about 45 w/w % to about 68 w/w % PEG 300, about 3 w/w % to about 28 w/w % of a sodium salt of a compound of Formula Ia or Ib, and about 0.6 w/w % to about 7 w/w % of poloxamer 188. In some embodiments, the solution comprises about 21.9 w/w % to about 30.1 w/w % water, about 46.8 w/w % to about 64.4 w/w % PEG 300, about 4.7 w/w % to about 26.5 w/w % of a sodium salt of a compound of Formula Ia or Ib, and about 0.9 w/w % to about 4.8 w/w % of poloxamer 188. In some embodiments, the solution comprises about 21.87 w/w % to about 30.07 w/w % water, about 46.84 w/w % to about 64.40 w/w % PEG 300, about 4.68 w/w % to about 26.47 w/w % of a sodium salt of a compound of Formula Ia or Ib, and about 0.85 w/w % to about 4.82 w/w % of poloxamer 188.

In some embodiments, the solution comprises about 30.1 w/w % water, about 64.4 w/w % PEG 300, about 4.7 w/w % of a sodium salt of a compound of Formula Ia or Ib, and about 0.9 w/w % of poloxamer 188. In some embodiments, the solution comprises about 30.07 w/w % water, about 64.40 w/w % PEG 300, about 4.68 w/w % of a sodium salt of a compound of Formula Ia or Ib, and about 0.85 w/w % of poloxamer 188. In some embodiments, the solution comprises about 30.1 w/w % water, about 64.4 w/w % PEG 300, about 4.7 w/w % of a sodium salt of a compound of Formula Ib, and about 0.9 w/w % of poloxamer 188. In some embodiments, the solution comprises about 30.07 w/w % water, about 64.40 w/w % PEG 300, about 4.68 w/w % of a sodium salt of a compound of Formula Ib, and about 0.85 w/w % of poloxamer 188.

In some embodiments, the solution comprises about 29.2 w/w % water, about 62.6 w/w % PEG 300, about 7 w/w % of a sodium salt of a compound of Formula Ia or Ib, and about 1.3 w/w % of poloxamer 188. In some embodiments, the solution comprises about 29.21 w/w % water, about 62.55 w/w % PEG 300, about 6.97 w/w % of a sodium salt of a compound of Formula Ia or Ib, and about 1.27 w/w % of poloxamer 188. In some embodiments, the solution comprises about 29.2 w/w % water, about 62.6 w/w % PEG 300, about 7 w/w % of a sodium salt of a compound of Formula Ib, and about 1.3 w/w % of poloxamer 188. In some embodiments, the solution comprises about 29.21 w/w % water, about 62.55 w/w % PEG 300, about 6.97 w/w % of a sodium salt of a compound of Formula Ib, and about 1.27 w/w % of poloxamer 188.

In some embodiments, the solution comprises about 28.4 w/w % water, about 60.7 w/w % PEG 300, about 9.2 w/w % of a sodium salt of a compound of Formula Ia or Ib, and about 1.7 w/w % of poloxamer 188. In some embodiments, the solution comprises about 28.36 w/w % water, about 60.73 w/w % PEG 300, about 9.23 w/w % of a sodium salt of a compound of Formula Ia or Ib, and about 1.68 w/w % of poloxamer 188. In some embodiments, the solution comprises about 28.4 w/w % water, about 60.7 w/w % PEG 300, about 9.2 w/w % of a sodium salt of a compound of Formula Ib, and about 1.7 w/w % of poloxamer 188. In some embodiments, the solution comprises about 28.36 w/w % water, about 60.73 w/w % PEG 300, about 9.23 w/w % of a sodium salt of a compound of Formula Ib, and about 1.68 w/w % of poloxamer 188.

In some embodiments, the solution comprises about 27.5 w/w % water, about 58.9 w/w % PEG 300, about 11.5 w/w % of a sodium salt of a compound of Formula Ia or Ib, and about 2.1 w/w % of poloxamer 188. In some embodiments, the solution comprises about 27.51 w/w % water, about 58.92 w/w % PEG 300, about 11.48 w/w % of a sodium salt of a compound of Formula Ia or Ib, and about 2.09 w/w % of poloxamer 188. In some embodiments, the solution comprises about 27.5 w/w % water, about 58.9 w/w % PEG 300, about 11.5 w/w % of a sodium salt of a compound of Formula Ib, and about 2.1 w/w % of poloxamer 188. In some embodiments, the solution comprises about 27.51 w/w % water, about 58.92 w/w % PEG 300, about 11.48 w/w % of a sodium salt of a compound of Formula Ib, and about 2.09 w/w % of poloxamer 188.

In some embodiments, the solution comprises about 26.7 w/w % water, about 57.1 w/w % PEG 300, about 13.7 w/w % of a sodium salt of a compound of Formula Ia or Ib, and about 2.5 w/w % of poloxamer 188. In some embodiments, the solution comprises about 26.68 w/w % water, about 57.13 w/w % PEG 300, about 13.70 w/w % of a sodium salt of a compound of Formula Ia or Ib, and about 2.49 w/w % of poloxamer 188. In some embodiments, the solution comprises about 26.7 w/w % water, about 57.1 w/w % PEG 300, about 13.7 w/w % of a sodium salt of a compound of Formula Ib, and about 2.5 w/w % of poloxamer 188. In some embodiments, the solution comprises about 26.68 w/w % water, about 57.13 w/w % PEG 300, about 13.70 w/w % of a sodium salt of a compound of Formula Ib, and about 2.49 w/w % of poloxamer 188.

In some embodiments, the solution comprises about 21.9 w/w % water, about 46.8 w/w % PEG 300, about 26.5 w/w % of a sodium salt of a compound of Formula Ia or Ib, and about 4.8 w/w % of poloxamer 188. In some embodiments, the solution comprises about 21.87 w/w % water, about 46.84 w/w % PEG 300, about 26.47 w/w % of a sodium salt of a compound of Formula Ia or Ib, and about 4.82 w/w % of poloxamer 188. In some embodiments, the solution comprises about 21.9 w/w % water, about 46.8 w/w % PEG 300, about 26.5 w/w % of a sodium salt of a compound of Formula Ib, and about 4.8 w/w % of poloxamer 188. In some embodiments, the solution comprises about 21.87 w/w % water, about 46.84 w/w % PEG 300, about 26.47 w/w % of a sodium salt of a compound of Formula Ib, and about 4.82 w/w % of poloxamer 188.

In some embodiments, the solution comprises about 19.2 w/w % to about 30.1 w/w % water, about 41.1 w/w % to about 64.4 w/w % PEG 300, about 4.7 w/w % to about 33.6 w/w % of a sodium salt of a compound of Formula Ia or Ib, and about 0.9 w/w % to about 6.1 w/w % of poloxamer 188. In some embodiments, the solution comprises about 19.18 w/w % to about 30.07 w/w % water, about 41.09 w/w % to about 64.40 w/w % PEG 300, about 4.68 w/w % to about 33.61 w/w % of a sodium salt of a compound of Formula Ia or Ib, and about 0.85 w/w % to about 6.12 w/w % of poloxamer 188.

In some embodiments, the solution comprises about 28.4 w/w % water, about 60.9 w/w % PEG 300, about 9.0 w/w % of a sodium salt of a compound of Formula Ia or Ib, and about 1.6 w/w % of poloxamer 188. In some embodiments, the solution comprises about 28.43 w/w % water, about 60.90 w/w % PEG 300, about 9.03 w/w % of a sodium salt of a compound of Formula Ia or Ib, and about 1.64 w/w % of poloxamer 188. In some embodiments, the solution comprises about 28.4 w/w % water, about 60.9 w/w % PEG 300, about 9.0 w/w % of a sodium salt of a compound of Formula Ib, and about 1.6 w/w % of poloxamer 188. In some embodiments, the solution comprises about 28.43 w/w % water, about 60.90 w/w % PEG 300, about 9.03 w/w % of a sodium salt of a compound of Formula Ib, and about 1.64 w/w % of poloxamer 188.

In some embodiments, the solution comprises about 27.6 w/w % water, about 59.1 w/w % PEG 300, about 11.2 w/w % of a sodium salt of a compound of Formula Ia or Ib, and about 2.0 w/w % of poloxamer 188. In some embodiments, the solution comprises about 27.61 w/w % water, about 59.13 w/w % PEG 300, about 11.22 w/w % of a sodium salt of a compound of Formula Ia or Ib, and about 2.04 w/w % of poloxamer 188. In some embodiments, the solution comprises about 27.6 w/w % water, about 59.1 w/w % PEG 300, about 11.2 w/w % of a sodium salt of a compound of Formula Ib, and about 2.0 w/w % of poloxamer 188. In some embodiments, the solution comprises about 27.61 w/w % water, about 59.13 w/w % PEG 300, about 11.22 w/w % of a sodium salt of a compound of Formula Ib, and about 2.04 w/w % of poloxamer 188.

In some embodiments, the solution comprises about 26.8 w/w % water, about 57.4 w/w % PEG 300, about 13.4 w/w % of a sodium salt of a compound of Formula Ia or Ib, and about 2.4 w/w % of poloxamer 188. In some embodiments, the solution comprises about 26.79 w/w % water, about 57.38 w/w % PEG 300, about 13.39 w/w % of a sodium salt of a compound of Formula Ia or Ib, and about 2.44 w/w % of poloxamer 188. In some embodiments, the solution comprises about 26.8 w/w % water, about 57.4 w/w % PEG 300, about 13.4 w/w % of a sodium salt of a compound of Formula Ib, and about 2.4 w/w % of poloxamer 188. In some embodiments, the solution comprises about 26.79 w/w % water, about 57.38 w/w % PEG 300, about 13.39 w/w % of a sodium salt of a compound of Formula Ib, and about 2.44 w/w % of poloxamer 188.

In some embodiments, the solution comprises about 23.2 w/w % water, about 49.7 w/w % PEG 300, about 25.9 w/w % of a sodium salt of a compound of Formula Ia or Ib, and about 1.2 w/w % of poloxamer 188. In some embodiments, the solution comprises about 23.22 w/w % water, about 49.73 w/w % PEG 300, about 25.87 w/w % of a sodium salt of a compound of Formula Ia or Ib, and about 1.18 w/w % of poloxamer 188. In some embodiments, the solution comprises about 23.2 w/w % water, about 49.7 w/w % PEG 300, about 25.9 w/w % of a sodium salt of a compound of Formula Ib, and about 1.2 w/w % of poloxamer 188. In some embodiments, the solution comprises about 23.22 w/w % water, about 49.73 w/w % PEG 300, about 25.87 w/w % of a sodium salt of a compound of Formula Ib, and about 1.18 w/w % of poloxamer 188.

In some embodiments, the solution comprises about 22.9 w/w % water, about 48.9 w/w % PEG 300, about 25.9 w/w % of a sodium salt of a compound of Formula Ia or Ib, and about 2.4 w/w % of poloxamer 188. In some embodiments, the solution comprises about 22.85 w/w % water, about 48.94 w/w % PEG 300, about 25.85 w/w % of a sodium salt of a compound of Formula Ia or Ib, and about 2.36 w/w % of poloxamer 188. In some embodiments, the solution comprises about 22.9 w/w % water, about 48.9 w/w % PEG 300, about 25.9 w/w % of a sodium salt of a compound of Formula Ib, and about 2.4 w/w % of poloxamer 188. In some embodiments, the solution comprises about 22.85 w/w % water, about 48.94 w/w % PEG 300, about 25.85 w/w % of a sodium salt of a compound of Formula Ib, and about 2.36 w/w % of poloxamer 188.

In some embodiments, the solution comprises about 22.5 w/w % water, about 48.1 w/w % PEG 300, about 25.9 w/w % of a sodium salt of a compound of Formula Ia or Ib, and about 3.5 w/w % of poloxamer 188. In some embodiments, the solution comprises about 22.48 w/w % water, about 48.13 w/w % PEG 300, about 25.85 w/w % of a sodium salt of a compound of Formula Ia or Ib, and about 3.54 w/w % of poloxamer 188. In some embodiments, the solution comprises about 22.5 w/w % water, about 48.1 w/w % PEG 300, about 25.9 w/w % of a sodium salt of a compound of Formula Ib, and about 3.5 w/w % of poloxamer 188. In some embodiments, the solution comprises about 22.48 w/w % water, about 48.13 w/w % PEG 300, about 25.85 w/w % of a sodium salt of a compound of Formula Ib, and about 3.54 w/w % of poloxamer 188.

In some embodiments, the solution comprises about 22.1 w/w % water, about 47.3 w/w % PEG 300, about 25.9 w/w % of a sodium salt of a compound of Formula Ia or Ib, and about 4.7 w/w % of poloxamer 188. In some embodiments, the solution comprises about 22.10 w/w % water, about 47.33 w/w % PEG 300, about 25.85 w/w % of a sodium salt of a compound of Formula Ia or Ib, and about 4.72 w/w % of poloxamer 188. In some embodiments, the solution comprises about 22.1 w/w % water, about 47.3 w/w % PEG 300, about 25.9 w/w % of a sodium salt of a compound of Formula Ib, and about 4.7 w/w % of poloxamer 188. In some embodiments, the solution comprises about 22.10 w/w % water, about 47.33 w/w % PEG 300, about 25.85 w/w % of a sodium salt of a compound of Formula Ib, and about 4.72 w/w % of poloxamer 188.

In some embodiments, the solution comprises about 20.2 w/w % water, about 43.2 w/w % PEG 300, about 33.6 w/w % of a sodium salt of a compound of Formula Ia or Ib, and about 3.1 w/w % of poloxamer 188. In some embodiments, the solution comprises about 20.16 w/w % water, about 43.17 w/w % PEG 300, about 33.61 w/w % of a sodium salt of a compound of Formula Ia or Ib, and about 3.06 w/w % of poloxamer 188. In some embodiments, the solution comprises about 20.2 w/w % water, about 43.2 w/w % PEG 300, about 33.6 w/w % of a sodium salt of a compound of Formula Ib, and about 3.1 w/w % of poloxamer 188. In some embodiments, the solution comprises about 20.16 w/w % water, about 43.17 w/w % PEG 300, about 33.61 w/w % of a sodium salt of a compound of Formula Ib, and about 3.06 w/w % of poloxamer 188.

In some embodiments, the solution comprises about 19.2 w/w % water, about 41.1 w/w % PEG 300, about 33.6 w/w % of a sodium salt of a compound of Formula Ia or Ib, and about 6.1 w/w % of poloxamer 188. In some embodiments, the solution comprises about 19.18 w/w % water, about 41.09 w/w % PEG 300, about 33.61 w/w % of a sodium salt of a compound of Formula Ia or Ib, and about 6.12 w/w % of poloxamer 188. In some embodiments, the solution comprises about 19.2 w/w % water, about 41.1 w/w % PEG 300, about 33.6 w/w % of a sodium salt of a compound of Formula Ib, and about 6.1 w/w % of poloxamer 188. In some embodiments, the solution comprises about 19.18 w/w % water, about 41.09 w/w % PEG 300, about 33.61 w/w % of a sodium salt of a compound of Formula Ib, and about 6.12 w/w % of poloxamer 188.

In some embodiments, the solution comprises a compound of Formula Ia or Ib, or a pharmaceutically acceptable salt thereof, PEG 300, water, poloxamer 188, and ethanol. In some embodiments, the solution comprises a compound of Formula Ia, or a pharmaceutically acceptable salt thereof, PEG 300, water, poloxamer 188, and ethanol. In some embodiments, the solution comprises a compound of Formula Ia, PEG 300, water, poloxamer 188, and ethanol. In some embodiments, the solution comprises a sodium salt of the compound of Formula Ia, PEG 300, water, poloxamer 188, and ethanol. In some embodiments, the solution comprises a compound of Formula Ib, or a pharmaceutically acceptable salt thereof, PEG 300, water, poloxamer 188, and ethanol. In some embodiments, the solution comprises a compound of Formula Ib, or a pharmaceutically acceptable salt thereof, PEG 300, water, poloxamer 188, and ethanol. In some embodiments, the solution comprises a sodium salt of the compound of Formula Ib, or a pharmaceutically acceptable salt thereof, PEG 300, water, poloxamer 188, and ethanol.

In some embodiments, the solution comprises a sodium salt of the compound of Formula Ia or Ib, PEG 300, water, poloxamer 188, and ethanol. In some embodiments, the concentration of the compound of Formula Ia or Ib in the solution comprising a sodium salt of the compound of Formula Ia or Ib, PEG 300, water, poloxamer 188, and ethanol is about 50 mg/ml to about 600 mg/ml. In some embodiments, the concentration of the compound of Formula Ia or Ib in the solution comprising a sodium salt of the compound of Formula Ia or Ib, PEG 300, water, poloxamer 188, and ethanol is about 50 mg/ml to about 500 mg/ml. In some embodiments, the concentration of the compound of Formula Ia or Ib in the solution comprising a sodium salt of the compound of Formula Ia or Ib, PEG 300, water, poloxamer 188, and ethanol is about 200 mg/ml to about 600 mg/ml. In some embodiments, the concentration of the compound of Formula Ia or Ib in the solution comprising a sodium salt of the compound of Formula Ia or Ib, PEG 300, water, poloxamer 188, and ethanol is about 300 mg/ml to about 600 mg/ml. In some embodiments, the concentration of the compound of Formula Ia or Ib in the solution comprising a sodium salt of the compound of Formula Ia or Ib, PEG 300, water, poloxamer 188, and ethanol is about 300 mg/ml to about 500 mg/ml. In some embodiments, the concentration of the compound of Formula Ia or Ib in the solution comprising a sodium salt of the compound of Formula Ia or Ib, PEG 300, water, poloxamer 188, and ethanol is about 50 mg/ml. In some embodiments, the concentration of the compound of Formula Ia or Ib in the solution comprising a sodium salt of the compound of Formula Ia or Ib, PEG 300, water, poloxamer 188, and ethanol is about 100 mg/ml. In some embodiments, the concentration of the compound of Formula Ia or Ib in the solution comprising a sodium salt of the compound of Formula Ia or Ib, PEG 300, water, poloxamer 188, and ethanol is about 150 mg/ml. In some embodiments, the concentration of the compound of Formula Ia or Ib in the solution comprising a sodium salt of the compound of Formula Ia or Ib, PEG 300, water, poloxamer 188, and ethanol is about 200 mg/ml. In some embodiments, the concentration of the compound of Formula Ia or Ib in the solution comprising a sodium salt of the compound of Formula Ia or Ib, PEG 300, water, poloxamer 188, and ethanol is about 250 mg/ml. In some embodiments, the concentration of the compound of Formula Ia or Ib in the solution comprising a sodium salt of the compound of Formula Ia or Ib, PEG 300, water, poloxamer 188, and ethanol is about 300 mg/ml. In some embodiments, the concentration of the compound of Formula Ia or Ib in the solution comprising a sodium salt of the compound of Formula Ia or Ib, PEG 300, water, poloxamer 188, and ethanol is about 350 mg/ml. In some embodiments, the concentration of the compound of Formula Ia or Ib in the solution comprising a sodium salt of the compound of Formula Ia or Ib, PEG 300, water, poloxamer 188, and ethanol is about 400 mg/ml. In some embodiments, the concentration of the compound of Formula Ia or Ib in the solution comprising a sodium salt of the compound of Formula Ia or Ib, PEG 300, water, poloxamer 188, and ethanol is about 450 mg/ml. In some embodiments, the concentration of the compound of Formula Ia or Ib in the solution comprising a sodium salt of the compound of Formula Ia or Ib, PEG 300, water, poloxamer 188, and ethanol is about 500 mg/ml. In some embodiments, the concentration of the compound of Formula Ia or Ib in the solution comprising a sodium salt of the compound of Formula Ia or Ib, PEG 300, water, poloxamer 188, and ethanol is about 550 mg/ml. In some embodiments, the concentration of the compound of Formula Ia or Ib in the solution comprising a sodium salt of the compound of Formula Ia or Ib, PEG 300, water, poloxamer 188, and ethanol is about 600 mg/ml.

In some embodiments, the amount of water in the solution comprising a sodium salt of the compound of Formula Ia or Ib, PEG 300, water, poloxamer 188, and ethanol is about 10 w/w % to about 40 w/w %. In some embodiments, the amount of water in the solution comprising a sodium salt of the compound of Formula Ia or Ib, PEG 300, water, poloxamer 188, and ethanol is about 10 w/w % to about 20 w/w %. In some embodiments, the amount of water in the solution comprising a sodium salt of the compound of Formula Ia or Ib, PEG 300, water, poloxamer 188, and ethanol is about 10 w/w % to about 19 w/w %. In some embodiments, the amount of water in the solution comprising a sodium salt of the compound of Formula Ia or Ib, PEG 300, water, poloxamer 188, and ethanol is about 14.50 w/w % to about 17.26 w/w %. In some embodiments, the amount of water in the solution comprising a sodium salt of the compound of Formula Ia or Ib, PEG 300, water, poloxamer 188, and ethanol is about 14.5 w/w % to about 17.3 w/w %. In some embodiments, the amount of water in the solution comprising a sodium salt of the compound of Formula Ia or Ib, PEG 300, water, poloxamer 188, and ethanol is about 14.50 w/w %. In some embodiments, the amount of water in the solution comprising a sodium salt of the compound of Formula Ia or Ib, PEG 300, water, poloxamer 188, and ethanol is about 14.5 w/w %. In some embodiments, the amount of water in the solution comprising a sodium salt of the compound of Formula Ia or Ib, PEG 300, water, poloxamer 188, and ethanol is about 14.57 w/w %. In some embodiments, the amount of water in the solution comprising a sodium salt of the compound of Formula Ia or Ib, PEG 300, water, poloxamer 188, and ethanol is about 14.6 w/w %. In some embodiments, the amount of water in the solution comprising a sodium salt of the compound of Formula Ia or Ib, PEG 300, water, poloxamer 188, and ethanol is about 15.71 w/w %. In some embodiments, the amount of water in the solution comprising a sodium salt of the compound of Formula Ia or Ib, PEG 300, water, poloxamer 188, and ethanol is about 15.7 w/w %. In some embodiments, the amount of water in the solution comprising a sodium salt of the compound of Formula Ia or Ib, PEG 300, water, poloxamer 188, and ethanol is about 17.26 w/w %. In some embodiments, the amount of water in the solution comprising a sodium salt of the compound of Formula Ia or Ib, PEG 300, water, poloxamer 188, and ethanol is about 17.3 w/w %.

In some embodiments, the amount of PEG 300 in the solution comprising a sodium salt of the compound of Formula Ia or Ib, PEG 300, water, poloxamer 188, and ethanol is about 20 w/w % to about 75 w/w %. In some embodiments, the amount of PEG 300 in the solution comprising a sodium salt of the compound of Formula Ia or Ib, PEG 300, water, poloxamer 188, and ethanol is about 25 w/w % to about 40 w/w %. In some embodiments, the amount of PEG 300 in the solution comprising a sodium salt of the compound of Formula Ia or Ib, PEG 300, water, poloxamer 188, and ethanol is about 27 w/w % to about 37 w/w %. In some embodiments, the amount of PEG 300 in the solution comprising a sodium salt of the compound of Formula Ia or Ib, PEG 300, water, poloxamer 188, and ethanol is about 31.04 w/w % to about 36.96 w/w %. In some embodiments, the amount of PEG 300 in the solution comprising a sodium salt of the compound of Formula Ia or Ib, PEG 300, water, poloxamer 188, and ethanol is about 31.0 w/w % to about 37.0 w/w %. In some embodiments, the amount of PEG 300 in the solution comprising a sodium salt of the compound of Formula Ia or Ib, PEG 300, water, poloxamer 188, and ethanol is about 31.04 w/w %. In some embodiments, the amount of PEG 300 in the solution comprising a sodium salt of the compound of Formula Ia or Ib, PEG 300, water, poloxamer 188, and ethanol is about 31.0 w/w %. In some embodiments, the amount of PEG 300 in the solution comprising a sodium salt of the compound of Formula Ia or Ib, PEG 300, water, poloxamer 188, and ethanol is about 31.21 w/w %. In some embodiments, the amount of PEG 300 in the solution comprising a sodium salt of the compound of Formula Ia or Ib, PEG 300, water, poloxamer 188, and ethanol is about 31.2 w/w %. In some embodiments, the amount of PEG 300 in the solution comprising a sodium salt of the compound of Formula Ia or Ib, PEG 300, water, poloxamer 188, and ethanol is about 33.63 w/w %. In some embodiments, the amount of PEG 300 in the solution comprising a sodium salt of the compound of Formula Ia or Ib, PEG 300, water, poloxamer 188, and ethanol is about 33.6 w/w %. In some embodiments, the amount of PEG 300 in the solution comprising a sodium salt of the compound of Formula Ia or Ib, PEG 300, water, poloxamer 188, and ethanol is about 36.96 w/w %. In some embodiments, the amount of PEG 300 in the solution comprising a sodium salt of the compound of Formula Ia or Ib, PEG 300, water, poloxamer 188, and ethanol is about 37.0 w/w %.

In some embodiments, the amount of the sodium salt of the compound of Formula Ia or Ib in the solution comprising a sodium salt of the compound of Formula Ia or Ib, PEG 300, water, poloxamer 188, and ethanol is about 10 w/w % to about 70 w/w %. In some embodiments, the amount of the sodium salt of the compound of Formula Ia or Ib in the solution comprising a sodium salt of the compound of Formula Ia or Ib, PEG 300, water, poloxamer 188, and ethanol is about 30 w/w % to about 45 w/w %. In some embodiments, the amount of the sodium salt of the compound of Formula Ia or Ib in the solution comprising a sodium salt of the compound of Formula Ia or Ib, PEG 300, water, poloxamer 188, and ethanol is about 32 w/w % to about 45 w/w %. In some embodiments, the amount of the sodium salt of the compound of Formula Ia or Ib in the solution comprising a sodium salt of the compound of Formula Ia or Ib, PEG 300, water, poloxamer 188, and ethanol is about 34.50 w/w % to about 41.85 w/w %. In some embodiments, the amount of the sodium salt of the compound of Formula Ia or Ib in the solution comprising a sodium salt of the compound of Formula Ia or Ib, PEG 300, water, poloxamer 188, and ethanol is about 34.5 w/w % to about 41.9 w/w %. In some embodiments, the amount of the sodium salt of the compound of Formula Ia or Ib in the solution comprising a sodium salt of the compound of Formula Ia or Ib, PEG 300, water, poloxamer 188, and ethanol is about 34.50 w/w %. In some embodiments, the amount of the sodium salt of the compound of Formula Ia or Ib in the solution comprising a sodium salt of the compound of Formula Ia or Ib, PEG 300, water, poloxamer 188, and ethanol is about 34.5 w/w %. In some embodiments, the amount of the sodium salt of the compound of Formula Ia or Ib in the solution comprising a sodium salt of the compound of Formula Ia or Ib, PEG 300, water, poloxamer 188, and ethanol is about 41.64 w/w %. In some embodiments, the amount of the sodium salt of the compound of Formula Ia or Ib in the solution comprising a sodium salt of the compound of Formula Ia or Ib, PEG 300, water, poloxamer 188, and ethanol is about 41.6 w/w %. In some embodiments, the amount of the sodium salt of the compound of Formula Ia or Ib in the solution comprising a sodium salt of the compound of Formula Ia or Ib, PEG 300, water, poloxamer 188, and ethanol is about 41.85 w/w %. In some embodiments, the amount of the sodium salt of the compound of Formula Ia or Ib in the solution comprising a sodium salt of the compound of Formula Ia or Ib, PEG 300, water, poloxamer 188, and ethanol is about 41.9 w/w %.

In some embodiments, the amount of poloxamer 188 in the solution comprising a sodium salt of the compound of Formula Ia or Ib, PEG 300, water, poloxamer 188, and ethanol is about 0.5 w/w % to about 20 w/w %. In some embodiments, the amount of poloxamer 188 in the solution comprising a sodium salt of the compound of Formula Ia or Ib, PEG 300, water, poloxamer 188, and ethanol is about 0.5 w/w % to about 12 w/w %. In some embodiments, the amount of poloxamer 188 in the solution comprising a sodium salt of the compound of Formula Ia or Ib, PEG 300, water, poloxamer 188, and ethanol is about 1 w/w % to about 11 w/w %. In some embodiments, the amount of poloxamer 188 in the solution comprising a sodium salt of the compound of Formula Ia or Ib, PEG 300, water, poloxamer 188, and ethanol is about 3.81 w/w % to about 7.61 w/w %. In some embodiments, the amount of poloxamer 188 in the solution comprising a sodium salt of the compound of Formula Ia or Ib, PEG 300, water, poloxamer 188, and ethanol is about 3.8 w/w % to about 7.6 w/w %. In some embodiments, the amount of poloxamer 188 in the solution comprising a sodium salt of the compound of Formula Ia or Ib, PEG 300, water, poloxamer 188, and ethanol is about 3.81 w/w %. In some embodiments, the amount of poloxamer 188 in the solution comprising a sodium salt of the compound of Formula Ia or Ib, PEG 300, water, poloxamer 188, and ethanol is about 3.8 w/w %. In some embodiments, the amount of poloxamer 188 in the solution comprising a sodium salt of the compound of Formula Ia or Ib, PEG 300, water, poloxamer 188, and ethanol is about 6.28 w/w %. In some embodiments, the amount of poloxamer 188 in the solution comprising a sodium salt of the compound of Formula Ia or Ib, PEG 300, water, poloxamer 188, and ethanol is about 6.3 w/w %. In some embodiments, the amount of poloxamer 188 in the solution comprising a sodium salt of the compound of Formula Ia or Ib, PEG 300, water, poloxamer 188, and ethanol is about 7.58 w/w %. In some embodiments, the amount of poloxamer 188 in the solution comprising a sodium salt of the compound of Formula Ia or Ib, PEG 300, water, poloxamer 188, and ethanol is about 7.61 w/w %. In some embodiments, the amount of poloxamer 188 in the solution comprising a sodium salt of the compound of Formula Ia or Ib, PEG 300, water, poloxamer 188, and ethanol is about 7.6 w/w %.

In some embodiments, the amount of ethanol in the solution comprising a sodium salt of the compound of Formula Ia or Ib, PEG 300, water, poloxamer 188, and ethanol is about 0.1 w/w % to about 10 w/w %. In some embodiments, the amount of ethanol in the solution comprising a sodium salt of the compound of Formula Ia or Ib, PEG 300, water, poloxamer 188, and ethanol is about 1 w/w % to about 10 w/w %. In some embodiments, the amount of ethanol in the solution comprising a sodium salt of the compound of Formula Ia or Ib, PEG 300, water, poloxamer 188, and ethanol is about 1 w/w % to about 9 w/w %. In some embodiments, the amount of ethanol in the solution comprising a sodium salt of the compound of Formula Ia or Ib, PEG 300, water, poloxamer 188, and ethanol is about 3 w/w % to about 8 w/w %. In some embodiments, the amount of ethanol in the solution comprising a sodium salt of the compound of Formula Ia or Ib, PEG 300, water, poloxamer 188, and ethanol is about 5.00 w/w %. In some embodiments, the amount of ethanol in the solution comprising a sodium salt of the compound of Formula Ia or Ib, PEG 300, water, poloxamer 188, and ethanol is about 5.0 w/w %.

In some embodiments, the solution comprises about 10 w/w % to about 40 w/w % water, about 20 w/w % to about 75 w/w % PEG 300, about 10 w/w % to about 70 w/w % of a sodium salt of the compound of Formula Ia or Ib, about 0.5 w/w % to about 20 w/w % poloxamer 188, and about 1 w/w % to about 10 w/w % of ethanol. In some embodiments, the solution comprises about 10 w/w % to about 20 w/w % water, about 25 w/w % to about 40 w/w % PEG 300, about 30 w/w % to about 45 w/w % of a sodium salt of the compound of Formula Ia or Ib, about 1 w/w % to about 11 w/w % of poloxamer 188, and about 3 w/w % to about 8 w/w % of ethanol.

In some embodiments, the solution comprises about 17.26 w/w % water, about 36.96 w/w % PEG 300, about 34.50 w/w % of a sodium salt of the compound of Formula Ia or Ib, about 6.28 w/w % poloxamer 188, and about 5.00 w/w % ethanol. In some embodiments, the solution comprises about 17.3 w/w % water, about 37.0 w/w % PEG 300, about 34.5 w/w % of a sodium salt of the compound of Formula Ia or Ib, about 6.3 w/w % poloxamer 188, and about 5.0 w/w % ethanol. In some embodiments, the solution comprises about 17.26 w/w % water, about 36.96 w/w % PEG 300, about 34.50 w/w % of a sodium salt of the compound of Formula Ib, about 6.28 w/w % poloxamer 188, and about 5.00 w/w % ethanol. In some embodiments, the solution comprises about 17.3 w/w % water, about 37.0 w/w % PEG 300, about 34.5 w/w % of a sodium salt of the compound of Formula Ib, about 6.3 w/w % poloxamer 188, and about 5.0 w/w % ethanol.

In some embodiments, the solution comprises about 15.71 w/w % water, about 33.63 w/w % PEG 300, about 41.85 w/w % of a sodium salt of the compound of Formula Ia or Ib, about 3.81 w/w % poloxamer 188, and about 5.00 w/w % ethanol. In some embodiments, the solution comprises about 15.7 w/w % water, about 33.6 w/w % PEG 300, about 41.9 w/w % of a sodium salt of the compound of Formula Ia or Ib, about 3.8 w/w % poloxamer 188, and about 5.0 w/w % ethanol. In some embodiments, the solution comprises about 15.71 w/w % water, about 33.63 w/w % PEG 300, about 41.85 w/w % of a sodium salt of the compound of Formula Ib, about 3.81 w/w % poloxamer 188, and about 5.00 w/w % ethanol. In some embodiments, the solution comprises about 15.7 w/w % water, about 33.6 w/w % PEG 300, about 41.9 w/w % of a sodium salt of the compound of Formula Ib, about 3.8 w/w % poloxamer 188, and about 5.0 w/w % ethanol.

In some embodiments, the solution comprises about 14.57 w/w % water, about 31.21 w/w % PEG 300, about 41.64 w/w % of a sodium salt of the compound of Formula Ia or Ib, about 7.58 w/w % poloxamer 188, and about 5.00 w/w % ethanol. In some embodiments, the solution comprises about 14.6 w/w % water, about 31.2 w/w % PEG 300, about 41.6 w/w % of a sodium salt of the compound of Formula Ia or Ib, about 7.6 w/w % poloxamer 188, and about 5.0 w/w % ethanol. In some embodiments, the solution comprises about 14.57 w/w % water, about 31.21 w/w % PEG 300, about 41.64 w/w % of a sodium salt of the compound of Formula Ib, about 7.58 w/w % poloxamer 188, and about 5.00 w/w % ethanol. In some embodiments, the solution comprises about 14.6 w/w % water, about 31.2 w/w % PEG 300, about 41.6 w/w % of a sodium salt of the compound of Formula Ib, about 7.6 w/w % poloxamer 188, and about 5.0 w/w % ethanol.

In some embodiments, the solution comprises about 14.50 w/w % water, about 31.04 w/w % PEG 300, about 41.85 w/w % of a sodium salt of the compound of Formula Ia or Ib, about 7.61 w/w % poloxamer 188, and about 5.00 w/w % ethanol. In some embodiments, the solution comprises about 14.5 w/w % water, about 31.0 w/w % PEG 300, about 41.9 w/w % of a sodium salt of the compound of Formula Ia or Ib, about 7.6 w/w % poloxamer 188, and about 5.0 w/w % ethanol. In some embodiments, the solution comprises about 14.50 w/w % water, about 31.04 w/w % PEG 300, about 41.85 w/w % of a sodium salt of the compound of Formula Ib, about 7.61 w/w % poloxamer 188, and about 5.00 w/w % ethanol. In some embodiments, the solution comprises about 14.5 w/w % water, about 31.0 w/w % PEG 300, about 41.9 w/w % of a sodium salt of the compound of Formula Ib, about 7.6 w/w % poloxamer 188, and about 5.0 w/w % ethanol.

In some embodiments of the methods herein, the compound of Formula Ia or Ib, or a pharmaceutically acceptable salt thereof is administered as a parenteral formulation (for example, for SC or IM administration), which is an aqueous suspension. In some embodiments, the parenteral formulation (for example, an SC or IM formulation is an aqueous suspension that includes a compound of Formula Ia or Ib, or a pharmaceutically acceptable salt thereof, and saline. In some embodiments, the parenteral formulation (for example, an SC or IM formulation) is an aqueous suspension that comprises a compound of Formula Ia or Ib, or a pharmaceutically acceptable salt thereof, saline, and a suspending agent. In some embodiments, the parenteral formulation (for example, an SC or IM formulation) is an aqueous suspension that comprises a compound of Formula Ia or Ib, or a pharmaceutically acceptable salt thereof, saline, and a poloxamer (such as poloxamer 338, 188, or 207).

In some embodiments, a suspension comprising a compound of Formula Ia or Ib, or a pharmaceutically acceptable salt thereof, in a poloxamer and saline is provided. In some embodiments, the concentration of poloxamer in saline is from about 0.1% to about 20%. In some embodiments, the concentration of poloxamer in saline is from about 0.1% to about 10%. In some embodiments, the concentration of poloxamer in saline is about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%. In certain embodiments, the concentration of poloxamer in saline is about 2%. In certain embodiments, the poloxamer is poloxamer 188. In certain embodiments, the compound is a compound of Formula Ia or Ib, or a pharmaceutically acceptable salt thereof. In certain embodiments, the compound is a compound of Formula Ia. In certain embodiments, the compound is a sodium salt of the compound of Formula Ia. In certain embodiments, the compound is a compound of Formula Ib, or a pharmaceutically acceptable salt thereof. In certain embodiments, the compound is a compound of Formula Ib. In certain embodiments, the compound is a sodium salt of the compound of Formula Ib.

In some embodiments, a suspension comprising a compound of Formula Ia or Ib, or a pharmaceutically acceptable salt thereof, in a poloxamer and mannitol is provided. In some embodiments, the concentration of poloxamer in mannitol is from about 0.1% to about 20%. In some embodiments, the concentration of poloxamer in mannitol is from about 0.1% to about 10%. In some in embodiments, the concentration of poloxamer in mannitol is about 0.1%, about 0.2%, about 0.3%, about 0.4%, about 0.5%, about 0.6%, about 0.7%, about 0.8%, about 0.9%, about 1%, about 2, %, about 3%, about 4%, about 5%, about 6%, about 7%, about 8%, about 9%, or about 10%. In certain embodiments, the concentration of poloxamer in mannitol is about 2%. In certain embodiments, the poloxamer is poloxamer 188. In certain embodiments, the compound is a compound of Formula Ia, or a pharmaceutically acceptable salt thereof. In certain embodiments, the compound is a compound of Formula Ia. In certain embodiments, the compound is a sodium salt of the compound of Formula Ia. In certain embodiments, the compound is a compound of Formula Ib, or a pharmaceutically acceptable salt thereof. In certain embodiments, the compound is a compound of Formula Ib. In certain embodiments, the compound is a sodium salt of the compound of Formula Ib.

In certain embodiments, the composition is formulated as a solid dosage form. In some embodiments, the solid dosage form is a solid injectable dosage form, such as a solid depot form.

In certain embodiments, the active ingredient (for example, a compound of Formula Ia or Ib) is present as a free acid. In certain embodiments, the active ingredient (for example, a compound of Formula Ia or Ib) is present as a sodium salt. In some embodiments, the active ingredient is a compound of Formula Ia. In some embodiments, the active ingredient is a compound of Formula Ib.

In certain embodiments, the pharmaceutical composition is a parenteral formulation. In certain embodiments, the formulation is administered subcutaneously to a patient in need thereof. In certain embodiments, the formulation is administered intramuscularly to a patient in need thereof.

In certain embodiments, the parenteral formulation comprises N-methyl-2-pyrrolidone (NMP). In certain embodiments, the parenteral formulation consists essentially of N-methyl-2-pyrrolidone. In certain embodiments, the parenteral formulation comprises dimethyl sulfoxide (DMSO). In some embodiments, the parenteral formulation comprises polyethylene glycol (PEG) or glycofurol. In some embodiments, the solution comprises PEG 200, ethanol, and water. In some embodiments, the solution comprises PEG 300 and water. In some embodiments, the solution comprises poloxamer in saline. In some embodiments, the solution comprises 2% poloxamer 188 in normal saline.

In certain embodiments, the parenteral formulation comprises a compound of Formula Ia or Ib, or a pharmaceutically acceptable salt thereof, and water. In certain embodiments, the parenteral formulation comprises a compound of Formula Ia, or a pharmaceutically acceptable salt thereof, and water. In certain embodiments, the parenteral formulation comprises a compound of Formula Ib, or a pharmaceutically acceptable salt thereof, and water. In certain embodiments, the parenteral formulation further contains an alcohol. In certain embodiments, the alcohol is ethanol. In certain embodiments, the parenteral formulation further contains polyethylene glycol. In certain embodiments, the polyethylene glycol has an average molecular weight of about 200 g/mol (for example, polyethylene glycol 200). In certain embodiments, the parenteral formulation further contains an inorganic base. In certain embodiments, the inorganic base is sodium hydroxide (NaOH). In certain embodiments, the inorganic base is sodium ethoxide (NaOEt). In certain embodiments, the formulation comprises from about 0.1 molar equivalents to about 1.5 molar equivalents of the inorganic base. In certain embodiments, the formulation comprises from about 0.5 molar equivalents to about 1.5 molar equivalents of the inorganic base. In certain embodiments, the formulation comprises from about 0.75 molar equivalents to about 1.2 molar equivalents of the inorganic base. In certain embodiments, the formulation comprises about 1.0 molar equivalents inorganic base. In certain embodiments, the formulation comprises about 1.2 molar equivalents inorganic base. In some embodiments, the inorganic base is NaOH or NaOEt.

In certain embodiments, the parenteral formulation comprises a compound of Formula Ia, or a pharmaceutically acceptable salt thereof, water and polyethylene glycol PEG 300. In certain embodiments, the parenteral formulation comprises a compound of Formula Ib, or a pharmaceutically acceptable salt thereof, water, and polyethylene glycol PEG 300.

In certain embodiments, the parenteral formulation comprises a compound of Formula Ia or Ib, or a pharmaceutically acceptable salt thereof, water, and polyethylene glycol PEG 300 (polyethylene glycol with an average molecular weight of 300 g/mol), and NaOH. In certain embodiments, the parenteral formulation comprises a compound of Formula Ia or Ib, or a pharmaceutically acceptable salt thereof, water, and polyethylene glycol (PEG) 300, and NaOEt. In certain embodiments, the formulation includes from about 0.1 molar equivalents to about 1.5 molar equivalents of NaOH or NaOEt. In certain embodiments, the formulation includes from about 0.5 molar equivalents to about 1.5 molar equivalents of NaOH or NaOEt. In certain embodiments, the formulation includes from about 0.75 molar equivalents to about 1.2 molar equivalents of NaOH or NaOEt. In certain embodiments, the formulation comprises about 1.0 molar equivalents of NaOH or NaOEt. In certain embodiments, the formulation includes about 1.2 molar equivalents of NaOH or NaOEt.

In certain embodiments, the parenteral formulation is a solution formulation that includes a mixture of ethanol, water, and polyethylene glycol. In certain embodiments, the parenteral formulation is a solution formulation that includes a mixture of ethanol, water, and PEG 200. In certain embodiments, the solution formulation includes about 5% to about 20% ethanol, about 5% to about 20% water, and about 60% to about 90% PEG 200. In certain embodiments, the solution formulation comprises about 10% to about 15% ethanol, about 10% to about 15% water, and about 70% to about 80% PEG 200. In certain embodiments, the solution formulation includes about 10% ethanol, about 12% water, and about 78% PEG 200. In certain embodiments, the solution formulation further includes an inorganic base. In certain embodiments, the solution formulation includes about 10% ethanol, about 13% water, and about 77% PEG 200. In certain embodiments, the solution formulation further includes an inorganic base. In certain embodiments, the formulation includes from about 0.1 molar equivalents to about 1.5 molar equivalents of the inorganic base. In certain embodiments, the formulation comprises from about 0.5 molar equivalents to about 1.5 molar equivalents of the inorganic base. In certain embodiments, the formulation comprises from about 1.0 molar equivalents to about 1.2 molar equivalents of the inorganic base. In certain embodiments, the formulation comprises about 1.2 molar equivalents inorganic base. In certain embodiments, the inorganic base is sodium hydroxide or sodium ethoxide. In certain embodiments, the inorganic base is sodium hydroxide.

In certain embodiments, the parenteral formulation is a solution formulation that includes a mixture of water and polyethylene glycol. In certain embodiments, the parenteral formulation is a solution formulation that includes a mixture of water and PEG 300. In certain embodiments, the solution formulation includes about 5% w/w to about 25% w/w water, and about 75% w/w to about 95% w/w PEG 300. In some embodiments, the solution formulation further includes an inorganic base. In certain embodiments, the formulation includes from about 0.1 molar equivalents to about 1.5 molar equivalents of the inorganic base. In certain embodiments, the formulation comprises from about 0.5 molar equivalents to about 1.5 molar equivalents of the inorganic base. In certain embodiments, the formulation comprises from about 0.75 molar equivalents to about 1.2 molar equivalents of the inorganic base. In certain embodiments, the formulation comprises about 1.0 molar equivalents inorganic base. In certain embodiments, the formulation comprises about 1.2 molar equivalents inorganic base. In certain embodiments, the inorganic base is sodium hydroxide or sodium ethoxide. In certain embodiments, the inorganic base is sodium hydroxide.

In some embodiments, the solution comprises a compound of Formula Ia or Ib, or a pharmaceutically acceptable salt thereof, PEG 300, sodium hydroxide, and water. In some embodiments, the solution comprises a compound of Formula Ia or a pharmaceutically acceptable salt thereof, PEG 300, sodium hydroxide, and water. In some embodiments, the solution comprises a compound of Formula Ib, or a pharmaceutically acceptable salt thereof, PEG 300, sodium hydroxide, and water. In some embodiments, the solution comprises a compound of Formula Ia, PEG 300, sodium hydroxide, and water. In some embodiments, the solution comprises a sodium salt of the compound of Formula Ia, PEG 300, sodium hydroxide, and water. In some embodiments, the solution comprises a compound of Formula Ib, PEG 300, sodium hydroxide, and water. In some embodiments, the solution comprises a sodium salt of the compound of Formula Ib, PEG 300, sodium hydroxide, and water.

In some embodiments, the solution comprises a compound of Formula Ia or Ib, PEG 300, sodium hydroxide, and water. In some embodiments, the concentration of the compound of Formula Ia or Ib in the solution comprising a compound of Formula Ia or Ib, PEG 300, sodium hydroxide, and water is about 50 mg/ml to about 600 mg/ml. In some embodiments, the concentration of the compound of Formula Ia or Ib in the solution comprising a compound of Formula Ia or Ib, PEG 300, sodium hydroxide, and water is about 50 mg/ml to about 500 mg/ml. In some embodiments, the concentration of the compound of Formula Ia or Ib in the solution comprising a compound of Formula Ia or Ib, PEG 300, sodium hydroxide, and water is about 50 mg/ml to about 400 mg/ml. In some embodiments, the concentration of the compound of Formula Ia or Ib in the solution comprising a compound of Formula Ia or Ib, PEG 300, sodium hydroxide, and water is about 50 mg/ml to about 300 mg/ml. In some embodiments, the concentration of the compound of Formula Ia or Ib in the solution comprising a compound of Formula Ia or Ib, PEG 300, sodium hydroxide, and water is about 75 mg/ml to about 300 mg/ml. In some embodiments, the concentration of the compound of Formula Ia or Ib in the solution comprising a compound of Formula Ia or Ib, PEG 300, sodium hydroxide, and water is about 50 mg/ml. In some embodiments, the concentration of the compound of Formula Ia or Ib in the solution comprising a compound of Formula Ia or Ib, PEG 300, sodium hydroxide, and water is about 75 mg/ml. In some embodiments, the concentration of the compound of Formula Ia or Ib in the solution comprising a compound of Formula Ia or Ib, PEG 300, sodium hydroxide, and water is about 100 mg/ml. In some embodiments, the concentration of the compound of Formula Ia or Ib in the solution comprising a compound of Formula Ia or Ib, PEG 300, sodium hydroxide, and water is about 125 mg/ml. In some embodiments, the concentration of the compound of Formula Ia or Ib in the solution comprising a compound of Formula Ia or Ib, PEG 300, sodium hydroxide, and water is about 150 mg/ml. In some embodiments, the concentration of the compound of Formula Ia or Ib in the solution comprising a compound of Formula Ia or Ib, PEG 300, sodium hydroxide, and water is about 175 mg/ml. In some embodiments, the concentration of the compound of Formula Ia or Ib in the solution comprising a compound of Formula Ia or Ib, PEG 300, sodium hydroxide, and water is about 200 mg/ml. In some embodiments, the concentration of the compound of Formula Ia or Ib in the solution comprising a compound of Formula Ia or Ib, PEG 300, sodium hydroxide, and water is about 225 mg/ml. In some embodiments, the concentration of the compound of Formula Ia or Ib in the solution comprising a compound of Formula Ia or Ib, PEG 300, sodium hydroxide, and water is about 250 mg/ml. In some embodiments, the concentration of the compound of Formula Ia or Ib in the solution comprising a compound of Formula Ia or Ib, PEG 300, sodium hydroxide, and water is about 275 mg/ml. In some embodiments, the concentration of the compound of Formula Ia or Ib in the solution comprising a compound of Formula Ia or Ib, PEG 300, sodium hydroxide, and water is about 300 mg/ml. In some embodiments, the concentration of the compound of Formula Ia or Ib in the solution comprising a compound of Formula Ia or Ib, PEG 300, sodium hydroxide, and water is about 325 mg/ml. In some embodiments, the concentration of the compound of Formula Ia or Ib in the solution comprising a compound of Formula Ia or Ib, PEG 300, sodium hydroxide, and water is about 350 mg/ml. In some embodiments, the concentration of the compound of Formula Ia or Ib in the solution comprising a compound of Formula Ia or Ib, PEG 300, sodium hydroxide, and water is about 375 mg/ml. In some embodiments, the concentration of the compound of Formula Ia or Ib in the solution comprising a compound of Formula Ia or Ib, PEG 300, sodium hydroxide, and water is about 400 mg/ml. In some embodiments, the concentration of the compound of Formula Ia or Ib in the solution comprising a compound of Formula Ia or Ib, PEG 300, sodium hydroxide, and water is about 425 mg/ml. In some embodiments, the concentration of the compound of Formula Ia or Ib in the solution comprising a compound of Formula Ia or Ib, PEG 300, sodium hydroxide, and water is about 450 mg/ml. In some embodiments, the concentration of the compound of Formula Ia or Ib in the solution comprising a compound of Formula Ia or Ib, PEG 300, sodium hydroxide, and water is about 475 mg/ml. In some embodiments, the concentration of the compound of Formula Ia or Ib in the solution comprising a compound of Formula Ia or Ib, PEG 300, sodium hydroxide, and water is about 500 mg/ml. In some embodiments, the concentration of the compound of Formula Ia or Ib in the solution comprising a compound of Formula Ia or Ib, PEG 300, sodium hydroxide, and water is about 550 mg/ml. In some embodiments, the concentration of the compound of Formula Ia or Ib in the solution comprising a compound of Formula Ia or Ib, PEG 300, sodium hydroxide, and water is about 600 mg/ml.

In some embodiments, the amount of water in the solution comprising a compound of Formula Ia or Ib, PEG 300, sodium hydroxide, and water is about 10 w/w % to about 40 w/w %. In some embodiments, the amount of water in the solution comprising a compound of Formula Ia or Ib, PEG 300, sodium hydroxide, and water is about 15 w/w % to about 35 w/w %. In some embodiments, the amount of water in the solution comprising a compound of Formula Ia or Ib, PEG 300, sodium hydroxide, and water is about 20 w/w % to about 30 w/w %. In some embodiments, the amount of water in the solution comprising a compound of Formula Ia or Ib, PEG 300, sodium hydroxide, and water is about 21 w/w % to about 29 w/w %. In some embodiments, the amount of water in the solution comprising a compound of Formula Ia or Ib, PEG 300, sodium hydroxide, and water is about 23.2 w/w % to about 27.9 w/w %. In some embodiments, the amount of water in the solution comprising a compound of Formula Ia or Ib, PEG 300, sodium hydroxide, and water is about 23.2 w/w % to about 27.92 w/w %. In some embodiments, the amount of water in the solution comprising a compound of Formula Ia or Ib, PEG 300, sodium hydroxide, and water is about 22.0 w/w %, about 22.1 w/w %, about 22.2 w/w %, about 22.3 w/w %, about 22.4 w/w %, about 22.5 w/w %, about 22.6 w/w %, about 22.7 w/w %, about 22.8 w/w %, about 22.9 w/w %, about 23.0 w/w %, about 23.1 w/w %, about 23.2 w/w %, about 23.3 w/w %, about 23.4 w/w %, about 23.5 w/w %, about 23.6 w/w %, about 23.7 w/w %, about 23.8 w/w %, about 23.9 w/w %, about 24.0 w/w %, about 24.1 w/w %, about 24.2 w/w %, about 24.3 w/w %, about 24.4 w/w %, about 24.5 w/w %, about 24.6 w/w %, about 24.7 w/w %, about 24.8 w/w %, about 24.9 w/w %, about 25.0 w/w %, about 25.1 w/w %, about 25.2 w/w %, about 25.3 w/w %, about 25.4 w/w %, about 25.5 w/w %, about 25.6 w/w %, about 25.7 w/w %, about 25.8 w/w %, about 25.9 w/w %, about 26.0 w/w %, about 26.1 w/w %, about 26.2 w/w %, about 26.3 w/w %, about 26.4 w/w %, about 26.5 w/w %, about 26.6 w/w %, about 26.7 w/w %, about 26.8 w/w %, about 26.9 w/w %, about 27.0 w/w %, about 27.1 w/w %, about 27.2 w/w %, about 27.3 w/w %, about 27.4 w/w %, about 27.5 w/w %, about 27.6 w/w %, about 27.7 w/w %, about 27.8 w/w %, about 27.9 w/w %, about 28.0 w/w %, about 28.1 w/w %, about 28.2 w/w %, about 28.3 w/w %, about 28.4 w/w %, about 28.5 w/w %, about 28.6 w/w %, about 28.7 w/w %, about 28.8 w/w %, about 28.9 w/w %, or about 29.0 w/w %. In some embodiments, the amount of water in the solution comprising a compound of Formula Ia or Ib, PEG 300, sodium hydroxide, and water is about 23.2 w/w %. In some embodiments, the amount of water in the solution comprising a compound of Formula Ia or Ib, PEG 300, sodium hydroxide, and water is about 27.9 w/w %. In some embodiments, the amount of water in the solution comprising a compound of Formula Ia or Ib, PEG 300, sodium hydroxide, and water is about 27.92 w/w %.

In some embodiments, the amount of PEG 300 in the solution comprising compound of Formula Ia or Ib, PEG 300, sodium hydroxide, and water is about 35 w/w % to about 75 w/w %. In some embodiments, the amount of PEG 300 in the solution comprising a compound of Formula Ia or Ib, PEG 300, sodium hydroxide, and water is about 45 w/w % to about 65 w/w %. In some embodiments, the amount of PEG 300 in the solution comprising a compound of Formula Ia or Ib, PEG 300, sodium hydroxide, and water is about 48 w/w % to about 60 w/w %. In some embodiments, the amount of PEG 300 in the solution comprising a compound of Formula Ia or Ib, PEG 300, sodium hydroxide, and water is about 49 w/w % to about 59 w/w %. In some embodiments, the amount of PEG 300 in the solution comprising a compound of Formula Ia or Ib, PEG 300, sodium hydroxide, and water is about 50.0 w/w % to about 58.0 w/w %. In some embodiments, the amount of PEG 300 in the solution comprising a compound of Formula Ia or Ib, PEG 300, sodium hydroxide, and water is about 50.0 w/w % to about 58.04 w/w %. In some embodiments, the amount of PEG 300 in the solution comprising a compound of Formula Ia or Ib, PEG 300, sodium hydroxide, and water is about 45 w/w %, about 46 w/w %, about 47 w/w %, about 48 w/w %, about 49 w/w %, about 50 w/w %, about 51 w/w %, about 52 w/w %, about 53 w/w %, about 54 w/w %, about 55 w/w %, about 56 w/w %, about 57 w/w %, about 58 w/w %, about 59 w/w %, about 60 w/w %, about 61 w/w %, about 62 w/w %, about 63 w/w %, about 64 w/w %, or about 65 w/w %. In some embodiments, the amount of PEG 300 in the solution comprising a compound of Formula Ia or Ib, PEG 300, sodium hydroxide, and water is about 50.0 w/w %. In some embodiments, the amount of PEG 300 in the solution comprising a compound of Formula Ia or Ib, PEG 300, sodium hydroxide, and water is about 58.0 w/w %. In some embodiments, the amount of PEG 300 in the solution comprising a compound of Formula Ia or Ib, PEG 300, sodium hydroxide, and water is about 58.04 w/w %.

In some embodiments, the amount of the compound of Formula Ia or Ib in the solution comprising a compound of Formula Ia or Ib, PEG 300, sodium hydroxide, and water is about 5 w/w % to about 35 w/w %. In some embodiments, the amount of the compound of Formula Ia or Ib in the solution comprising a compound of Formula Ia or Ib, PEG 300, sodium hydroxide, and water is about 10 w/w % to about 30 w/w %. In some embodiments, the amount of the compound of Formula Ia or Ib in the solution comprising a compound of Formula Ia or Ib, PEG 300, sodium hydroxide, and water is about 11 w/w % to about 28 w/w %. In some embodiments, the amount of the compound of Formula Ia or Ib in the solution comprising a compound of Formula Ia or Ib, PEG 300, sodium hydroxide, and water is about 13 w/w % to about 27 w/w %. In some embodiments, the amount of the compound of Formula Ia or Ib in the solution comprising a compound of Formula Ia or Ib, PEG 300, sodium hydroxide, and water is about 13.5 w/w % to about 25.7 w/w %. In some embodiments, the amount of the compound of Formula Ia or Ib in the solution comprising a compound of Formula Ia or Ib, PEG 300, sodium hydroxide, and water is about 13.47 w/w % to about 25.7 w/w %. In some embodiments, the amount of the compound of Formula Ia or Ib in the solution comprising a compound of Formula Ia or Ib, PEG 300, sodium hydroxide, and water is about 13.0 w/w %, about 13.1 w/w %, about 13.2 w/w %, about 13.3 w/w %, about 13.4 w/w %, about 13.5 w/w %, about 13.6 w/w %, about 13.7 w/w %, about 13.8 w/w %, about 13.9 w/w %, about 14.0 w/w %, about 14.1 w/w %, about 14.2 w/w %, about 14.3 w/w %, about 14.4 w/w %, about 14.5 w/w %, about 14.6 w/w %, about 14.7 w/w %, about 14.8 w/w %, about 14.9 w/w %, about 15.0 w/w %, about 15.1 w/w %, about 15.2 w/w %, about 15.3 w/w %, about 15.4 w/w %, about 15.5 w/w %, about 15.6 w/w %, about 15.7 w/w %, about 15.8 w/w %, about 15.9 w/w %, about 16.0 w/w %, about 16.1 w/w %, about 16.2 w/w %, about 16.3 w/w %, about 16.4 w/w %, about 16.5 w/w %, about 16.6 w/w %, about 16.7 w/w %, about 16.8 w/w %, about 16.9 w/w %, about 17.0 w/w %, about 17.1 w/w %, about 17.2 w/w %, about 17.3 w/w %, about 17.4 w/w %, about 17.5 w/w %, about 17.6 w/w %, about 17.7 w/w %, about 17.8 w/w %, about 17.9 w/w %, about 18.0 w/w %, about 18.1 w/w %, about 18.2 w/w %, about 18.3 w/w %, about 18.4 w/w %, about 18.5 w/w %, about 18.6 w/w %, about 18.7 w/w %, about 18.8 w/w %, about 18.9 w/w %, about 19.0 w/w %, about 19.1 w/w %, about 19.2 w/w %, about 19.3 w/w %, about 19.4 w/w %, about 19.5 w/w %, about 19.6 w/w %, about 19.7 w/w %, about 19.8 w/w %, about 19.9 w/w %, about 20.0 w/w %, about 21.1 w/w %, about 21.2 w/w %, about 21.3 w/w %, about 21.4 w/w %, about 21.5 w/w %, about 21.6 w/w %, about 21.7 w/w %, about 21.8 w/w %, about 21.9 w/w %, about 22.0 w/w %, about 22.1 w/w %, about 22.2 w/w %, about 22.3 w/w %, about 22.4 w/w %, about 22.5 w/w %, about 22.6 w/w %, about 22.7 w/w %, about 22.8 w/w %, about 22.9 w/w %, about 23.0 w/w %, about 23.1 w/w %, about 23.2 w/w %, about 23.3 w/w %, about 23.4 w/w %, about 23.5 w/w %, about 23.6 w/w %, about 23.7 w/w %, about 23.8 w/w %, about 23.9 w/w %, about 24.0 w/w %, about 24.1 w/w %, about 24.2 w/w %, about 24.3 w/w %, about 24.4 w/w %, about 24.5 w/w %, about 24.6 w/w %, about 24.7 w/w %, about 24.8 w/w %, about 24.9 w/w %, about 25.0 w/w %, about 25.1 w/w %, about 25.2 w/w %, about 25.3 w/w %, about 25.4 w/w %, about 25.5 w/w %, about 25.6 w/w %, about 25.7 w/w %, about 25.8 w/w %, about 25.9 w/w %, about 26.0 w/w %, about 26.1 w/w %, about 26.2 w/w %, about 26.3 w/w %, about 26.4 w/w %, about 26.5 w/w %, about 26.6 w/w %, about 26.7 w/w %, about 26.8 w/w %, about 26.9 w/w %, about 27.0 w/w %, about 27.1 w/w %, about 27.2 w/w %, about 27.3 w/w %, about 27.4 w/w %, about 27.5 w/w %, about 27.6 w/w %, about 27.7 w/w %, about 27.8 w/w %, about 27.9 w/w %, about 28.0 w/w %, about 28.1 w/w %, about 28.2 w/w %, about 28.3 w/w %, about 28.4 w/w %, about 28.5 w/w %, about 28.6 w/w %, about 28.7 w/w %, about 28.8 w/w %, about 28.9 w/w %, or about 29.0 w/w %. In some embodiments, the amount of the compound of Formula Ia or Ib in the solution comprising a compound of Formula Ia or Ib, PEG 300, sodium hydroxide, and water is about 13.47 w/w %. In some embodiments, the amount of the compound of Formula Ia or Ib in the solution comprising a compound of Formula Ia or Ib, PEG 300, sodium hydroxide, and water is about 13.5 w/w %. In some embodiments, the amount of the compound of Formula Ia or Ib in the solution comprising a compound of Formula Ia or Ib, PEG 300, sodium hydroxide, and water is about 25.7 w/w %.

In some embodiments, the amount of sodium hydroxide in the solution comprising a compound of Formula Ia or Ib, PEG 300, sodium hydroxide, and water is about 0.05 w/w % to about 2 w/w %. In some embodiments, the amount of sodium hydroxide in the solution comprising a compound of Formula Ia or Ib, PEG 300, sodium hydroxide, and water is about 0.1 w/w % to about 1.5 w/w %. In some embodiments, the amount of sodium hydroxide in the solution comprising a compound of Formula Ia or Ib, PEG 300, sodium hydroxide, and water is about 0.3 w/w % to about 1.3 w/w %. In some embodiments, the amount of sodium hydroxide in the solution comprising a compound of Formula Ia or Ib, PEG 300, sodium hydroxide, and water is about 0.5 w/w % to about 1.2 w/w %. In some embodiments, the amount of sodium hydroxide in the solution comprising a compound of Formula Ia or Ib, PEG 300, sodium hydroxide, and water is about 0.6 w/w % to about 1.1 w/w %. In some embodiments, the amount of sodium hydroxide in the solution comprising a compound of Formula Ia or Ib, PEG 300, sodium hydroxide, and water is about 0.58 w/w % to about 1.1 w/w %. In some embodiments, the amount of sodium hydroxide in the solution comprising a compound of Formula Ia or Ib, PEG 300, sodium hydroxide, and water is about 0.1 w/w %, about 0.2 w/w %, about 0.3 w/w %, about 0.4 w/w %, about 0.5 w/w %, about 0.6 w/w %, about 0.7 w/w %, about 0.8 w/w %, about 0.9 w/w %, about 1.0 w/w %, about 1.1 w/w %, about 1.2 w/w %, about 1.3 w/w %, about 1.4 w/w %, about 1.5 w/w %, about 1.6 w/w %, about 1.7 w/w %, about 1.8 w/w %, about 1.9 w/w %, or about 2.0 w/w %. In some embodiments, the amount of sodium hydroxide in the solution comprising a compound of Formula Ia or Ib, PEG 300, sodium hydroxide, and water is about 0.58 w/w %. In some embodiments, the amount of sodium hydroxide in the solution comprising a compound of Formula Ia or Ib, PEG 300, sodium hydroxide, and water is about 0.6 w/w %. In some embodiments, the amount of sodium hydroxide in the solution comprising a compound of Formula Ia or Ib, PEG 300, sodium hydroxide, and water is about 1.1 w/w %.

In some embodiments, the solution comprises about 10 w/w % to about 40 w/w % water, about 35 w/w % to about 75 w/w % PEG 300, about 5 w/w % to about 35 w/w % of a compound of Formula Ia or Ib, and about 0.05 w/w % to about 2 w/w % of sodium hydroxide. In some embodiments, the solution comprises about 15 w/w % to about 35 w/w % water, about 45 w/w % to about 65 w/w % PEG 300, about 10 w/w % to about 30 w/w % of a compound of Formula Ia or Ib, and about 0.1 w/w % to about 1.5 w/w % of sodium hydroxide. In some embodiments, the solution comprises about 20 w/w % to about 30 w/w % water, about 48 w/w % to about 60 w/w % PEG 300, about 11 w/w % to about 28 w/w % of a compound of Formula Ia or Ib, and about 0.3 w/w % to about 1.3 w/w % of sodium hydroxide. In some embodiments, the solution comprises about 21 w/w % to about 29 w/w % water, about 49 w/w % to about 59 w/w % PEG 300, about 13 w/w % to about 27 w/w % of a compound of Formula Ia or Ib, and about 0.5 w/w % to about 1.2 w/w % of sodium hydroxide. In some embodiments, the solution comprises about 23.2 w/w % to about 27.9 w/w % water, about 50 w/w % to about 58 w/w % PEG 300, about 13.5 w/w % to about 25.7 w/w % of a compound of Formula Ia or Ib, and about 0.6 w/w % to about 1.1 w/w % of sodium hydroxide. In some embodiments, the solution comprises about 23.2 w/w % to about 27.92 w/w % water, about 50.0 w/w % to about 58.04 w/w % PEG 300, about 13.47 w/w % to about 25.7 w/w % of a compound of Formula Ia or Ib, and about 0.58 w/w % to about 1.1 w/w % of sodium hydroxide.

In some embodiments, the solution comprises about 27.9 w/w % water, about 58 w/w % PEG 300, about 13.5 w/w % of a compound of Formula Ia or Ib, and about 0.6 w/w % of sodium hydroxide. In some embodiments, the solution comprises about 27.92 w/w % water, about 58.04 w/w % PEG 300, about 13.47 w/w % of a compound of Formula Ia or Ib, and about 0.58 w/w % of sodium hydroxide. In some embodiments, the solution comprises about 27.9 w/w % water, about 58 w/w % PEG 300, about 13.5 w/w % of a compound of Formula Ib, and about 0.6 w/w % of sodium hydroxide. In some embodiments, the solution comprises about 27.92 w/w % water, about 58.04 w/w % PEG 300, about 13.47 w/w % of a compound of Formula Ib, and about 0.58 w/w % of sodium hydroxide.

In some embodiments, the solution comprises about 23.2 w/w % water, about 50.0 w/w % PEG 300, about 25.7 w/w % of a compound of Formula Ia or Ib, and about 1.1 w/w % of sodium hydroxide. In some embodiments, the solution comprises about 23.2 w/w % water, about 50.0 w/w % PEG 300, about 25.7 w/w % of a compound of Formula Ib, and about 1.1 w/w % of sodium hydroxide.

In some embodiments, the compound of Formula Ia or Ib becomes ionized in situ to a sodium salt of the compound of Formula Ia or Ib in the presence of sodium hydroxide in the solution comprising a compound of Formula Ia or Ib, PEG 300, sodium hydroxide, and water. Thus, in some embodiments, the compound of Formula Ia or Ib is present as a sodium salt of the compound of Formula Ia or Ib in the solution comprising a compound of Formula Ia or Ib, PEG 300, sodium hydroxide, and water.

In some embodiments, the pharmaceutical compositions comprise a compound of Formula Ia or Ib, or a pharmaceutically acceptable salt thereof, a poloxamer, and a pharmaceutically acceptable excipient. In some embodiments, the pharmaceutical compositions comprise a compound of Formula Ia or Ib, or a pharmaceutically acceptable salt thereof, poloxamer 188, and a pharmaceutically acceptable excipient. In some embodiments, the pharmaceutical compositions are solutions that comprise a compound of Formula Ia or Ib, or a pharmaceutically acceptable salt thereof, a poloxamer, and a pharmaceutically acceptable excipient. In some embodiments, the pharmaceutical compositions are solutions that comprise a compound of Formula Ia or Ib, or a pharmaceutically acceptable salt thereof, poloxamer 188, and a pharmaceutically acceptable excipient.

In some embodiments, the solution comprises a compound of Formula Ia or Ib, or a pharmaceutically acceptable salt thereof, PEG 300, sodium hydroxide, poloxamer 188, and water. In some embodiments, the solution comprises a compound of Formula Ia or a pharmaceutically acceptable salt thereof, PEG 300, sodium hydroxide, poloxamer 188, and water. In some embodiments, the solution comprises a compound of Formula Ib, or a pharmaceutically acceptable salt thereof, PEG 300, sodium hydroxide, poloxamer 188, and water. In some embodiments, the solution comprises a sodium salt of the compound of Formula Ia, PEG 300, sodium hydroxide, poloxamer 188, and water. In some embodiments, the solution comprises a sodium salt of the compound of Formula Ib, PEG 300, sodium hydroxide, poloxamer 188, and water.

In some embodiments, the solution comprises a compound of Formula Ia or Ib, PEG 300, sodium hydroxide, poloxamer 188, and water. In some embodiments, the concentration of the compound of Formula Ia or Ib in the solution comprising a compound of Formula Ia or Ib, PEG 300, sodium hydroxide, poloxamer 188, and water is about 50 mg/ml to about 600 mg/ml. In some embodiments, the concentration of the compound of Formula Ia or Ib in the solution comprising a compound of Formula Ia or Ib, PEG 300, sodium hydroxide, poloxamer 188, and water is about 50 mg/ml to about 500 mg/ml. In some embodiments, the concentration of the compound of Formula Ia or Ib in the solution comprising a compound of Formula Ia or Ib, PEG 300, sodium hydroxide, poloxamer 188, and water is about 50 mg/ml to about 400 mg/ml. In some embodiments, the concentration of the compound of Formula Ia or Ib in the solution comprising a compound of Formula Ia or Ib, PEG 300, sodium hydroxide, poloxamer 188, and water is about 50 mg/ml to about 300 mg/ml. In some embodiments, the concentration of the compound of Formula Ia or Ib in the solution comprising a compound of Formula Ia or Ib, PEG 300, sodium hydroxide, poloxamer 188, and water is about 75 mg/ml to about 300 mg/ml. In some embodiments, the concentration of the compound of Formula Ia or Ib in the solution comprising a compound of Formula Ia or Ib, PEG 300, sodium hydroxide, poloxamer 188, and water is about 50 mg/ml. In some embodiments, the concentration of the compound of Formula Ia or Ib in the solution comprising a compound of Formula Ia or Ib, PEG 300, sodium hydroxide, poloxamer 188, and water is about 75 mg/ml. In some embodiments, the concentration of the compound of Formula Ia or Ib in the solution comprising a compound of Formula Ia or Ib, PEG 300, sodium hydroxide, poloxamer 188, and water is about 100 mg/ml. In some embodiments, the concentration of the compound of Formula Ia or Ib in the solution comprising a compound of Formula Ia or Ib, PEG 300, sodium hydroxide, poloxamer 188, and water is about 125 mg/ml. In some embodiments, the concentration of the compound of Formula Ia or Ib in the solution comprising a compound of Formula Ia or Ib, PEG 300, sodium hydroxide, poloxamer 188, and water is about 150 mg/ml. In some embodiments, the concentration of the compound of Formula Ia or Ib in the solution comprising a compound of Formula Ia or Ib, PEG 300, sodium hydroxide, poloxamer 188, and water is about 175 mg/ml. In some embodiments, the concentration of the compound of Formula Ia or Ib in the solution comprising a compound of Formula Ia or Ib, PEG 300, sodium hydroxide, poloxamer 188, and water is about 200 mg/ml. In some embodiments, the concentration of the compound of Formula Ia or Ib in the solution comprising a compound of Formula Ia or Ib, PEG 300, sodium hydroxide, poloxamer 188, and water is about 225 mg/ml. In some embodiments, the concentration of the compound of Formula Ia or Ib in the solution comprising a compound of Formula Ia or Ib, PEG 300, sodium hydroxide, poloxamer 188, and water is about 250 mg/ml. In some embodiments, the concentration of the compound of Formula Ia or Ib in the solution comprising a compound of Formula Ia or Ib, PEG 300, sodium hydroxide, poloxamer 188, and water is about 275 mg/ml. In some embodiments, the concentration of the compound of Formula Ia or Ib in the solution comprising a compound of Formula Ia or Ib, PEG 300, sodium hydroxide, poloxamer 188, and water is about 300 mg/ml. In some embodiments, the concentration of the compound of Formula Ia or Ib in the solution comprising a compound of Formula Ia or Ib, PEG 300, sodium hydroxide, poloxamer 188, and water is about 325 mg/ml. In some embodiments, the concentration of the compound of Formula Ia or Ib in the solution comprising a compound of Formula Ia or Ib, PEG 300, sodium hydroxide, poloxamer 188, and water is about 350 mg/ml. In some embodiments, the concentration of the compound of Formula Ia or Ib in the solution comprising a compound of Formula Ia or Ib, PEG 300, sodium hydroxide, poloxamer 188, and water is about 375 mg/ml. In some embodiments, the concentration of the compound of Formula Ia or Ib in the solution comprising a compound of Formula Ia or Ib, PEG 300, sodium hydroxide, poloxamer 188, and water is about 400 mg/ml. In some embodiments, the concentration of the compound of Formula Ia or Ib in the solution comprising a compound of Formula Ia or Ib, PEG 300, sodium hydroxide, poloxamer 188, and water is about 425 mg/ml. In some embodiments, the concentration of the compound of Formula Ia or Ib in the solution comprising a compound of Formula Ia or Ib, PEG 300, sodium hydroxide, poloxamer 188, and water is about 450 mg/ml. In some embodiments, the concentration of the compound of Formula Ia or Ib in the solution comprising a compound of Formula Ia or Ib, PEG 300, sodium hydroxide, poloxamer 188, and water is about 475 mg/ml. In some embodiments, the concentration of the compound of Formula Ia or Ib in the solution comprising a compound of Formula Ia or Ib, PEG 300, sodium hydroxide, poloxamer 188, and water is about 500 mg/ml. In some embodiments, the concentration of the compound of Formula Ia or Ib in the solution comprising a compound of Formula Ia or Ib, PEG 300, sodium hydroxide, poloxamer 188, and water is about 550 mg/ml. In some embodiments, the concentration of the compound of Formula Ia or Ib in the solution comprising a compound of Formula Ia or Ib, PEG 300, sodium hydroxide, poloxamer 188, and water is about 600 mg/ml.

In some embodiments, the amount of water in the solution comprising a compound of Formula Ia or Ib, PEG 300, sodium hydroxide, poloxamer 188, and water is about 10 w/w % to about 45 w/w %. In some embodiments, the amount of water in the solution comprising a of Formula Ia or Ib, PEG 300, sodium hydroxide, poloxamer 188, and water is about 15 w/w % to about 35 w/w %. In some embodiments, the amount of water in the solution comprising a compound of Formula Ia or Ib, PEG 300, sodium hydroxide, poloxamer 188, and water is about 20 w/w % to about 35 w/w %. In some embodiments, the amount of water in the solution comprising a compound of Formula Ia or Ib, PEG 300, sodium hydroxide, poloxamer 188, and water is about 20 w/w % to about 31 w/w %. In some embodiments, the amount of water in the solution comprising a compound of Formula Ia or Ib, PEG 300, sodium hydroxide, poloxamer 188, and water is about 22 w/w % to about 30.1 w/w %. In some embodiments, the amount of water in the solution comprising a compound of Formula Ia or Ib, PEG 300, sodium hydroxide, poloxamer 188, and water is about 21.97 w/w % to about 30.07 w/w %. In some embodiments, the amount of water in the solution comprising a compound of Formula Ia or Ib, PEG 300, sodium hydroxide, poloxamer 188, and water is about 19.0 w/w %, about 19.1 w/w %, about 19.2 w/w %, about 19.3 w/w %, about 19.4 w/w %, about 19.5 w/w %, about 19.6 w/w %, about 19.7 w/w %, about 19.8 w/w %, about 19.9 w/w %, about 20.0 w/w %, about 20.1 w/w %, about 20.2 w/w %, about 20.3 w/w %, about 20.4 w/w %, about 20.5 w/w %, about 20.6 w/w %, about 20.7 w/w %, about 20.8 w/w %, about 20.9 w/w %, about 21.0 w/w %, about 21.1 w/w %, about 21.2 w/w %, about 21.3 w/w %, about 21.4 w/w %, about 21.5 w/w %, about 21.6 w/w %, about 21.7 w/w %, about 21.8 w/w %, about 21.9 w/w %, about 22.0 w/w %, about 22.1 w/w %, about 22.2 w/w %, about 22.3 w/w %, about 22.4 w/w %, about 22.5 w/w %, about 22.6 w/w %, about 22.7 w/w %, about 22.8 w/w %, about 22.9 w/w %, about 23.0 w/w %, about 23.1 w/w %, about 23.2 w/w %, about 23.3 w/w %, about 23.4 w/w %, about 23.5 w/w %, about 23.6 w/w %, about 23.7 w/w %, about 23.8 w/w %, about 23.9 w/w %, about 24.0 w/w %, about 24.1 w/w %, about 24.2 w/w %, about 24.3 w/w %, about 24.4 w/w %, about 24.5 w/w %, about 24.6 w/w %, about 24.7 w/w %, about 24.8 w/w %, about 24.9 w/w %, about 25.0 w/w %, about 25.1 w/w %, about 25.2 w/w %, about 25.3 w/w %, about 25.4 w/w %, about 25.5 w/w %, about 25.6 w/w %, about 25.7 w/w %, about 25.8 w/w %, about 25.9 w/w %, about 26.0 w/w %, about 26.1 w/w %, about 26.2 w/w %, about 26.3 w/w %, about 26.4 w/w %, about 26.5 w/w %, about 26.6 w/w %, about 26.7 w/w %, about 26.8 w/w %, about 26.9 w/w %, about 27.0 w/w %, about 27.1 w/w %, about 27.2 w/w %, about 27.3 w/w %, about 27.4 w/w %, about 27.5 w/w %, about 27.6 w/w %, about 27.7 w/w %, about 27.8 w/w %, about 27.9 w/w %, about 28.0 w/w %, about 28.1 w/w %, about 28.2 w/w %, about 28.3 w/w %, about 28.4 w/w %, about 28.5 w/w %, about 28.6 w/w %, about 28.7 w/w %, about 28.8 w/w %, about 28.9 w/w %, about 29.0 w/w %, about 29.1 w/w %, about 29.2 w/w %, about 29.3 w/w %, about 29.4 w/w %, about 29.5 w/w %, about 29.6 w/w %, about 29.7 w/w %, about 29.8 w/w %, about 29.9 w/w %, about 30.0 w/w %, about 30.1 w/w %, about 30.2 w/w %, about 30.3 w/w %, about 30.4 w/w %, about 30.5 w/w %, about 30.6 w/w %, about 30.7 w/w %, about 30.8 w/w %, about 30.9 w/w %, about 31.0 w/w %, about 31.1 w/w %, about 31.2 w/w %, about 31.3 w/w %, about 31.4 w/w %, about 31.5 w/w %, about 31.6 w/w %, about 31.7 w/w %, about 31.8 w/w %, about 31.9 w/w %, about 32.0 w/w %, about 32.1 w/w %, about 32.2 w/w %, about 32.3 w/w %, about 32.4 w/w %, about 32.5 w/w %, about 32.6 w/w %, about 32.7 w/w %, about 32.8 w/w %, about 32.9 w/w %, or about 33.0 w/w %. In some embodiments, the amount of water in the solution comprising a compound of Formula Ia or Ib, PEG 300, sodium hydroxide, poloxamer 188, and water is about 21.97 w/w %. In some embodiments, the amount of water in the solution comprising a compound of Formula Ia or Ib, PEG 300, sodium hydroxide, poloxamer 188, and water is about 22 w/w %. In some embodiments, the amount of water in the solution comprising a compound of Formula Ia or Ib, PEG 300, sodium hydroxide, poloxamer 188, and water is about 30.07 w/w %. In some embodiments, the amount of water in the solution comprising a compound of Formula Ia or Ib, PEG 300, sodium hydroxide, poloxamer 188, and water is about 30.1 w/w %.

In some embodiments, the amount of PEG 300 in the solution comprising a compound of Formula Ia or Ib, PEG 300, sodium hydroxide, poloxamer 188, and water is about 30 w/w % to about 85 w/w %. In some embodiments, the amount of PEG 300 in the solution comprising a compound of Formula Ia or Ib, PEG 300, sodium hydroxide, poloxamer 188, and water is about 35 w/w % to about 75 w/w %. In some embodiments, the amount of PEG 300 in the solution comprising a compound of Formula Ia or Ib, PEG 300, sodium hydroxide, poloxamer 188, and water is about 40 w/w % to about 70 w/w %. In some embodiments, the amount of PEG 300 in the solution comprising a compound of Formula Ia or Ib, PEG 300, sodium hydroxide, poloxamer 188, and water is about 45 w/w % to about 68 w/w %. In some embodiments, the amount of PEG 300 in the solution comprising a compound of Formula Ia or Ib, PEG 300, sodium hydroxide, poloxamer 188, and water is about 47.1 w/w % to about 64.4 w/w %. In some embodiments, the amount of PEG 300 in the solution comprising a compound of Formula Ia or Ib, PEG 300, sodium hydroxide, poloxamer 188, and water is about 47.05 w/w % to about 64.41 w/w %. In some embodiments, the amount of PEG 300 in the solution comprising a compound of Formula Ia or Ib, PEG 300, sodium hydroxide, poloxamer 188, and water is about 40 w/w %, about 41 w/w %, about 42 w/w %, about 43 w/w %, about 44 w/w %, about 45 w/w %, about 46 w/w %, about 47 w/w %, about 48 w/w %, about 49 w/w %, about 50 w/w %, about 51 w/w %, about 52 w/w %, about 53 w/w %, about 54 w/w %, about 55 w/w %, about 56 w/w %, about 57 w/w %, about 58 w/w %, about 59 w/w %, about 60 w/w %, about 61 w/w %, about 62 w/w %, about 63 w/w %, about 64 w/w %, about 65 w/w %, about 66 w/w %, about 67 w/w %, about 68 w/w %, about 69 w/w %, or about 70 w/w %. In some embodiments, the amount of PEG 300 in the solution comprising a compound of Formula Ia or Ib, PEG 300, sodium hydroxide, poloxamer 188, and water is about 47.05 w/w %. In some embodiments, the amount of PEG 300 in the solution comprising a compound of Formula Ia or Ib, PEG 300, sodium hydroxide, poloxamer 188, and water is about 47.1 w/w %. In some embodiments, the amount of PEG 300 in the solution comprising a compound of Formula Ia or Ib, PEG 300, sodium hydroxide, poloxamer 188, and water is about 64.4 w/w %. In some embodiments, the amount of PEG 300 in the solution comprising a compound of Formula Ia or Ib, PEG 300, sodium hydroxide, poloxamer 188, and water is about 64.41 w/w %.

In some embodiments, the amount of the compound of Formula Ia or Ib in the solution comprising a compound of Formula Ia or Ib, PEG 300, sodium hydroxide, poloxamer 188, and water is about 0.5 w/w % to about 40 w/w %. In some embodiments, the amount of the compound of Formula Ia or Ib in the solution comprising a compound of Formula Ia or Ib, PEG 300, sodium hydroxide, poloxamer 188, and water is about 1 w/w % to about 35 w/w %. In some embodiments, the amount of the compound of Formula Ia or Ib in the solution comprising a compound of Formula Ia or Ib, PEG 300, sodium hydroxide, poloxamer 188, and water is about 1 w/w % to about 30 w/w %. In some embodiments, the amount of the compound of Formula Ia or Ib in the solution comprising a compound of Formula Ia or Ib, PEG 300, sodium hydroxide, poloxamer 188, and water is about 3 w/w % to about 28 w/w %. In some embodiments, the amount of the compound of Formula Ia or Ib in the solution comprising a compound of Formula Ia or Ib, PEG 300, sodium hydroxide, poloxamer 188, and water is about 4 w/w % to about 26 w/w %. In some embodiments, the amount of the compound of Formula Ia or Ib in the solution comprising a compound of Formula Ia or Ib, PEG 300, sodium hydroxide, poloxamer 188, and water is about 4.6 w/w % to about 25.2 w/w %. In some embodiments, the amount of the compound of Formula Ia or Ib in the solution comprising a compound of Formula Ia or Ib, PEG 300, sodium hydroxide, poloxamer 188, and water is about 4.57 w/w % to about 25.21 w/w %. In some embodiments, the amount of the compound of Formula Ia or Ib in the solution comprising a compound of Formula Ia or Ib, PEG 300, sodium hydroxide, poloxamer 188, and water is about 3.0 w/w %, about 3.1 w/w %, about 3.2 w/w %, about 3.3 w/w %, about 3.4 w/w %, about 3.5 w/w %, about 3.6 w/w %, about 3.7 w/w %, about 3.8 w/w %, about 3.9 w/w %, about 4.0 w/w %, about 4.1 w/w %, about 4.2 w/w %, about 4.3 w/w %, about 4.4 w/w %, about 4.5 w/w %, about 4.6 w/w %, about 4.7 w/w %, about 4.8 w/w %, about 4.9 w/w %, about 5.0 w/w %, about 5.1 w/w %, about 5.2 w/w %, about 5.3 w/w %, about 5.4 w/w %, about 5.5 w/w %, about 5.6 w/w %, about 5.7 w/w %, about 5.8 w/w %, about 5.9 w/w %, about 6.0 w/w %, about 6.1 w/w %, about 6.2 w/w %, about 6.3 w/w %, about 6.4 w/w %, about 6.5 w/w %, about 6.6 w/w %, about 6.7 w/w %, about 6.8 w/w %, about 6.9 w/w %, about 7.0 w/w %, about 7.1 w/w %, about 7.2 w/w %, about 7.3 w/w %, about 7.4 w/w %, about 7.5 w/w %, about 7.6 w/w %, about 7.7 w/w %, about 7.8 w/w %, about 7.9 w/w %, about 8.0 w/w %, about 8.1 w/w %, about 8.2 w/w %, about 8.3 w/w %, about 8.4 w/w %, about 8.5 w/w %, about 8.6 w/w %, about 8.7 w/w %, about 8.8 w/w %, about 8.9 w/w %, about 9.0 w/w %, about 9.1 w/w %, about 9.2 w/w %, about 9.3 w/w %, about 9.4 w/w %, about 9.5 w/w %, about 9.6 w/w %, about 9.7 w/w %, about 9.8 w/w %, about 9.9 w/w %, about 10.0 w/w %, about 10.1 w/w %, about 10.2 w/w %, about 10.3 w/w %, about 10.4 w/w %, about 10.5 w/w %, about 10.6 w/w %, about 10.7 w/w %, about 10.8 w/w %, about 10.9 w/w %, about 11.0 w/w %, about 11.1 w/w %, about 11.2 w/w %, about 11.3 w/w %, about 11.4 w/w %, about 11.5 w/w %, about 11.6 w/w %, about 11.7 w/w %, about 11.8 w/w %, about 11.9 w/w %, about 12.0 w/w %, about 12.1 w/w %, about 12.2 w/w %, about 12.3 w/w %, about 12.4 w/w %, about 12.5 w/w %, about 12.6 w/w %, about 12.7 w/w %, about 12.8 w/w %, about 12.9 w/w %, about 13.0 w/w %, about 13.1 w/w %, about 13.2 w/w %, about 13.3 w/w %, about 13.4 w/w %, about 13.5 w/w %, about 13.6 w/w %, about 13.7 w/w %, about 13.8 w/w %, about 13.9 w/w %, about 14.0 w/w %, about 14.1 w/w %, about 14.2 w/w %, about 14.3 w/w %, about 14.4 w/w %, about 14.5 w/w %, about 14.6 w/w %, about 14.7 w/w %, about 14.8 w/w %, about 14.9 w/w %, about 15.0 w/w %, about 15.1 w/w %, about 15.2 w/w %, about 15.3 w/w %, about 15.4 w/w %, about 15.5 w/w %, about 15.6 w/w %, about 15.7 w/w %, about 15.8 w/w %, about 15.9 w/w %, about 16.0 w/w %, about 16.1 w/w %, about 16.2 w/w %, about 16.3 w/w %, about 16.4 w/w %, about 16.5 w/w %, about 16.6 w/w %, about 16.7 w/w %, about 16.8 w/w %, about 16.9 w/w %, about 17.0 w/w %, about 17.1 w/w %, about 17.2 w/w %, about 17.3 w/w %, about 17.4 w/w %, about 17.5 w/w %, about 17.6 w/w %, about 17.7 w/w %, about 17.8 w/w %, about 17.9 w/w %, about 18.0 w/w %, about 18.1 w/w %, about 18.2 w/w %, about 18.3 w/w %, about 18.4 w/w %, about 18.5 w/w %, about 18.6 w/w %, about 18.7 w/w %, about 18.8 w/w %, about 18.9 w/w %, about 19.0 w/w %, about 19.1 w/w %, about 19.2 w/w %, about 19.3 w/w %, about 19.4 w/w %, about 19.5 w/w %, about 19.6 w/w %, about 19.7 w/w %, about 19.8 w/w %, about 19.9 w/w %, about 20.0 w/w %, about 21.1 w/w %, about 21.2 w/w %, about 21.3 w/w %, about 21.4 w/w %, about 21.5 w/w %, about 21.6 w/w %, about 21.7 w/w %, about 21.8 w/w %, about 21.9 w/w %, about 22.0 w/w %, about 22.1 w/w %, about 22.2 w/w %, about 22.3 w/w %, about 22.4 w/w %, about 22.5 w/w %, about 22.6 w/w %, about 22.7 w/w %, about 22.8 w/w %, about 22.9 w/w %, about 23.0 w/w %, about 23.1 w/w %, about 23.2 w/w %, about 23.3 w/w %, about 23.4 w/w %, about 23.5 w/w %, about 23.6 w/w %, about 23.7 w/w %, about 23.8 w/w %, about 23.9 w/w %, about 24.0 w/w %, about 24.1 w/w %, about 24.2 w/w %, about 24.3 w/w %, about 24.4 w/w %, about 24.5 w/w %, about 24.6 w/w %, about 24.7 w/w %, about 24.8 w/w %, about 24.9 w/w %, about 25.0 w/w %, about 25.1 w/w %, about 25.2 w/w %, about 25.3 w/w %, about 25.4 w/w %, about 25.5 w/w %, about 25.6 w/w %, about 25.7 w/w %, about 25.8 w/w %, about 25.9 w/w %, about 26.0 w/w %, about 26.1 w/w %, about 26.2 w/w %, about 26.3 w/w %, about 26.4 w/w %, about 26.5 w/w %, about 26.6 w/w %, about 26.7 w/w %, about 26.8 w/w %, about 26.9 w/w %, about 27.0 w/w %, about 27.1 w/w %, about 27.2 w/w %, about 27.3 w/w %, about 27.4 w/w %, about 27.5 w/w %, about 27.6 w/w %, about 27.7 w/w %, about 27.8 w/w %, about 27.9 w/w %, about 28.0 w/w %, about 28.1 w/w %, about 28.2 w/w %, about 28.3 w/w %, about 28.4 w/w %, about 28.5 w/w %, about 28.6 w/w %, about 28.7 w/w %, about 28.8 w/w %, about 28.9 w/w %, or about 29.0 w/w %. In some embodiments, the amount of the compound of Formula Ia or Ib in the solution comprising a compound of Formula Ia or Ib, PEG 300, sodium hydroxide, poloxamer 188, and water is about 4.57 w/w %. In some embodiments, the amount of the compound of Formula Ia or Ib in the solution comprising a compound of Formula Ia or Ib, PEG 300, sodium hydroxide, poloxamer 188, and water is about 4.6 w/w %. In some embodiments, the amount of the compound of Formula Ia or Ib in the solution comprising a compound of Formula Ia or Ib, PEG 300, sodium hydroxide, poloxamer 188, and water is about 25.2 w/w %. In some embodiments, the amount of the compound of Formula Ia or Ib in the solution comprising a compound of Formula Ia or Ib, PEG 300, sodium hydroxide, poloxamer 188, and water is about 25.21 w/w %.

In some embodiments, the amount of sodium hydroxide in the solution comprising a compound of Formula Ia or Ib, PEG 300, sodium hydroxide, poloxamer 188, and water is about 0.01 w/w % to about 3.0 w/w %. In some embodiments, the amount of sodium hydroxide in the solution comprising a compound of Formula Ia or Ib, PEG 300, sodium hydroxide, poloxamer 188, and water is about 0.01 w/w % to about 2.0 w/w %. In some embodiments, the amount of sodium hydroxide in the solution comprising a compound of Formula Ia or Ib, PEG 300, sodium hydroxide, poloxamer 188, and water is about 0.05 w/w % to about 1.5 w/w %. In some embodiments, the amount of sodium hydroxide in the solution comprising a compound of Formula Ia or Ib, PEG 300, sodium hydroxide, poloxamer 188, and water is about 0.05 w/w % to about 1.2 w/w %. In some embodiments, the amount of sodium hydroxide in the solution comprising a compound of Formula Ia or Ib, PEG 300, sodium hydroxide, poloxamer 188, and water is about 0.1 w/w % to about 1.1 w/w %. In some embodiments, the amount of sodium hydroxide in the solution comprising a compound of Formula Ia or Ib, PEG 300, sodium hydroxide, poloxamer 188, and water is about 0.10 w/w % to about 1.08 w/w %. In some embodiments, the amount of sodium hydroxide in the solution comprising a compound of Formula Ia or Ib, PEG 300, sodium hydroxide, poloxamer 188, and water is about 0.01 w/w %, about 0.02 w/w %, about 0.03 w/w %, about 0.04 w/w %, about 0.05 w/w %, about 0.06 w/w %, about 0.07 w/w %, about 0.08 w/w %, about 0.09 w/w %, about 0.1 w/w %, about 0.2 w/w %, about 0.3 w/w %, about 0.4 w/w %, about 0.5 w/w %, about 0.6 w/w %, about 0.7 w/w %, about 0.8 w/w %, about 0.9 w/w %, about 1.0 w/w %, about 1.1 w/w %, about 1.2 w/w %, about 1.3 w/w %, about 1.4 w/w %, about 1.5 w/w %, about 1.6 w/w %, about 1.7 w/w %, about 1.8 w/w %, about 1.9 w/w %, or about 2.0 w/w %. In some embodiments, the amount of sodium hydroxide in the solution comprising a compound of Formula Ia or Ib, PEG 300, sodium hydroxide, poloxamer 188, and water is about 0.1 w/w %. In some embodiments, the amount of sodium hydroxide in the solution comprising a compound of Formula Ia or Ib, PEG 300, sodium hydroxide, poloxamer 188, and water is about 0.10 w/w %. In some embodiments, the amount of sodium hydroxide in the solution comprising a compound of Formula Ia or Ib, PEG 300, sodium hydroxide, poloxamer 188, and water is about 1.08 w/w %. In some embodiments, the amount of sodium hydroxide in the solution comprising a compound of Formula Ia or Ib, PEG 300, sodium hydroxide, poloxamer 188, and water is about 1.1 w/w %.

In some embodiments, the amount of poloxamer 188 in the solution comprising a compound of Formula Ia or Ib, PEG 300, sodium hydroxide, poloxamer 188, and water is about 0.1 w/w % to about 10 w/w %. In some embodiments, the amount of poloxamer 188 in the solution comprising a compound of Formula Ia or Ib, PEG 300, sodium hydroxide, poloxamer 188, and water is about 0.3 w/w % to about 8 w/w %. In some embodiments, the amount of poloxamer 188 in the solution comprising a compound of Formula Ia or Ib, PEG 300, sodium hydroxide, poloxamer 188, and water is about 0.5 w/w % to about 7 w/w %. In some embodiments, the amount of poloxamer 188 in the solution comprising a compound of Formula Ia or Ib, PEG 300, sodium hydroxide, poloxamer 188, and water is about 0.6 w/w % to about 7 w/w %. In some embodiments, the amount of poloxamer 188 in the solution comprising a compound of Formula Ia or Ib, PEG 300, sodium hydroxide, poloxamer 188, and water is about 0.85 w/w % to about 4.69 w/w %. In some embodiments, the amount of poloxamer 188 in the solution comprising a compound of Formula Ia or Ib, PEG 300, sodium hydroxide, poloxamer 188, and water is about 0.9 w/w % to about 4.7 w/w %. In some embodiments, the amount of poloxamer 188 in the solution comprising a compound of Formula Ia or Ib, PEG 300, sodium hydroxide, poloxamer 188, and water is about 0.5 w/w %, about 0.6 w/w %, about 0.7 w/w %, about 0.8 w/w %, about 0.9 w/w %, about 1.0 w/w %, about 1.1 w/w %, about 1.2 w/w %, about 1.3 w/w %, about 1.4 w/w %, about 1.5 w/w %, about 1.6 w/w %, about 1.7 w/w %, about 1.8 w/w %, about 1.9 w/w %, about 2.0 w/w %, about 2.1 w/w %, about 2.2 w/w %, about 2.3 w/w %, about 2.4 w/w %, about 2.5 w/w %, about 2.6 w/w %, about 2.7 w/w %, about 2.8 w/w %, about 2.9 w/w %, about 3.0 w/w %, about 3.1 w/w %, about 3.2 w/w %, about 3.3 w/w %, about 3.4 w/w %, about 3.5 w/w %, about 3.6 w/w %, about 3.7 w/w %, about 3.8 w/w %, about 3.9 w/w %, about 4.0 w/w %, about 4.1 w/w %, about 4.2 w/w %, about 4.3 w/w %, about 4.4 w/w %, about 4.5 w/w %, about 4.6 w/w %, about 4.7 w/w %, about 4.8 w/w %, about 4.9 w/w %, about 5.0 w/w %, about 5.1 w/w %, about 5.2 w/w %, about 5.3 w/w %, about 5.4 w/w %, about 5.5 w/w %, about 5.6 w/w %, about 5.7 w/w %, about 5.8 w/w %, about 5.9 w/w %, about 6.0 w/w %, about 6.1 w/w %, about 6.2 w/w %, about 6.3 w/w %, about 6.4 w/w %, about 6.5 w/w %, about 6.6 w/w %, about 6.7 w/w %, about 6.8 w/w %, about 6.9 w/w %, or about 7.0 w/w %. In some embodiments, the amount of poloxamer 188 in the solution comprising a compound of Formula Ia or Ib, PEG 300, sodium hydroxide, poloxamer 188, and water is about 0.85 w/w %. In some embodiments, the amount of poloxamer 188 in the solution comprising a compound of Formula Ia or Ib, PEG 300, sodium hydroxide, poloxamer 188, and water is about 0.9 w/w %. In some embodiments, the amount of poloxamer 188 in the solution comprising a compound of Formula Ia or Ib, PEG 300, sodium hydroxide, poloxamer 188, and water is about 4.69 w/w %. In some embodiments, the amount of poloxamer 188 in the solution comprising a compound of Formula Ia or Ib, PEG 300, sodium hydroxide, poloxamer 188, and water is about 4.7 w/w %.

In some embodiments, the solution comprises about 10 w/w % to about 45 w/w % water, about 30 w/w % to about 85 w/w % PEG 300, about 0.5 w/w % to about 40 w/w % of a compound of Formula Ia or Ib, about 0.01 w/w % to about 3.0 w/w % of sodium hydroxide, and about 0.1 w/w % to about 10 w/w % of poloxamer 188. In some embodiments, the solution comprises about 15 w/w % to about 35 w/w % water, about 35 w/w % to about 75 w/w % PEG 300, about 1 w/w % to about 35 w/w % of a compound of Formula Ia or Ib, about 0.01 w/w % to about 2.0 w/w % of sodium hydroxide, and about 0.3 w/w % to about 8 w/w % of poloxamer 188. In some embodiments, the solution comprises about 20 w/w % to about 35 w/w % water, about 40 w/w % to about 70 w/w % PEG 300, about 1 w/w % to about 30 w/w % of a compound of Formula Ia or Ib, about 0.05 w/w % to about 1.5 w/w % of sodium hydroxide, and about 0.5 w/w % to about 7 w/w % of poloxamer 188. In some embodiments, the solution comprises about 20 w/w % to about 31 w/w % water, about 45 w/w % to about 68 w/w % PEG 300, about 3 w/w % to about 28 w/w % of a compound of Formula Ia or Ib, about 0.05 w/w % to about 1.2 w/w % of sodium hydroxide, and about 0.6 w/w % to about 7 w/w % of poloxamer 188.

In some embodiments, the solution comprises about 22 w/w % to about 30.1 w/w % water, about 47.1 w/w % to about 64.4 w/w % PEG 300, about 4.6 w/w % to about 25.2 w/w % of a compound of Formula Ia or Ib, about 0.1 w/w % to about 1.1 w/w % of sodium hydroxide, and about 0.9 w/w % to about 4.7 w/w % of poloxamer 188. In some embodiments, the solution comprises about 21.97 w/w % to about 30.07 w/w % water, about 47.05 w/w % to about 64.41 w/w % PEG 300, about 4.57 w/w % to about 25.21 w/w % of a compound of Formula Ia or Ib, about 0.10 w/w % to about 1.08 w/w % of sodium hydroxide, and about 0.85 w/w % to about 4.69 w/w % of poloxamer 188.

In some embodiments, the solution comprises about 30.1 w/w % water, about 64.4 w/w % PEG 300, about 4.6 w/w % of a compound of Formula Ia or Ib, about 0.1 w/w % of sodium hydroxide, and about 0.9 w/w % of poloxamer 188. In some embodiments, the solution comprises about 30.07 w/w % water, about 64.41 w/w % PEG 300, about 4.57 w/w % of a compound of Formula Ia or Ib, about 0.10 w/w % of sodium hydroxide, and about 0.85 w/w % of poloxamer 188. some embodiments, the solution comprises about 30.1 w/w % water, about 64.4 w/w % PEG 300, about 4.6 w/w % of a compound of Formula Ib, about 0.1 w/w % of sodium hydroxide, and about 0.9 w/w % of poloxamer 188. In some embodiments, the solution comprises about 30.07 w/w % water, about 64.41 w/w % PEG 300, about 4.57 w/w % of a compound of Formula Ib, about 0.10 w/w % of sodium hydroxide, and about 0.85 w/w % of poloxamer 188.

In some embodiments, the solution comprises about 22 w/w % water, about 47.1 w/w % PEG 300, about 25.2 w/w % of a compound of Formula Ia or Ib, about 1.1 w/w % of sodium hydroxide, and about 4.7 w/w % of poloxamer 188. In some embodiments, the solution comprises about 21.97 w/w % water, about 47.05 w/w % PEG 300, about 25.21 w/w % of a compound of Formula Ia or Ib, about 1.08 w/w % of sodium hydroxide, and about 4.69 w/w % of poloxamer 188. In some embodiments, the solution comprises about 22 w/w % water, about 47.1 w/w % PEG 300, about 25.2 w/w % of a compound of Formula Ib, about 1.1 w/w % of sodium hydroxide, and about 4.7 w/w % of poloxamer 188. In some embodiments, the solution comprises about 21.97 w/w % water, about 47.05 w/w % PEG 300, about 25.21 w/w % of a compound of Formula Ib, about 1.08 w/w % of sodium hydroxide, and about 4.69 w/w % of poloxamer 188.

In some embodiments, the compound of Formula Ia or Ib becomes ionized in situ to a sodium salt of the compound of Formula Ia or Ib in the presence of sodium hydroxide in the solution comprising a compound of Formula Ia or Ib, PEG 300, sodium hydroxide, poloxamer 188, and water. Thus, in some embodiments, the compound of Formula Ia or Ib is present as a sodium salt of the compound of Formula Ia or Ib in the solution comprising a compound of Formula Ia or Ib, PEG 300, sodium hydroxide, poloxamer 188, and water.

In some embodiments, the PEG 300 of the solutions disclosed herein can be replaced with PEG 400 or PEG 600 in the same amount as the PEG 300. In some embodiments, the PEG 300 of the solutions disclosed herein can be replaced with PEG 400 in the same amount as the PEG 300. In some embodiments, the PEG 300 of the solutions disclosed herein can be replaced with PEG 600 in the same amount as the PEG 300.

In some embodiments, the molar ratio of sodium to a compound of Formula Ia or Ib is about 0:1 to about 1.5:1. In some embodiments, the molar ratio of sodium to a compound of Formula Ia or Ib is about 0:1 to about 1.5:1. In some embodiments, the molar ratio of sodium to a compound of Formula Ia or Ib is about 0:1 to about 1.2:1. In some embodiments, the molar ratio of sodium to a compound of Formula Ia or Ib is about 0:1. In some embodiments, the molar ratio of sodium to a compound of Formula Ia or Ib is about 0.5:1. In some embodiments, the molar ratio of sodium to a compound of Formula Ia or Ib is about 1:1. In some embodiments, the molar ratio of sodium to a compound of Formula Ia or Ib is about 1.2:1.

In some embodiments, the solutions are administered through subcutaneous injection. In some embodiments, the solutions are administered through intramuscular injection.

In some embodiments of the methods disclosed herein, the compound of Formula Ia or Ib, or a pharmaceutically acceptable salt thereof, is administered subcutaneously at a concentration of about 309 mg/mL.

In some embodiments of the methods disclosed herein, a solution of the sodium salt of the compound of Formula Ia or Ib is administered subcutaneously, wherein the solution comprises about 20 w/w % to about 30 w/w % water, about 48 w/w % to about 60 w/w % PEG 300, and about 11 w/w % to about 28 w/w % of a sodium salt of the compound of Formula Ia or Ib.

In some embodiments of the methods disclosed herein, a solution of the sodium salt of the compound of Formula Ia or Ib is administered subcutaneously, wherein the solution comprises about 23.41 w/w % water, about 50.13 w/w % PEG 300, and about 26.46 w/w % of the sodium salt of the compound of Formula Ia or Ib. In some embodiments of the methods disclosed herein, a solution of the sodium salt of the compound of Formula Ib is administered subcutaneously, wherein the solution comprises about 23.41 w/w % water, about 50.13 w/w % PEG 300, and about 26.46 w/w % of the sodium salt of the compound of Formula Ib.

In some embodiments of the methods disclosed herein, the compound of Formula Ia or Ib, or a pharmaceutically acceptable salt thereof, is administered intramuscularly.

In some embodiments of the methods disclosed herein, the compound of Formula Ia or Ib, or a pharmaceutically acceptable salt thereof, is administered intramuscularly at a concentration of about 400 mg/mL to about 600 mg/mL. In some embodiments of the methods disclosed herein, the compound of Formula Ia or Ib, or a pharmaceutically acceptable salt thereof, is administered intramuscularly at a concentration of about 400 mg/mL to about 500 mg/mL. In some embodiments of the methods disclosed herein, the compound of Formula Ia or Ib, or a pharmaceutically acceptable salt thereof, is administered intramuscularly at a concentration of about 400 mg/mL. In some embodiments of the methods disclosed herein, the compound of Formula Ia or Ib, or a pharmaceutically acceptable salt thereof, is administered intramuscularly at a concentration of about 500 mg/mL. In some embodiments of the methods disclosed herein, the compound of Formula Ia or Ib, or a pharmaceutically acceptable salt thereof, is administered intramuscularly at a concentration of about 600 mg/mL.

In some embodiments, the compound of Formula Ia or Ib, or a pharmaceutically acceptable salt thereof, is administered orally.

In certain embodiments, an oral formulation of a compound of Formula Ia or Ib, or a pharmaceutically acceptable salt thereof, and at least one excipient is provided. Excipients can include ethanol, medium chain triglycerides, vitamin E d-a-tocopheryl polyethylene glycol 1000 succinate (TPGS), glycerol monocaprylocaprate, glycerin, and/or pharmaceutically acceptable oils. Examples of suitable medium chain triglycerides include, but are not limited to, MIGLYOL 810, MIGLYOL 821, and MIGLYOL 840. Examples of suitable pharmaceutically acceptable oils include, but are not limited to, sesame oil, castor oil, safflower oil, vegetable oil, and soybean oil. Oral formulations can include any combination of one or more suitable excipients. Excipients taken together can be present in greater than about 65% by weight of the total oral formulation, greater than about 70% by weight of the total oral formulation, greater than about 80% by weight of the total oral formulation, greater than about 90% by weight of the total oral formulation, or greater than about 95% by weight of the total oral formulation.

In some embodiments, the oral formulation of a compound of Formula Ia or Ib, or a pharmaceutically acceptable salt thereof, and at least one excipient as is prepared in hard or soft capsules. In some embodiments, the hard or soft capsules comprise a compound of Formula Ia or Ib, or a pharmaceutically acceptable salt thereof, and one or more excipients selected from the group consisting of ethanol, propylene glycol, glycerine, d-a-tocopheryl polyethylene glycol 1000 succinate (TPGS), polyoxyl 35 castor oil (e.g., Kolliphor® EL), glycerol monocapryolocaprate (e.g., Capmul® MCM), PEG 400 caprylic/capric glycerides (e.g., Labrasol®), PEG 400, diethylene glycol monoethyl ether (e.g., Transcutol®), propylene glycol monocaprylate, Type II (e.g., Capryol® 90), glyceryl monooleate, Type 40 (e.g., Peceol™), medium chain triglycerides (e.g., Miglyol® 812N), glyceryl monolinoleate (e.g., Maisine®), and polysorbate 80. In some embodiments, the hard or soft capsules comprise a compound of Formula Ia or Ib, or a pharmaceutically acceptable salt thereof, and one or more excipients selected from the group consisting of ethanol, medium chain triglycerides (e.g., Miglyol® 812N), d-a-tocopheryl polyethylene glycol 1000 succinate (TPGS), glycerol monocapryolocaprate (e.g., Capmul® MCM), PEG 400 caprylic/capric glycerides (e.g., Labrasol®), and PEG 400.

In some embodiments, the hard or soft capsules further comprise a capsule shell. In some embodiments, the capsule shell comprises one or more pharmaceutically acceptable excipients. In some embodiments, the one or more pharmaceutically acceptable excipients of the capsule shell is selected from the group consisting of a gelatin shell, a plasticizer, an opacifier, and a colorant. Plasticizers, opacifiers, and colorants are well-known in the art. In some embodiments, the capsule shell comprises one or more pharmaceutically acceptable excipients selected from the group consisting of gelatin, glycerin, titanium dioxide, and iron oxide. In some embodiments, the iron oxide comprises iron oxide (yellow).

In some embodiments, an oral formulation of a compound of Formula Ia or Ib, or a pharmaceutically acceptable salt thereof, is provided. In certain embodiments, the oral formulation comprises a compound of Formula Ia or Ib, or a pharmaceutically acceptable salt thereof, and glycerol monocaprylocaprate. In certain embodiments, the oral formulation includes a compound of Formula Ia or Ib, or a pharmaceutically acceptable salt thereof, about 5% to about 20% ethanol, about 10% to about 30% vitamin E TPGS, and about 50% to about 85% MIGLYOL 812. In some embodiments, the oral formulation includes a compound of Formula Ia or Ib, or a pharmaceutically acceptable salt thereof, about 8% to about 15% ethanol, about 15% to about 25% vitamin E TPGS, and about 60% to about 77% MIGLYOL 812. In certain embodiments, the oral formulation includes a compound of Formula Ia or Ib, or a pharmaceutically acceptable salt thereof, in about 10% ethanol, about 20% vitamin E TPGS, and about 70% MIGLYOL 812. In certain embodiments, the oral formulation is prepared in hard gelatin capsules. In certain embodiments, the oral formulation is prepared in soft gelatin capsules.

In some embodiments, the hard gelatin or soft gelatin capsule comprises a compound of Formula Ia or Ib, or a pharmaceutically acceptable salt thereof, and a pharmaceutically acceptable excipient. In some embodiments, the hard gelatin or soft gelatin capsule comprises a compound of Formula Ia or Ib, or a pharmaceutically acceptable salt thereof, and glycerol monocaprylocaprate. In some embodiments, the concentration of a compound of Formula Ia or Ib, or a pharmaceutically acceptable salt thereof, in the hard gelatin or soft gelatin capsule is about 10 mg/ml to about 500 mg/ml. In some embodiments, the concentration of a compound of Formula Ia or Ib, or a pharmaceutically acceptable salt thereof, in the hard gelatin or soft gelatin capsule is about 10 mg/ml to about 400 mg/ml. In some embodiments, the concentration of a compound of Formula Ia or Ib, or a pharmaceutically acceptable salt thereof, in the hard gelatin or soft gelatin capsule is about 50 mg/ml to about 300 mg/ml. In some embodiments, the concentration of a compound of Formula Ia or Ib, or a pharmaceutically acceptable salt thereof, in the hard gelatin or soft gelatin capsule is about 50 mg/ml to about 200 mg/ml. In some embodiments, the concentration of a compound of Formula Ia or Ib, or a pharmaceutically acceptable salt thereof, in the hard gelatin or soft gelatin capsule is about 50 mg/ml to about 100 mg/ml. In some embodiments, the concentration of a compound of Formula Ia or Ib, or a pharmaceutically acceptable salt thereof, in the hard gelatin or soft gelatin capsule is about 10 mg/ml, about 15 mg/ml, about 20 mg/ml, about 25 mg/ml, about 30 mg/ml, about 30 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 105 mg/ml, about 110 mg/ml, about 115 mg/ml, about 120 mg/ml, about 125 mg/ml, about 130 mg/ml, about 135 mg/ml, about 140 mg/ml, about 145 mg/ml, about 150 mg/ml, about 155 mg/ml, about 160 mg/ml, about 165 mg/ml, about 170 mg/ml, about 175 mg/ml, about 180 mg/ml, about 185 mg/ml, about 190 mg/ml, about 195 mg/ml, about 200 mg/ml, 205 mg/ml, about 210 mg/ml, about 215 mg/ml, about 220 mg/ml, about 225 mg/ml, about 230 mg/ml, about 235 mg/ml, about 240 mg/ml, about 245 mg/ml, about 250 mg/ml, about 255 mg/ml, about 260 mg/ml, about 265 mg/ml, about 270 mg/ml, about 275 mg/ml, about 280 mg/ml, about 285 mg/ml, about 290 mg/ml, about 295 mg/ml, about 300 mg/ml, about 305 mg/ml, about 310 mg/ml, about 315 mg/ml, about 320 mg/ml, about 325 mg/ml, about 330 mg/ml, about 335 mg/ml, about 340 mg/ml, about 345 mg/ml, about 350 mg/ml, about 355 mg/ml, about 360 mg/ml, about 365 mg/ml, about 370 mg/ml, about 375 mg/ml, about 380 mg/ml, about 385 mg/ml, about 390 mg/ml, about 395 mg/ml, about 400 mg/ml, about 405 mg/ml, about 410 mg/ml, about 415 mg/ml, about 420 mg/ml, about 425 mg/ml, about 430 mg/ml, about 435 mg/ml, about 440 mg/ml, about 445 mg/ml, about 450 mg/ml, about 455 mg/ml, about 460 mg/ml, about 465 mg/ml, about 470 mg/ml, about 475 mg/ml, about 480 mg/ml, about 485 mg/ml, about 490 mg/ml, about 495 mg/ml, or about 500 mg/ml. In some embodiments, the concentration of a compound of Formula Ia or Ib, or a pharmaceutically acceptable salt thereof, in the hard gelatin or soft gelatin capsule is about 10 mg/ml. In some embodiments, the concentration of a compound of Formula Ia or Ib, or a pharmaceutically acceptable salt thereof, in the hard gelatin or soft gelatin capsule is about 20 mg/ml. In some embodiments, the concentration of a compound of Formula Ia or Ib, or a pharmaceutically acceptable salt thereof, in the hard gelatin or soft gelatin capsule is about 30 mg/ml. In some embodiments, the concentration of a compound of Formula Ia or Ib, or a pharmaceutically acceptable salt thereof, in the hard gelatin or soft gelatin capsule is about 40 mg/ml. In some embodiments, the concentration of a compound of Formula Ia or Ib, or a pharmaceutically acceptable salt thereof, in the hard gelatin or soft gelatin capsule is about 50 mg/ml. In some embodiments, the concentration of a compound of Formula Ia or Ib, or a pharmaceutically acceptable salt thereof, in the hard gelatin or soft gelatin capsule is about 75 mg/ml. In some embodiments, the concentration of a compound of Formula Ia or Ib, or a pharmaceutically acceptable salt thereof, in the hard gelatin or soft gelatin capsule is about 100 mg/ml. In some embodiments, the concentration of a compound of Formula Ia or Ib, or a pharmaceutically acceptable salt thereof, in the hard gelatin or soft gelatin capsule is about 125 mg/ml. In some embodiments, the concentration of a compound of Formula Ia or Ib, or a pharmaceutically acceptable salt thereof, in the hard gelatin or soft gelatin capsule is about 150 mg/ml. In some embodiments, the concentration of a compound of Formula Ia or Ib, or a pharmaceutically acceptable salt thereof, in the hard gelatin or soft gelatin capsule is about 175 mg/ml. In some embodiments, the concentration of a compound of Formula Ia or Ib, or a pharmaceutically acceptable salt thereof, in the hard gelatin or soft gelatin capsule is about 200 mg/ml. In some embodiments, the concentration of a compound of Formula Ia or Ib, or a pharmaceutically acceptable salt thereof, in the hard gelatin or soft gelatin capsule is about 225 mg/ml. In some embodiments, the concentration of a compound of Formula Ia or Ib, or a pharmaceutically acceptable salt thereof, in the hard gelatin or soft gelatin capsule is about 250 mg/ml. In some embodiments, the concentration of a compound of Formula Ia or Ib, or a pharmaceutically acceptable salt thereof, in the hard gelatin or soft gelatin capsule is about 275 mg/ml. In some embodiments, the concentration of a compound of Formula Ia or Ib, or a pharmaceutically acceptable salt thereof, in the hard gelatin or soft gelatin capsule is about 300 mg/ml. In some embodiments, the concentration of a compound of Formula Ia or Ib, or a pharmaceutically acceptable salt thereof, in the hard gelatin or soft gelatin capsule is about 325 mg/ml. In some embodiments, the concentration of a compound of Formula Ia or Ib, or a pharmaceutically acceptable salt thereof, in the hard gelatin or soft gelatin capsule is about 350 mg/ml. In some embodiments, the concentration of a compound of Formula Ia or Ib, or a pharmaceutically acceptable salt thereof, in the hard gelatin or soft gelatin capsule is about 375 mg/ml. In some embodiments, the concentration of a compound of Formula Ia or Ib, or a pharmaceutically acceptable salt thereof, in the hard gelatin or soft gelatin capsule is about 400 mg/ml. In some embodiments, the concentration of a compound of Formula Ia or Ib, or a pharmaceutically acceptable salt thereof, in the hard gelatin or soft gelatin capsule is about 425 mg/ml. In some embodiments, the concentration of a compound of Formula Ia or Ib, or a pharmaceutically acceptable salt thereof, in the hard gelatin or soft gelatin capsule is about 450 mg/ml. In some embodiments, the concentration of a compound of Formula Ia or Ib, or a pharmaceutically acceptable salt thereof, in the hard gelatin or soft gelatin capsule is about 475 mg/ml. In some embodiments, the concentration of a compound of Formula Ia or Ib, or a pharmaceutically acceptable salt thereof, in the hard gelatin or soft gelatin capsule is about 500 mg/ml.

In some embodiments, the amount of glycerol monocaprylocaprate in the hard gelatin or soft gelatin capsule comprising a compound of Formula Ia or Ib and glycerol monocaprylocaprate is about 30 w/w % to about 85 w/w %. In some embodiments, the amount of glycerol monocaprylocaprate in the hard gelatin or soft gelatin capsule comprising a compound of Formula Ia or Ib and glycerol monocaprylocaprate is about 40 w/w % to about 80 w/w %. In some embodiments, the amount of glycerol monocaprylocaprate in the hard gelatin or soft gelatin capsule comprising a compound of Formula Ia or Ib and glycerol monocaprylocaprate is about 50 w/w % to about 80 w/w %. In some embodiments, the amount of glycerol monocaprylocaprate in the hard gelatin or soft gelatin capsule comprising a compound of Formula Ia or Ib and glycerol monocaprylocaprate is about 60 w/w % to about 70 w/w %. In some embodiments, the amount of glycerol monocaprylocaprate in the hard gelatin or soft gelatin capsule comprising a compound of Formula Ia or Ib and glycerol monocaprylocaprate is about 65.9 w/w %. In some embodiments, the amount of glycerol monocaprylocaprate in the hard gelatin or soft gelatin capsule comprising a compound of Formula Ia or Ib and glycerol monocaprylocaprate is about 65.94 w/w %.

In some embodiments, the amount of the compound of Formula Ia or Ib in the hard gelatin or soft gelatin capsule comprising a compound of Formula Ia or Ib and glycerol monocaprylocaprate is about 1 w/w % to about 40 w/w %. In some embodiments, the amount of the compound of Formula Ia or Ib in the hard gelatin or soft gelatin capsule comprising a compound of Formula Ia or Ib and glycerol monocaprylocaprate is about 1 w/w % to about 35 w/w %. In some embodiments, the amount of the compound of Formula Ia or Ib in the hard gelatin or soft gelatin capsule comprising a compound of Formula Ia or Ib and glycerol monocaprylocaprate is about 2 w/w % to about 30 w/w %. In some embodiments, the amount of the compound of Formula Ia or Ib in the hard gelatin or soft gelatin capsule comprising a compound of Formula Ia or Ib and glycerol monocaprylocaprate is about 3 w/w % to about 28 w/w %. In some embodiments, the amount of the compound of Formula Ia or Ib in the hard gelatin or soft gelatin capsule comprising a compound of Formula Ia or Ib and glycerol monocaprylocaprate is about 3.4 w/w %. In some embodiments, the amount of the compound of Formula Ia or Ib in the hard gelatin or soft gelatin capsule comprising a compound of Formula Ia or Ib and glycerol monocaprylocaprate is about 3.42 w/w %.

In some embodiments, the hard gelatin or soft gelatin capsule comprising a compound of Formula Ia or Ib and glycerol monocaprylocaprate further comprises a capsule shell. In some embodiments, the capsule shell comprises one or more pharmaceutically acceptable excipients. In some embodiments, the one or more pharmaceutically acceptable excipients of the capsule shell is selected from the group consisting of a gelatin shell, a plasticizer, an opacifier, and a colorant. In some embodiments, the hard gelatin or soft gelatin capsule comprising a compound of Formula Ia or Ib and glycerol monocaprylocaprate further comprises one or more of a pharmaceutically acceptable excipient selected from the group consisting of gelatin, glycerin, titanium dioxide, and iron oxide. In some embodiments, the hard gelatin or soft gelatin capsule comprising a compound of Formula Ia or Ib and glycerol monocaprylocaprate further comprises gelatin. In some embodiments, the hard gelatin or soft gelatin capsule comprising a compound of Formula Ia or Ib and glycerol monocaprylocaprate further comprises glycerin. In some embodiments, the hard gelatin or soft gelatin capsule comprising a compound of Formula Ia or Ib and glycerol monocaprylocaprate further comprises titanium dioxide. In some embodiments, the hard gelatin or soft gelatin capsule comprising a compound of Formula Ia or Ib and glycerol monocaprylocaprate further comprises iron oxide. In some embodiments, the hard gelatin or soft gelatin capsule comprising a compound of Formula Ia or Ib and glycerol monocaprylocaprate further comprises gelatin and glycerin. In some embodiments, the hard gelatin or soft gelatin capsule comprising a compound of Formula Ia or Ib and glycerol monocaprylocaprate further comprises gelatin, glycerin, and titanium dioxide. In some embodiments, the hard gelatin or soft gelatin capsule comprising a compound of Formula Ia or Ib and glycerol monocaprylocaprate further comprises gelatin, glycerin, titanium dioxide, and iron oxide. In some embodiments, the iron oxide of the hard gelatin or soft gelatin capsule comprising a compound of Formula Ia or Ib, glycerol monocaprylocaprate, gelatin, glycerin, titanium dioxide, and iron oxide comprises iron oxide (yellow).

In some embodiments, the amount of gelatin in the hard gelatin or soft gelatin capsule comprising a compound of Formula Ia or Ib, glycerol monocaprylocaprate, gelatin, glycerin, titanium dioxide, and iron oxide is about 10 w/w % to about 40 w/w %. In some embodiments, the amount of gelatin in the hard gelatin or soft gelatin capsule comprising a compound of Formula Ia or Ib, glycerol monocaprylocaprate, gelatin, glycerin, titanium dioxide, and iron oxide is about 10 w/w % to about 30 w/w %. In some embodiments, the amount of gelatin in the hard gelatin or soft gelatin capsule comprising a compound of Formula Ia or Ib, glycerol monocaprylocaprate, gelatin, glycerin, titanium dioxide, and iron oxide is about 15 w/w % to about 25 w/w %. In some embodiments, the amount of gelatin in the hard gelatin or soft gelatin capsule comprising a compound of Formula Ia or Ib, glycerol monocaprylocaprate, gelatin, glycerin, titanium dioxide, and iron oxide is about 18 w/w % to about 22 w/w %. In some embodiments, the amount of gelatin in the hard gelatin or soft gelatin capsule comprising a compound of Formula Ia or Ib, glycerol monocaprylocaprate, gelatin, glycerin, titanium dioxide, and iron oxide is about 19.6 w/w %. In some embodiments, the amount of gelatin in the hard gelatin or soft gelatin capsule comprising a compound of Formula Ia or Ib, glycerol monocaprylocaprate, gelatin, glycerin, titanium dioxide, and iron oxide is about 19.60 w/w %.

In some embodiments, the amount of glycerin in the hard gelatin or soft gelatin capsule comprising a compound of Formula Ia or Ib, glycerol monocaprylocaprate, gelatin, glycerin, titanium dioxide, and iron oxide is about 3 w/w % to about 25 w/w %. In some embodiments, the amount of glycerin in the hard gelatin or soft gelatin capsule comprising a compound of Formula Ia or Ib, glycerol monocaprylocaprate, gelatin, glycerin, titanium dioxide, and iron oxide is about 5 w/w % to about 20 w/w %. In some embodiments, the amount of glycerin in the hard gelatin or soft gelatin capsule comprising a compound of Formula Ia or Ib, glycerol monocaprylocaprate, gelatin, glycerin, titanium dioxide, and iron oxide is about 5 w/w % to about 15 w/w %. In some embodiments, the amount of glycerin in the hard gelatin or soft gelatin capsule comprising a compound of Formula Ia or Ib, glycerol monocaprylocaprate, gelatin, glycerin, titanium dioxide, and iron oxide is about 8 w/w % to about 12 w/w %. In some embodiments, the amount of glycerin in the hard gelatin or soft gelatin capsule comprising a compound of Formula Ia or Ib, glycerol monocaprylocaprate, gelatin, glycerin, titanium dioxide, and iron oxide is about 10.8 w/w %. In some embodiments, the amount of glycerin in the hard gelatin or soft gelatin capsule comprising a compound of Formula Ia or Ib, glycerol monocaprylocaprate, gelatin, glycerin, titanium dioxide, and iron oxide is about 10.80 w/w %.

In some embodiments, the amount of titanium dioxide in the hard gelatin or soft gelatin capsule comprising a compound of Formula Ia or Ib, glycerol monocaprylocaprate, gelatin, glycerin, titanium dioxide, and iron oxide is about 0.01 w/w % to about 2 w/w %. In some embodiments, the amount of titanium dioxide in the hard gelatin or soft gelatin capsule comprising a compound of Formula Ia or Ib, glycerol monocaprylocaprate, gelatin, glycerin, titanium dioxide, and iron oxide is about 0.05 w/w % to about 1.5 w/w %. In some embodiments, the amount of titanium dioxide in the hard gelatin or soft gelatin capsule comprising a compound of Formula Ia or Ib, glycerol monocaprylocaprate, gelatin, glycerin, titanium dioxide, and iron oxide is about 0.1 w/w % to about 1.0 w/w %. In some embodiments, the amount of titanium dioxide in the hard gelatin or soft gelatin capsule comprising a compound of Formula Ia or Ib, glycerol monocaprylocaprate, gelatin, glycerin, titanium dioxide, and iron oxide is about 0.1 w/w % to about 0.5 w/w %. In some embodiments, the amount of titanium dioxide in the hard gelatin or soft gelatin capsule comprising a compound of Formula Ia or Ib, glycerol monocaprylocaprate, gelatin, glycerin, titanium dioxide, and iron oxide is about 0.2 w/w %. In some embodiments, the amount of titanium dioxide in the hard gelatin or soft gelatin capsule comprising a compound of Formula Ia or Ib, glycerol monocaprylocaprate, gelatin, glycerin, titanium dioxide, and iron oxide is about 0.22 w/w %.

In some embodiments, the amount of iron oxide in the hard gelatin or soft gelatin capsule comprising a compound of Formula Ia or Ib, glycerol monocaprylocaprate, gelatin, glycerin, titanium dioxide, and iron oxide is about 0.01 w/w % to about 1 w/w %. In some embodiments, the amount of iron oxide in the hard gelatin or soft gelatin capsule comprising a compound of Formula Ia or Ib, glycerol monocaprylocaprate, gelatin, glycerin, titanium dioxide, and iron oxide is about 0.01 w/w % to about 0.5 w/w %. In some embodiments, the amount of iron oxide in the hard gelatin or soft gelatin capsule comprising a compound of Formula Ia or Ib, glycerol monocaprylocaprate, gelatin, glycerin, titanium dioxide, and iron oxide is about 0.01 w/w % to about 0.15 w/w %. In some embodiments, the amount of iron oxide in the hard gelatin or soft gelatin capsule comprising a compound of Formula Ia or Ib, glycerol monocaprylocaprate, gelatin, glycerin, titanium dioxide, and iron oxide is about 0.01 w/w % to about 0.1 w/w %. In some embodiments, the amount of iron oxide in the hard gelatin or soft gelatin capsule comprising a compound of Formula Ia or Ib, glycerol monocaprylocaprate, gelatin, glycerin, titanium dioxide, and iron oxide is about 0.02 w/w %.

In some embodiments, the hard gelatin or soft capsule comprises about 30 w/w % to about 85 w/w % of glycerol monocaprylocaprate and about 1 w/w % to about 40 w/w % of a compound of Formula Ia or Ib. In some embodiments, the hard gelatin or soft capsule comprises about 40 w/w % to about 80 w/w % of glycerol monocaprylocaprate and about 1 w/w % to about 35 w/w % of a compound of Formula Ia or Ib. In some embodiments, the hard gelatin or soft capsule comprises about 50 w/w % to about 80 w/w % of glycerol monocaprylocaprate and about 2 w/w % to about 30 w/w % of a compound of Formula Ia or Ib. In some embodiments, the hard gelatin or soft capsule comprises about 60 w/w % to about 70 w/w % of glycerol monocaprylocaprate and about 3 w/w % to about 28 w/w % of a compound of Formula Ia or Ib. In some embodiments, the hard gelatin or soft capsule comprises about 65.9 w/w % of glycerol monocaprylocaprate and about 3.4 w/w % of a compound of Formula Ia or Ib. In some embodiments, the hard gelatin or soft capsule comprises about 65.94 w/w % of glycerol monocaprylocaprate and about 3.42 w/w % of a compound of Formula Ia or Ib.

In some embodiments, the hard gelatin or soft capsule comprises about 30 w/w % to about 85 w/w % of glycerol monocaprylocaprate, about 1 w/w % to about 40 w/w % of a compound of Formula Ia or Ib, about 10 w/w % to about 40 w/w % of gelatin, about 3 w/w % to about 25 w/w % of glycerin, about 0.01 w/w % to about 2 w/w % of titanium dioxide, and about 0.01 w/w % to about 1 w/w % of iron oxide. In some embodiments, the hard gelatin or soft capsule comprises about 40 w/w % to about 80 w/w % of glycerol monocaprylocaprate, about 1 w/w % to about 35 w/w % of a compound of Formula Ia or Ib, about 10 w/w % to about 30 w/w % of gelatin, about 5 w/w % to about 20 w/w % of glycerin, about 0.05 w/w % to about 1.5 w/w % of titanium dioxide, and about 0.01 w/w % to about 0.5 w/w % of iron oxide. In some embodiments, the hard gelatin or soft capsule comprises about 50 w/w % to about 80 w/w % of glycerol monocaprylocaprate, about 2 w/w % to about 30 w/w % of a compound of Formula Ia or Ib, about 15 w/w % to about 25 w/w % of gelatin, about 5 w/w % to about 15 w/w % of glycerin, about 0.1 w/w % to about 1.0 w/w % of titanium dioxide, and about 0.01 w/w % to about 0.15 w/w % of iron oxide. In some embodiments, the hard gelatin or soft capsule comprises about 60 w/w % to about 70 w/w % of glycerol monocaprylocaprate, about 3 w/w % to about 28 w/w % of a compound of Formula Ia or Ib, about 18 w/w % to about 22 w/w % of gelatin, about 8 w/w % to about 12 w/w % of glycerin, about 0.1 w/w % to about 0.5 w/w % of titanium dioxide, and about 0.01 w/w % to about 0.1 w/w % of iron oxide. In some embodiments, the hard gelatin or soft capsule comprises about 65.9 w/w % of glycerol monocaprylocaprate, about 3.4 w/w % of a compound of Formula Ia or Ib, about 19.6 w/w % of gelatin, about 10.8 w/w % of glycerin, about 0.2 w/w % of titanium dioxide, and about 0.02 w/w % of iron oxide. In some embodiments, the hard gelatin or soft capsule comprises about 65.94 w/w % of glycerol monocaprylocaprate, about 3.42 w/w % of a compound of Formula Ia or Ib, about 19.60 w/w % of gelatin, about 10.80 w/w % of glycerin, about 0.22 w/w % of titanium dioxide, and about 0.02 w/w % of iron oxide.

In some embodiments, the iron oxide in the hard gelatin or soft gelatin capsule comprising a compound of Formula Ia or Ib, glycerol monocaprylocaprate, gelatin, glycerin, titanium dioxide, and iron oxide comprises iron oxide (yellow). In some embodiments, the hard gelatin or soft capsule comprises about 65.9 w/w % of glycerol monocaprylocaprate, about 3.4 w/w % of a compound of Formula Ia or Ib, about 19.6 w/w % of gelatin, about 10.8 w/w % of glycerin, about 0.2 w/w % of titanium dioxide, and about 0.02 w/w % of iron oxide (yellow). In some embodiments, the hard gelatin or soft capsule comprises about 65.94 w/w % of glycerol monocaprylocaprate, about 3.42 w/w % of a compound of Formula Ia or Ib, about 19.60 w/w % of gelatin, about 10.80 w/w % of glycerin, about 0.22 w/w % of titanium dioxide, and about 0.02 w/w % of iron oxide (yellow).

In some embodiments, the hard gelatin or soft gelatin capsule comprises a sodium salt of the compound of Formula Ia or Ib and glycerol monocaprylocaprate. In some embodiments, the concentration of the compound of Formula Ia or Ib in the hard gelatin or soft gelatin capsule comprising a sodium salt of the compound of Formula Ia or Ib and glycerol monocaprylocaprate is about 10 mg/ml to about 500 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 10 mg/ml to about 400 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 50 mg/ml to about 300 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 75 mg/ml to about 300 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 10 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 20 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 30 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 40 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 50 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 75 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 100 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 125 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 150 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 175 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 200 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 225 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 250 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 275 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 300 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 325 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 350 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 375 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 400 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 425 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 450 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 475 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 500 mg/ml.

In some embodiments, the amount of glycerol monocaprylocaprate in the hard gelatin or soft gelatin capsule comprising a sodium salt of the compound of Formula Ia or Ib and glycerol monocaprylocaprate is about 30 w/w % to about 99 w/w %. In some embodiments, the amount of glycerol monocaprylocaprate in the hard gelatin or soft gelatin capsule comprising a sodium salt of the compound of Formula Ia or Ib and glycerol monocaprylocaprate is about 50 w/w % to about 99 w/w %. In some embodiments, the amount of glycerol monocaprylocaprate in the hard gelatin or soft gelatin capsule comprising a sodium salt of the compound of Formula Ia or Ib and glycerol monocaprylocaprate is about 60 w/w % to about 99 w/w %. In some embodiments, the amount of glycerol monocaprylocaprate in the hard gelatin or soft gelatin capsule comprising a sodium salt of the compound of Formula Ia or Ib and glycerol monocaprylocaprate is about 75 w/w % to about 98 w/w %. In some embodiments, the amount of glycerol monocaprylocaprate in the hard gelatin or soft gelatin capsule comprising a sodium salt of the compound of Formula Ia or Ib and glycerol monocaprylocaprate is about 80.09 w/w % to about 94.85 w/w %. In some embodiments, the amount of glycerol monocaprylocaprate in the hard gelatin or soft gelatin capsule comprising a sodium salt of the compound of Formula Ia or Ib and glycerol monocaprylocaprate is about 80.1 w/w % to about 94.9 w/w %. In some embodiments, the amount of glycerol monocaprylocaprate in the hard gelatin or soft gelatin capsule comprising a sodium salt of the compound of Formula Ia or Ib and glycerol monocaprylocaprate is about 80.09 w/w %. In some embodiments, the amount of glycerol monocaprylocaprate in the hard gelatin or soft gelatin capsule comprising a sodium salt of the compound of Formula Ia or Ib and glycerol monocaprylocaprate is about 80.1 w/w %. In some embodiments, the amount of glycerol monocaprylocaprate in the hard gelatin or soft gelatin capsule comprising a sodium salt of the compound of Formula Ia or Ib and glycerol monocaprylocaprate is about 94.85 w/w %. In some embodiments, the amount of glycerol monocaprylocaprate in the hard gelatin or soft gelatin capsule comprising a sodium salt of the compound of Formula Ia or Ib and glycerol monocaprylocaprate is about 94.9 w/w %.

In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 1 w/w % to about 40 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 1 w/w % to about 35 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 2 w/w % to about 30 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 3 w/w % to about 28 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 5.15 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 5.2 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 19.9 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 19.91 w/w %.

In some embodiments, the hard gelatin or soft gelatin capsule comprising a sodium salt of the compound of Formula Ia or Ib and glycerol monocaprylocaprate further comprises a capsule shell. In some embodiments, the capsule shell comprises one or more pharmaceutically acceptable excipients. In some embodiments, the one or more pharmaceutically acceptable excipients of the capsule shell is selected from the group consisting of a gelatin shell, a plasticizer, an opacifier, and a colorant. In some embodiments, the hard gelatin or soft gelatin capsule comprising a sodium salt of the compound of Formula Ia or Ib and glycerol monocaprylocaprate further comprises one or more of a pharmaceutically acceptable excipient selected from the group consisting of gelatin, glycerin, titanium dioxide, and iron oxide. In some embodiments, the hard gelatin or soft gelatin capsule comprising a sodium salt of the compound of Formula Ia or Ib and glycerol monocaprylocaprate further comprises gelatin. In some embodiments, the hard gelatin or soft gelatin capsule comprising a sodium salt of the compound of Formula Ia or Ib and glycerol monocaprylocaprate further comprises glycerin. In some embodiments, the hard gelatin or soft gelatin capsule comprising a sodium salt of the compound of Formula Ia or Ib and glycerol monocaprylocaprate further comprises titanium dioxide. In some embodiments, the hard gelatin or soft gelatin capsule comprising a sodium salt of the compound of Formula Ia or Ib and glycerol monocaprylocaprate further comprises iron oxide. In some embodiments, the hard gelatin or soft gelatin capsule comprising a sodium salt of the compound of Formula Ia or Ib and glycerol monocaprylocaprate further comprises gelatin and glycerin. In some embodiments, the hard gelatin or soft gelatin capsule comprising a sodium salt of the compound of Formula Ia or Ib and glycerol monocaprylocaprate further comprises gelatin, glycerin, and titanium dioxide. In some embodiments, the hard gelatin or soft gelatin capsule comprising a sodium salt of the compound of Formula Ia or Ib and glycerol monocaprylocaprate further comprises gelatin, glycerin, titanium dioxide, and iron oxide. In some embodiments, the iron oxide of the hard gelatin or soft gelatin capsule comprising a sodium salt of the compound of Formula Ia or Ib, glycerol monocaprylocaprate, gelatin, glycerin, titanium dioxide, and iron oxide comprises iron oxide (yellow).

In some embodiments, the amount of gelatin in the hard gelatin or soft gelatin capsule comprising a sodium salt of the compound of Formula Ia or Ib, glycerol monocaprylocaprate, gelatin, glycerin, titanium dioxide, and iron oxide is about 10 w/w % to about 40 w/w %. In some embodiments, the amount of gelatin in the hard gelatin or soft gelatin capsule comprising a sodium salt of the compound of Formula Ia or Ib, glycerol monocaprylocaprate, gelatin, glycerin, titanium dioxide, and iron oxide is about 10 w/w % to about 30 w/w %. In some embodiments, the amount of gelatin in the hard gelatin or soft gelatin capsule comprising a sodium salt of the compound of Formula Ia or Ib, glycerol monocaprylocaprate, gelatin, glycerin, titanium dioxide, and iron oxide is about 15 w/w % to about 25 w/w %. In some embodiments, the amount of gelatin in the hard gelatin or soft gelatin capsule comprising a sodium salt of the compound of Formula Ia or Ib, glycerol monocaprylocaprate, gelatin, glycerin, titanium dioxide, and iron oxide is about 18 w/w % to about 22 w/w %. In some embodiments, the amount of gelatin in the hard gelatin or soft gelatin capsule comprising a sodium salt of the compound of Formula Ia or Ib, glycerol monocaprylocaprate, gelatin, glycerin, titanium dioxide, and iron oxide is about 19.6 w/w %. In some embodiments, the amount of gelatin in the hard gelatin or soft gelatin capsule comprising a sodium salt of the compound of Formula Ia or Ib, glycerol monocaprylocaprate, gelatin, glycerin, titanium dioxide, and iron oxide is about 19.60 w/w %.

In some embodiments, the amount of glycerin in the hard gelatin or soft gelatin capsule comprising a sodium salt of the compound of Formula Ia or Ib, glycerol monocaprylocaprate, gelatin, glycerin, titanium dioxide, and iron oxide is about 3 w/w % to about 25 w/w %. In some embodiments, the amount of glycerin in the hard gelatin or soft gelatin capsule comprising a sodium salt of the compound of Formula Ia or Ib, glycerol monocaprylocaprate, gelatin, glycerin, titanium dioxide, and iron oxide is about 5 w/w % to about 20 w/w %. In some embodiments, the amount of glycerin in the hard gelatin or soft gelatin capsule comprising a sodium salt of the compound of Formula Ia or Ib, glycerol monocaprylocaprate, gelatin, glycerin, titanium dioxide, and iron oxide is about 5 w/w % to about 15 w/w %. In some embodiments, the amount of glycerin in the hard gelatin or soft gelatin capsule comprising a sodium salt of the compound of Formula Ia or Ib, glycerol monocaprylocaprate, gelatin, glycerin, titanium dioxide, and iron oxide is about 8 w/w % to about 12 w/w %. In some embodiments, the amount of glycerin in the hard gelatin or soft gelatin capsule comprising a sodium salt of the compound of Formula Ia or Ib, glycerol monocaprylocaprate, gelatin, glycerin, titanium dioxide, and iron oxide is about 10.8 w/w %. In some embodiments, the amount of glycerin in the hard gelatin or soft gelatin capsule comprising a sodium salt of the compound of Formula Ia or Ib, glycerol monocaprylocaprate, gelatin, glycerin, titanium dioxide, and iron oxide is about 10.80 w/w %.

In some embodiments, the amount of titanium dioxide in the hard gelatin or soft gelatin capsule comprising a sodium salt of the compound of Formula Ia or Ib, glycerol monocaprylocaprate, gelatin, glycerin, titanium dioxide, and iron oxide is about 0.01 w/w % to about 2 w/w %. In some embodiments, the amount of titanium dioxide in the hard gelatin or soft gelatin capsule comprising a sodium salt of the compound of Formula Ia or Ib, glycerol monocaprylocaprate, gelatin, glycerin, titanium dioxide, and iron oxide is about 0.05 w/w % to about 1.5 w/w %. In some embodiments, the amount of titanium dioxide in the hard gelatin or soft gelatin capsule comprising a sodium salt of the compound of Formula Ia or Ib, glycerol monocaprylocaprate, gelatin, glycerin, titanium dioxide, and iron oxide is about 0.1 w/w % to about 1.0 w/w %. In some embodiments, the amount of titanium dioxide in the hard gelatin or soft gelatin capsule comprising a sodium salt of the compound of Formula Ia or Ib, glycerol monocaprylocaprate, gelatin, glycerin, titanium dioxide, and iron oxide is about 0.1 w/w % to about 0.5 w/w %. In some embodiments, the amount of titanium dioxide in the hard gelatin or soft gelatin capsule comprising a sodium salt of the compound of Formula Ia or Ib, glycerol monocaprylocaprate, gelatin, glycerin, titanium dioxide, and iron oxide is about 0.2 w/w %. In some embodiments, the amount of titanium dioxide in the hard gelatin or soft gelatin capsule comprising a sodium salt of the compound of Formula Ia or Ib, glycerol monocaprylocaprate, gelatin, glycerin, titanium dioxide, and iron oxide is about 0.22 w/w %.

In some embodiments, the amount of iron oxide in the hard gelatin or soft gelatin capsule comprising a sodium salt of the compound of Formula Ia or Ib, glycerol monocaprylocaprate, gelatin, glycerin, titanium dioxide, and iron oxide is about 0.01 w/w % to about 1 w/w %. In some embodiments, the amount of iron oxide in the hard gelatin or soft gelatin capsule comprising a sodium salt of the compound of Formula Ia or Ib, glycerol monocaprylocaprate, gelatin, glycerin, titanium dioxide, and iron oxide is about 0.01 w/w % to about 0.5 w/w %. In some embodiments, the amount of iron oxide in the hard gelatin or soft gelatin capsule comprising a sodium salt of the compound of Formula Ia or Ib, glycerol monocaprylocaprate, gelatin, glycerin, titanium dioxide, and iron oxide is about 0.01 w/w % to about 0.15 w/w %. In some embodiments, the amount of iron oxide in the hard gelatin or soft gelatin capsule comprising a sodium salt of the compound of Formula Ia or Ib, glycerol monocaprylocaprate, gelatin, glycerin, titanium dioxide, and iron oxide is about 0.01 w/w % to about 0.1 w/w %. In some embodiments, the amount of iron oxide in the hard gelatin or soft gelatin capsule comprising a sodium salt of the compound of Formula Ia or Ib, glycerol monocaprylocaprate, gelatin, glycerin, titanium dioxide, and iron oxide is about 0.02 w/w %.

In some embodiments, the hard gelatin or soft capsule disclosed herein comprises about 30 w/w % to about 99 w/w % of glycerol monocaprylocaprate and about 1 w/w % to about 40 w/w % of a sodium salt of the compound of Formula Ia or Ib. In some embodiments, the hard gelatin or soft capsule comprises about 50 w/w % to about 99 w/w % of glycerol monocaprylocaprate and about 1 w/w % to about 35 w/w % of a sodium salt of the compound of Formula Ia or Ib. In some embodiments, the hard gelatin or soft capsule comprises about 60 w/w % to about 99 w/w % of glycerol monocaprylocaprate and about 2 w/w % to about 30 w/w % of a sodium salt of the compound of Formula Ia or Ib. In some embodiments, the hard gelatin or soft capsule comprises about 75 w/w % to about 98 w/w % of glycerol monocaprylocaprate and about 3 w/w % to about 28 w/w % of a sodium salt of the compound of Formula Ia or Ib. In some embodiments, the hard gelatin or soft capsule comprises about 80.09 w/w % to about 94.85 w/w % of glycerol monocaprylocaprate and about 5.15 w/w % to about 19.91 w/w % of a sodium salt of the compound of Formula Ia or Ib. In some embodiments, the hard gelatin or soft capsule comprises about 80.1 w/w % to about 94.9 w/w % of glycerol monocaprylocaprate and about 5.2 w/w % to about 19.9 w/w % of a sodium salt of the compound of Formula Ia or Ib. In some embodiments, the hard gelatin or soft capsule comprises about 94.85 w/w % of glycerol monocaprylocaprate and about 5.15 w/w % of a sodium salt of the compound of Formula Ia or Ib. In some embodiments, the hard gelatin or soft capsule comprises about 94.9 w/w % of glycerol monocaprylocaprate and about 5.2 w/w % of a sodium salt of the compound of Formula Ia or Ib.

In some embodiments, the hard gelatin or soft capsule comprises about 80.09 w/w % of glycerol monocaprylocaprate and about 19.91 w/w % of a sodium salt of the compound of Formula Ia or Ib. In some embodiments, the hard gelatin or soft capsule comprises about 80.1 w/w % of glycerol monocaprylocaprate and about 19.9 w/w % of a sodium salt of the compound of Formula Ia or Ib.

In some embodiments, the iron oxide in the hard gelatin or soft gelatin capsule comprising a sodium salt of the compound of Formula Ia or Ib, glycerol monocaprylocaprate, gelatin, glycerin, titanium dioxide, and iron oxide comprises iron oxide (yellow).

In some embodiments, the oral formulation of a compound of Formula Ia or Ib, or a pharmaceutically acceptable salt thereof, is a tablet. In some embodiments, the tablet is prepared from a spray-dried dispersion of a compound of Formula Ia or Ib, or a pharmaceutically acceptable salt thereof. In some embodiments, the amount of a compound of Formula Ia or Ib, or a pharmaceutically acceptable salt thereof, in the tablet is about 5 mg to about 500 mg. In some embodiments, the amount of a compound of Formula Ia or Ib, or a pharmaceutically acceptable salt thereof, in the tablet is about 25 mg to about 500 mg. In some embodiments, the amount of a compound of Formula Ia or Ib, or a pharmaceutically acceptable salt thereof, in the tablet is about 25 mg to about 400 mg. In some embodiments, the amount of a compound of Formula Ia or Ib, or a pharmaceutically acceptable salt thereof, in the tablet is about 25 mg to about 300 mg. In some embodiments, the amount of a compound of Formula Ia or Ib, or a pharmaceutically acceptable salt thereof, in the tablet is about 50 mg to about 500 mg. In some embodiments, the amount of a compound of Formula Ia or Ib, or a pharmaceutically acceptable salt thereof, in the tablet is about 75 mg to about 500 mg. In some embodiments, the amount of a compound of Formula Ia or Ib, or a pharmaceutically acceptable salt thereof, in the tablet is about 50 mg to about 400 mg. In some embodiments, the amount of a compound of Formula Ia or Ib, or a pharmaceutically acceptable salt thereof, in the tablet is about 50 mg to about 300 mg. In some embodiments, the amount of a compound of Formula Ia or Ib, or a pharmaceutically acceptable salt thereof, in the tablet is about 75 mg to about 400 mg. In some embodiments, the amount of a compound of Formula Ia or Ib, or a pharmaceutically acceptable salt thereof, in the tablet is about 75 mg to about 300 mg. In some embodiments, the amount of a compound of Formula Ia or Ib, or a pharmaceutically acceptable salt thereof, in the tablet is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, or about 500 mg. In some embodiments, the amount of a compound of Formula Ia or Ib, or a pharmaceutically acceptable salt thereof, in the tablet is about 5 mg. In some embodiments, the amount of a compound of Formula Ia or Ib, or a pharmaceutically acceptable salt thereof, in the tablet is about 10 mg. In some embodiments, the amount of a compound of Formula Ia or Ib, or a pharmaceutically acceptable salt thereof, in the tablet is about 20 mg. In some embodiments, the amount of a compound of Formula Ia or Ib, or a pharmaceutically acceptable salt thereof, in the tablet is about 25 mg. In some embodiments, the amount of a compound of Formula Ia or Ib, or a pharmaceutically acceptable salt thereof, in the tablet is about 30 mg. In some embodiments, the amount of a compound of Formula Ia or Ib, or a pharmaceutically acceptable salt thereof, in the tablet is about 40 mg. In some embodiments, the amount of a compound of Formula Ia or Ib, or a pharmaceutically acceptable salt thereof, in the tablet is about 50 mg. In some embodiments, the amount of a compound of Formula Ia or Ib, or a pharmaceutically acceptable salt thereof, in the tablet is about 75 mg. In some embodiments, the amount of a compound of Formula Ia or Ib, or a pharmaceutically acceptable salt thereof, in the tablet is about 100 mg. In some embodiments, the amount of a compound of Formula Ia or Ib, or a pharmaceutically acceptable salt thereof, in the tablet is about 125 mg. In some embodiments, the amount of a compound of Formula Ia or Ib, or a pharmaceutically acceptable salt thereof, in the tablet is about 150 mg. In some embodiments, the amount of a compound of Formula Ia or Ib, or a pharmaceutically acceptable salt thereof, in the tablet is about 175 mg. In some embodiments, the amount of a compound of Formula Ia or Ib, or a pharmaceutically acceptable salt thereof, in the tablet is about 200 mg. In some embodiments, the amount of a compound of Formula Ia or Ib, or a pharmaceutically acceptable salt thereof, in the tablet is about 225 mg. In some embodiments, the amount of a compound of Formula Ia or Ib, or a pharmaceutically acceptable salt thereof, in the tablet is about 250 mg. In some embodiments, the amount of a compound of Formula Ia or Ib, or a pharmaceutically acceptable salt thereof, in the tablet is about 275 mg. In some embodiments, the amount of a compound of Formula Ia or Ib, or a pharmaceutically acceptable salt thereof, in the tablet is about 300 mg.

In some embodiments, the amount of a compound of Formula Ia or Ib, or a pharmaceutically acceptable salt thereof, in the tablet is about 325 mg. In some embodiments, the amount of a compound of Formula Ia or Ib, or a pharmaceutically acceptable salt thereof, in the tablet is about 350 mg. In some embodiments, the amount of a compound of Formula Ia or Ib, or a pharmaceutically acceptable salt thereof, in the tablet is about 375 mg. In some embodiments, the amount of a compound of Formula Ia or Ib, or a pharmaceutically acceptable salt thereof, in the tablet is about 400 mg. In some embodiments, the amount of a compound of Formula Ia or Ib, or a pharmaceutically acceptable salt thereof, in the tablet is about 425 mg. In some embodiments, the amount of a compound of Formula Ia or Ib, or a pharmaceutically acceptable salt thereof, in the tablet is about 450 mg. In some embodiments, the amount of a compound of Formula Ia or Ib, or a pharmaceutically acceptable salt thereof, in the tablet is about 475 mg. In some embodiments, the amount of a compound of Formula Ia or Ib, or a pharmaceutically acceptable salt thereof, in the tablet is about 500 mg.

In some embodiments, the tablet herein comprises a compound of Formula Ia or Ib, or a pharmaceutically acceptable salt thereof, and one or more pharmaceutically acceptable excipients. In some embodiments, the tablet comprises a compound of Formula Ia or a pharmaceutically acceptable salt thereof, and one or more pharmaceutically acceptable excipients. In some embodiments, the tablet comprises a compound of Formula Ia and one or more pharmaceutically acceptable excipients. In some embodiments, the tablet comprises a sodium salt of the compound of Formula Ia and one or more pharmaceutically acceptable excipients. In some embodiments, the tablet comprises a compound of Formula Ib or a pharmaceutically acceptable salt thereof, and one or more pharmaceutically acceptable excipients. In some embodiments, the tablet comprises a compound of Formula Ib and one or more pharmaceutically acceptable excipients. In some embodiments, the tablet comprises a sodium salt of the compound of Formula Ib and one or more pharmaceutically acceptable excipients.

The pharmaceutically acceptable excipients of the tablets disclosed herein should be compatible with the other ingredients of the formulation and physiologically innocuous to the recipient thereof. Examples of suitable excipients are well known to the person skilled in the art of tablet formulation and may be found e.g. in Handbook of Pharmaceutical Excipients (eds. Rowe, Sheskey & Quinn), 6th edition 2009. As used herein the term “excipients” is intended to refer to inter alia basifying agents, solubilisers, glidants, fillers, binders, lubricant, diluents, preservatives, surface active agents, dispersing agents and the like. The term also includes agents such as sweetening agents, flavoring agents, coloring agents and preserving agents. Such components will generally be present in admixture within the tablet.

Examples of solubilisers include, but are not limited to, surfactants (including both ionic and non-ionic surfactants) such as sodium lauryl sulphate, cetyltrimethylammonium bromide, polysorbates (such as polysorbate 20 or 80), poloxamers (such as poloxamer 188 or 207), and macrogols. Examples of lubricants, glidants and flow aids include, but are not limited to, magnesium stearate, calcium stearate, stearic acid, hydrogenated vegetable oil, glyceryl palmitostearate, glyceryl behenate, sodium stearyl fumarate, colloidal silicon dioxide, and talc.

Examples of disintegrants include, but are not limited to, starches, celluloses, cross-linked PVP, sodium starch glycolate, croscarmellose sodium, and the like. Examples of fillers (also known as bulking agents or diluents) include, but are not limited to, starches, maltodextrins, polyols (such as lactose), and celluloses. Examples of binders include, but are not limited to, cross-linked PVP, HPMC, microcrystalline cellulose, sucrose, starches, and the like.

In some embodiments, the tablets disclosed herein comprise a compound of Formula Ia or Ib, or a pharmaceutically acceptable salt thereof, and one or more pharmaceutically acceptable excipients selected from the group consisting of a matrix former, a surfactant, a filler, a disintegrant, and a lubricant. In some embodiments, the tablet comprises about 1 w/w % to about 10 w/w % of a matrix former. In some embodiments, the matrix former comprises copovidone. In some embodiments, the tablet comprises about 0.01 w/w % to about 10 w/w % of a surfactant. In some embodiments, the surfactant comprises poloxamer 407. In some embodiments, the tablet comprises about 25-85 w/w % of one or more fillers. In some embodiments, the one or more fillers comprises microcrystalline cellulose and/or mannitol. In some embodiments, the tablet comprises about 1 w/w % to about 30 w/w % of a disintegrant. In some embodiments, the disintegrant comprises croscarmellose sodium. In some embodiments, the tablet comprises about 0.01 w/w % to about 10 w/w % of a lubricant. In some embodiments, the lubricant comprises magnesium stearate.

The tablets disclosed herein may be uncoated or coated (in which case they include an outer film coat). Although uncoated tablets may be used, it is more usual to provide a coated tablet, in which case a conventional non-enteric coating may be used. Film coatings are known in the art and can be composed of hydrophilic polymer materials, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though the water-soluble material included in the film coating of the tablets may include a single polymer material, it may also be formed using a mixture of more than one polymer. The coating may be white or colored. Suitable coatings include, but are not limited to, polymeric film coatings such as those comprising polyvinyl alcohol e.g. ‘Opadry® II’ (which includes part-hydrolysed PVA, titanium dioxide, macrogol 3350 and talc, with optional colouring such as iron oxide or indigo carmine or iron oxide yellow or FD&C yellow #6). The amount of coating will generally be between about 1-8% of the uncoated tablet's weight.

In some embodiments, the tablet disclosed herein comprises a compound of Formula Ia or Ib or a pharmaceutically acceptable salt thereof, and one or more pharmaceutically acceptable excipients. In some embodiments, the one or more pharmaceutically acceptable excipients is selected from the group consisting of copovidone, poloxamer 407, microcrystalline cellulose, mannitol, croscarmellose sodium, and magnesium stearate. In some embodiments, the one or more pharmaceutically acceptable excipient comprises copovidone. In some embodiments, the one or more pharmaceutically acceptable excipient comprises poloxamer 407. In some embodiments, the one or more pharmaceutically acceptable excipient comprises microcrystalline cellulose. In some embodiments, the one or more pharmaceutically acceptable excipient comprises mannitol. In some embodiments, the one or more pharmaceutically acceptable excipient comprises croscarmellose sodium. In some embodiments, the one or more pharmaceutically acceptable excipient comprises magnesium stearate. In some embodiments, the one or more pharmaceutically acceptable excipient comprises copovidone and poloxamer 407. In some embodiments, the one or more pharmaceutically acceptable excipient comprises copovidone, poloxamer 407, and microcrystalline cellulose. In some embodiments, the one or more pharmaceutically acceptable excipient comprises copovidone, poloxamer 407, microcrystalline cellulose, and mannitol. In some embodiments, the one or more pharmaceutically acceptable excipient comprises copovidone, poloxamer 407, microcrystalline cellulose, mannitol, and croscarmellose sodium. In some embodiments, the one or more pharmaceutically acceptable excipient comprises copovidone, poloxamer 407, microcrystalline cellulose, mannitol, croscarmellose sodium, and magnesium stearate.

In some embodiments, the tablet disclosed herein comprises a compound of Formula Ia or Ib or a pharmaceutically acceptable salt thereof, copovidone, poloxamer 407, microcrystalline cellulose, mannitol, croscarmellose sodium, and magnesium stearate. In some embodiments, the tablet comprises a compound of Formula Ia, or a pharmaceutically acceptable salt thereof, copovidone, poloxamer 407, microcrystalline cellulose, mannitol, croscarmellose sodium, and magnesium stearate. In some embodiments, the tablet comprises a compound of Formula Ib, or a pharmaceutically acceptable salt thereof, copovidone, poloxamer 407, microcrystalline cellulose, mannitol, croscarmellose sodium, and magnesium stearate. In some embodiments, the tablet comprises a compound of Formula Ia, copovidone, poloxamer 407, microcrystalline cellulose, mannitol, croscarmellose sodium, and magnesium stearate. In some embodiments, the tablet comprises a sodium salt of the compound of Formula Ia, copovidone, poloxamer 407, microcrystalline cellulose, mannitol, croscarmellose sodium, and magnesium stearate. In some embodiments, the tablet comprises a compound of Formula Ib, copovidone, poloxamer 407, microcrystalline cellulose, mannitol, croscarmellose sodium, and magnesium stearate. In some embodiments, the tablet comprises a sodium salt of the compound of Formula Ib, copovidone, poloxamer 407, microcrystalline cellulose, mannitol, croscarmellose sodium, and magnesium stearate.

In some embodiments, the tablet disclosed herein comprises a sodium salt of the compound of Formula Ia or Ib, copovidone, poloxamer 407, microcrystalline cellulose, mannitol, croscarmellose sodium, and magnesium stearate. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 5 mg to about 500 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 25 mg to about 500 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 25 mg to about 400 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 25 mg to about 300 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 50 mg to about 300 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 5 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 10 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 20 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 25 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 30 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 40 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 50 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 75 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 100 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 125 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 150 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 175 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 200 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 225 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 250 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 275 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 300 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 325 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 350 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 375 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 400 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 425 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 450 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 475 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 500 mg.

In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 5 w/w % to about 45 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 10 w/w % to about 40 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 15 w/w % to about 35 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 15 w/w % to about 25 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 20.46 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 20.5 w/w %.

In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 1 w/w % to about 10 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 2 w/w % to about 10 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 3 w/w % to about 8 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 3 w/w % to about 6 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 4.88 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 4.9 w/w %.

In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 0.01 w/w % to about 10 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 0.05 w/w % to about 8 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 0.5 w/w % to about 4 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 0.5 w/w % to about 3.0 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 1.3 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 1.33 w/w %.

In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 5 w/w % to about 45 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 10 w/w % to about 40 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 15 w/w % to about 35 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 18 w/w % to about 30 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 21.28 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 21.3 w/w %.

In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 15 w/w % to about 70 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 20 w/w % to about 60 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 30 w/w % to about 55 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 40 w/w % to about 50 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 42.55 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 42.6 w/w %.

In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 1 w/w % to about 30 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 1 w/w % to about 20 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 4 w/w % to about 16 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 6 w/w % to about 10 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 8.0 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 8.00 w/w %.

In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 0.01 w/w % to about 10 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 0.05 w/w % to about 8 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 0.5 w/w % to about 4 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 1.0 w/w % to about 3.0 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 1.5 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 1.50 w/w %.

In some embodiments, the tablet disclosed herein comprises about 5 w/w % to about 45 w/w % of a sodium salt of the compound of Formula Ia or Ib, about 1 w/w % to about 10 w/w % of copovidone, about 0.01 w/w % to about 10 w/w % of poloxamer 407, about 5 w/w % to about 45 w/w % of microcrystalline cellulose, about 15 w/w % to about 70 w/w % of mannitol, about 1 w/w % to about 30 w/w % of croscarmellose sodium, about 0.01 w/w % to about 10 w/w % of magnesium stearate, and one or more pharmaceutically acceptable excipients. In some embodiments, the tablet disclosed herein comprises about 5 w/w % to about 45 w/w % of a sodium salt of the compound of Formula Ia or Ib, about 1 w/w % to about 10 w/w % of copovidone, about 0.01 w/w % to about 10 w/w % of poloxamer 407, about 5 w/w % to about 45 w/w % of microcrystalline cellulose, about 15 w/w % to about 70 w/w % of mannitol, about 1 w/w % to about 30 w/w % of croscarmellose sodium, and about 0.01 w/w % to about 10 w/w % of magnesium stearate. In some embodiments, the tablet comprises about 10 w/w % to about 40 w/w % of a sodium salt of the compound of Formula Ia or Ib, about 2 w/w % to about 10 w/w % of copovidone, about 0.05 w/w % to about 8 w/w % of poloxamer 407, about 10 w/w % to about 40 w/w % of microcrystalline cellulose, about 20 w/w % to about 60 w/w % of mannitol, about 1 w/w % to about 20 w/w % of croscarmellose sodium, and about 0.05 w/w % to about 8 w/w % of magnesium stearate. In some embodiments, the tablet comprises about 15 w/w % to about 35 w/w % of a sodium salt of the compound of Formula Ia or Ib, about 3 w/w % to about 8 w/w % of copovidone, about 0.5 w/w % to about 4 w/w % of poloxamer 407, about 15 w/w % to about 35 w/w % of microcrystalline cellulose, about 30 w/w % to about 55 w/w % of mannitol, about 4 w/w % to about 16 w/w % of croscarmellose sodium, and about 0.5 w/w % to about 4 w/w % of magnesium stearate. In some embodiments, the tablet comprises about 15 w/w % to about 25 w/w % of a sodium salt of the compound of Formula Ia or Ib, about 3 w/w % to about 6 w/w % of copovidone, about 0.5 w/w % to about 3.0 w/w % of poloxamer 407, about 18 w/w % to about 30 w/w % of microcrystalline cellulose, about 40 w/w % to about 50 w/w % of mannitol, about 6 w/w % to about 10 w/w % of croscarmellose sodium, and about 1.0 w/w % to about 3.0 w/w % of magnesium stearate.

In some embodiments, the tablet comprises about 20.5 w/w % of a sodium salt of the compound of Formula Ia or Ib, about 4.9 w/w % of copovidone, about 1.3 w/w % of poloxamer 407, about 21.3 w/w % of microcrystalline cellulose, about 42.6 w/w % of mannitol, about 8.0 w/w % of croscarmellose sodium, and about 1.5 w/w % of magnesium stearate. In some embodiments, the tablet comprises about 20.46 w/w % of a sodium salt of the compound of Formula Ia or Ib, about 4.88 w/w % of copovidone, about 1.33 w/w % of poloxamer 407, about 21.28 w/w % of microcrystalline cellulose, about 42.55 w/w % of mannitol, about 8.00 w/w % of croscarmellose sodium, and about 1.50 w/w % of magnesium stearate. In some embodiments, the tablet comprises about 20.5 w/w % of a sodium salt of the compound of Formula Ib, about 4.9 w/w % of copovidone, about 1.3 w/w % of poloxamer 407, about 21.3 w/w % of microcrystalline cellulose, about 42.6 w/w % of mannitol, about 8.0 w/w % of croscarmellose sodium, and about 1.5 w/w % of magnesium stearate. In some embodiments, the tablet comprises about 20.46 w/w % of a sodium salt of the compound of Formula Ib, about 4.88 w/w % of copovidone, about 1.33 w/w % of poloxamer 407, about 21.28 w/w % of microcrystalline cellulose, about 42.55 w/w % of mannitol, about 8.00 w/w % of croscarmellose sodium, and about 1.50 w/w % of magnesium stearate.

In some embodiments, the tablet disclosed herein further comprises an outer film coat. In some embodiments, the tablet comprising a sodium salt of the compound of Formula Ia or Ib, copovidone, poloxamer 407, microcrystalline cellulose, mannitol, croscarmellose sodium, and magnesium stearate further comprises an outer film coat. In some embodiments, the outer film coat provides from about 1% to about 8% weight gain based on the uncoated tablet. In some embodiments, the outer film coat provides from about 2% to about 6% weight gain based on the uncoated tablet. In some embodiments, the outer film coat provides from about 2% to about 4% weight gain based on the uncoated tablet. In some embodiments, the outer film coat provides from about 4% to about 6% weight gain based on the uncoated tablet. In some embodiments, the outer film coat provides about 1%, about 2%, about 3%, about 4%, about 5%, about 6%, about 7%, or about 8% weight gain based on the uncoated tablet. In some embodiments, the outer film coat provides about 1% weight gain based on the uncoated tablet. In some embodiments, the outer film coat provides about 2% weight gain based on the uncoated tablet. In some embodiments, the outer film coat provides about 3% weight gain based on the uncoated tablet. In some embodiments, the outer film coat provides about 4% weight gain based on the uncoated tablet. In some embodiments, the outer film coat provides about 5% weight gain based on the uncoated tablet. In some embodiments, the outer film coat provides about 6% weight gain based on the uncoated tablet. In some embodiments, the outer film coat provides about 7% weight gain based on the uncoated tablet. In some embodiments, the outer film coat provides about 8% weight gain based on the uncoated tablet. In some embodiments, the outer film coat comprises Opadry® II. In some embodiments, the outer film coat comprises Opadry® II White. In some embodiments, the outer film coat comprises Opadry® II White 85F18422.

In some embodiments, the outer film coat comprises Opadry® II Green. In some embodiments, the outer film coat comprises Opadry® II Green 85F110187. In some embodiments, the outer film coat comprises Opadry® II Green 85F110186.

In some embodiments, the tablet comprises about 20.5 w/w % of a sodium salt of a compound of Formula Ia or Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 3% weight gain from Opadry® II White 85F18422, wherein the weight gain is based on the uncoated tablet. In some embodiments, the tablet comprises about 20.46 w/w % of a sodium salt of a compound of Formula Ia or Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 3.0% weight gain from Opadry® II White 85F18422, wherein the weight gain is based on the uncoated tablet. In some embodiments, the tablet comprises about 20.5 w/w % of a sodium salt of a compound of Formula Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 3% weight gain from Opadry® II White 85F18422, wherein the weight gain is based on the uncoated tablet. In some embodiments, the tablet comprises about 20.46 w/w % of a sodium salt of a compound of Formula Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 3.0% weight gain from Opadry® II White 85F18422, wherein the weight gain is based on the uncoated tablet.

In some embodiments, the tablet comprises about 20.5 w/w % of a sodium salt of a compound of Formula Ia or Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 4% weight gain from Opadry® II Green 85F110187, wherein the weight gain is based on the uncoated tablet. In some embodiments, the tablet comprises about 20.46 w/w % of a sodium salt of a compound of Formula Ia or Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 4.0% weight gain from Opadry® II Green 85F110187, wherein the weight gain is based on the uncoated tablet. In some embodiments, the tablet comprises about 20.5 w/w % of a sodium salt of a compound of Formula Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 4% weight gain from Opadry® II Green 85F110187, wherein the weight gain is based on the uncoated tablet. In some embodiments, the tablet comprises about 20.46 w/w % of a sodium salt of a compound of Formula Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 4.0% weight gain from Opadry® II Green 85F110187, wherein the weight gain is based on the uncoated tablet.

In some embodiments, the tablet comprises about 20.5 w/w % of a sodium salt of a compound of Formula Ia or Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 4% weight gain from Opadry® II Green 85F110186, wherein the weight gain is based on the uncoated tablet. In some embodiments, the tablet comprises about 20.46 w/w % of a sodium salt of a compound of Formula Ia or Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 4.0% weight gain from Opadry® II Green 85F110186, wherein the weight gain is based on the uncoated tablet. In some embodiments, the tablet comprises about 20.5 w/w % of a sodium salt of a compound of Formula Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 4% weight gain from Opadry® II Green 85F110186, wherein the weight gain is based on the uncoated tablet. In some embodiments, the tablet comprises about 20.46 w/w % of a sodium salt of a compound of Formula Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 4.0% weight gain from Opadry® II Green 85F110186, wherein the weight gain is based on the uncoated tablet.

The pharmaceutical compositions disclosed herein can be also prepared by other methodologies known in the pharmaceutical art. For example, in certain embodiments, a pharmaceutical composition intended to be administered by injection can prepared by combining the compound of Formula Ia or Ib, or a pharmaceutically acceptable salt thereof, with sterile, distilled water so as to form a solution. In some embodiments, a surfactant is added to facilitate the formation of a homogeneous solution or suspension. Surfactants are compounds that non-covalently interact with the compound of Formula Ia or Ib, or a pharmaceutically acceptable salt thereof, so as to facilitate dissolution or homogeneous suspension of the compound in the aqueous delivery system.

The terms “effective amount” or “therapeutically effective amount” refer to an amount of the compound of Formula Ia or Ib, or other anti-HIV agent, or a pharmaceutically acceptable salt thereof, which when administered to a patient in need thereof, is sufficient to effect preventing an HIV infection or reducing the risk of contracting HIV infection, as described herein. Such an amount would be sufficient to elicit the biological or medical response of a tissue system, or patient that is sought by a researcher or clinician. The amount of the compound of Formula Ia or Ib or other anti-HIV agent which constitutes a therapeutically effective amount will vary depending on such factors as the compound, salt, or composition used for administration, the time of administration, the route of administration, the rate of excretion of the compound, the duration of the treatment, the type of disease-state or disorder being treated and its severity, drugs used in combination with or coincidentally with the compound of Formula Ia or Ib, and the age, body weight, general health, sex and diet of the patient. Such a therapeutically effective amount can be determined routinely by one of ordinary skill in the art having regard to their own knowledge, the state of the art, and this disclosure.

In some embodiments of the methods provided herein, each subcutaneous administration is administered as a solution comprising the sodium salt of the compound of Formula Ia or Ib.

In some embodiments of the methods provided herein, each solution comprises about 20 w/w % to about 30 w/w % water, about 48 w/w % to about 60 w/w % PEG 300, and about 11 w/w % to about 28 w/w % of a sodium salt of the compound of Formula Ia or Ib.

In some embodiments of the methods provided herein, each solution comprises about 23.41 w/w % water, about 50.13 w/w % PEG 300, and about 26.46 w/w % of the sodium salt of the compound of Formula Ia or Ib.

In some embodiments of the methods provided herein, each solution comprises about 309 mg/mL of the compound of Formula Ia or Ib, or a pharmaceutically acceptable salt thereof.

In some embodiments of the methods provided herein, each solution comprises about 315.4 mg/mL of the sodium salt of the compound of Formula Ia or Ib.

In some embodiments of the methods provided herein, each oral administration is administered as a tablet comprising the sodium salt of the compound of Formula Ia or Ib.

In some embodiments of the methods provided herein, each tablet is prepared from a spray-dried dispersion technology.

In some embodiments of the methods provided herein, each tablet comprises about 5 w/w % to about 45 w/w % of the sodium salt of the compound of Formula Ia or Ib, about 1 w/w % to about 10 w/w % of copovidone, about 0.01 w/w % to about 10 w/w % of poloxamer 407, about 5 w/w % to about 45 w/w % of microcrystalline cellulose, about 15 w/w % to about 70 w/w % of mannitol, about 1 w/w % to about 30 w/w % of croscarmellose sodium, and about 0.01 w/w % to about 10 w/w % of magnesium stearate, and one or more pharmaceutically acceptable excipients.

In some embodiments of the methods provided herein, each tablet comprises about 15 w/w % to about 25 w/w % of the sodium salt of the compound of Formula Ia or Ib, about 3 w/w % to about 6 w/w % of copovidone, about 0.5 w/w % to about 3.0 w/w % of poloxamer 407, about 18 w/w % to about 30 w/w % of microcrystalline cellulose, about 40 w/w % to about 50 w/w % of mannitol, about 6 w/w % to about 10 w/w % of croscarmellose sodium, and about 1.0 w/w % to about 3.0 w/w % magnesium stearate.

In some embodiments of the methods provided herein, each tablet comprises about 300 mg of the compound of Formula Ia, or a pharmaceutically acceptable salt thereof.

In some embodiments of the methods provided herein, each tablet comprises about 306.8 mg of the sodium salt of the compound of Formula Ia or Ib.

In some embodiments of the methods provided herein, each tablet further comprises an outer film coat.

In some embodiments of the methods provided herein, the outer film coat provides from about 1% to about 8% weight gain based on the uncoated tablet.

In some embodiments of the methods provided herein, wherein the outer film coat provides about 4% weight gain based on the uncoated tablet.

### Combination Therapies

Patients being treated by administration of the compound of Formula Ia or Ib, or a pharmaceutically acceptable salt thereof, often exhibit diseases or conditions that benefit from treatment with other therapeutic agents, including agents that are therapeutic for Retroviridae infections, including an HIV infection. In some embodiments, the other therapeutic agent is an agent that is therapeutic for an HIV infection. Thus, one aspect of the disclosure is a method of treating an HIV infection comprising administering a compound of Formula Ia or Ib, or a pharmaceutically acceptable salt thereof, in combination with one or more compounds useful for the treatment of an HIV infection to a patient in need thereof.

In some embodiments, a compound of Formula Ia or Ib, or a pharmaceutically acceptable salt thereof, is combined with one, two, three, four or more additional therapeutic agents. In some embodiments, a compound of Formula Ia or Ib, or a pharmaceutically acceptable salt thereof, is combined with two additional therapeutic agents. In some embodiments, a compound of Formula Ia or Ib, or a pharmaceutically acceptable salt thereof, is combined with three additional therapeutic agents. In some embodiments, a compound of Formula Ia or Ib, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In some embodiments, when a compound of Formula Ia or Ib, or a pharmaceutically acceptable salt thereof, is combined with one or more additional therapeutic agents as described herein, the components of the composition are administered as a simultaneous or sequential regimen.

In some embodiments, a compound of Formula Ia or Ib, or a pharmaceutically acceptable salt thereof, and the additional therapeutic agents are administered simultaneously.

In some embodiments, a compound of Formula Ia or Ib, or a pharmaceutically acceptable salt thereof, and the additional therapeutic agents are administered sequentially. When administered sequentially, the combination may be administered in two or more administrations.

In some embodiments, a compound of Formula Ia or Ib, or a pharmaceutically acceptable salt thereof, is combined with one or more additional therapeutic agents in a unitary dosage form for simultaneous administration to a patient.

In some embodiments, a compound of Formula Ia or Ib, or a pharmaceutically acceptable salt thereof, is co-administered with one or more additional therapeutic agents.

Co-administration includes administration of unit dosages of the compounds provided herein, or pharmaceutically acceptable salts thereof, before or after administration of unit dosages of one or more additional therapeutic agents. The compound of Formula Ia or Ib, or pharmaceutically acceptable salts thereof, may be administered within seconds, minutes, or hours of the administration of one or more additional therapeutic agents. For example, in some embodiments, a unit dose of a compound of Formula Ia or Ib, or a pharmaceutically acceptable salt thereof, is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of Formula Ia or Ib, or a pharmaceutically acceptable salt thereof, within seconds or minutes. In some embodiments, a unit dose of a compound of Formula Ia or Ib, or a pharmaceutically acceptable salt thereof, is administered first, followed, after a period of hours (i.e., 1-12 hours), by administration of a unit dose of one or more additional therapeutic agents. In other embodiments, a unit dose of one or more additional therapeutic agents is administered first, followed, after a period of hours (i.e., 1-12 hours), by administration of a unit dose of a compound of Formula Ia or Ib, or a pharmaceutically acceptable salt thereof.

### HIV Combination Therapy

In the above embodiments, the additional therapeutic agent or agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, anti-HIV peptides, and combinations thereof.

In some embodiments, the additional therapeutic agent or agents are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are chosen from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents any are chosen from dolutegravir, cabotegravir, darunavir, bictegravir, elsulfavirine, rilpivirine, and lenacapavir, and combinations thereof.

### HIV Combination Drugs

Examples of combination drugs include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, and dual-target HIV-1 reverse transcriptase/nucleocapsid protein 7 inhibitors.

### Other HIV Drugs

Examples of other drugs for treating HIV include, but are not limited to, aspernigrin C, acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ, AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937, GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558 November-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, x1-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169, BL-008, MK-8122, TMB-607, GRL-02031, and TMC-310911. Additional examples of HIV protease inhibitors are described, e.g., in U.S. Pat. No. 10,294,234, and U.S. Patent Application Publication Nos. US2020030327 and US2019210978.

### HIV Gag Protein Inhibitors

Examples of HIV Gag protein inhibitors include, but are not limited to, HRF-10071.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, efavirenz, etravirine, lentinan, nevirapine, rilpivirine, ACC-007, ACC-008, AIC-292, F-18, KM-023, PC-1005, M1-TFV, M2-TFV, VM-1500A-LAI, PF-3450074, elsulfavirine (sustained release oral, HIV infection), elsulfavirine (long acting injectable nanosuspension, HIV infection), and elsulfavirine (VM-1500). Additional non-limiting examples of non-nucleoside or non-nucleotide inhibitors of reverse transcriptase include the compounds disclosed in U.S. Pat. No. 10,548,898.

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, MK-8583, VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T169, STP-0404, VM-3500, XVIR-110, and ACC-017. Additional non-limiting examples of HIV integrase inhibitors include the compounds disclosed in U.S. Pat. No. 11,084,832.

Examples of HIV non-catalytic site, or allosteric, integrase inhibitors (NCINI) include, but are not limited to, CX-05045, CX-05168, and CX-14442.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

Examples of gp41 inhibitors include, but are not limited to, albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, C13hmAb, lipuvirtide, PIE-12 trimer and sifuvirtide.

Examples of CD4 attachment inhibitors include, but are not limited to, ibalizumab and CADA analogs.

Examples of gp120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188, N15 peptide, and vMIP (Haimipu).

### HIV Maturation Inhibitors

Examples of HIV maturation inhibitors include, but are not limited to, BMS-955176, GSK-3640254 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242), SMAC mimetics (including TL32711, LCL161, GDC-0917, HGS1029, AT-406, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B, and ubiquitin inhibitors such as largazole analogs, APH-0812, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, AVI-621, AVI-101, AVI-201, AVI-301, and AVI-CAN1-15 series, PF-3450074, HIV-1 capsid inhibitors (HIV-1 infection, Shandong University), and compounds described in (GSK WO2019/087016).

Additional examples of capsid inhibitors include, but not limited to, those described in U.S. Patent Application Publication Nos. US2018051005 and US2016108030.

Additional examples of HIV capsid inhibitors include, but are not limited to, those described in U.S. Patent Application Publication Nos. US2014221356 and US2016016973.

### Cytochrome P450 3 Inhibitors

Examples of Cytochrome P450 3 inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18 (11): 671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL 16 binding protein 1 (ULBP1); UL 16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18 (11): 671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (INFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi- and Tri-Specific Natural Killer (NK)-Cell Engagers

In various embodiments, the agents as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346; Fang et al., Semin Immunol. (2017) 31:37-54. Examples of trispecific NK cell engagers (TRIKE) include, but are not limited to, OXS-3550, HIV-TriKE, and CD16-IL-15-B7H3 Trike.

### Indoleamine-Pyrrole-2,3-Dioxygenase (ID) (1) Inhibitors

In various embodiments, the agents as described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the agents as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. TLR4 agonists include, but are not limited to, G-100 and GSK-1795091.

### CDK Inhibitors or Antagonists

In some embodiments, the agents described herein are combined with an inhibitor or antagonist of CDK. In some embodiments, the CDK inhibitor or antagonist is selected from the group consisting of VS2-370.

### STING Agonists, RIG-I and NOD2 Modulators

In some embodiments, the agents described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

### LAG-3 and TIM-3 Inhibitors

In certain embodiments, the agents as described herein are combined with an anti-TIM-3 antibody, such as TSR-022, LY-3321367, MBG-453, INCAGN-2390.

In certain embodiments, the antibodies or antigen-binding fragments described herein are combined with an anti LAG-3 (Lymphocyte-activation) antibody, such as relatlimab (ONO-4482), LAG-525, MK-4280, REGN-3767, INCAGN2385.

### Interleukin Agonists

In certain embodiments, the agents described herein are combined with an interleukin agonist, such as IL-2, IL-7, IL-15, IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO-C101, IL-15 Synthorin (pegylated Il-15), P-22339, and a IL-15-PD-1 fusion protein N-809; examples of IL-7 include without limitation CYT-107.

Examples of additional immune-based therapies that can be combined with an agent of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, peginterferon alfa-2b, and RPI-MN.

### Phosphatidylinositol 3-Kinase (PI3K) Inhibitors

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

alpha-4 beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, ibalizumab, ibalizumab (second generation), Immuglo, MB-66, clone 3 human monoclonal antibody targeting KLIC (HIV infection), GS-9721, BG-HIV, VRC-HIVMAB091-00-AB.

Various bNAbs may be used. Examples include, but are not limited to, those described in U.S. Pat. Nos. 8,673,307, 9,493,549, 9,783,594, 10,239,935, US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, IB2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492 (7427): 118-22 (2012), Horwitz et al., Proc Natl Acad Sci USA, 110 (41): 16538-43 (2013), Scheid et al., Science, 333: 1633-1637 (2011), Scheid et al., Nature, 458:636-640 (2009), Eroshkin et al, Nucleic Acids Res., 42 (Database issue): DI 133-9 (2014), Mascola et al., Immunol Rev., 254 (1): 225-44 (2013), such as 2F5, 4E10, M66.6, CAP206-CH12, 10E81 (all of which bind the MPER of gp41); PG9, PG16, CH01-04 (all of which bind V1V2-glycan), 2G12 (which binds to outer domain glycan); b12, HJ16, CH103-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173 (7): 1783-1795; Sajadi et al., J Infect Dis. (2016) 213 (1): 156-64; Klein et al., Nature, 492 (7427): 118-22 (2012), Horwitz et al., Proc Natl Acad Sci USA, 110 (41): 16538-43 (2013), Scheid et al., Science, 333:1633-1637 (2011), Scheid et al., Nature, 458:636-640 (2009), Eroshkin et al., Nucleic Acids Res., 42 (Database issue): Dl 133-9 (2014), Mascola et al., Immunol Rev., 254 (1): 225-44 (2013), such as 2F5, 4E10, M66.6, CAP206-CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1, BiIA-SG, CH01, CH59, C2F5, C4E10, C2F5+C2G12+C4E10, CAP256V2LS, 3BNC117, 3BNC117-LS, 3BNC60, DH270.1, DH270.6, D1D2, 10-1074-LS, C13hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7, VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include without limitation MGD014, B12BiTe, BiIA-SG, TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US20140221356 (Gilead Sciences), US20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AE-298p, anti-CD40.Env-gp140 vaccine, Ad4-EnvC150, BG505 SOSIP.664 gp140 adjuvanted vaccine, BG505 SOSIP.GT1.1 gp140 adjuvanted vaccine, ChAdOx1.tHIVconsv1 vaccine, CMV-MVA triplex vaccine, ChAdOx1.HTI, Chimigen HIV vaccine, ConM SOSIP.v7 gp140, ALVAC HIV (vCP1521), AIDSVAX B/E (gp120), monomeric gp 120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140 [delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PTI, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, PreVax Tat, AE-H, MYM-V101, CombiHIVvac, ADVAX, MYM-V201, MVA-CMDR, Maga Vax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp 120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; eOD-GT8 60 mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, arenavirus vector-based vaccines (Vaxwave, TheraT), MVA-BN HIV-1 vaccine regimen, mRNA based prophylactic vaccines, VPI-211, multimeric HIV gp120 vaccine (Fred Hutchinson cancer center), TBL-1203HI, CH505 TF chTrimer, CD40.HIVRI.Env vaccine, Drep-HIV-PT-1, mRNA-1644, and mRNA-1574.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a compound disclosed herein, or a pharmaceutically acceptable salt thereof, is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, an agent disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, an agent disclosed herein, or a pharmaceutical composition thereof, is combined with a first additional therapeutic agent chosen from dolutegravir, cabotegravir, darunavir, bictegravir, elsulfavirine, rilpivirine, and lenacapavir and a second additional therapeutic agent chosen from emtricitabine and lamivudine.

In some embodiments, an agent disclosed herein, or a pharmaceutical composition thereof, is combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the agents described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-B01, HSPCs overexpressing ALDH1 (LV-800, HIV infection), AGT103-T, and SupT1 cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the agents described herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T Cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp 120, N glycan on gp 120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR T-Cell Therapy

In certain embodiments, the agents described herein are combined with a population of TCR-T-cells. TCR-T-cells are engineered to target HIV derived peptides present on the surface of virus-infected cells, for example, ImmTAV.

### B-Cell Therapy

In certain embodiments, the antibodies or antigen-binding fragments described herein are combined with a population of B cells genetically modified to express broadly neutralizing antibodies, such as 3BNC117 (Hartweger et al., J. Exp. Med. 2019, 1301, Moffett et al., Sci. Immunol. 4, eaax0644 (2019) 17 May 2019.

A compound of Formula Ia or Ib, or a pharmaceutically acceptable salt thereof, may be combined with one, two, three, or four additional therapeutic agents in any dosage amount of the compound of Formula Ia or Ib, or a pharmaceutically acceptable salt thereof.

In one embodiment, kits comprising a compound disclosed herein, or a pharmaceutically acceptable salt thereof, in combination with one or more (e.g., one, two, three, one or two, or one to three) additional therapeutic agents are provided.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MLK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In a specific embodiment, the kit includes a compound disclosed herein, or a pharmaceutically acceptable salt thereof, and an HIV nucleoside or nucleotide inhibitor of reverse transcriptase. In a specific embodiment, the kit includes a compound disclosed herein, or a pharmaceutically acceptable salt thereof, and an HIV nucleoside or nucleotide inhibitor of reverse transcriptase and an HIV non-nucleoside inhibitor of reverse transcriptase. In another specific embodiment, the kit includes a compound disclosed herein, or a pharmaceutically acceptable salt thereof, and an HIV nucleoside or nucleotide inhibitor of reverse transcriptase, and an HIV protease inhibiting compound. In an additional embodiment, the kit includes a compound disclosed herein, or a pharmaceutically acceptable salt thereof, an HIV nucleoside or nucleotide inhibitor of reverse transcriptase, an HIV non-nucleoside inhibitor of reverse transcriptase, and a pharmacokinetic enhancer. In certain embodiments, the kit includes a compound disclosed herein, or a pharmaceutically acceptable salt thereof, at least one HIV nucleoside inhibitor of reverse transcriptase, an integrase inhibitor, and a pharmacokinetic enhancer. In another embodiment, the kit includes a compound disclosed herein, or a pharmaceutically acceptable salt thereof, and two HIV nucleoside or nucleotide inhibitors of reverse transcriptase. In a specific embodiment, the kit includes a compound disclosed herein, or a pharmaceutically acceptable salt thereof, an HIV nucleoside or nucleotide inhibitor of reverse transcriptase and an HIV capsid inhibitor. In a specific embodiment, the kit includes a compound disclosed herein, or a pharmaceutically acceptable salt thereof, an HIV nucleoside inhibitor of reverse transcriptase and an HIV capsid inhibitor. In a specific embodiment, the kit includes a compound disclosed herein, or a pharmaceutically acceptable salt thereof, and an HIV capsid inhibitor. In a specific embodiment, the kit includes a compound disclosed herein, or a pharmaceutically acceptable salt thereof, and one, two, three or four HIV bNAbs. In a specific embodiment, the kit includes a compound disclosed herein, or a pharmaceutically acceptable salt thereof, one, two, three or four HIV bNAbs and an HIV capsid inhibitor. In a specific embodiment, the kit includes a compound disclosed herein, or a pharmaceutically acceptable salt thereof, one, two, three or four HIV bNAbs, an HIV capsid inhibitor, and an HIV nucleoside inhibitor of reverse transcriptase.

### HIV Long Acting Therapy

Examples of drugs that are being developed as long acting regimens include, but are not limited to, cabotegravir, rilpivirine, any integrase LA, VM-1500 LAI, maraviroc (LAI), tenofovir implant, doravirine, raltegravir, and long acting dolutegravir.

In some embodiments, the methods provided herein further comprise administering one, two, three, or four additional therapeutic agents to the patient.

In some embodiments of the methods provided herein, the additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof.

In some embodiments of the methods provided herein, the additional therapeutic agents are selected from the group consisting of HIV protease inhibiting compounds, HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, pharmacokinetic enhancers, and other drugs for treating HIV, or any combinations thereof.

In some embodiments of the methods provided herein, the additional therapeutic agents are selected from the group consisting of bictegravir or a pharmaceutically acceptable salt thereof, abacavir sulfate, tenofovir, tenofovir disoproxil, tenofovir disoproxil fumarate, tenofovir disoproxil hemifumarate, tenofovir alafenamide, and tenofovir alafenamide hemifumarate.

In some embodiments of the methods provided herein, the additional therapeutic agents are selected from the group consisting of bictegravir or a pharmaceutically acceptable salt thereof, tenofovir alafenamide, tenofovir alafenamide fumarate, and tenofovir alafenamide hemifumarate.

### EXAMPLES

The disclosure will be described in greater detail by way of specific examples. The following examples are offered for illustrative purposes, and are not intended to limit the disclosure in any manner. Those of skill in the art will readily recognize a variety of noncritical parameters which can be changed or modified to yield essentially the same results.

### Example 1. Dosing and Administration of Oral Weekly Bridging Dose of Lenacapavir

Lenacapavir (LEN) is currently approved for multidrug-resistant HIV-1 infection in combination with other antiretrovirals for heavily treatment-experienced individuals. Lenapcapavir can be administered to patients according to the protocol in Table 1:

TABLE 1
Initiation Option 1

| | |
|---|---|
| Day 1 | 927 mg by subcutaneous injection |
| | (2 × 1.5 mL injections) |
| | 600 mg orally (2 × 300 mg tablets) |
| Day 2 | 600 mg orally (2 × 300 mg tablets) |

Initiation Option 2

| | |
|---|---|
| Day 1 | 600 mg orally (2 × 300 mg tablets) |
| Day 2 | 600 mg orally (2 × 300 mg tablets) |
| Day 8 | 300 mg orally (1 × 300 mg tablets) |
| Day 15 | 927 mg by subcutaneous injection |
| | (2 × 1.5 mL injections) |

Maintenance
927 mg by subcutaneous (SC) injection (2 × 1.5 mL injections)
every 6 months (26 weeks) from the date of the last
injection +/−2 weeks. Two 1.5 mL subcutaneous
injections are required for complete dose.

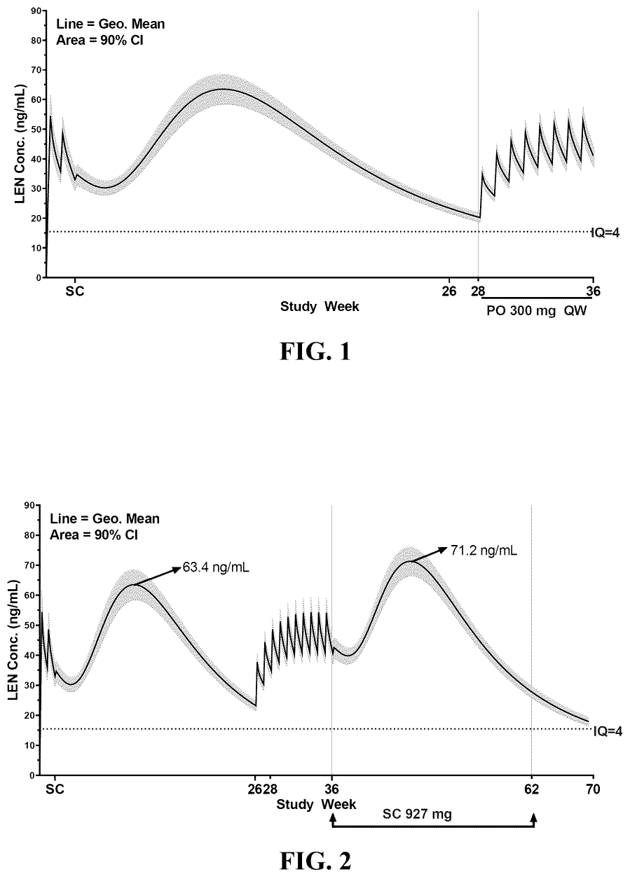

Oral bridging of lenacapavir is supported by antiviral activity, PK, and safety data from a Phase 1b proof of concept study, ongoing Phase 2 and 2/3 studies, as well as pharmacokinetic (PK) and safety data from 2 Phase 1 studies in healthy volunteers. Phenotypic analyses and pharmacokinetic/pharmacodynamic (PK/PD) modeling indicate that a lenacapavir plasma concentration of 15.5 ng/mL, corresponding to an inhibitory quotient (IQ) of 4 or higher, would provide near maximal antiviral activity. An oral weekly bridging dose, even when started as late as 28 weeks after the last lenacapavir SC injection, is believed to immediately maintain the lower bound of the 90% confidence interval (CI) of arithmetic mean for lenacapavir Cₜₐᵤ above IQ4 (i.e., even before reaching steady state), as shown in FIG. 1. As long as the oral weekly bridging is initiated between 26 to 28 weeks after the last lenacapavir SC injection, the PK profile upon resuming SC injection is believed to be comparable with that of the prior SC dose and within the target range regardless of when SC injection is resumed, as shown in FIG. 2.

Patients may require an oral weekly bridging dosage of lenacapavir if an SC injection of lenacapavir cannot be administered for any reason within the protocol visit window, as shown in Table 2. If 26 to 28 weeks elapses since the last SC injection and if clinically appropriate to continue lenacapavir, start oral lenacapavir 300 mg (1 tablet) once a week at oral bridging visits and continue weekly dosing on the same day of the week. Participants may continue receiving oral weekly lenacapavir 300 mg until they can receive their next SC lenacapavir injection.

TABLE 2

| 26 to 28 Weeks of Elapse | |
|---|---|
| Since the Last SC Injection | Lenacapavir |
| Treatment time from Oral Start | Dose |
| Day 1 | Oral 300 mg |
| | (1 × 300 mg tablets) |
| Day 8; | Oral 300 mg |
| continue oral weekly lenacapavir on the | (1 × 300 mg tablets) |
| same day of the week as first dose until | |
| lenacapavir SC is administered | |
| SC administration restart | SC 927 mg |
| (within one week of the oral weekly | every 6 months (2 × 1.5 mL |
| 300 mg dose [as early as after Day | injections) |
| 1 dosing]) | |

N/A = not applicable;
SC = subcutaneous

Missed dose recommendations for the oral weekly bridging dosages are provided in Table 3. The scheduled dosing day of the week should not change due to the missed dose of lenacapavir.

TABLE 3

| Number of Days Since | | |
|---|---|---|
| Initial Missed Schedule | | |
| Dose | Recommendation | Example |
| 1 to 6 days (1 missed | Take 300 mg (1 tablet) as | Participant forgets to take |
| weekly dose) | soon as possible, then | dose on Monday |
| | resume normal schedule, | (scheduled) but remembers |
| | taking 300 mg (1 tablet) on | before the next scheduled |
| | the next scheduled dosing | dose day (ie, Tuesday- |
| | day and weekly thereafter | Sunday). Take 1 dose as |
| | | soon as possible, then take |
| | | 1 dose on the following |
| | | Monday as scheduled. |
| 7 to 14 days (1-2 missed | Take 600 mg (2 tablets) as | Participant forgets to take |
| weekly doses) | soon as possible, then | dose on Monday |
| | resume weekly dosing by | (scheduled) but remembers |
| | taking 300 mg (1 tablet) on | the following Monday. |
| | the next scheduled weekly | Take 2 doses on the second |
| | dosing day and weekly | Monday and resume dosing |
| | thereafter. If participant | schedule (1 dose on |
| | remembers on scheduled | Mondays). |
| | dosing day, then take 600 | Participant forgets to take |
| | mg (2 tablets) only. Never | dose on 2 consecutive |
| | take greater than 600 mg | Mondays (scheduled) but |
| | on a single day. | remembers a few days later |
| | | (e.g., Tuesday-Sunday |
| | | following second missed |
| | | dose) before the third |
| | | Monday. Take 2 doses as |
| | | soon as possible, then take |
| | | 1 dose the next Monday as |
| | | scheduled. |
| | | Participant forgets to take |
| | | dose on 2 consecutive |
| | | Mondays (scheduled) but |
| | | remembers on the third |
| | | Monday. Take 2 doses on |
| | | the third Monday and |
| | | resume dosing schedule (1 |
| | | dose on Mondays). Never |
| | | take 3 doses on the same |
| | | day. |
| More than 14 days (3 or | Assess whether clinically | Participant forgets to take |
| more missed weekly doses) | appropriate to continue oral | dose on 3 consecutive |
| | weekly bridging. Consider | Mondays (scheduled). |
| | checking HIV-1 RNA. | Clinical assessment |
| | | needed. |

### Example 2. Pharmacokinetic Bridging with Lenacapavir Oral 300-Mg Once-Weekly Regimen for Missed Subcutaneous Q6M Dosing

Lenacapavir (LEN) is currently approved for multidrug-resistant HIV-1 infection in combination with other antiretrovirals for heavily treatment-experienced individuals. In ongoing Phase 2/3 studies (CAPELLA and CALIBRATE), participants receive oral LEN loading (600 mg on Days 1 and 2; 300 mg on Day 8) followed by 927 mg subcutaneous (SC) administration every six months (Q6M) starting from Day 15. In these studies, mean trough concentrations>15.5 ng/mL, which is inhibitory quotient-4 (IQ4; i.e., 4-fold in-vitro protein binding-adjusted 95% effective concentration), are associated with high rates of virologic suppression. In participants temporarily unable to receive SC LEN during its clinical hold, oral bridging (300 mg QW) was used to bridge LEN concentrations until SC dosing was resumed.

The objective of this study was to evaluate the pharmacokinetics (PK) of LEN during oral bridging period (OBP) to assess the adequacy of 300-mg PO once-weekly (QW) regimen for maintaining therapeutic concentrations between missed SC LEN doses. During OBP, sparse PK samples were collected at the start of OBP and every ˜10-12 weeks (without regard to a prespecified time since dose) until SC LEN was resumed. LEN plasma concentrations were summarized during OBP in the CAPELLA (N=57) and CALIBRATE (N=82) studies.

In both studies, mean LEN concentration and the lower-bound 90% confidence interval (CI) were maintained above the efficacy target of IQ4 at all OBP visits, as shown in Table 4). At the time of SC LEN resumption, mean (lower-bound 90% CI) predose concentrations in CAPELLA (74.4 ng/ml [56.2 ng/mL]) and CALIBRATE (50.7 [43.6]) exceeded IQ4. Mean LEN concentrations and the lower-bound 90% CIs were maintained above IQ4 from the first PO LEN bridging visits until SC LEN was resumed in both studies.

These results indicate that LEN 300 mg PO QW provides adequate concentrations to bridge LEN dosing in participants who may miss their Q6M SC injection.

TABLE 4

| | | OBP | OBP | OBP | OBP |
|---|---|---|---|---|---|
| Study | Concentration | Day 1 | Week 10 | Week 20 | Week 30 |
| CAPELLA | Mean, ng/mL | 46.1 | 76.2 | 74.8 | 41.7 |
| | | (n = 56) | (n = 57) | (n = 36) | (n = 39) |
| | 90% CI | 40.3, 51.9 | 66.1, 86.3 | 50.4, 99.3 | 29.9, 53.5 |
| CALIBRATE | Mean, ng/mL | 27.8 | 54.9 | 52.5 | 50.1 |
| | | (n = 76) | (n = 68) | (n = 60) | (n = 6) |
| | 90% CI | 25.3, 30.4 | 47.1, 62.8 | 43.7, 61.4 | 24.4, 75.7 |

### Example 3. Oral Bridging (300 mg QW) Maintains Efficacy with a Similar Safety Profile when SC LEN Cannot be Administered

Subcutaneous (SC) LEN provides a Q6M treatment option for HIV-1; however, potential SC treatment interruptions may lead to management challenges due to SC treatment gaps. This subanalysis investigated the efficacy and safety of an oral bridging (OB) regimen (LEN 300 mg PO QW) in participants with MDR HIV-1 as well as treatment-naïve people with HIV-1 (PWH) enrolled in the CAPELLA and CALIBRATE studies, respectively, when SC LEN dosing was interrupted.

Virologic suppression, CD4+ cell counts, and safety outcomes were assessed from available data at baseline (time of initiation of OB) and until SC resumption or early discontinuation from OB during a clinical hold period (December 2021-May 2022). Of 72 participants who received SC LEN in CAPELLA, 57 received OB (79%) and were included in this analysis. Of 105 participants who received SC LEN in CALIBRATE, 82 received OB (78%). In both studies, demographic and baseline characteristics were similar between OB and overall analysis sets. Median OB exposure was 19 weeks, and OB adherence (by pill count) was ≥95% in most participants.

Results were consistent across both subanalyses. High virologic suppression rates were maintained among those already suppressed (HIV-1 RNA<50 copies/mL) at OB baseline in CAPELLA and in all participants in CALIBRATE, as shown in Table 5. CD4 (abs/%) remained stable or increased from OB baseline. One participant (CAPELLA) who missed 2 non-consecutive oral LEN doses did not maintain virologic suppression during OB. Treatment-emergent adverse events (AEs) were similar to SC LEN. Two participants in CAPELLA (3.5%) and 1 participant in CALIBRATE (1.2%) experienced treatment-related diarrhea.

High rates of virologic suppression, and stable or increased CD4, support efficacy of OB in PWH, including those with MDR HIV-1 whose SC LEN treatment was interrupted. During OB, LEN was generally safe and well tolerated.

TABLE 5
Number and Proportion of Participants With HIV-1 RNA <50
copies/mL by Visit, Missing = Excluded

| n/N (%) | CAPELLA^{a,b} | CALIBRATE^{b} |
|---|---|---|
| OB Baseline | 46/46 (100) | 82/82 (100) |
| OB Week 10 | 44/45 (98) | 77/77 (100) |
| OB Week 20 | 30/31 (97) | 58/58 (100) |
| OB Week 30 | 10/10 (100) | 5/5 (100) |

^{a}Participants had virologic suppression at OB Baseline.
^{b}Denominators reflect participants who reached the specified duration of OB.

### Example 4. Oral Bridging of Lenacapavir in Combination with an Optimized Background Regimen in Heavily Treatment Experienced People Living with HIV-1 Infection with Multidrug Resistance

This Example describes analysis of oral LEN bridging administered during a Phase 2/3, global, randomized, placebo-controlled multicenter study of lenacapavir (LEN) together with an optimized background regimen (OBR) in people with HIV (PWH) with multidrug resistance (MDR) (to >2 antiretroviral (ARV) medications from each of ≥3 of the 4 main classes) and who are failing their current regimen (defined as plasma HIV-1 RNA≥400 copies/mL).

During a partial clinical hold, oral LEN (300 mg once weekly) was allowed to be administered as an alternative option for subcutaneous (SC) LEN. Oral bridging with 300-mg oral LEN once weekly was implemented following the clinical hold. When the partial hold was lifted, participants were able to resume SC LEN. This Example describes the oral bridging interim analysis that was conducted after all participants who received oral bridging of LEN during the clinical hold had resumed their SC LEN injection or prematurely discontinued the study drug.

### Study Design

This Example describes a Phase 2/3, global, randomized, placebo-controlled multicenter study of LEN together with an OBR in PWH with MDR (to ≥2 ARV medications from each of ≥3 of the 4 main classes) and who are failing their current regimen (defined as plasma HIV-1 RNA≥400 copies/mL). Participants required oral bridging if an SC injection of LEN was not administered within the protocol visit window.

Participants were enrolled in either of the following two cohorts:
Cohort 1: Randomized in a 2:1 ratio to receive oral LEN or placebo for 14 days starting on Day 1, while they continued their existing regimen; or
Cohort 2: Enrolled to receive oral LEN together with an OBR.

Oral bridging of LEN is supported by antiviral activity, pharmacokinetics (PK), and safety data from a prior Phase 1b, proof-of-concept study, two ongoing Phase 2 and 2/3 studies described in Examples 4-5, as well as PK and safety data from Phase 1 studies in healthy volunteers. Phenotypic analyses and PK-pharmacodynamic modeling indicate that a mean trough concentration of 15.5 ng/mL, which is inhibitory quotient 4 (IQ4; 4-fold greater than the in vitro protein-adjusted 95% effective concentration derived from MT-4 cells) has been associated with high rates of virologic suppression in clinical studies.

Lenacapavir has been administered orally at doses up to 1800 mg. Safety data from all completed and ongoing clinical studies indicated that LEN was generally safe and well tolerated at the intended exposures. Oral LEN was administered without regard to food.

### Inclusion Criteria

Inclusion criteria for the study were as follows:
1) Was willing and able to provide written informed consent (participants≥18 years of age) and assent (participants≥12 and <18 years of age) prior to performing study procedures. For participants≥12 and <18 years of age, the parent or legal guardian was willing and able to provide written informed consent prior to performing study procedures as required by local law.
2) Adult aged ≥8 years (at all sites) or adolescent aged ≥12 and weighing ≥35 kg (at sites in North America and Dominican Republic).
3) Was receiving a stable failing ARV regimen for >8 weeks before screening and willing to continue the regimen until Day 1. Participants in Cohort 1 must have also been willing to continue the failing regimen until completing the Functional Monotherapy Period (Day 1 to Day 14).
4) Had HIV-1 RNA≥400 copies/mL at screening.
5) Had screening or available historical HIV resistance reports showing resistance to ≥2 ARV medications from each of ≥3 of the 4 main classes of ARV medications (nucleoside/nucleotide reverse transcriptase inhibitor [NRTI/NtRTI], nonnucleoside/nucleotide reverse transcriptase inhibitor [NNRTI], protease inhibitor [PI], integrase strand-transfer inhibitor [INSTI]). Resistance to emtricitabine or lamivudine associated with the presence of the M184V/I reverse transcriptase mutation could not be used for the purpose of determining this eligibility criterion.
6) Had ≤2 fully active ARV remaining from the 4 main classes that can be effectively combined to form a viable regimen in the opinion of the investigator based on resistance, tolerability, contraindication, safety, drug access, or acceptability to the participant.
7) Was willing and able to receive an OBR together with LEN. Participants with an OBR without a fully active agent could be enrolled if the investigator considered that there was a favorable risk-benefit ratio for the participant.
8) If female, had a negative serum pregnancy test at screening.
9) Participants of childbearing potential who engaged in heterosexual intercourse must have agreed to use protocol-specified method(s) of contraception as described in the clinical study protocol.
10) Lactating women must have agreed to discontinue nursing before administration of LEN.

### Exclusion Criteria

Exclusion criteria for the study were as follows:
1) Had an opportunistic illness requiring acute therapy within the 30 days prior to screening.
2) Had active, serious infections (other than HIV-1 infection) requiring parenteral antibiotic or antifungal therapy within 30 days before screening.
3) Had active tuberculosis infection.
4) Had acute hepatitis within 30 days prior to screening visit.
5) Had untreated or newly treated (<3 months prior to screening) hepatitis B virus (HBV) infection. Participants could be enrolled regardless of the HBV serology criteria below if they were receiving treatment with anti-HBV activity and planned to continue the treatment during the study. Hepatitis B infection was defined as screening results showing either or both of:
   a. Positive HBV surface antigen.
   b. Positive HBV core antibody and negative HBV surface antibody. Participants could be enrolled with these findings if they had HBV DNA than the lower limit of quantitation (LLOQ).
6) Had hepatitis C virus (HCV) antibody positive and HCV RNA greater than LLOQ.
7) Had a history of or current clinical decompensated liver cirrhosis (e.g., ascites, encephalopathy, or variceal bleeding).
8) Had treatment within 3 months prior to screening, or anticipated treatment during the study period with immunosuppressant therapies, hydroxyurea, foscarnet, radiation, or cytotoxic chemotherapeutic agents without prior approval prior to randomization.
9) Had active malignancy requiring acute therapy (with the exception of local cutaneous Kaposi's sarcoma).
10) Had current alcohol or substance use judged by the investigator to potentially interfere with the participant's study compliance.
11) Had clinically significant abnormal electrocardiogram (ECG) at the screening visit.
12) Had any of the following laboratory values at screening:
   a. Estimated glomerular filtration rate (eGFR)≤50 mL/min using Cockcroft-Gault formula for participants≥18 years of age (see e.g., Cockcroft & Gault, Nephron, 1976; 16:31-41) and Schwartz Formula for participants<18 years of age for creatinine clearance.
   b. Alanine aminotransferase (ALT)>5× upper limit of normal (ULN).
   c. Direct bilirubin>1.5×ULN.
   d. Platelets<50,000/mm³.
   e. Hemoglobin<8.0 g/dL.
13) Had participated or planned to participate in any other clinical trial (including observational trials) without prior approval from the sponsor throughout the study.
14) Had prior use of, or exposure to, LEN.
15) Had known hypersensitivity to LEN, the metabolites, or formulation excipient.
16) Had used or planned to use of exclusionary medications defined in the clinical study protocol.
17) Had any other clinical condition or prior therapy that, in the opinion of the investigator, would make the participant unsuitable for the study or unable to comply with dosing requirements.

### Oral Bridging Treatment Administration

Participants required oral weekly bridging if an SC injection of LEN could not be administered for any reason within the protocol visit window. If 26 to 28 weeks elapsed since the last SC injection, and if clinically appropriate to continue LEN, participants started oral LEN 300 mg (1 tablet) once a week at the oral bridging visit(s) and continued weekly dosing on the same day of the week. If more than 28 weeks elapsed since the last SC injection, and if clinically appropriate to continue LEN, participants restarted the oral lead-in at the oral bridging visits and continued oral weekly dosing on the same day of the week, until SC LEN was administered, as shown in Table 6.

TABLE 6
Dosing Schedule for Oral Weekly Bridging and Resumption of SC LEN Injection

| Elapsed 26 to 28 Weeks | More than 28 Weeks Elapsed | |
|---|---|---|
| Since the Last SC Injection | Since the Last SC Injection | LEN Dose |
| Treatment Time from Oral Start | Treatment Time from Oral Start | Dose |
| NA | Day 1 | Oral 600 mg |
| | | (2 × 300 mg tablets) |
| NA | Day 2 | Oral 600 mg |
| | | (2 × 300 mg tablets) |
| Day 1 | Day 8: | Oral 300 mg |
| | continued oral weekly LEN | (1 × 300 mg tablets) |
| | on the same day of the week | |
| | as first dose until LEN SC is | |
| | adminsitered | |
| Day 8; | NA | Oral 600 mg |
| continued oral weekly LEN | | (2 × 300 mg tablets) |
| on the same day of the week | | once a week |
| as the first dose until SC LEN | | |
| is administered | | |
| SC administration restarted | SC administration restarted | SC 927 mg |
| (within 1 week of the oral | (within 1 week of the oral | every 6 months |
| weekly 300 mg dose (as early | weekly 300 mg dose (as early | |
| as after Day 1 dosing) | as after Day 8 dosing) | |

The oral bridging dose of LEN 300 mg was administered once weekly starting 26 to 28 weeks after the last SC LEN injection. This oral weekly bridging dose, even when started as late as 28 weeks after the last SC LEN injection, was predicted to immediately maintain the lower bound of the 90% CI of arithmetic mean for LEN Cₜₐᵤ above IQ4 (i.e., even before reaching steady state). As long as the oral weekly bridging is initiated between 26 to 28 weeks after the last LEN SC injection, the PK profile upon resuming SC injection was predicted to be comparable with that of the prior SC dose and within the target range regardless of when SC injection is resumed. Oral bridging day was calculated from first dose date of Oral Bridging.

### Adverse Events

An adverse event (AE) was any untoward medical occurrence in a clinical study participant administered a study drug, which did not necessarily have a causal relationship with the treatment. An AE could, therefore, be any unfavorable and/or unintended sign, symptom, or disease temporally associated with the use of a study drug, whether or not considered related to the study drug. Adverse events also included pretreatment or posttreatment complications that occurred as a result of protocol-specified procedures, lack of efficacy (if applicable to study), overdose, drug abuse/misuse reports, or occupational exposure. Preexisting events that increased in severity or changed in nature during or as a consequence of participation in the clinical study were also considered AEs.

The relationship or association of the AE to a study treatment was assessed using clinical judgment and the following considerations:
Not related: Evidence existed that the AE had an etiology other than the study drug. For serious adverse events (SAEs), an alternative causality must have been provided (e.g., preexisting condition, underlying disease, intercurrent illness, or concomitant medication).
Related: There was reasonable possibility that the event was caused by the study drug.

The relationship to study procedures (e.g., invasive procedures such as venipuncture or biopsy) was assessed using the following considerations:
Not related: Evidence existed that the AE had an etiology other than the study procedure.
Related: The AE occurred as a result of protocol procedures (e.g., venipuncture).

### Serious Adverse Events

A serious adverse event (SAE) was defined as an event that, at any dose, resulted in any of the following outcomes:
Death.
Life-threatening situation (participant was at immediate risk of death).
Inpatient hospitalization or prolongation of existing hospitalization.
Persistent or significant disability/incapacity.
A congenital anomaly/birth defect.
Other medically significant events that based upon appropriate medical judgment may have jeopardized the participant or may have required medical or surgical intervention to prevent one of the outcomes listed above.

### Summary of Efficacy Results

Mean (SD) oral bridging baseline HIV-1 RNA values for participants who received oral bridging was 1.74 (0.947) log₁₀ copies/mL. At Weeks 10 and 20, mean (SD) change from oral bridging baseline in HIV-1 RNA was-0.11 (0.597) and −0.16 (0.476) log 10 copies/mL, respectively.

Change from Baseline in HIV-1 RNA in Participants with Oral Bridging Baseline HIV-1 RNA-50 Copies mL and ≥50 Copies mL

Mean (SD) baseline HIV-1 RNA values for participants with oral bridging baseline HIV-1 RNA<50 copies/mL was 1.34 (0.121) log₁₀ copies/mL. At Weeks 10 and 20, mean (SD) change from oral bridging baseline in HIV-1 RNA was 0.00 (0.250) and −0.01 (0.096) log₁₀ copies/mL, respectively.

Mean (SD) oral bridging baseline HIV-1 RNA values of participants with oral bridging baseline HIV-1 RNA≥50 copies/mL was 3.43 (1.035) log₁₀ copies/mL. At Weeks 10 and 20, mean (SD) change from oral bridging baseline in HIV-1 RNA was −0.58 (1.188) and −1.05 (0.867) log₁₀ copies/mL, respectively.

### Change From Baseline in CD4 Cell Count

Mean (SD) oral bridging baseline CD4 cell count values of participants who received oral bridging was 356 (261.1) cells/μL. At Weeks 10 and 20, mean (SD) change from oral bridging baseline in CD4 cell count was −16 (114.2) and 12 (71.8) cells/μL, respectively.

### Change From Baseline in CD4 Percentage

Mean (SD) oral bridging baseline CD4 percentage in participants who received oral bridging was 17.0% (9.02). At Weeks 10 and 20, mean (SD) change from oral bridging baseline in CD4 percentage was 0.2% (2.69) and 1.0% (2.48), respectively.

### HIV-1 RNA 50 Copies/mL Using Imputation Methods (M=F and M=E)

At Weeks 10 and 20, mean (SD) change from oral bridging baseline in CD4 percentage was 0.2% (2.69) and 1.0% (2.48), respectively. At Weeks 10 and 20 for the Missing=Failure (M=F) analysis, the percentage of participants who received oral bridging with HIV-1 RNA<50 copies/mL was 83.9% (47 of 56 participants) and 84.2% (32 of 38 participants), respectively. The percentage of participants with oral bridging baseline HIV-1 RNA<50 copies/mL who maintained HIV-1 RNA<50 copies/mL at Weeks 10 and 20 was 97.8% (44 of 45 participants) and 90.9% (30 of 33 participants), respectively. The percentage of participants with oral bridging baseline HIV-1 RNA≥50 copies/mL who achieved HIV-1 RNA<50 copies/mL at Weeks 10 and 20 was 27.3% (3 of 11 participants) and 40.0% (2 of 5 participants), respectively.

At Weeks 10 and 20 for the Missing=Excluded (M=E) analysis, the percentage of participants who received oral bridging with HIV-1 RNA<50 copies/mL was 83.9% (47 of 56 participants) and 91.4% (32 of 35 participants), respectively. The percentage of participants with oral bridging baseline HIV-1 RNA<50 copies/mL who maintained HIV-1 RNA<50 copies/mL at Weeks 10 and 20 was 97.8% (44 of 45 participants) and 96.8% (30 of 31 participants), respectively. Of the 46 participants with oral bridging baseline HIV-1 RNA<50 copies/mL, there was 1 participant who did not maintain HIV-1 RNA<50 copies/mL during the oral bridging period at Weeks 10 and 20. The participant missed 2, nonconsecutive doses of oral LEN prior to the elevated HIV-1 RNA results. The percentage of participants with oral bridging baseline HIV-1 RNA≥50 copies/mL who achieved HIV-1 RNA<50 copies/mL at Weeks 10 and 20 was 27.3% (3 of 11 participants) and 50.0% (2 of 4 participants), respectively.

The majority of participants who were virologically suppressed at oral bridging baseline (HIV-1 RNA<50 copies/mL) remained virologically suppressed throughout the Oral Bridging Period.

### Summary of Pharmacokinetics

### LEN Plasma Concentrations

Mean LEN concentration and the lower bound 90% CI were maintained above the efficacy target of IQ4 (15.5 ng/mL) from the first oral LEN bridging visit through the SC LEN resumption visit.

For the oral bridging period, mean (% CV) predose concentration and the lower bound 90% CI at oral bridging Day 1 [46.1 ng/ml (56.3%); lower 90% CI=40.3 ng/mL], at oral bridging Week 10 [76.2 ng/ml (59.6%); lower 90% CI=66.1 ng/mL], at oral bridging Week 20 [74.8 ng/mL (116.1%); lower 90% CI=50.4 ng/mL], at oral bridging Week 30 [41.7 ng/ml (45.7%); lower 90% CI=29.9 ng/mL] exceeded IQ4, as shown in Table 7. At the SC resumption visit, mean (% CV) predose concentration and the lower bound 90% CI [74.4 ng/ml (105.1%); lower 90% CI=56.2 ng/mL] also exceeded IQ4.

TABLE 7
Summary of Plasma LEN Concentration During Oral
Bridging Visits Using 300-mg Once Weekly Dose

| | Oral | Oral | Oral | Oral |
|---|---|---|---|---|
| | Bridging | Bridging | Bridging | Bridging |
| Parameter, | Day 1 | Day 10 | Day 20 | Day 30 |
| Mean (% CV) | (N = 56) | (N = 57) | (N = 36) | (N = 9) |
| Concentration | 46.1 (56.3) | 76.2 (59.6) | 74.8 (116.1) | 41.7 (45.7 |
| Lower 90% CI | 40.3 | 66.1 | 50.4 | 29.9 |
| of Concentration | | | | |
| (ng/mL) | | | | |

% CV = percentage coefficient of variation;
CI = confidence interval

During the oral bridging period, mean LEN concentration and the lower bound of 90% CI sustained above efficacy target of IQ4 (15.5 ng/mL) from the first oral LEN bridging visit through the SC LEN resumption visit.

### Summary of Safety Evaluation

Median (Q1, Q3) duration of exposure to oral bridging of LEN was 19.0 (11.0, 21.9) weeks. A total of 7 (12.3%) of 57 participants had at least 28 weeks of exposure. During the oral bridging period, 49.1% (28 of 57 participants) experienced TEAEs. The 4 most commonly reported TEAEs by participants who received oral bridging were COVID-19 (7.0%, 4 of 57 participants), cough, diarrhea, and upper respiratory tract infection (5.3%, 3 of 57 participants each). The majority of TEAEs were Grade 1 (13 participants, 22.8%) or Grade 2 (14 participants, 24.6%) in severity. There was 1 Grade 5 TEAE of death (SAE, Grade 3 or higher TEAE; and TEAE leading to premature discontinuation of the study, in the same participant [1.8%]) during the oral bridging period, which was considered unrelated to the study drug.

Overall, 3.5% (2 of 57 participants) experienced treatment-related TEAEs, all of which were Grade 1 in severity. The reported treatment-related TEAEs were diarrhea (3.5%, 2 participants) and nausea and asthenia (1.8% each, reported in the same participant). No participant discontinued study drug due to a TEAE. One participant (1.8%) had an AE of death, which led to discontinuation of the study, as described above.

There were no clinically relevant changes from baseline in hematology and clinical chemistry parameters in the oral bridging period. The median values were generally within reference ranges. There were no clinically relevant changes from baseline in vital signs.

### Overall Study Conclusions

A summary of conclusions from the oral bridging study described in this Example are as follows:
Consistent with data reported in the Week 26 and Week 52 interim analyses, high rates of virologic suppression continued to be maintained throughout the oral bridging period.
   The majority of participants who were virologically suppressed at oral bridging baseline (HIV-1 RNA<50 copies/mL) remained virologically suppressed throughout the oral bridging period.
Consistent with the data reported in the Week 26 and Week 52 interim analyses, CD4 cell count and CD4 percentage continued to increase from oral bridging baseline during the oral bridging period.
During the oral bridging period, mean LEN concentration and the lower bound 90% CI were sustained above IQ4 (15.5 ng/ml) from the first oral LEN bridging visit onwards and until the SC LEN resumption visit (approximately 10 to 30 weeks later). Oral bridging with LEN 300 mg once weekly maintained efficacious LEN levels throughout the oral bridging period.
Oral LEN remained generally safe and well tolerated.

These results indicated that oral LEN 300 mg once weekly provided adequate plasma concentrations to bridge SC LEN dosing in PWH who may miss their LEN 6-monthly SC injection.

### Example 5. Oral Bridging of Lenacapavir in Combination with Other Antiretroviral Agents in People Living With HIV

This Example describes results from a Phase 2 clinical study evaluating the safety and efficacy of LEN in combination with other antiretroviral (ARV) agents in ARV-naive people with HIV (PWH).

As described in Example 4, oral bridging with 300 mg oral LEN once weekly (QW) was implemented following a clinical hold imposed by the Food & Drug Administration. Due to the LEN clinical hold, participants receiving subcutaneous (SC) LEN may have missed their SC injection window (within 26 to 28 weeks of the previous SC LEN dose). If a missed dose was anticipated, participants received oral bridging until they could receive their next SC injection. After removal of the full clinical hold, a partial hold was imposed, which allowed oral LEN (300 mg QW) to be administered as an alternative option for SC LEN as described in Example 4. Once the partial hold was lifted, participants were able to resume SC LEN.

Oral bridging of LEN was supported by antiviral activity, pharmacokinetics (PK), and safety data from a Phase 1b proof of concept study, two ongoing Phase 2 and Phase 2/3 studies, described in Examples 4-5, as well as oral PK and safety data from two Phase 1 studies in healthy volunteers. Clinical data indicated that a LEN plasma concentration of 15.5 ng/ml, corresponding to inhibitory quotient (IQ) of 4 or higher provides near maximal antiviral activity.

### Study Design

This Example describes a Phase 2, randomized, open-label, active-controlled, multicenter study. Participants in Treatment Groups 1 and 2 required oral bridging following a clinical hold imposed by the Food & Drug Administration on injectable LEN as the SC injection of LEN could not be administered within the protocol visit window. An Oral Bridging visit was completed every 10 to 12 weeks. Participants had to have met all of the inclusion criteria and none of the exclusion criteria to be included in the study.

### Inclusion Criteria

Inclusion criteria for the study were as follows:
1) Willing and able to provide written informed consent prior to performing study procedures.
2) Aged ≥18 years.
3) ARV-naive with no use of any ARV within 1 month of screening. Use of preexposure prophylaxis (any duration), postexposure prophylaxis (any duration), or HIV-1 treatment (<10 days therapy total)>1 month prior to screening was permitted.
4) Plasma HIV-1 RNA≥200 copies/mL at screening.
5) CD4 cell count≥200 cells/μL at screening.
6) A negative serum pregnancy test was required for all women at screening.
7) Participants of childbearing potential who engage in heterosexual intercourse had to agree to use protocol-specified method(s) of contraception as defined in the clinical study protocol.
8) Lactating women had to agree to discontinue nursing before the study drug(s) was administered.

### Exclusion Criteria

Exclusion criteria for the study were as follows:
1) An opportunistic illness requiring acute therapy within 30 days prior to screening.
2) Active, serious infections (other than HIV-1 infection) requiring parenteral antibiotic or antifungal therapy within 30 days before screening.
3) Active tuberculosis infection.
4) Acute hepatitis within 30 days prior to screening.
5) Hepatitis B virus (HBV) infection, defined as screening results showing either or both of:
   a) Positive HBV surface antigen.
   b) Positive HBV core antibody and negative HBV surface antibody.
6) Hepatitis C virus (HCV) antibody positive and HCV RNA greater than lower limit of quantitation (LLOQ).
7) A history of or current clinical decompensated liver cirrhosis (e.g., ascites, encephalopathy, or variceal bleeding).
8) Treatment within 3 months prior to screening, or anticipated treatment during the study period with immunosuppressant therapies, hydroxyurea, foscarnet, radiation, or cytotoxic chemotherapeutic agents without prior approval from sponsor prior to randomization.
9) Active malignancy requiring acute therapy (with the exception of local cutaneous Kaposi sarcoma).
10) Current alcohol or substance use judged by the investigator to potentially interfere with the participant's study compliance.
11) Clinically significant abnormal electrocardiogram (ECG) at the screening visit.
12) Any of the following laboratory values at screening:
   a) Estimated glomerular filtration rate (eGFR)≤50 mL/min according to the Cockcroft-Gault formula for creatinine clearance (see e.g., Cockcroft & Gault, Nephron, 1976; 16:31-41).
   b) Alanine aminotransferase (ALT)>5× upper limit of normal (ULN).
   c) Direct bilirubin>1.5×ULN.
   d) Platelets<50,000/mm³.
   e) Hemoglobin<8.0 g/dL.
13) Participation or planned participation in any other clinical study (including observational studies) without prior approval throughout the study.
14) Prior use of, or exposure to, LEN.
15) Known hypersensitivity to the study drug(s), the metabolites, or formulation excipient.
16) Use or planned use of exclusionary medications.
17) Any other clinical condition or prior therapy that, in the opinion of the investigator, would make the participant unsuitable for the study or unable to comply with dosing requirements.

### Oral Bridging Treatment Administration

Study treatments were administered as shown in Table 8.

TABLE 8
Study Treatments

| Treatment | Time | Study |
|---|---|---|
| Group | Period | Treatments |
| 1 | Induction | Oral LEN 600 mg (2 × 300 mg tablet) on |
| | (Day 1 through | Days 1 and 2; 300 mg (1 × 300 mg tablet) |
| | Week 27) | on Day 8 |
| | | Oral daily DVY (F/TAF 200/25 mg) from |
| | | Day 1 onwards for a total of 28 weeks^{a} |
| | | SC LEN 927 mg 2 × 1.5 mL) |
| | | (309 mg/mL; |
| | | on Day 15 |
| | Maintenance | SC LEN 927 mg (309 mg/mL; |
| | | 2 × 1.5 mL) |
| | (Week 28 through | at Week 28 and every 6 months |
| | Week 80) | (26 weeks) thereafter |
| | | Oral daily TAF (25 mg) |
| 2 | Induction | Oral LEN 600 mg (2 × 300 mg tablet) on |
| | (Day 1 through | Days 1 and 2; 300 mg (1 × 300 mg tablet) |
| | Week 27) | on Day 8 |
| | | Oral daily DVY (F/TAF 200/25 mg) from |
| | | Day 1 onwards for a total of 28 weeks^{a} |
| | | SC LEN 927 mg (309 mg/ml; |
| | | 2 × 1.5 mL) |
| | | on Day 15 |
| | Maintenance | SC LEN 927 mg (309 mg/mL; |
| | (Week 28 through | 2 × 1.5 mL) |
| | Week 80) | at Week 28 and every 6 months |
| | | (26 weeks) thereafter |
| | | Oral daily BIC (75 mg) |

BIC = bictegravir;
DVY = emtricitibine/tenofovir alafenamide (F/TAF; coformulated; Descovy);
LEN = lenacapavir;
TAF = tenofovir alafenamide

Oral daily TAF, and DVY were taken at approximately same time each day during the applicable treatment period. All study drugs were taken without regard to food; the date and time of the last meal before dosing were collected.

Participants in Treatment Groups 1 and 2 required oral weekly bridging when SC injection of LEN could not be administered due to the LEN clinical hold.
If 26 to 28 weeks elapsed since the last SC injection and if clinically appropriate to continue LEN, oral LEN 300 mg (1 tablet) once a week was started at oral bridging visits and continued weekly dosing on the same day of the week.
If more than 28 weeks elapsed since the last SC injection and if clinically appropriate to continue LEN, the oral lead-in was restarted at the oral bridging visit and the participant continued oral weekly dosing on the same day of the week, until SC LEN was administered, as shown in Table 9.
Participants continued receiving oral weekly LEN 300 mg until they received their next SC LEN injection.
Oral bridging visits were completed every 10-12 weeks. Additional visits during the oral weekly administration were conducted perd investigator's discretion.

TABLE 9
Dosing Schedule for Oral Weekly Bridging and Resumption of SC LEN Injection

| Elapsed 26 to 28 Weeks | More than 28 Weeks Elapsed | |
|---|---|---|
| Since the Last SC Injection | Since the Last SC Injection | |
| Treatment Time from Oral | Treatment Time from Oral | LEN Dose |
| Start | Start | Dose |
| NA | Day 1 | Oral 600 mg |
| | | (2 × 300 mg tablets) |
| NA | Day 2 | Oral 600 mg |
| | | (2 × 300 mg tablets) |
| Day 1 | Day 8: | Oral 300 mg |
| | continued oral weekly LEN | (1 × 300 mg tablets) |
| | on the same day of the week | |
| | as first dose until LEN SC is | |
| | adminsitered | |
| Day 8; | NA | Oral 600 mg |
| continued oral weekly LEN | | (2 × 300 mg tablets) |
| on the same day of the week | | once a week |
| as the first dose until SC LEN | | |
| is administered | | |
| SC administration restarted | SC administration restarted | SC 927 mg |
| (within 1 week of the oral | (within 1 week of the oral | every 6 months |
| weekly 300 mg dose (as early | weekly 300 mg dose (as early | |
| as after Day 1 dosing) | as after Day 8 dosing) | |

The oral bridging dose of LEN was 300 mg administered QW starting 26 to 28 weeks after the last SC LEN injection. This oral weekly bridging dose, even when started as late as 28 weeks after the last SC LEN injection, was predicted to immediately maintain the lower bound of the 90% CI of arithmetic mean for LEN Cₜₐᵤ above IQ4 (i.e., even before reaching steady state). As long as the oral weekly bridging was initiated between 26 to 28 weeks after the last SC LEN injection, the PK profile upon resuming SC injection was predicted to be comparable with that of the prior SC dose and within the target range regardless of when SC injection was resumed. Lenacapavir has been administered orally at doses up to 1800 mg. Safety data from all completed and ongoing clinical studies indicate that LEN is generally safe and well tolerated at the intended exposures.

### Adverse Events

An adverse event (AE) was any untoward medical occurrence in a clinical study participant administered a study drug that did not necessarily have a causal relationship with the treatment. An AE could, therefore, be any unfavorable and/or unintended sign, symptom, or disease temporally associated with the use of a study drug, whether or not considered related to the study drug. Adverse events also included pretreatment or posttreatment complications that occurred as a result of protocol-specified procedures, lack of efficacy, overdose, drug abuse/misuse reports, or occupational exposure.

Preexisting events that increased in severity or changed in nature during or as a consequence of participation in the clinical study were also considered AEs. The relationship or association of the AE to a study treatment was assessed using clinical judgment and the following considerations:
Not related: Evidence existed that the AE had an etiology other than the study drug product. For serious adverse events (SAEs), an alternative causality must have been provided (e.g., preexisting condition, underlying disease, intercurrent illness, or concomitant medication).
Related: There was reasonable possibility that the AE was caused by the study drug.

The relationship to study procedures (e.g., invasive procedures such as venipuncture or biopsy) was assessed using the following considerations:
Not related: Evidence existed that the AE had an etiology other than the study procedure.
Related: The AE occurred as a result of protocol procedures (e.g., venipuncture).

### Serious Adverse Events

A serious adverse event (SAE) was defined as an event that, at any dose, resulted in any of the following outcomes:
Death.
Life-threatening situation (participant was at immediate risk of death).
Inpatient hospitalization or prolongation of existing hospitalization.
Persistent or significant disability/incapacity.
A congenital anomaly/birth defect.
Other medically significant events that based upon appropriate medical judgment may have jeopardized the participant or may have required medical or surgical intervention to prevent one of the outcomes listed above.

### Summary of Efficacy Results

All participants who switched from SC to oral LEN during the period of oral bridging remained virologically suppressed (HIV-1 RNA<50 copies/mL) with oral dosing and there were no virologic failures. Following switching to oral bridging treatment, CD4 count and CD4% results were generally stable or slightly increasing.

### HIV-1 RNA 50 Copies/mL Using Imputation Methods (M=F and M=E)

At oral bridging baseline, 120 of 121 (99.2%) participants in the LEN total group had HIV-1 RNA<50 copies/mL (SC LEN total group: 82 of 82 participants [100%]; oral LEN group: 38 of 39 participants [97.4%]). A single participant in the oral LEN group had HIV-1 RNA 200 to <400 copies/mL.

In the Missing=Excluded (M=E) analysis, at all subsequent oral bridging time points, all participants (100%) in all treatment groups (LEN total, SC LEN total, and oral LEN groups) had HIV-1 RNA<50 copies/mL.

In the Missing=Failure (M=F) analysis, at Week 10, 115 of 118 participants (97.5%) (SC LEN total group: 77 of 79 participants [97.5%]; oral LEN group: 38 of 39 participants [97.4%]) had HIV-1 RNA<50 copies/mL. At Week 20, 77 of 77 participants (100%) (SC LEN total group: 58 of 58 participants [100%]; oral LEN group: 19 of 19 participants [100%]) had HIV-1 RNA<50 copies/mL.

### Change From Baseline in CD4 Cell Count

At oral bridging baseline, the mean (SD) CD4 cell count in the LEN total group (N=121) was 762 (285.2)/μL (SC LEN total group [N=82]: 786 [299.3]/μL; oral LEN group [N=39]: 711 [248.9]/μL).

At Week 10 (N=121), the mean (SD) change from oral bridging baseline in CD4 cell count in the LEN total group was 35/μL (212.8/μL) (SC LEN total group [N=82]: 26/μL [204.0/μL]; oral LEN group [N=39]: 55/μL [231.7/μL]). At Week 20 (N=79), the mean (SD) change from oral bridging baseline in CD4 cell count in the LEN total group was 0/μL (206.3/μL) (SC LEN total group [N=61]: −31/μL [196.6/μL]; oral LEN group [N=18]: 106/μL [208.1/μL]).

No notable trends in change from oral bridging baseline in CD4 count results were observed between treatment groups.

### Change From Baseline in CD4%

At oral bridging baseline, the mean (SD) CD4% in the LEN total group (N=121) was 34% (7.7%; SC LEN total group [N=82]: 35% [7.3%]; oral LEN group (N=39): 33% [8.4%]).

At Week 10 (N=121), the mean (SD) change from oral bridging baseline in CD4% in the LEN total group was 1% (4.0%) (SC LEN total group [N=82]: 0% [4.0%]; oral LEN group [N=39]: 1% [3.9%]). At Week 20 (N=79), the mean (SD) change from oral bridging baseline in CD4% in the LEN total group was 0% (3.6%) (SC LEN total group [N=61]: 0% [3.3%]; oral LEN group [N=18]: 1% [4.4%]).

Similar mean change from oral bridging baseline in CD4% results were observed across treatment groups.

### Summary of Pharmacokinetic and Pharmacodynamic Evaluation

For Treatment Groups 1 and 2 (separately and combined), mean LEN concentrations and the lower bound 90% CI (when reportable) were consistently maintained above IQ4 (15.5 ng/mL) from the first oral LEN bridging visit and through the SC LEN resumption visit based on this interim analysis, as shown in Table 10.

For Treatment Group 1 (SC LEN+ [DVY→TAF]), mean (% CV) concentrations on Day 1 (28.8 ng/mL [47.7%]; lower bound 90% CI: 25.2 ng/ml), Week 10 (50.9 ng/ml [67.0%]; lower bound 90% CI: 41.1 ng/mL), and Week 20 (53.1 ng/mL [84.5%]; lower bound 90% CI: 39.6 ng/ml), exceeded IQ4. At Week 30, the mean (% CV) concentration was 43.7 ng/ml (74.7%), but a lower bound 90% CI was not reported due to sample size of <5. At the SC LEN resumption visit, the mean (% CV) concentration was 49.4 ng/mL (84.6%), with a lower bound 90% CI of 38.6 ng/ml.

For Treatment Group 2 (SC LEN+ [DVY→BIC]), mean (% CV) concentrations on Day 1 (26.7 ng/mL [47.3%]; lower bound 90% CI: 23.0 ng/ml), Week 10 (59.3 ng/ml [73.6%]; lower bound 90% CI: 46.4 ng/ml), and Week 20 (51.9 ng/mL [70.2%]; lower bound 90% CI: 40.2 ng/ml) exceeded IQ4. At Week 30, the mean (% CV) concentration was 56.5 ng/mL (62.5%), but a lower bound 90% CI was not reported due to sample size of <5. At the SC LEN resumption visit, the mean (% CV) concentration was 52.2 ng/ml (66.6%), with a lower bound 90% CI of 42.7 ng/mL.

For Treatment groups 1 and 2 combined (SC LEN total), mean (% CV) concentrations on Day 1 (27.8 ng/mL [47.4%]; lower bound 90% CI: 25.3 ng/mL), Week 10 (54.9 ng/mL [70.8%]; lower bound 90% CI: 47.1 ng/ml), Week 20 (52.5 ng/ml [77.7%]; lower bound 90% CI: 43.7 ng/ml), and Week 30 (50.1 ng/mL [62.3%]; lower bound 90% CI: 24.4 ng/mL) exceeded IQ4. At the SC LEN resumption visit, the mean (% CV) concentration was 50.7 ng/ml (75.7%), with a lower bound 90% CI of 43.6 ng/mL.

TABLE 10
Plasma LEN Concentrations for Treatment Groups Following
Oral Bridging Using 300 mg Once Weekly Oral LEN
SC LEN + [DVY →TAF] Treatment Group 1

| PK Parameter | Day 1 | Week 10 | Week 20 | Week 30 |
|---|---|---|---|---|
| Mean (% CV) | (N = 41) | (N = 35) | (N = 32) | (N = 3) |
| Concentration | 28.8 (47.7) | 50.9 (67.0) | 53.1 (84.5) | 43.7 (74.7) |
| (ng/mL) | | | | |
| Lower 90% CI | 25.2 | 41.1 | 39.6 | NR |
| of Concentration | | | | |
| (ng/mL) | | | | |

SC LEN + [DVY →BIC] Treatment Group 2

| PK Parameter | Day 1 | Week 10 | Week 20 | Week 30 |
|---|---|---|---|---|
| Mean (% CV) | (N = 35) | (N = 33) | (N = 28) | (N = 3) |
| Concentration | 26.7 (47.3) | 59.3 (73.6) | 51.9 (70.2) | 56.5 (62.5) |
| (ng/mL) | | | | |
| Lower 90% CI | 23.0 | 46.4 | 40.2 | NR |
| of Concentration | | | | |
| (ng/mL) | | | | |

SC LEN TOTAL: SC LEN + [DVY →TAF] and SC LEN + [DVY →BIC]

| PK Parameter | Day 1 | Week 10 | Week 20 | Week 30 |
|---|---|---|---|---|
| Mean (% CV) | (N = 76) | (N = 68) | (N = 60) | (N = 6) |
| Concentration | 27.8 (47.4) | 54.9 (70.8) | 52.5 (77.7) | 50.1 (62.3) |
| (ng/mL) | | | | |
| Lower 90% CI | 25.3 | 47.1 | 43.7 | 24.4 |
| of Concentration | | | | |
| (ng/mL) | | | | |

% CV = percentage coefficient of variation;
BIC = bictegravir;
CI = confidence interval;
DVY = emtricitabine/tenofovir alafenamide (F/TAF, coformulated; Descovy);
LEN = lenacapavir;
N = number of participants;
SC = subcutaneous;
TAF = tenofovir alafenamide;
NR = not reported due to sample size <5

Mean LEN concentrations and the lower bound 90% CI were consistently maintained above IQ4 (15.5 ng/mL) from the first oral LEN bridging visit and through the SC LEN resumption visit of the oral bridging period based on this interim analysis. There were few participants with LEN concentrations at Week 30 of the oral bridging period; however, when Treatment Groups 1 and 2 were combined, the lower bound 90% CI was above IQ4 at Week 30 (n=6).

### Summary of Safety Results

During the oral bridging, 60.3% of participants in the LEN total group (73 of 121 participants; SC LEN total group: 37 [45.1%] participants; oral LEN group: 36 [92.3%] participants) had been exposed to oral LEN for at least 20 weeks. With the exception of injection site reactions (ISRs), the treatment-emergent adverse events (TEAEs) were similar in pattern and frequency to those observed when participants received SC LEN. Treatment with oral LEN was generally safe and well tolerated.

Overall, 62.8% of participants in the LEN total group (76 of 121 participants) had any AE during oral bridging (SC LEN total group: 53 [64.6%] participants; oral LEN group: 23 [59.0%] participants). The most commonly reported TEAEs in the LEN total group were as follows: influenza (9 [7.4%] participants; SC LEN total group: 7 [8.5%] participants; oral LEN group: 2 [5.1%] participants), COVID-19 (7 [5.8%] participants; SC LEN total group: 4 [4.9%] participants; oral LEN group: 3 [7.7%] participants), nasopharyngitis (6 [5.0%] participants; SC LEN total group: 4 [4.9%] participants; oral LEN group: 2 [5.1%] participants), and syphilis (6 [5.0%] participants; SC LEN total group: 4 [4.9%] participants; oral LEN group: 2 [5.1%]).

The majority of all TEAEs reported in all treatment groups were Grade 1 or 2 in severity. The percentage of participants with TEAEs of Grade 3 or higher in the LEN total group was 5.0% (LEN total: 6 participants; SC LEN total group: 3 [3.7%] participants; oral LEN group: 3 [7.7%] participants); 3 (2.5%) participants had Grade 3 events (SC LEN total group: 1 [1.2%] participants; oral LEN group: 2 [5.1%] participant) and 3 (2.5%) participants had Grade 4 events (SC LEN total group: 2 [2.4%] participants; oral LEN group: 1 [2.6%] participant). The only AE of Grade 3 or higher reported for >1 participant was blood creatine phosphokinase increased (1.7%, 2 participants; both in oral LEN group). The percentage of participants with TEAEs related to study drug in the LEN total group was 3.3% (4 participants; SC LEN total group: 3 [3.7%] participants; oral LEN group: 1 [2.6%] participant). No TEAEs related to study drug were reported for >1 participant in any treatment group and all were Grade 1.

Serious AEs were reported for 2.5% of participants in the LEN total group (3 participants; SC LEN total group: 2 [2.4%] participants [cholecystitis, deep vein thrombosis, and diabetic ketoacidosis]; oral LEN group: 1 [2.6%] participant [bipolar disorder]). None of the SAEs were reported for >1 participant in any treatment group, and all were considered not related to study drug.

During the oral bridging period no TEAEs which led to a premature discontinuation of study drug or from the study were reported. No deaths were reported during the oral bridging period.

There were no clinically relevant changes from oral bridging baseline in median values for hematology or clinical chemistry parameters (including metabolic parameters) in any treatment group, and median values were generally within reference ranges.

There were no clinically relevant changes from oral bridging baseline within each treatment group or differences among the treatment groups in median values for systolic blood pressure, diastolic blood pressure, pulse, respiration rate, or body temperature. One female participant in the oral LEN group had a confirmed pregnancy during the oral bridging period, this pregnancy had an outcome of live birth.

### Study Conclusions

A summary of conclusions from the oral bridging study described in this Example are as follows:
High rates of virologic suppression continued to be maintained and no participant who received oral bridging had virologic failure (HIV-1 RNA≥50 copies/mL) during the oral bridging period.
CD4 cell count and CD4% increased or remained stable from oral bridging baseline for participants who received oral bridging during the oral bridging period.
For participants who received oral bridging (SC LEN total group), mean LEN concentrations and the lower bound 90% CI were consistently maintained above IQ4 (15.5 ng/mL) from the first oral LEN bridging visit and through the SC LEN resumption visit. Oral bridging with 300 mg QW LEN maintained efficacious LEN levels throughout the oral bridging period.
During the oral bridging period, orally administered LEN in participants who received oral bridging was generally safe and well tolerated.

### Example 6. Post Hoc Analysis Assessing Efficacy of LEN Oral Bridging

This example provides an overview of data from a post hoc analysis assessing the clinical pharmacology, efficacy, and safety of oral LEN (300 mg) administered weekly in MDR and treatment-naive PWH described in Examples 4-5, respectively, when SC LEN dosing was interrupted.

### Virology

### Capsid Resistance Emergence During Oral Bridging Period

In HTE participants (Example 4), the resistance analysis population (RAP) during the oral bridging period included 9 participants (16%, 9/57) who met the criteria for resistance analysis, including 4 participants also present in the previous analysis. Six of the 9 participants in the RAP developed resistance to LEN: 3 participants developed the Q67H or Q67Q/H LEN resistance mutation that was associated with a mean 5.6-fold decrease in LEN susceptibility; 1 participant developed the N74D LEN resistance mutation with no phenotypic data (assay failure); 1 participant developed K70N+N74K that was associated with a 289-fold decrease in LEN susceptibility; 1 participant developed Q67H+K70R+T107T/N that was associated with a 393-fold decrease in LEN susceptibility.

No treatment-naive participants from the study described in Example 4 experienced virologic failure during the oral bridging period resulting in no capsid resistance data for that study.

### Clinical Pharmacokinetics

### Observed Multiple-Dose PK of the LEN Oral Tablet Formulation

Multiple-dose PK of oral 300 mg once weekly LEN for oral bridging was evaluated in the studies described in Examples 4-5. The mean LEN concentrations and the lower bound 90% CIs were maintained above inhibitory quotient of 4 (IQ4=15.5 ng/ml) from the first oral LEN bridging visit until SC LEN was resumed approximately 10 to 30 weeks later. These results indicate that oral LEN 300 mg once weekly provided adequate plasma concentrations to bridge SC LEN dosing in PWH who may miss their LEN 6-monthly SC injection.

### Pharmacokinetics/Pharmacodynamics

### Rationale for Proposed Dosing Regimen

The proposed dosing schedule for missed maintenance injections is as follows: “during the maintenance period, if a patient plans to miss a scheduled 6-month injection visit by more than 2 weeks, SUNLENCA tablets may be used for oral bridging on an interim basis for up to 6 months until injections resume. The recommended dosage is 300 mg taken orally once every 7 days. Resume the maintenance injection dosage within 7 days after the last oral dose.” Population-PK simulations in HTE PWH indicate that the oral weekly bridging dose, even when started as late as 28 weeks after the last SC LEN injection, is predicted to immediately maintain the lower bound of the 90% CI of the arithmetic mean for LEN C_{trough} above IQ4 (i.e., 15.5 ng/mL) even before reaching steady state.

### Exposure-Response Analysis for Efficacy

Exposure-response relationships for LEN were evaluated in HTE PWH with HIV-1 RNA<50 copies/mL at the oral bridging baseline in the Phase 2/3 study described in Example 4, using LEN exposure parameters (C_{trough}, C_{avg}, and Cₘₐₓ) derived from PopPK modeling.

A total of 39 of 40 participants had HIV-1 RNA<50 copies/mL at the time SC LEN was resumed. No trend in post hoc C_{trough} was observed for participants with HIV-1 RNA<50 copies/mL compared with participants with HIV-1 RNA≥50 copies/mL. For 1 participant with HIV-1 RNA≥50 copies/mL, observed LEN concentrations were within the range from other participants. It is suspected that the virologic failure in this participant was due to a lack of adherence to the optimized background regimen (OBR) or oral LEN during visits at which PK sampling was not conducted. This participant was resuppressed after SC resumption.

### Exposure-Response Analysis for Safety

Exposure-safety relationships for LEN were evaluated in the HTE PWH studies described in Example 4 (N=55), and in treatment-naive PWH in the study described in Example 5 (N=82) who received the oral bridging LEN regimen. Exposure-safety evaluations used LEN exposure metrics (C_{avg}, C_{trough}, and Cₘₐₓ) derived from PopPK analysis and the 5 most common treatment-emergent adverse events (TEAEs) were evaluated in relation to presence/absence in the LEN group across the entire oral bridging period in the two studies separately and combined (excluding infections and infestations AEs and injection site reactions). Whether analyzed separately or combined, LEN exposures (C_{avg}, C_{trough}, and Cₘₐₓ), were similar, regardless of the presence or absence of the evaluated AEs, indicating a lack of association between LEN exposure and the incidence of common AEs.

### Pharmacokinetic and Pharmacokinetic-Pharmacodynamic Conclusions

During the oral bridging period, oral LEN 300 mg once weekly administration, maintained efficacious LEN levels from first oral LEN bridging visit onwards and until the SC LEN resumption visit in both studies described in Example 4-5. These results indicate that oral LEN 300 mg once weekly provided adequate plasma concentrations to bridge SC LEN dosing in PWH who may miss their LEN 6-monthly SC injection.

Population-PK analysis suggested that the effects of HTE status (i.e., HTE status versus healthy participants), pharmacoenhancers, baseline body weight, and fed or fasting conditions on LEN exposures from oral bridging were not considered clinically meaningful and therefore do not warrant dose adjustment.

Exposure-response analysis for efficacy during the oral bridging period indicated overlap in concentrations for participants with HIV-1 RNA≥50 copies/mL (N=1) versus <50 copies/mL (N=39). It is suspected that virologic failure may have been related to lack of adherence to the OBR or oral LEN during visits at which PK sampling was not conducted.

LEN exposures (C_{avg}, C_{trough}, and Cₘₐₓ) in the studies described in Examples 4-5 were similar, regardless of the presence or absence of evaluated AEs, indicating a lack of association between LEN exposure from oral bridging and the incidence of common AEs.

### Efficacy

Of participants with oral bridging baseline HIV-1 RNA<50 copies/mL at the time of switch to oral bridging, only 1 did not maintain HIV-1 RNA<50 copies/mL during this period at Week 10 and 20 (Week 10 values: 920 copies/mL; Week 20 values: 57 copies/mL). The participant developed a LEN resistance-associated mutation (N74D) and had missed 2 nonconsecutive doses of oral LEN prior to the elevated HIV-1 RNA results. However, the participant resuppressed after receiving SC LEN with no OBR change.

In addition, the percentage of participants with oral bridging baseline HIV-1 RNA≥50 copies/mL who achieved HIV-1 RNA<50 copies/mL at Weeks 10 and 20 was 27.3% (3 of 11 participants) and 50.0% (2 of 4 participants), respectively.

Following switching to oral bridging treatment, CD4 count and CD4% results continued to increase from oral bridging baseline during the oral bridging period.

### Efficacy Discussion and Conclusions

The efficacy conclusions from the studies described in Examples 4-5 were as follows:

For HTE PWH participants and consistent with data reported in the Week 26 and Week 52 interim analyses, high rates of virologic suppression continued to be maintained throughout the oral bridging period. The majority of participants who were virologically suppressed at oral bridging baseline (HIV-1 RNA<50 copies/mL) remained virologically suppressed throughout the oral bridging period.

For treatment-naive participants, high rates of virologic suppression continued to be maintained and no participant who received oral bridging or continued oral therapy had virologic failure (HIV-1 RNA≥50 copies/mL) during the oral bridging period.

For HTE PWH participants and consistent with the data reported in the Week 26 and Week 52 interim analyses, CD4 cell count and CD4% continued to increase from oral bridging baseline during the oral bridging period.

For treatment-naive participants, CD4 cell count and CD4% increased or remained stable from oral bridging baseline for participants who received oral bridging or continued oral therapy during the oral bridging period.

### Safety Analysis

### Primary Study Supporting the Safety of LEN

The percentage of participants who received oral bridging during the study described in Example 4 and experienced TEAEs was 49.1% (28 of 57 participants). There was 1 Grade 5 TEAE of death (serious adverse event [SAE], Grade 3 or higher TEAE; and TEAE leading to premature discontinuation of the study, in the same participant [1.8%]) during the oral bridging period, which was considered unrelated to the study drug. A total of 15 participants (26.3%) experienced Grade 2 or higher TEAEs. The 4 most commonly reported TEAEs by participants who received oral bridging were COVID-19 (7.0%, 4 of 57 participants), cough, diarrhea, and upper respiratory tract infection (5.3%, 3 of 57 participants each). The majority of AEs were Grade 1 (13 participants, 22.8%) or Grade 2 (14 participants, 24.6%) in severity. Overall, 3.5% (2 of 57 participants) experienced treatment-related TEAEs, all of which were Grade 1 in severity. The reported treatment-related TEAEs were diarrhea (3.5%, 2 participants) and nausea and asthenia (1.8% each, reported in the same participant).

No participant discontinued study drug due to a TEAE. One participant had an AE of death, which was considered not related to study drug, and which led to discontinuation of the study.

There were no clinically relevant changes from baseline in median values for hematology and clinical chemistry (including metabolic assessments) during the Oral Bridging Period.

### Additional Data Supporting the Safety of LEN (Study GS-US-200-4334)

Within the LEN total group described in Example 5, a total of 76 (62.8%) participants had any AE (SC LEN total group: 53 [64.6%] participants; oral LEN group 23 [59.0%] participants) and 6 (5.0%) participants had Grade 3 or higher AEs (SC LEN total group: 3 [3.7%] participants; oral LEN group 3 [7.7%] participants). Serious AEs were reported for 3 (2.5%) participants in the LEN total group (SC LEN total group: 2 [2.4%] participants; oral LEN group: 1 [2.6%] participant). There were 4 (3.3%) participants in the LEN total group with AEs related to study drug (SC LEN total group: 3 [3.7%] participants; oral LEN group 1 [2.6%] participant), all of these related AEs were Grade 1.

The most commonly reported (>2 participants in the LEN total group) AEs in participants who received oral LEN were as follows:

SC LEN total group: influenza (7 [8.5%] participants), COVID-19 (4 [4.9%] participants), nasopharyngitis (4 [4.9%] participants), syphilis (4 [4.9%] participants), oropharyngeal pain (4 [4.9%] participants).

Oral LEN group: COVID-19 (3 [7.7%] participants), influenza (2 [5.1%] participants), nasopharyngitis (2 [5.1%] participants), syphilis (2 [5.1%] participants), arthralgia (2 [5.1%] participants), blood creatine phosphokinase increase (2 [5.1%] participants), dyslipidemia (2 [5.1%] participants), and oropharyngeal gonococcal infection (2 [5.1%] participants) The majority of the AEs reported in all treatment groups were Grade 1 or 2 in severity. The percentage of participants with Grade 3 or higher AEs in the LEN total group was 5.0% (6 of 121 participants).

One participant had Grade 4 deep vein thrombosis who had medical history of recurrent deep vein thrombosis events for which the participant was receiving warfarin. This participant also experienced a Grade 3 hematoma event.

One participant had Grade 4 events of diabetes mellitus and diabetic ketoacidosis, who was assessed as having uncontrolled diabetes at baseline. This participant also experienced a Grade 3 cholecystitis event.

None of these AE were considered related to study drug. The percentage of participants with AEs related to study drug in the LEN total group was 3.3% (4 of 121 participants); 3 [3.7%] participants in the SC LEN total group, and 1 [2.6%] participant in the oral LEN group. No AEs related to study drug were reported for >1 participant in any treatment group. No AEs of Grade 2 or higher related to study drug were reported for any treatment group.

No deaths were reported during the oral bridging period. Serious AEs were reported for 3 (2.5%) participants in the LEN total group: 2 (2.4%) participants in the SC LEN total group, and 1 (2.6%) participant in the oral LEN group. None of the SAEs were reported for >1 participant in any treatment group. All SAEs reported during oral bridging were considered not related to study drug by the investigator and pre-existing conditions provided a likely alternative etiology.

During the oral bridging period, no AEs that led to premature discontinuation of study drug or from the study were reported.

### Conclusions on LEN Safety

In summary, the safety results from the studies described in Examples 4-5, during the oral bridging period demonstrated the following:
Consistent with the data reported in the interim analyses, treatment with LEN during the oral bridging period weeks remained generally safe and well tolerated. There were no study drug-related SAEs.
The most common study drug-related AEs were of diarrhea; all of which were Grade 1 in severity.
Incidence of Grade 3 or higher laboratory abnormalities remained low. Most were transient, improved on subsequent visits despite continued exposure to the study drug, or participants had alternative etiologies.
LEN continues to have a favorable hepatic and renal safety profile, based on the low frequency of Grade 3 or higher laboratory abnormalities, and no clinically relevant changes from baseline in hepatic and renal parameters.

In conclusion, oral bridging LEN (300 mg) administered weekly in HTE and treatment-naive PWH was generally safe and well tolerated.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A method of treating or preventing HIV in a patient, comprising administering to the patient a compound of Formula Ia: 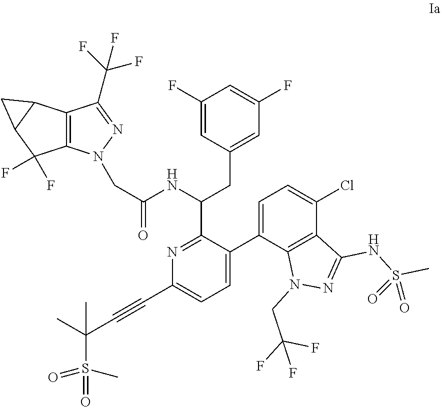 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]
or a pharmaceutically acceptable salt thereof, the method comprising:
(a) administering to the patient an initiation dosage of the compound of Formula Ia, or a pharmaceutically acceptable salt thereof, for a first period of time; and
(b) administering to the patient one or more maintenance dosages of the compound of Formula Ia, or a pharmaceutically acceptable salt thereof, for a second period of time, wherein the second period of time occurs after the first period of time; and
wherein if the patient misses or will miss a maintenance dosage, the method further comprises orally administering to the patient a bridging dosage of about 250 mg to about 650 mg of the compound of Formula Ia, or a pharmaceutically acceptable salt thereof, once per week until the patient resumes administration of the one or more maintenance dosages.

2. The method of claim 1, wherein the first period of time is two days.

3. The method of claim 1, wherein the initiation dosage comprises:
subcutaneously administering the compound of Formula Ia, or a pharmaceutically acceptable salt thereof, at a concentration of about 309 mg/mL, and orally administering about 500 mg to about 700 mg of the compound of Formula Ia, or a pharmaceutically acceptable salt thereof, on the first day; and
orally administering about 500 mg to about 700 mg of the compound of Formula Ia, or a pharmaceutically acceptable salt thereof, on the second day.

4. The method of claim 3, wherein the subcutaneous administration is administered as two subcutaneous injections of about 309 mg/mL, on the first day.

5. The method of claim 3, wherein the subcutaneous administration comprises administering about 927 mg of the compound of Formula Ia, or a pharmaceutically acceptable salt thereof, as two subcutaneous injections of about 309 mg/mL.

6. The method of claim 3, wherein the oral administrations are administered as two tablets, each comprising about 200 to about 400 mg of the compound of Formula Ia, or a pharmaceutically acceptable salt thereof, on the first day and on the second day.

7. The method of claim 3, wherein the oral administrations are administered as two tablets, each comprising about 300 mg of the compound of Formula Ia, or a pharmaceutically acceptable salt thereof, on the first day and on the second day.

8. The method of claim 1, wherein the initiation dosage comprises:
subcutaneously administering 927 mg of the compound of Formula Ia, or a pharmaceutically acceptable salt thereof, and orally administering about 600 mg of the compound of Formula Ia, or a pharmaceutically acceptable salt thereof, on the first day; and
orally administering about 600 mg of the compound of Formula Ia, or a pharmaceutically acceptable salt thereof, on the second day.

9. The method of claim 1, wherein the first period of time is fifteen days.

10. The method of claim 9, wherein the initiation dosage comprises:
orally administering about 500 mg to about 700 mg of the compound of Formula Ia, or a pharmaceutically acceptable salt thereof, on the first day and on the second day;
orally administering about 200 mg to about 400 mg of the compound of Formula Ia, or a pharmaceutically acceptable salt thereof, on the eighth day; and
subcutaneously administering the compound of Formula Ia, or a pharmaceutically acceptable salt thereof, at a concentration of about 309 mg/mL, on the fifteenth day.

11. The method of claim 10, wherein the oral administrations of the first and second days are each administered as two tablets, each comprising about 200 to about 400 mg of the compound of Formula Ia, or a pharmaceutically acceptable salt thereof.

12. The method of claim 10, wherein the oral administration of the eighth day is administered as one tablet comprising about 200 to about 400 mg of the compound of Formula Ia, or a pharmaceutically acceptable salt thereof.

13. The method of claim 10, wherein the oral administrations of the first and second days are administered as two tablets, each comprising about 200 to about 400 mg of the compound of Formula Ia, or a pharmaceutically acceptable salt thereof; and
the oral administration of the eighth day is administered as one tablet comprising about 200 to about 400 mg of the compound of Formula Ia, or a pharmaceutically acceptable salt thereof.

14. The method of claim 10, wherein the oral administrations of the first and second days are administered as two tablets, each comprising about 300 mg of the compound of Formula Ia, or a pharmaceutically acceptable salt thereof; and
the oral administration of the eighth day is administered as one tablet comprising about 300 mg of the compound of Formula Ia, or a pharmaceutically acceptable salt thereof.

15. The method of claim 10, wherein the subcutaneous administration is administered as two subcutaneous injections of about 309 mg/mL, on the fifteenth day.

16. The method of claim 10, wherein the subcutaneous administration comprises administering about 927 mg of the compound of Formula Ia, or a pharmaceutically acceptable salt thereof, as two subcutaneous injections of about 309 mg/mL.

17. The method of claim 1, wherein the initiation dosage comprises:
orally administering about 600 mg of the compound of Formula Ia, or a pharmaceutically acceptable salt thereof, on the first day and on the second day;
orally administering about 300 mg of the compound of Formula Ia, or a pharmaceutically acceptable salt thereof, on the eighth day; and
subcutaneously administering 927 mg of the compound of Formula Ia, or a pharmaceutically acceptable salt thereof, on the fifteenth day.

18. The method of claim 3, wherein the second period of time begins about 24 to about 28 weeks after the final subcutaneous administration of the initiation dosage.

19. The method of claim 3, wherein the second period of time begins about 26 weeks after the final subcutaneous administration of the initiation dosage.

20. The method of claim 1, wherein each maintenance dosage comprises subcutaneously administering the compound of Formula Ia, or a pharmaceutically acceptable salt thereof, at a concentration of about 309 mg/mL, every 26 weeks.

21. The method of claim 20, wherein each maintenance dosage comprises two subcutaneous injections of about 309 mg/mL, every 26 weeks.

22. The method of claim 1, wherein each maintenance dosage comprises subcutaneously administering 927 mg of the compound of Formula Ia, or a pharmaceutically acceptable salt thereof, every 26 weeks.

23. The method of claim 1, wherein each maintenance dosage comprises subcutaneously administering about 927 mg of the compound of Formula Ia, or a pharmaceutically acceptable salt thereof, as two subcutaneous injections of about 309 mg/mL, every 26 weeks.

24. (canceled)

25. The method of claim 1, wherein the bridging dosage is administered for up to 6 months.

26-28. (canceled)

29. A method of treating or preventing HIV in a patient, comprising administering to the patient a compound of Formula Ia: 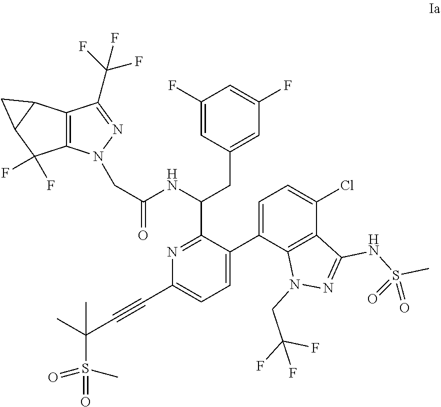 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]
or a pharmaceutically acceptable salt thereof, the method comprising:
(a) administering to the patient an initiation dosage comprising:
(i) subcutaneously administering to the patient a dosage of 927 mg of the compound of Formula Ia, or a pharmaceutically acceptable salt thereof, and orally administering to the patient a dosage of 600 mg of the compound of Formula Ia, or a pharmaceutically acceptable salt thereof, on the first day;
(ii) orally administering to the patient a dosage of 600 mg of the compound of Formula Ia, or a pharmaceutically acceptable salt thereof, on the second day; and
(b) administering to the patient one or more maintenance dosages of the compound of Formula Ia, or a pharmaceutically acceptable salt thereof, wherein each maintenance dosage comprises:
(i) subcutaneously administering to the patient a dosage of 927 mg of the compound of Formula Ia, or a pharmaceutically acceptable salt thereof, once every 24 to 28 weeks from the date of the last subcutaneous administration;
wherein if the patient misses or will miss a maintenance dosage of step (b) (i), the method further comprises orally administering to the patient a bridging dosage of about 300 mg of the compound of Formula Ia, or a pharmaceutically acceptable salt thereof, once per week until the patient resumes administration of the one or more maintenance dosages.

30. A method of treating or preventing HIV in a patient, comprising administering to the patient a compound of Formula Ia: 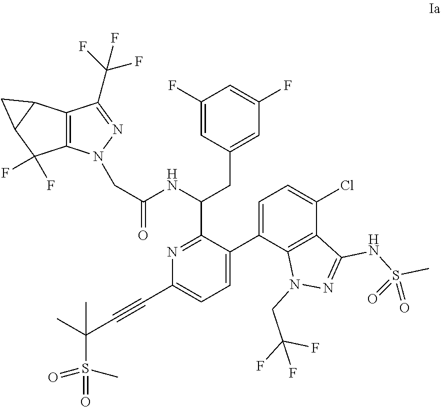 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]
or a pharmaceutically acceptable salt thereof, the method comprising:
(a) administering to the patient an initiation dosage comprising:
(i) orally administering to the patient a dosage of 600 mg of the compound of Formula Ia, or a pharmaceutically acceptable salt thereof, on the first day and on the second day;
(ii) orally administering to the patient a dosage of 300 mg of the compound of Formula Ia, or a pharmaceutically acceptable salt thereof, on the eighth day, wherein step (ii) occurs after step (i);
(iii) subcutaneously administering to the patient a dosage of 927 mg of the compound of Formula Ia, or a pharmaceutically acceptable salt thereof, on the fifteenth day; and
(b) administering to the patient one or more maintenance dosages of the compound of Formula Ia, or a pharmaceutically acceptable salt thereof, wherein each maintenance dosage comprises:
(i) subcutaneously administering to the patient a dosage of 927 mg of the compound of Formula Ia, or a pharmaceutically acceptable salt thereof, once every 24 to 28 weeks from the date of the last subcutaneous administration;
wherein if the patient misses or will miss a maintenance dosage of step (b) (1), the method further comprising orally administering to the patient a bridging dosage of about 300 mg of the compound of Formula Ia, or a pharmaceutically acceptable salt thereof, once per week until the patient resumes administration of the one or more maintenance dosages.

31. The method of claim 1, wherein the compound of Formula Ia is administered as a sodium salt.

32. The method of claim 1, wherein each subcutaneous administration is administered as a solution comprising the sodium salt of the compound of Formula Ia.

33. The method of claim 32, wherein each solution comprises about 20 w/w % to about 30 w/w % water, about 48 w/w % to about 60 w/w % PEG 300, and about 11 w/w % to about 28 w/w % of a sodium salt of the compound of Formula Ia.

34. The method of claim 32, wherein each solution comprises about 23.41 w/w % water, about 50.13 w/w % PEG 300, and about 26.46 w/w % of the sodium salt of the compound of Formula Ia.

35. The method of claim 32, wherein each solution comprises about 309 mg/mL of the compound of Formula Ia, or a pharmaceutically acceptable salt thereof.

36. The method of claim 32, wherein each solution comprises about 315.4 mg/mL of the sodium salt of the compound of Formula Ia.

37. The method of claim 1, wherein each oral administration is administered as a tablet comprising the sodium salt of the compound of Formula Ia.

38. (canceled)

39. The method of claim 37, wherein each tablet comprises about 5 w/w % to about 45 w/w % of the sodium salt of the compound of Formula Ia, about 1 w/w % to about 10 w/w % of copovidone, about 0.01 w/w % to about 10 w/w % of poloxamer 407, about 5 w/w % to about 45 w/w % of microcrystalline cellulose, about 15 w/w % to about 70 w/w % of mannitol, about 1 w/w % to about 30 w/w % of croscarmellose sodium, and about 0.01 w/w % to about 10 w/w % of magnesium stearate, and one or more pharmaceutically acceptable excipients.

40. The method of claim 37, wherein each tablet comprises about 15 w/w % to about 25 w/w % of the sodium salt of the compound of Formula Ia, about 3 w/w % to about 6 w/w % of copovidone, about 0.5 w/w % to about 3.0 w/w % of poloxamer 407, about 18 w/w % to about 30 w/w % of microcrystalline cellulose, about 40 w/w % to about 50 w/w % of mannitol, about 6 w/w % to about 10 w/w % of croscarmellose sodium, and about 1.0 w/w % to about 3.0 w/w % magnesium stearate.

41. The method of claim 37, wherein each tablet comprises about 300 mg of the compound of Formula Ia, or a pharmaceutically acceptable salt thereof.

42. The method of claim 37, wherein each tablet comprises about 306.8 mg of the sodium salt of the compound of Formula Ia.

43. The method of claim 37, wherein each tablet further comprises an outer film coat.

44. The method of claim 43, wherein the outer film coat provides from about 1% to about 8% weight gain based on the uncoated tablet.

45. The method of claim 43, wherein the outer film coat provides about 4% weight gain based on the uncoated tablet.

46-59. (canceled)

60. The method of claim 1, wherein the patient is a heavily treatment-experienced patient.

61-86. (canceled)

87. The method of claim 1, wherein the method further comprises administering one, two, three or four additional therapeutic agents to the patient.

88. The method of claim 87, wherein the additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof.

89. (canceled)

90. The method of claim 87, wherein the additional therapeutic agents are selected from the group consisting of bictegravir or a pharmaceutically acceptable salt thereof, abacavir sulfate, tenofovir, tenofovir disoproxil, tenofovir disoproxil fumarate, tenofovir disoproxil hemifumarate, tenofovir alafenamide, and tenofovir alafenamide hemifumarate.

91-94. (canceled)

95. The method of claim 1, wherein the compound of Formula Ia, or a pharmaceutically acceptable salt thereof, is a compound of Formula Ib: 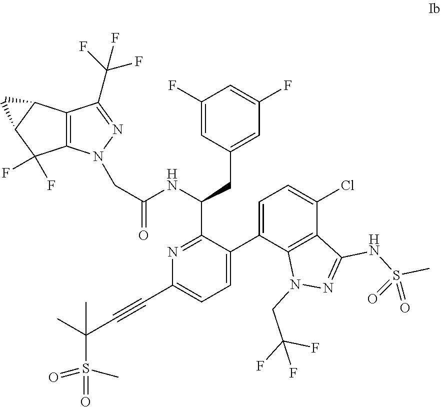 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]
or a pharmaceutically acceptable salt thereof.

96. The method of claim 95, wherein the compound of Formula Ib is administered as the sodium salt.
